SEQUENCE LISTING (17 sequences)

SEQ ID NO: 1 - protein, 624 aa, Homo Sapien
LSQRFPKYLYEIARYLYEIARFQNALLVRFQNALLVRQTALVELVKKYLYEIARLDELRDEGKYLYEIARRKQTALVELVKCCTESLVNRHPDYSVVLLLRAVMDDFAAFVEKAVMDDFAAFVEKAAFTECCQAADKYICENQDSISSKRHPDYSVVLLLRVPQVSTPTLVEVSRLKECCEKPLLEKCCAAADPHECYAKCCAAADPHECYAKADDKETCFAEEGQKKVPQVSTPTLVEVSRKVPQVSTPTLVEVSRAEFAEVSKLVTDLTKQNCELFEQLGEYKYICENQDSISSKLKQEPERNECFLQHKEQLKAVMDDFAAFVEKRPCFSALEVDETYVPKRPCFSALEVDETYVPKNECFLQHKDDNPNLPRVFDEFKPLVEEPQNLIKVFDEFKPLVEEPQNLIKAAFTECCQAADKAACLLPKEFNAETFTFHADICTLSEKEFNAETFTFHADICTLSEKERVHTECCHGDLLECADDRADLAKVHTECCHGDLLECADDRADLAKQNCELFEQLGEYKFQNALLVRLVRPEVDVMCTAFHDNEETFLKLVRPEVDVMCTAFHDNEETFLKLVRPEVDVMCTAFHDNEETFLKKLVRPEVDVMCTAFHDNEETFLKK

SEQ ID NO: 2 - protein, 193 aa, Homo Sapien
DYAEVGRGSFPVIQAKGSFPWCAKVGYVSGVIGRVGYVSGWGRVTSEQDWVQKDIAPILTLYVGKSCAVAEYGVYVKYVNILPVADQDQCRTMLPVADQDQCIRVMPICIPSKENADIGRVMPICIPSKDYABIGRSPVGVNLNEHTFCAGMSKSPVGVQPILNEHTFCAGMSKSPVGVQPLNEHTFCAGMSK

SEQ ID NO: 3 - protein, 622 aa, Homo Sapien
FQFLALLVRFQNALLVRQTALVELVKKYLYEIARLDELRDEGKCCTESLVNRLVIEVTEFAKHPDYSVVLLLRAVMDDFAAFVEKAVMDDFVFVEKFQNALLVRYTKAVMDDFIAFVEKAAFTECCQAADKYICENQDSISSIRHPDYSVVLLLRRHPDYSVVLLLRVPVSTPTLVEVSRLIECCEKPLLEICCAAADPHECYAKCCAAADPHECYAKDVFLGMFLYEYARADDKETCFAEEGQKKVPQVSTPTLVEVSRKVPQVSTPTLVEVSRAEFAEVSKLVTDLTKNCELFEQLGEYKYICEIQDSISSKLKHPYFYAPELLFFAKHPYFYAPELLFFAKRRHPYFYAPELLFFAIRPCFSALEVDETYVPKRPCFSLEVDETYVPKVPVSRPTLVEVSRNLGKVFDEFIPLVEEPQNLIIVFDEFKPLVEEPILIKVHTECCHGDLLECADDREFNAETFNHADICRLSEKEFNAETFTFHADICTLSEKERVHTECCHGDLLECADDRADLAKVHTECCHGDLLECADDRADLAKNCELFEQLGEYKFQNALLVRLVRPEVDVMCTIFHDNEELHKLVRPEVDVMCTAFHDNEETFLKKLVRPEVDVMCTIFHDNE

SEQ ID NO: 4 - protein, 71 aa, Homo Sapien
EGETKAVKLLELTGPKLLELRGPKSGLSTGWTQLSKSGLSTGWTLSKHQFLLTGDTQGRHQFLLTGDLTGR

SEQ ID NO: 5 - protein, 69 aa, Homo Sapien
NYYKLRNYYKLRGSFPWQAKLRTEGDGVYTLNNEKAVGDKLPECEAVCGKPKAVGDKLPECEAVCGKPK

SEQ ID NO: 6 - protein, 277 aa, Homo Sapien
DTLMISRALPFPIEKALPAPIEKDTLMSRNQVSLTCLVKNQVSLTCLVKGPSVFPLAPSSKEPQVYTLPPSREPQVYTLPPSRQTIPDYRRMIGQGAFIWYVDGVEVHIAKFTNVYVDGVEVHTAKEPQVYTLPPSRDELTKEPQYTLPPSRDELTKTPEVTCVVVDVSHEDPETVKTPEVTCVVVDVSHEDPEVKTPEVTCVVVDVSHEDPEVKGFYPSDIAVEWESNGQPENNYKWQQGIVFSCSIRVHEALHNHYTQKWQQGNVFSCSVMHEAL

SEQ ID NO: 7 - protein, 494 aa, Homo Sapien
RPCFSALEVDETYVPKRPCFSALEVDETYVPKNECFLQHKDDNPNLPRVFDEFKPLVEEPQNLIKVFDEFKPLVEEPQNLIKAFTECCQADKAACLLPKEFNAETFTFHADCTLSEKEFNAETFNHIDICRLSEKERVHRECCHGDLLECADDRADLAKVHTECCHGDLLECADDRADLAKQNCELFEQLGEYKFQNALLVRLVRPEVDVMCTAFHDNEETFLILVRPEVDVMCRAFHDNEETFLKLVRPEVDVMCTAFHDNEETFLIKLVRPEVDVMCRAFHDNEETFLKINQVSLTCLVKNQVSLTCLVKEPQVYTLFPSREPQVYTLPPSREPQYTLPPSRDELTKEPQVYTLPPSRDELTKTTPPVLDSDGSFFLYSKGFYPSDIAVEWESIGQPEIIYKGFYPSDIAVEWESIGQPENNYKWQGIVFSCSVMHEALHNHYTQKWQGNVFSCSVILHEALHNHYTQKWQQGRVFSCSVMHEALHRHYTQK

SEQ ID NO: 8 - protein, 899 aa, Homo Sapien
RPDPWAKDSAHGFLKSCHTFVGRAPNHAVITRASYLDCIRYLGEEYVKKSCHTAVGRKASYLDCIRHQTVPQTTGGKWCALSHHERDSGFQMNQLRDSGFQIVLNQLRSASDLTWDNLKSASDLTWDILKHSTIFENLANKEFQLFSSPHGKEGYYGYTGAFREGYRGYTGAFRWCAVSEHEATKKDSGFQMNQLRKDSGFQMIQLRDYELLCLDGTRKSASDLWVDNLKSVIPSDGPSVACVKMYLGYEYVTAIRSKEFQLFSSPHGKSKEFQLFSSPHGKMYLGYEVTAIRLKCDEWSVNSVGKCSTSSLLEACTFRCSTSSLLEACTFRDQYELLCLDITRDLLFRDDTVCLKDLLFRDDTVCLAKFDEFFSEGCAPGSKKPVEEYANCHLARKPVEEYANGHLARTAGWNIPMGLLYNKHSTIFENLNKADREDPTFYYAVAVVKKCSTSSLLEACTFRDCHLAQVPSHTVVARFDEFFSEGCAPGSIKLCMGSGLNLCEPNNIIEGVSAETTEDGIAKEGTCPEAPTDECKPVKADRDQYELLCLDITRADRDQYELLCLDTTRNLNEKDYELLCLDGTRKPVDEYIDCHLAQVPSHTVVARRPCFSALEVDETYVPKRPCFSALEVDETYVPKNECFLQHKDDNPNLPRVFDEFKPLVEEPQNLIKVFDEFKPLVEEPQNLIKAFTECCQADKAACLLPKEFNAETFTFHADCTLSEKEFNAETFNHIDICRLSEKERVHRECCHGDLLECADDRADLAKVHTECCHGDLLECADDRADLAKQNCELFEQLGEYKFQNALLVRLVRPEVDVMCTAFHDNEETFLILVRPEVDVMCRAFHDNEETFLKLVRPEVDVMCTAFHDNEETFLIKLVRPEVDVMCRAFHDNEETFLKI

SEQ ID NO: 9 - protein, 210 aa, Homo Sapien
ATVVYQGERATVVYQGEREHSSLAFWKEHSSLAFWKKATVVYQGERVCPFAGILENGAVRVCPFAGILENGAVRTCPKPDDLPFSTVVPLKTCPKPDDLPFSTVVPLKCSYTEDAQCIDGTIEVPKTFYEPGEEITYSGKPGYVSRTFYEPGEEITYSCKPGYVSRATFGCHDGYSLDGPEEIECTKGPFPSRPDNGFVNYPAKPTLYYK

SEQ ID NO: 10 - protein, 165 aa, Homo Sapien
ATVVYQGERATVIYQGEREHSSLAFWKEHSSLAFWKKATVVYQGERVCPFAGILENGAVRICPFAGILENGAVRTCPKPDDLPFSTVIPLKTCPKPDDLPFSTIVPLKCSYTEDAQCIDGTIEVPKTFYEPGEEITYSCKPGYSRTFYEPGEEITYSGKPGYVSR

SEQ ID NO: 11 - protein, 143 aa, Homo Sapien
GVGHPYRRYGIDWASGRYGIDWASGRYVSEAHKQDGSVDFFRQDGSVDFFRTFAHYATFRTFAHYATFRGEPGDPVNLLRRQDGSVDFFRRQDGSVDFFRLLGEVDHYQLALGKLLGEVDHYQLALGKMGPKGEPGDPVNLLR

SEQ ID NO: 12 - protein, 772 aa, Homo Sapein
TMMSVSRTMMSVSRNASKVANKGAASRFVKNALWEPKLDPLEQAKLDPLEQAKFVKNALWEPKEINFYKQKQQSAKQKQQSAKRTMFQKRTMFQKDEEILLKHHLEFGKMKVQEEKAFLNCDRMSLFMYRFHIPQEALVREPLDESGWMIKHHLEFGKTLLRHHLEFGKTLLREINFYKVIDYILHGKVKQMFRDSMLGDRMMSTRRVENTAKRRVENTAKVKQMFRVSGPGVRRSQKPGRRRTASAPTKVKQALGLKFLQVEQKSKSNPNLRLSHSLGLPGGTRRRLSHSLGLPGGTRYLMAGISDEDSLARTSSSTQLKSEGSGGTQLKMQKSEGSGGTQLKMQKSEGSGGTQLKTINPFGEQSRVPSAFAAIYSKTSSSTQLKSEGSGGTQLKMQKSEGSGGTQLKMQKSEGSGGTQLKTINPFGEQSRVPSAFAAIYSKTSSSTQLKSEGSGGTQLKMQKSEGSGGTQLKMQKSEGSGGTQLKTINPVHSDDEVFERVKQMFRDSMLGDRMMSTRRVENTAKRRVENTAKVKQMFRVSGPGVRRSQKPGRRRTASAPTKVKQALGLKFLQVEQKSKSNPNLRLSHSLGLPGGTRRRLSHSLGLPGGTRYLMAGISDEDSLARTIYLCRSSADSSRKKFASALSKVWTSQLKLPYEQWKDLRVWTSQLKYQKQQKSGRSPAPRVWFQNRMPSPRPRLTENQVRAQLAARLKAQLAARLKELDRERELDRERMGGTTSTRMGGTTSTRGGTTSTRRSEEERAKQAKELDR

SEQ ID NO: 13 - protein, 2360 aa, Homo Sapien
KSSMVALKYKLLESKNLEVEHRLRQQAEIKLRQQAEIKESSLSRQSKNYEALKQRQKGQLEDLEKQKGQLEDLEKETEVLQTDHKQASEYESLISKDLEDRYNQLLKDLEDRYNQLLKSVSGKTPGDFYDRSSSQENLLDEVMKTLVTLREDLVSEKLIEVERNNATLQAEKKTEDTYFISSAGKPTPGTQGKTLLEQNMESKDLFHVEQRTLLEQNMESKDLFHVEQRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRAAQIAGAVRATPAHRARGNMRSCRGNMRSCRLTDFGFGRSCRVLLHMRSCRVLLHMRGHQGGGPAASAPGLRGAPGHPLRPQEVRCENVLLSPDERRLEAGWFQPFLQPRALGQGGARLMALGPIRLMALGPIRFVTISGQKDFTELQKKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRQLNLLQKRFSNIDKFALTAETKRNFSNMKSLTGYLEKSLTGYLEKAELEHWKIPAWWKKRLELWLRRLELWLRVNDLIEFRSVAMMVPDRSVAMMVPDRTWREMDIRNMKLIESKPKNMKLIESKPKAVLLIGEQGTAKLLSDIFIPALRLLSDIFIPALRVDTLHYAWEKTSIIDFTVTMKLKEASESVAALSKRLLSDIFIPALRLPNPAYTPEISARLVSVLSTIINSTKKNFSNMKMMLAEYQRERWTEQSQEFAAQTKELLSHFNHQNMDALLKVTREILNNHGKDAAPGASKLRDAAPGASKLRGVVDHLLLRGVVDHLLLRQPWHGKAMQRNNEFPVFDEFNNEFPVFDEFGGSPAVTLLISEKASEAGATAPKASARISAPNEFDVMFKLSRDDISTAAGMVKDDVDMLRDYDALRKLEQEYSRRENGQLLRRENGQLLRAHGPEVQAHNKAHGPEVQAHNKKAANEEMEALRLADVEQELSFKKLADVEQELSFKKDDNSATKTLSAAARLQTEVELAESKLKAANEEMEALRQIKVQKGSEPLGISIVSGEKDLNPGVKKDLNPGVKKMSLGQLQSARGTCSGFEPHSWRGVACLGCKGTCSGFEPH

SEQ ID NO: 14 - protein, 2811 aa, Homo Sapien
ESEDQKRMGGTTSTRRKSSMVALKYKLLESKNLEVEHRLRQQAEIKLRQQAEIKESSLSRQSKNYEALKQRQKGQLEDLEKQKGQLEDLEKETEVLQTDHKQASEYESLISKDLEDRYNQLLKDLEDRYNQLLKSVSGKTPGDFYDRSSSQENLLDEVMKTLVTLREDLVSEKLIEVERNNATLQAEKKTEDTYFISSAGKPTPGTQGKTLLEQNMESKDLFHVEQRTLLEQNMESKDLFHVEQRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRAAQIAGAVRATPAHRARGNMRSCRGNMRSCRLTDFGFGRSCRVLLHMRSCRVLLHMRGHQGGGPAASAPGLRGAPGHPLRPQEVRCENVLLSPDERRLEAGWFQPFLQPRALGQGGARLMALGPIRLMALGPIRFVTISGQKDFTELQKKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRQLNLLQKRFSNIDKFALTAETKRNFSNMKSLTGYLEKSLTGYLEKAELEHWKIPAWWKKRLELWLRRLELWLRVNDLIEFRSVAMMVPDRSVAMMVPDRTWREMDIRNMKLIESKPKNMKLIESKPKAVLLIGEQGTAKLLSDIFIPALRLLSDIFIPALRVDTLHYAWEKTSIIDFTVTMKLKEASESVAALSKRLLSDIFIPALRLPNPAYTPEISARLVSVLSTIINSTKKNFSNMKMMLAEYQRERWTEQSQEFAAQTKELLSHFNHQNMDALLKVTREAALTLPREAALTLPREILNNHGKDAAPGASKLRDAAPGASKLRGVVDHLLLRGVVDHLLLRQPWHGKAMQRNNEFPVFDEFNNEFPVFDEFGGSPAVTLLISEKASEAGATAPKASARISAPNEFDVMFKLSRDDISTAAGMVKDDVDMLRDYDALRKLEQEYSRRENGQLLRRENGQLLRAHGPEVQAHNKAHGPEVQAHNKKAANEEMEALRLADVEQELSFKKLADVEQELSFKKDDNSATKTLSAAARLQTEVELAESKLKAANEEMEALRQIKVQKGSEPLGISIVSGEKDLNPGVKKDLNPGVKKMSLGQLQSARGTCSGFEPHSWRGVACLGCKGTCSGFEPHRQAVQDLRADVVNKALREKMELEMRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRSWCQELEKRGEHGRLLSLWRVNVELQLQGDSAQGQKLMALGPIR

SEQ ID NO: 15 - protein, 2374 aa, Homo Sapien
RQAVQDLRADVVNKALREKMELEMRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRSWCQELEKRGEHGRLLSLWRVNVELQLQGDSAQGQKLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKSEQLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKLMALGPIRDFTELQKKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKTNFPYVRSDILGHLRSDILGHLRNIFKRPLGSRNIFKRPLGSRQSGVVPFIFQAKSGFPLLKEFVQRTLSGTPEVHSNKRTLSGTPEVHSNKRAAEGIPKLLVLITGGKYPPPGEMGASEVLLGAFSIKMKPLDGSALYTGSALDFVRSAGSRIEDGVLQFLVLLVAGRVDGPASNLKQSGVVPFIFQAKGNRVSPTMKDSLLELSPVERDSLLELSPVERYPGMFIALSKNGKYPGMFIALSKNGKRLYCNVGIGFHLQALPDGRRLYCNVGIGFHLQALPDGRMAGASPAVPHERARMAGASPAVPHERARLFEDMVTKLQALRALGVGGAGCGVQGLASLARTTGLSDQQVVCDLDHRAVEALLQAVRMNGEYRGRGGGNFSGKWRTSEQTPQFLLSTKELTEGGEVTNLIPDIATELRTLACMGLAIHQVLTKDLERDAPEKTLACMGLAIHQVL

SEQ ID NO: 16 - protein, 1858 aa, Homo Sapien
RQAVQDLRRQAVQDLREKMELEMRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLHMEKADVVNKHFLEMKSATDRLQSSQLQSCRVLKVNVELQLQGDSAQGQKLSGSLLTCCLRLTVGAQEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKSEQAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKSVAYMPYAEVKSVAYMPYAEVKSVAYMPYAEVKRSSTSPCGTSKSPNRSSTSPCGTSKSPNRRALEQEAQMHNTAARYSVPPVLQPAPHQVITNLPEWHVVSSKLMALGPIRYSEIQDRYSEIQDRQQVQFMLKQQVQFMLKVVKAQIQEQKNCPISAKLERQQLEETSEIRQISEQLNALNKQISEQLNALNKLLEVWIEFGRGDLRFVTISGQKQPVYDTTIRTGRQPVYDTTIRTGRARQEQLELTLGRTGSLEEMTQRLRNTISVKAVCDYRNTISVKAVCDYRLNDALDRLEELKFEQLCLQQQEKEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKHLFCPDLLRHLFCPDLLRDKVAFITGGGSGIGFRIAEIFMRAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKKLPASEGVGKKLPASEGVGKNVILMAHDGKFYMVSYYERFYMVSYYERWQDRCLEVRGPITAVAFAPDGRSLAALKNMAHHKGLQECFPAICRYSHNSLMVQAIKYSHNSLMVQAIKLPASCLPASDSFRVIIWTENGQSYIYKHTCKALTFLLLQPPSPKEHLLDDEEEDEEIMRQRYDQRYLISGGVDFSVIIWQGWSQLAAMHCVMLP

SEQ ID NO: 17 - protein, 2581 aa, Homo Sapien
KLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKKLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKEVQLVESGAEVKSDDTAVYYCARSDDTAVYYCARASGYTFTGYYMHWVRVTMTRDTTISTAYMELSRIAAAGDAFDIWGQGTMVTVSSLMALGPIRLMALGPIRYSEIQDRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKWADHEVRAMESTATAAVAAELVSADKMAMESTATAAVAAELVSADKAMESTATAAVAAELVSADKMSGRLMALGPIRLMALGPIRYSEIQDRQQVQFMLKQQVQFMLKVVKAQIQEQKNCPISAKLERNCPISAKLERMPPLIPAEVDKQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKLMALGPIRLMALGPIRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKSYAEELAKISYSLFTALRIVMNMNKSTRDKLDQVSSEIKDKLDQVSSEIKMININILSVCKMININILSVCKGVFVQSVLPYFVATKLAKMININILSVCKMTQLVLPGMVERMININILSVCKMTQLVLPGMVERYEDEINKEDLARLLRGFSSGSAVVSGGSRRSTSSFSCLSRHGGGGGGFGGGGFGSRHGGGGGGFGGGGFGSRGSSSGGGYSSGSSSYGSGGRQCKNVQDAIADAEQRSISISVAGGGGGFGAAGGFGGRDYQELMNVKLALDVEIATYRNPGSLRGRLETHPCRGSIGQSAIPRSPCPIRSPLPARASAPWASLSTRADSGLRMSPLETNKGSIGQSAIPRATSASLPQETPFALSVVWAPRR

(19) United States
(12) Patent Application Publication
(10) Pub. No.: US 2014/0093515 A1
(43) Pub. Date: Apr. 3, 2014

(54) PROCESS OF AFOD AND AFCC AND MANUFACTURING AND PURIFICATION PROCESSES OF PROTEINS

(71) Applicant: Kieu Hoang, Agoura Hills, CA (US)

(72) Inventor: Kieu Hoang, Agoura Hills, CA (US)

(21) Appl. No.: 13/756,478

(22) Filed: Jan. 31, 2013

Related U.S. Application Data
(60) Provisional application No. 61/593,164, filed on Jan. 31, 2012.
(60) Provisional application No. 61/593,183, filed on Jan. 31, 2012.
(60) Provisional application No. 61/593,196, filed on Jan. 31, 2012.
(60) Provisional application No. 61/648,281, filed on May 17, 2012.
(60) Provisional application No. 61/692,273, filed on Aug. 23, 2012.
(60) Provisional application No. 61/710,930, filed on Oct. 8, 2012.

(51) Int. Cl. A61K 39/395; A61K 38/17

(52) U.S. Cl. CPC A61K 39/3955; A61K 38/1709
USPC 424/158.1; 435/325; 514/1.9; 514/4.3; 435/366; 435/419

(57) ABSTRACT

Manufacturing and purification processes of proteins, KH 1-through KH-52, and more KH proteins are being discovered in good healthy cells—named KH CELLS. KH CELLS are good healthy cells in which the RNA synthesizes good proteins that: 1) Send signal to the damaged, sick, and bad cells that triggers that synthesis of good proteins that transform these cells to become GOOD healthy cells; 2) Send signal to the other currently undamaged cells to synthesis of good proteins to protect them from being damaged, infected and prone to DNA and other cellular alterations; and 3) Send signal to the body to produce new cells that are healthy and forbid them from being affected by intra- and extracellular damaging signals. The mechanism that governs these processes is that the KH good healthy cells provide innate good signals that make good proteins to boost the immune system.

Process of AFOD and AFCC and Manufacturing and Purification processes of existing discovered and newly discovered proteins, KH 1—through KH-52, and more KH proteins are being discovered in GOOD HEALTHY CELLs—named KH CELLS. KH CELLS are GOOD HEALTHY CELLS in which the RNA synthesizes good proteins that:
1—Send signal to the DAMAGED, SICK, AND BAD CELLS that triggers that synthesis of good proteins that transform these cells to become GOOD healthy cells.
2—Send signal to the other currently undamaged cells to synthesis of good proteins to protect them from being DAMAGED, INFECTED and PRONE to DNA and other cellular alterations.
3—Send signal to the body to produce new cells that are healthy and forbid them from being affected by intra- and extracellular damaging signals.

The mechanisms that govern these processes is the KH good healthy cells provide innate good signals that make good proteins to boost the immune system in order to CURE, TO PROTECT, and TO PREVENT diseases, viruses infections, bacteria infections, auto immune disease, neurological disorder, all type of solid and blood cancer, coagulation, diabetic, inhibitor, immune deficiency, muscle and nerve repair and restoration from Human, animal or substances by the method of fractionation, purification, recombinant DNA, monoclonal antibody, transgenic and expression of cells from the cultured GOOD HEALTHY CELLS.

### BRIEF DESCRIPTION OF THE DRAWINGS

FIG. 1. Process flow chart of the manufacturing of tile AFOD RAAS 101® from pool of the plasma to fraction V for further process into a human albumin containing ALB Uncharacterized protein, HPR 31 kDa protein, ALB Uncharacterized protein, A1BG Isoform 1 of Alpha-1B-glycoprotein, HPR Haptoglobin and KH51.

FIG. 2. Protein analysis of RAAS human albumin against human album import from other manufacturers. RAAS Albumin containing 1—ALB Uncharacterized protein, 2—HPR 31 kDa protein, 3—ALB Uncharacterized protein, 4—A1BG Isoform 1 of Alpha-1B-glycoprotein, 5-HPR Haptoglobin and 6-KH51 proteins

FIG. 2.1 Protein analysis of RAAS Human albumin containing the protein ACTC1 Actin, alpha cardiac muscle 1.

FIG. 3. Protein analysis of International import Company 1 I1uman albumin containing only HPR31 kDa protein.

FIG. 4. Protein analysis of International import Company 2 human albumin containing only HPR31 kDa and Albumin Uncharacterized proteins.

FIG. 5. Protein analysis of International import Company 3 human albumin containing only HPR31 kDa, Albumin Uncharacterized and A1BG Isoform 1 of Alpha-1B-glycoprotein proteins.

FIG. 6. Process flow chart of the manufacturing of the AFOD RAAS 102® from Fraction II+III paste.

FIG. 7. Protein analysis of Immunoglobulin from fraction II+III. Beside Immunoglobulin there are two other proteins 120/E19 IGHV4-31; IGHG1 44 kDa protein and 191/H18 IGHV4-31; IGHG1 32 kDa.

FIG. 7.1 Process analysis of Immunoglobulin containing the protein IGHV4-31; IGHG1 Putative uncharacterized protein DKFZp686G11190.

FIG. 8. Process flow chart of the manufacturing of tile AFOO RAAS 103® from fraction III paste

FIG. 9. Protein analysis of Immunoglobulin from fraction III containing 193/H20 TF serotransferrin, 194/H21 APOH beta2-glycoprotein 1, 195/H22 cDNA FLJ5165, moderately similar to beta-2-glycoprotein, 196/H23FCN3 isoform 1 of Ficolin-3.

FIG. 10. Process flow chart of the manufacturing of the AFOO RAAS 104® HBig purification process from Fraction II+III paste.

FIG. 11. Protein analysis of HBIG beside the Immunoglobulin proteins, containing the protein TF serotransferrin.

FIG. 12. Protein analysis comparison between Immunoglobulin from II+III paste vice versa Imn1unoglobulin produced frmn fraction III paste and Hepatitis B In1munoglobulin produced from fraction !!+III paste showing the different protein in each of the product bedsides the main Immunoglobulin protein analysis.

FIG. 13. Protein analysis for AFOO RAAS 102®, AFOO RAAS 103® and AFOD RAAS 104®

FIG. 14. Process flow chart for AFOD RAAS 105®

FIG. 14a. Process flow chart for AFOO RAAS 105®

FIG. 15. Process flow chart for AFOD RAAS 106®

FIG. 16. Process flow chart for purification process of AFOO RAAS 107® (CP98)

FIG. 17. 20 electropherosis of plasma derived protein CP 98 kDa

FIG. 18. Process flow chart for purification process of AFOO RAAS 108® (A1AT)

FIG. 19. 20 electropherosis of plasma derived protein A1AT

FIG. 20. Process flow chart for purification process of AFOO RAAS 109® (Transferrin)

FIG. 21. 20 electropherosis of plasma derived protein Transferrin

FIG. 22. Process flow chart for purification process of AFOO RAAS 110® (AntiThrombin !II)

FIG. 22a. Process flow chart for purification process of AFOO RAAS 110® (AntiThrombin III from fraction III)

FIG. 23. 20 electropherosis of plasma derived protein AntiThrombin IIL

FIG. 24. Process flow chart for purification process of AFOO RAAS 111® (Hun1an Albumin from fraction IV)

FIG. 25. 20 electropherosis of plasma derived protein Human Albumin from fraction IV

FIG. 26. Process flow chart for purification process of AFOO RAAS 112® (Human Albumin from Fraction !II)

FIG. 27—Photograph of Cryopaste and FVIII

FIG. 28. Process flow chart for purification process of AFCC RAAS 101® (Human Coagulation Factor VIII)

FIG. 29. 20 electropherosis of plasma derived protein Human coagulation Factor VIII

FIG. 30. Process flow chart for purification process of AFCC RAAS 102® (Human Fibrinogen)

FIG. 31. 20 electropherosis of plasma derived protein Human Fibrinogen

FIG. 32. Process flow chart for purification process of AFCC 103® (High Concentrate Human Fibrinogen)

FIG. 33. 20 electropherosis of plasma derived protein High Concentrate Human Fibrinogen

FIG. 34. Process flow chart for purification process of AFCC RAAS 104® (Human Thrombin)

FIG. 35. 20 electropherosis of plasma derived protein Human Thrombin

FIG. 36. Process flow chart for purification process of AFCC RAAS 105® (Human Prothrombin Complex)

FIG. 37. 20 electropherosis of plasma derived protein Human Prothrombin

FIG. 38. Process flowchart of AFCC RAAS 106® Purification process from Fr. IV1+IV4 paste

FIG. 38a. 20 electropherosis of AFCC from fraction IV.

FIG. 38b. 20 electropherosis of Anti Thrombin III.

FIG. 38c. 20 electropherosis of CP98.

FIG. 38d. 20 electropherosis of Transferrin.

FIG. 38e, 20 electropherosis of AlpI1a 1 Antitrypsin.

FIG. 38f. 20 electropherosis of Human Albumin.

FIG. 39. Process flowchart for Recombinant Factor VIII

FIG. 40. Process flowchart for Monoclonal Antibodies.

FIG. 41. Process flowchart for manufacturing of AFOD RAAS and AFCC RAAS products by using the direct cell from cell culture for expression to synthesize the desired already discovered or newly found proteins.

FIG. 42. Dose-dependent curves (by GraphPad Prism) showing AFCC KH has 100% percentage of inhibition of HIV virus like the reference compound.

FIG. 43. All products have shown a low percentage of inhibition.

FIG. 44. Log compound ug/mL showing inhibition of HCV in AFOD KH 70% and AFCC RAAS 1 50%, AFCC RAAS 4 40% to compare with Ribavirin which reach only 50%

FIG. 45. Log compound ug/mL showing inhibition of HCV in AFOD KH 70% and AFCC RAAS 1 50%, AFCC RAAS 4 40% to compare with Ribavirin which reach only 50%;

FIG. 46. CCK8 testing method. In vitro testing for Lung Cancer cells in RAAS current plasma derived products.

FIG. 47. CCK8 testing method. In vitro testing for Lung Cancer cells in RAAS new plasn1a derived products.

FIG. 47a. In vitro studies of the different proteins vs Lung Cancer at 0%, 2%) and 10% concentration of the product

FIG. 48. High concentration of rONA products with lung cancer cell.

FIG. 49. High concentration of rONA products with lung cancer ceiL

FIG. 50. Recombinant and monoclonal products in inhibiting lung cancer ceiL

FIG. 50a. In vitro studies of the different recombinant products vs Lung Cancer at 0%, 2% and 10% concentration of the product.

FIG. 50b. In vitro studies of the different recombinant products vs Lung Cancer at 0%, 2% and 10% concentration of the product.

FIG. 51. 5% samples from animal source with feta bovine serum, bovine albumin, bovine IVIG, pig thrombin and pig fibrinogen.

FIG. 52. 5% sample from animal source with feta bovine serum, bovine albumin, bovine IVIG, pig thrombin and pig fibrinogen with lung cancer cell.

FIG. 53. KH101 medium alone, KH101 medium consist of 50 g of paste of rice in 1 liter of water for injection.

FIG. 54. KH101 medium alone, KH101 medium consist of 50 g of paste of rice in 1 liter of water for injection with cell count analysis sI1owing nearly 20 million cells.

FIG. 55. Product AFCC alone showing nearly 8,000 cells.

FIG. 56. Product AFCC mixed with KH101 medium.

FIG. 57. Product AFCC mixed with KH101 medium after 5 days in bioreactor, which has reach 4.5 million cell count

FIG. 58. APOA1 product alone with cell count with nearly 20,000 cells.

FIG. 59. APOA1 product with KH101 medium.

FIG. 60. APOA1 with KH101 medium after 5 days in bioreactor which after cell analysis has reached 4 million cell count.

FIG. 61. AFOD Product alone with cell count with nearly 10,000 cells.

FIG. 62. AFOD Product with KH101 medium

FIG. 63. AFOD product with KH101 medium after 5 days in bioreactor which after cell analysis has reached 4.6 million cell count.

FIG. 64. Factor VIII alone with cell count with nearly 5,400 cells.

FIG. 65. Factor VIII with KH 101 medium.

FIG. 66. Factor VIII with KH101 medium after 5 days in bioreactor which after cell analysis has reached 3.4 million cell count.

FIG. 67. Liver fatty change of Rabbit after treatment with AFOD RAAS 101.

FIG. 68. Comparison of fat deposit on I1eart from vehicle rabbit and AFOD RAAS 101 treated rabbit.

FIG. 69. Comparison of atherosclerosis in aorta from vehicle rabbit and treated rabbit

FIG. 70. Pictures of aorta from vehicle control rabbit.

FIG. 71. Pictures of aorta from rabbit treated with a low dose of AFOD RAAS 101.

FIG. 72. Pictures of aorta from rabbit treated with a medium dose of AFOD RAAS 101.

FIG. 73. Pictures of aorta from rabbit treated witlla high dose of AFOD RAAS 101.

FIG. 74. Pictures of aorta from rabbit treated with a positive control (Lipitor)

FIG. 75. Analysis of body weight in 18 aPOe MICE.

FIG. 76. Blood plasma lipid profile at three time points in 18 Apo E(−/−) mice.

FIG. 77. Illustration of Aorta.

FIG. 78. Oil red staining procedure.

FIG. 79. image analysis and procedure of aorta.

FIG. 80. Aorta photos of vehicle, control and treated mice.

FIG. 81. Graph showing results of the sum area of atherosclerotic plaque. (mm2).

FIG. 81a. Area of atherosclerotic plaue on aorta.

FIG. 81b. Photos of treated and control aortas.

FIG. 81c. Results of the atherosclerotic plaque

FIG. 81d. Results of the mean density.

FIG. 81e. Results of the area percent

FIG. 82. Effect of APOA1 on body weight

FIG. 83. Effect of APOA1 on food intake.

FIG. 84. Comparison of the lipid profile of ApoE mice fed with common diet and high fat diet.

FIG. 85. Effect of RAAS antibody on total cholesterol.

FIG. 86. Net change of plasn1a total cholesterol after 8 weeks.

FIG. 87. Effect of RAAS antibody on triglyceride.

FIG. 88. Effect of RAAS antibody on High Density Lipoprotein.

FIG. 89. Effect of RAAS antibody on Low Density Lipoprotein.

FIG. 90. Effect of RAAS antibody on Atherosclerosis plaque lesion area.

FIG. 91. Effect of RAAS antibody on the percent of plaque area.

FIG. 92. Effect of RAAS antibody on the percent of plaque area after 2 weeks

FIG. 93. Analysis area of the aortic plaque.

FIG. 94. Analysis of tile root plaque area.

FIG. 95. Analysis of tile percent of the root plaque area.

FIG. 96. Analysis area of the artery.

FIG. 97. Analysis of plaque area from root to right renal area.

FIG. 98. Analysis of plaque area percentage from root to right renal area.

FIG. 99. The effect of the aortic inner lumen area

FIG. 100. The mean density of the effect of the aortic lumen area.

FIG. 101. The effect of RAAS antibody on liver weight.

FIG. 102. The effect of RAAS antibody on liver weight index.

FIG. 103. The effect of RAAS antibody on fasting overnight blood glucose

FIG. 104. Image of aorta red oil staining.

FIG. 105. Image of aorta red oil staining in different groups.

FIG. 106. Images of red stained aorta in negative control.

FIG. 107. Images of red stained aorta in vehicle control.

FIG. 108. Images of red stained aorta treated with APOA1 high dose.

FIG. 109. Images of red stained aorta treated with APOA1 medium dose.

FIG. 110. Images of red stained aorta treated with APOA1 low dose.

FIG. 111. Images of red stained aorta in positive control (Atorvastatin).

FIG. 112. Effect of AFOD on body weight.

FIG. 113. Effect of products on blood glucose (fasting 6 hrs)

FIG. 114. Effect of products o fasting overnight t of blood glucose.

FIG. 115. The effect of AFOD on plasma insulin.

FIG. 116. The effect of AFOD on HOMA-IR

FIG. 117. The effect of AFOD, AFCC, APOA1 on body weigI1t.

FIG. 118. The effect of AFOD, AFCC and APOA1 on fasted 6 hours of blood glucose.

FIG. 119. The effect of AFOD, AFCC and APOA1 on overnight fasted blood glucose.

FIG. 120. The effect of AFOD, AFCC and APOA1 on plasma insulin

FIG. 121. The effect of AFOD, AFCC and APOA1 on plasma HOMA-IR

FIG. 122. The effect of AFOD, AFCC and APOA1 on plasma lipid.

FIG. 123. The effect of AFOD, AFCC and APOA1 on liver weight.

FIG. 124. Plasma insulin level in db/db mice during two periods of study.

FIG. 125. Breast cancer 4T1-luc orthotopic model growth curve

FIG. 126. Breast cancer 4T1-luc orthotopic model growth curve for AFOD RAAS 1, 2, 3 and 4.

FIG. 127. Breast cancer 4T1-luc orthotopic model growth curve for AFOD RAAS 5 and 6.

FIG. 128. Breast cancer 4T1-luc ort11otopic model growth curve for AFOD RAAS 1, 2, 3, 4, 5 and 6 and AFOD KH and AFCC KH

FIG. 129. Breast cancer 4T1-luc orthotopic model growth curve for AFOD RAAS 1, 2, 3 and 4.

FIG. 130. Breast cancer 4T1-luc orthotopic model growth curve for AFOD RAAS 5 and 6 and AFOD KH and AFCC KH.

FIG. 131. Breast cancer 4T1-luc orthotopic model body weight change for AFOD RAAS 1, 2, 3 and 4.

FIG. 132. Breast cancer 4T1-luc orthotopic n1odel body weight change for AFOD RAAS 1, 2, 3 and 4.

FIG. 133. Breast cancer 4T1-luc orthotopic model body weight change for AFOD RAAS 5 and 6 and AFOD KH and AFCC KH.

FIG. 134. Fluorescence inlages of the whole body for vehicle, Gemcitabine, AFOD RAAS 1/8, AFOD RAAS2 and AFOD RAAS 3.

FIG. 135. Fluorescence images of the whole body for AFOD RAAS 4, AFOD RAAS 5, AFOD RAAS 6, AFOD KH and AFCC KH

FIG. 136. Anti-tumor efficacy of FS+AFOD in PDX model U-00-0117

FIG. 137. Weights oftumors on day 24 after treatment

FIG. 138. Photograph of each tumor for each group.

FIG. 139. Relative change of body weight(%) of different groups

FIG. 140. Photo of nude mice with MDA-MB-231-Luc tumor cells.

FIG. 141. Photo of 10 nude mice group 2-5 which have been implanted with tumor cells from the 2-5 mice positive control group using Docetaxel in another study done at another CRO lab.

FIG. 142. PI1oto of nude mice witIIMDA-MB-231-Luc tumor cells transferred from 2-5 positive control group using DocetaxeL

FIG. 143. Graph showing the tumor volume of Mice #6-10 from the study done from July until Nov. 11, 2011 when the dead body of mouse #6-10 was removed from one CRO lab to another one for further study.

FIG. 144. Pictures of mouse #6-10 taken from Aug. 23, 2011 to Nov. 3, 2011 showing the growth of the tumor which had been detached from the body was under recovery from breast cancer using AFCC proteins for treatment.

FIG. 145. The tissue from the area of mouse #6-10 where the tumor had been detached was used to implant in the 10 nude mice 66 days after re-implantations show no tumor growth.

FIG. 146. Table showing tumor growth of mouse #6-10 after second re-implantation.

FIG. 147. Graph showing tumor growth after re-implantation of various mice including 6-10.

FIG. 148. Photo of nude mice group #6-10 with mice $5-1 and #5-5 showing growth of the tumor.

FIG. 149. PI1oto of mice 6-10 after re-implantation, showing tumor growth which has been inhibited by using AFCC KH proteins from Feb. 29, 2012.

FIG. 150. Graph of mouse #4-6 recovery within 24 days.

FIG. 151. Mouse #4-6 grew the tumor on August 23rd and self-detached from the body Sep. 1, 2011.

FIG. 152. Photo of mouse #4-6 completely recovered.

FIG. 153. Photo of 10 mice in group #4-6

FIG. 154. Photo of nude mice #4-6 with no tumor growth.

FIG. 155. Photo of nude mice used as negative control with no tumor.

FIG. 156. Photo of nude mice C57BU6 used as negative control with no tumor.

FIG. 157. The percent of B cells in peripheral blood.

FIG. 158. The percent of activated B lymphocytes in peripheral blood.

FIG. 159. The percent of monocytes/macrophages in peripheral blood.

FIG. 160. The percent of mDC and pDC in peripheral blood.

FIG. 161. The percent of CD3 T cells in spleen.

FIG. 162. The percent of B cells in spleen.

FIG. 163. The percent of mDC and pDC in spleen.

FIG. 164. The percent of activated B lymphocytes in spleen.

FIG. 165. The percent of monocytes/macrophages in spleen.

FIG. 166. The percent of granulocytes in spleen.

FIG. 167. The percent of CD3 T cells in draining lymph nodes.

FIG. 168. The percent of B cells in draining lymph nodes.

FIG. 169. The percent of mDC and pDC in draining lymph nodes.

FIG. 170. The percent of granulocytes in draining lymph nodes.

FIG. 171. The percent of monocytes and macrophages in draining lymph nodes.

FIG. 172. The percent of activated B lymphocytes in draining lymph nodes.

FIG. 173. Effect of AFOD RAAS2 on HN1 caused mortality.

FIG. 174. The average body weight change in mice infected with H1N1 influenza.

FIG. 175. Effects of pretreatment of AFOD on the behavioral performance.

FIG. 176. Effects of pretreatment+post treatment of AFOD on the behavioral performance.

FIG. 177. TH staining of the SN. Rats were perfused and the brains were fixed for IHC study.

FIG. 178. Effects of daily injection of AFOD on adjusting step test.

FIG. 179. Effects of daily injection of AFOD on rotation

FIG. 180. TH staining of the SN.

FIG. 181. Body weight changes caused with AFCC treatment in mice.

FIG. 182. Efficacy of AFCC on H1N1 WSN-caused mouse death.

FIG. 183. Body weight change caused by AFCC in mice infected with H1N1 (WSN) influenza.

FIG. 184. Body weight change caused with AFCC treatment in mice infected with H1N1 (WSN) influenza.

FIG. 185. Body weight change caused with vehicle treatment in mice infected with H1N1 (WSN) influenza.

FIG. 186. Effect of AFCC on H1N1-caused mouse mortality.

FIG. 187. The average body weight change in mice infected with H1N1 influenza.

FIG. 188. The efficacy of AFOD on H1N1 WSN-caused mouse death.

FIG. 189. The efficacy of AFCC on HIN1 WSN-caused mouse deat11.

FIG. 190. Body weight changes caused by AFOD or Oseltamivir treatment in mice infected with HINI (WSN) influenza.

FIG. 191. Body weight changes caused by AFCC or Oseltamivir treatment in n1ice infected with H1N1 (WSN) influenza.

FIG. 192. Photos of mouse organs dissected in the end of the study RAAS-201110170.

FIG. 193. Day 1 if HBsAg level.

FIG. 194. Day 3 of HBsAg level.

FIG. 195. Efficacy of therapeutic treatment of prophylactic treatment of RAAS-8 or ETV on in vivo HBV replication in HBV mouse HOI model.

FIG. 196. Effect of propHylactic treatment or therapeutic treatment of RAAS 8 or ETV on the HBsAg in mouse blood.

FIG. 197. Effect of prophylactic treatment or therapeutic treatment of RAAS 8 or ETV on the intermediate HBV replication in the n1ouse livers by qPCR

FIG. 198. HBV DNA level in plasma effect of treatment or therapeutic treatent of RAAS 8 or ETV.

FIG. 199. Southern blot determination of intermediate HBV DNA in mouse livers.

FIG. 200. The body weights of mice treated with vehicle or indicated compounds during the course of experiment.

FIG. 201. Picture of n1 ouse 4-6 which grew hair on top of head.

FIG. 202. Picture of Fibrin Sealant inhibiting the growth of lung cancer cell.

FIG. 203. Picture of Lung cancer cell without Fibrin Sealant.

FIG. 204. Picture of Lung cancer cell with Fibrin Sealant.

FIG. 205. Picture of lung cancer cells in n1ediun1.

FIG. 206. Photos of peripheral nerve repair in Rhesus monkey.

FIG. 207. PI1otos of peripheral nerve repair in Rhesus monkey.

FIG. 208. Photos of peripheral nerve repair in Rhesus monkey.

FIG. 209. Peripheral nerve degradation and regeneration.

FIG. 210. Nerve conduit repair, goat common peroneal nerve.

FIG. 211. Goat distal nerve immunohistochemical staining.

FIG. 212. Pictures of goat after 7 days of operation and 16 months later.

FIG. 213. Pictures of nerve conduit group 16 months after operation and vehicle control.

FIG. 214. Picture of Goat after 7 days of operation and self graft group 16 moths later.

FIG. 215. Picture of nerve conduit group 16 months later and vehicle controL

FIG. 216—Picture of FRIII and AFCC KH

FIG. 217 APCC KH

FIG. 218-219—FRIII Process

FIG. 220—Flow chart OF AFCC 01 process FROM Frill PASTE

FIG. 221—Flow chart of AFCC02 PROCSS FROM Frill PASTE

FIG. 222—Flow chart of AFCC03 PROCSS FROM Frill PASTE

FIG. 223—Flow chart OF AFCC04 FROM Frill PASTE

FIG. 224—PROCESS OF AFCC05 FROM Frill PASTE

FIG. 225—Flow chart of AFCC 06 PROCSS FROM Frill PASTE

FIG. 226—Flow chart of AFCC 07 PROCSS FROM Frill PASTE

FIG. 227—Flow chart of AFCC 08 PROCSS FROM Frill PASTE

FIG. 228—Flow chart of AFCC 09 PROCSS FROM Frill PASTE

FIG. 229—Flow chart of AFCC 10 PROCSS FROM Frill PASTE

FIG. 230—Flow chart of AFCC 11 PROCSS FROM Frill PASTE

FIGS. 231A&B—Flow chart of AFCC 12 PROCSS FROM Frill PASTE

FIG. 232—Flow chart of AFCC 13 PROCSS FROM Frill PASTE

FIG. 233—Flow chart of AFCC 14 PROCSS FROM Frill PASTE

FIG. 234—Flow chart of AFCC 15 PROCSS FROM Frill PASTE

FIG. 235—Flow chart of AFCC 16 PROCSS FROM Frill PASTE

FIG. 236—AFOD KH & Fr. IV

FIG. 237—AFOD KH

FIG. 238—Flow chart of AFOD and PCC from FrIV1+1V4 ppt with chromatography method

FIG. 239—Flow chart of AFOD01 FROM FrIV1+IV4 PASTE

FIG. 240—Flow chart of AFOD02 FROM FrIV1+IV4 PASTE

FIG. 241—Flow chart of AFOD03 FROM FrIV1+IV4 PASTE

FIG. 242—Flow chart of AFOD 04 FROM FrIV1+IV4 PASTE

FIG. 243—Flow chart of AFOD 05 FROM FrIV1+IV4 PASTE

FIG. 244—Flow chart of AFOD 06 FROM FrIV1+IV4 PASTE

FIG. 245—Flow chart of AFOD 07 FROM FrIV1+IV4 PASTE

FIG. 246—Flow chart of AFOD 08 FROM FrIV1+IV4 PASTE

FIGS. 247A&B—Flow chart of AFOD 09 FROM FrIV1+IV4 PASTE

FIGS. 248A&B—Flow chart of AFOD 10 FROM FrIV1+IV4 PASTE

FIGS. 249A&B—Flow chart of AFOD 11 FROM FrIV1+IV4 PASTE

FIGS. 250A&B—Flow chart of AFOD 12 FROM FrIV1+IV4 PASTE

FIGS. 251A&B—Flow chart of AFOD 13 FROM FrIV1+IV4 PASTE

FIGS. 252A&B—Flow chart of AFOD 14 FROM FrIV1+IV4 PASTE

FIG. 253—Flow chart of AFOD 15 FROM FrIV1+IV4 PASTE

FIG. 254—Flow chart of AFOD 16 FROM FrIV1+IV4 PASTE

FIGS. 255-265—Photographs of Cryopaste and FVIII

### BACKGROUND

The discovery of the new proteins which are already in existence in all the plasma derived products from human source, animal source, recombinant DNA source, Monoclonal source, transgenic source, natural substance and the expression of cell from the cultured GOOD HEALTHY CELLS lead us to the discovery of a number of the following human plasma process:

### Human Blood Plasma

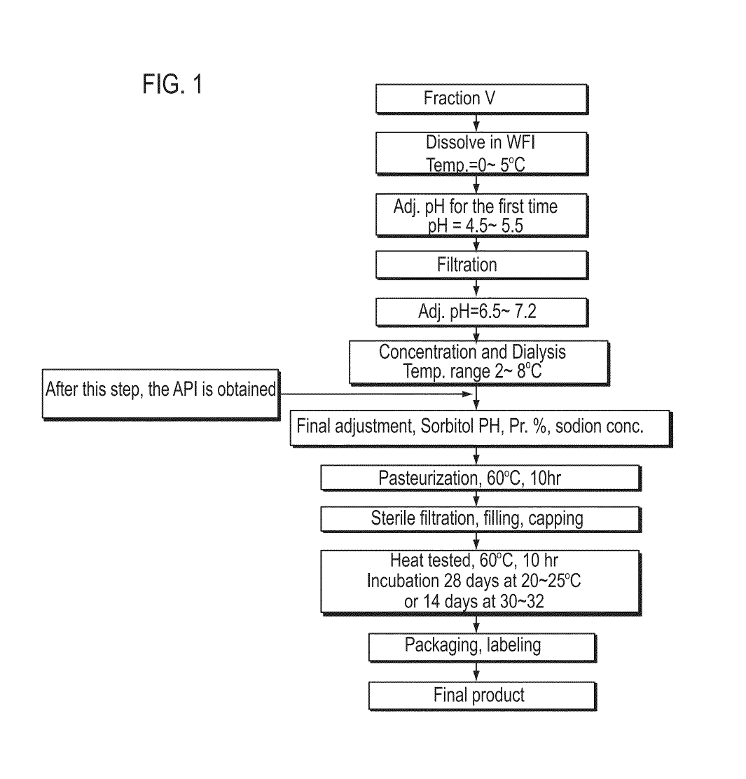

1) AFOD RAAS 101@contain protein ALB Uncharacterized protein, HPR 31 kDa protein, Albumin Uncharacterized protein, AIBG isoform 1 of Alpha-1B-glycoprotein, all of these proteins can be found in the import human albumin from the three different manufacturers but lack HPR haptoglobin, ACTC 1 Actin, alpha cardiac muscle and KH51 protein which can only be found in AlbuRAAS® and the concentration of Human Albumin containing all these proteins must be equal to 30% or higher to be effective. FIG. 1

Protein sequences of ALB Uncharacterized protein, HPR 3 lkDa protein, Albumin Uncharacterized protein, AIBG isoform 1 of Alpha-1B-glycoprotein HPR haptoglobin.

### Protein Sequence of M1, M2, M7, M9, M10

| 299/m1 | Instr./Gel Origin |
|---|---|
| [1] Sample Project | Instrument Sample Name |
| 20120517 | |

| Accession | | | |
|---|---|---|---|
| No. | Protein Name | Protein | Protein MW |
| PI00022434 | Tax ld, 9606 Gene_Symboi ALB | Pi | 738814 |
| | Uncharacterized | 6.33 | |
| | protein | | |

### Peptide Information

| | Obsrv. | | | Start | End | |
|---|---|---|---|---|---|---|
| Caic. Mass | Mass | ±da | ±ppm | Seq. | Seq. | Sequence |
| 875.5098 | 875.5258 | 0.016 | 18 | 243 | 249 | LSO.RFPK |
| 927.4934 | 927.5149 | 0.0215 | 23 | 162 | 168 | YLYEIAR |
| 927.4934 | 927.5149 | 0.0215 | 23 | 162 | 168 | YLYE1AR |
| 960.5625 | 960.5834 | 0.0209 | 22 | 427 | 434 | FQNALLVR |
| 960.5625 | 960.5834 | 0.0209 | 22 | 427 | 434 | FQNALLVR |
| 1000.6037 | 1000.612 | 0.0083 | 8 | 550 | 558 | QTALVELVK |
| 1055.5884 | 1055.6189 | 0.0305 | 29 | 161 | 168 | KYLYEAR |
| 1074.5426 | 1074.5758 | 0.0332 | 31 | 206 | 214 | LDELRDEGK |
| 1083.5946 | 1063.62 | 0.0254 | 23 | 162 | 169 | YLYE1ARR |
| 1128.6987 | 1128.7164 | 0.0177 | 16 | 549 | 558 | KOTALVELVK |
| 1138.498 | 1138.5211 | 0.0231 | 20 | 500 | 508 | CCIESLVNR |
| 1311.7419 | 1311.7593 | 0.0174 | 13 | 362 | 372 | HPDYSVV::!R |
| 1358.6298 | 1358.6437 | 0.0139 | 10 | 570 | 581 | AVMDDFAAFVEK |
| 1358.6298 | 1358.6437 | 0.0139 | 10 | 570 | 581 | AVMDDFAAFVEK |
| 1371.5668 | 1371.5905 | 0.0237 | 17 | 187 | 198 | AAFTECCQAADK |
| 1443.6421 | 1443.6641 | 0.022 | 15 | 287 | 298 | YICENQDSESSK |
| 1467.8431 | 1467.8513 | 0.0082 | 6 | 361 | 372 | RHPDYSWLLLR |
| 1511.8429 | 1511.8691 | 0.0262 | 17 | 439 | 452 | VPQVSIPILVEVSR |
| 1546.7968 | 1546.8112 | 0.0144 | 9 | 299 | 310 | LKECCEKPLLEK |
| 1552.5978 | 1552.62 | 0.0222 | 14 | 384 | 396 | CCAAAD PH ECYAK |
| 1552.5978 | 1552.62 | 0.0222 | 14 | 384 | 396 | CCAAADPHECYAK |
| 1627.6904 | 1627.745 | 0.0546 | 34 | 585 | 598 | ADDKEICFAEEGQK |
| 1639.9379 | 1639.9292 | −0.0087 | −5 | 433 | 452 | KVPQVSTPTLVEVSR |
| 1639.9379 | 1639.9292 | −0.0087 | 5 | 438 | 452 | KVPQVSTPILVEVSR |
| 1650.8949 | 1650.8706 | −0.0243 | −15 | 250 | 264 | AEFAEVSKLVTDLIK |
| 1657.7527 | 1657.7756 | 0.0229 | 14 | 414 | 426 | QNCE I FE QL GEYK |
| 1684.821 | 1684.9177 | 0.0967 | 57 | 287 | 300 | Y10ENQDSISSKLK |
| 1714.7966 | 1714.8048 | 0.0082 | 5 | 118 | 130 | QEPERNECFLQHK |
| 1856.9099 | 1856.8966 | −0.0133 | −7 | 566 | 581 | EQLKAVMDDFAAFVEK |
| 1910.9318 | 1910.9406 | 0.0088 | 5 | 509 | 524 | RPCFSALEVDETYWK |
| 1910.9318 | 1910.9406 | 0.0088 | 5 | 509 | 524 | RPCFSALEVDETYVPK |
| 1996.9294 | 1996.942 | 0.0126 | 6 | 123 | 138 | NECFLQHKDDNPNLPR |
| 2045.0955 | 2045.0938 | −0.0017 | | 397 | 413 | VFDEFKPLVEEPQNLEK |
| 2045.0955 | 2045.0938 | −0.0017 | −1 | 397 | 413 | VEDEFKPLVEEPQNLIK |
| 2124.9875 | 2124.9539 | −0.0336 | | 187 | 205 | AAFTECCQAADKAACLLp |
| | | | | | | K |
| 2260.0227 | 2260.0466 | 0.0239 | | 525 | 543 | EFNAETFTEHADICTLSEK |
| 2545.1665 | 2545.1492 | −0.0173 | | 525 | 545 | EFNAEIFITHADICILSEK |
| | | | | | | ER |
| 2585.1177 | 2585.0925 | −0.0252 | −10 | 265 | 286 | VHIECCHGDLLECADDR |
| | | | | | | ADLAK |
| 2585.1177 | 2585.0925 | −0.0252 | −10 | 265 | 286 | VHIECCHGDLLECADDR |
| | | | | | | ADLAK |
| 2599.2974 | 2599.1685 | −0.1289 | −50 | 414 | 434 | QNCELFEQLGEYKFONA |
| | | | | | | LLVR |
| 2650.2642 | 2650.1511 | −0.1131 | −43 | 139 | 160 | LVRPEVDVNICIAFFEDNE |
| | | | | | | ETFLK |
| 2666.259 | 2666.1682 | −0.0908 | −34 | 139 | 160 | LVRPEVDVMCIAFFEDNE |
| | | | | | | ETFLK |
| 2794.354 | 2794.2439 | −0.1101 | −39 | 139 | 161 | LVRPEVDVNICIAFFEDNE |
| | | | | | | ETFLKK |
| 2794.354 | 2794.2439 | −0.1101 | −39 | 139 | 161 | LVRPEVDVMCIAFFEDNE |
| | | | | | | ETFLKK |

### Protein Sequence of M1, M2, M7, M9, M10

| 300/m2 | Instr./Gel Origin |
|---|---|
| [1] Sample Project | Instrument Sample Name |
| 20120517 | |

| Accession | | | |
|---|---|---|---|
| No. | Protein Name | Protein | Protein MW |
| IPI00431645 | Tax ld 9606 Gene_Symbol f-IPR | Pi | 31673 |
| | 31 kDa protein | 8.48 | |

### Peptide Information

| | Obsrv. | | | Start | End | |
|---|---|---|---|---|---|---|
| Calc. Mass | Mass | ±da | ±ppm | Seq. | Seq. | Sequence |
| 809.3788 | 809.368 | −0.0108 | −13 | 146 | 152 | DYAEVGR |
| 920.4625 | 920.4637 | 0.0012 | 1 | 46 | 53 | GSFPV\IQAK |
| 920.4625 | 920.4637 | 0.0012 | 1 | 46 | 53 | GSFPWC)AK |
| 980.4948 | 960.4968 | 0.002 | 2 | 153 | 161 | VGYVSGV\IGR |
| 980.4948 | 980.4968 | 0.002 | 2 | 153 | 161 | VGYVSGWGR |
| 1203.6368 | 1203.6545 | 0.0177 | 15 | 267 | 276 | VT.SEQDWVQK |
| 1290.7305 | 1290.6764 | −0.0541 | −42 | 91 | 102 | DIAPILTLYVGK |
| 1345.6458 | 1345.6672 | 0.0214 | 16 | 255 | 266 | SCAVAEYGVYVK |
| 1723.8142 | 1723.8369 | 0.0227 | 13 | 173 | 186 | YVNILPVADQDQC!R |
| 1723.3142 | 1723.8369 | 0.0227 | 13 | 173 | 186 | 1 t/MLPVADQDQCIR |
| 1850.9139 | 1850.9366 | 0.0227 | 12 | 137 | 152 | VMPICI_PSKENADIGR |
| 1850.9139 | 1650.9366 | 0.0227 | 12 | 137 | 152 | VMPICIPSKDYABIGR |
| 2172.0576 | 2172.0862 | 0.0286 | 13 | 201 | 220 | SPVGVONLNEHTFCAG |
| | | | | | | MSK |
| 2172.0576 | 2172.0862 | 0.0286 | 13 | 201 | 220 | SPVGVQPILNEHTFCAG |
| | | | | | | MSK |
| 2188.0525 | 2188.0706 | 0.0181 | 8 | 201 | 220 | SPVGVQP1LNEHTFCAG |
| | | | | | | MSK |

| 305/M7 | Instr./Gel Origin |
|---|---|
| [1] Sample Project | Instrument Sample Name |
| 20120517 | |

| Accession | | | |
|---|---|---|---|
| No. | Protein Name | Protein | Protein MW |
| IPI00022434 | Tax ld 9606 Gene_Symbo! | Pi | 73881.4 |
| | f\LB protein | 6.33 | |
| | Uncharacterized | | |
| | protein | | |

### Peptide Information

| | Obsrv. | | | Start | End | |
|---|---|---|---|---|---|---|
| Calc. Mass | Mass | ±da | ±pp, | Seq. | Seq | Sequence |
| 927.4934 | 927 4874 | −0.006 | −6 | 162 | 168 | YLYEIAR |
| 927.4934 | 927.48?4 | ··0.006 | ·6 | 162 | 168 | YLYEIAR |
| 960.5625 | 960.5604 | −0.002″1 | −2 | 427 | 434 | FQf\1ALLVR |
| 950.5625 | 960.5604 | −0.002″1 | −2 | 427 | 434 | FQNALLVR |
| 1000.60:37 | 1000.5975 | −0.0062 | −6 | 550 | 558 | QTALVELVK |
| ″1055.5884 | 1055.5979 | 0.0095 | 9 | 161 | 168 | KYLYEiAR |
| ″10745426 | 1074.5447 | 0.0021 | 2 | 206 | 214 | LDELRDEGK |
| 1138.498 | 1138.5083 | O.Oi03 | 9 | 500 | 508 | CCTESLVNR |
| 1149.615 | 1149.6238 | 0.0088 | 8 | 66 | 75 | LVI\JEVTEFAK |
| 1311.7419 | 1:311.7579 | 0{116 | 12 | 362 | 372 | HPDYSVVLLLR |
| ″1342.6348 | 1342.6411 | 0.0063 | 5 | 510 | 581 | AVMDDFAAFVE:K |
| 1342.6348 | 1342.6411 | 0.0063 | 5 | 570 | 581 | AVMDDF/V\FVEK |
| 1352.″1686 | 1352.T791 | om·1·1 | 8 | 427 | 437 | FQNALL VRYTK |
| 1358.6298 | 1:358.6348 | 0{105 | 4 | 570 | 581 | AVMDDFi\AFVEK |
| ″137″1.5668 | 1371.5879 | 0.0211 | 15 | 181 | 198 | AAFTECCQAADK |
| 1443.6421 | 1443.6553 | 0.0132 | 9 | 287 | 298 | YICENQDSISSI< |
| 146″1.8431 | 1461.8514 | 0.0143 | ″1O | 36″1 | 31′2 | RHPDYSVVLLLR |
| 1467.84:31 | 14137.8574 | (1.(J143 | 10 | 361 | 372 | RHPDYSVVLLLR |
| ″15″1″1.8429 | 1511.8596 | O.o167 | 11 | 4: 9 | 452 | VPOVS TPTLVE:VSR |
| 1546.7968 | 1546.8142 | 0.0174 | 11 | 299 | :310 | LI<EC:CEKPLLEI< |
| 1552.5918 | 1552.6318 | 0.034 | 22 | 384 | 396 | CCAAADPHECYAK |
| 1552.5978 | 1552.13318 | (1.(J34 | 22 | 384 | 396 | CCAAADPHECYAK |
| ″1623.7876 | 1623.8319 | 0.0443 | 21 | :H8 | 360 | DVFLGMFLYE:YAR |
| 1627.6904 | 1627.7493 | 0.0589 | 36 | 585 | 598 | ADDKETC:FAEEGQK |
| 15: 9.9319 | 1639.9246 | −0.0133 | −8 | 438 | 452 | KVPQVSTPTLVE:VSR |
| 1639.9:379 | 113:39.92413 | −0.CJ133 | −8 | 438 | 452 | KVPQVSTPTLVEVSR |
| ″1650.8949 | 1650.8693 | 0.0256 | −16 | 250 | 264 | AEFAEVSKLVTDL TK |
| 1657.7527 | 1657.7588 | 0.0061 | 4 | 414 | 4213 | ONCELFEQLGEYK |
| 1684.821 | 1684.8501 | 0.029″1 | ″17 | 281′ | 300 | YICEI\JQDSISSKLK |
| 1742.8942 | 1742.91713 | (1.(J234 | 13 | 170 | 183 | HPYFYAPELLFFAK |
| 1898.9952 | 1899.0358 | 0.0406 | 21 | 110 | 184 | HPYFYAPE:LLFFAKR |
| 1898.9952 | 1899.0358 | 0.0406 | 21 | 169 | 183 | RHPYFYAPELLFFAI< |
| 1910.9318 | 1910.9614 | 0.0196 | ″10 | 509 | 524 | RPCFSALEVDE:TYVPK |
| 1910.9:318 | 1910.9514 | 0{1196 | 10 | 509 | 524 | RPCFS,<\LEVDETYVPK |
| ″1924.0863 | 1924.0873 | 0.001 | 1 | 4: 9 | 466 | VPOVSrPTLVE:VSRNL GK |
| 2045.0955 | 2045.0996 | 0.0041 | 2 | 397 | 413 | VFDEFI<PLVEEPQNLII< |
| 204S.G955 | 2046.0996 | 0.004″1 | 2 | 391′ | 413 | VFDE:FKPLVE:EPOI\JLIK |
| 2086.8:3713 | 20813.81394 | 0{1318 | 15 | 265 | 281 | VHTECC:HGDLLECADDR |
| 2260.0227 | 2260.0278 | 0.0051 | 2 | 525 | 643 | E:FNAE:TFn=HADICrL. SEK |
| 2545.1665 | 2545.1123 | −0.0542 | −21 | 525 | 545 | EFNAETFTFHADIC:TLSEK |
| | | | | | | ER |
| 2585.1177 | 2585.1113 | −0{1064 | −2 | 265 | 286 | VHTECC:HGDLLECADDR |
| | | | | | | ADLAK |
| 2585.1177 | 2585.1113 | −0{1064 | −2 | 265 | 286 | VHTECC:HGDLLECADDR |
| | | | | | | ADLAK |
| 2599.2974 | 2599.0598 | −0.2376 | −91 | 414 | 4:34 | ONCELFEQL GEYKFQNA |
| | | | | | | LLVR |
| 2650.21342 | 21350.21305 | −0.CJ037 | −1 | 139 | 160 | LVRPEVDVMCTi\FHDNE |
| | | | | | | E1H.K |
| 2778.3589 | 2778.3564 | −0.0025 | −1 | 139 | 1131 | LVRPEVDVMCTAFHDNE |
| | | | | | | ETFLKK |
| 2794.354 | 2794.3438 | −0 1102 | −4 | 139 | 161 | LVRPEVDVMCTi\FHDNE |

| 307/M9 | Instr./Gel Origin |
|---|---|
| [1] Sample Project | Instrument Sample Name |
| 20120517 | |

| Accession | | | |
|---|---|---|---|
| No. | Protein Name | Protein | Protein MW |
| IPI00022895 | Tax_Id = 9606 Gene Symboi = A18G | Pi | 5478B.8 |
| | Isofornl1 of Alpha-1 B-glycoprotein | 5.56 | |
| | protein | | |

### Peptide Information

| | Obsrv. | | | Start | End | |
|---|---|---|---|---|---|---|
| Calc. Mass | Mass | ±da | ±pp, | Seq. | Seq | Sequence |
| 861.46?6 | 8•61.4217 | −0.0459 | −53 | 437 | 444 | EGETKAVK |
| 870.5295 | 870.5177 | −0{1118 | −14 | 107 | 114 | LLELTGPK |
| 8705295 | 810.51T7 | −0.0118 | −14 | 10? | 114 | LLEL rGPK |
| 1264.6532 | 126413721 | 0.0189 | 15 | 95 | 106 | SGLSTGWTQLSK |
| 1264.65: 2 | •1264.6721 | 0.o-!89 | 15 | 95 | 106 | SGLSTGWTOLSK |
| 1372.6969 | 1372.7217 | (I.(J248 | 18 | 79 | 90 | HQFLLTGDTQGR |
| ″1372.6969 | 1:r12.1211 | 0.0248 | 18 | 79 | 90 | HQFLLTGDIT GR |

| 308/M10 | Instr./Gel Origin |
|---|---|
| [1] Sample Project | Instrument Sample Name |
| 20120517 | |

| Accession | | | |
|---|---|---|---|
| No. | Protein Name | Protein | Protein MW |
| IPI00641737 | Tax_Id = 9606 Gene Symboi = | Pi | 45860.8 |
| | HP; HPR Haptoglobin _ | 6.13 | |
| | protein | | |

### Peptide Information

| Calc. Mass | Obsrv. | ±da | ±pp, | Start | End | Sequence |
|---|---|---|---|---|---|---|
| 856.4675 | 856.4838 | 0.0163 | 19 | 113 | 118 | NYYKLR |
| 855.4615 | 856.41B8 | 0.0163 | 19 | 54 | 59 | NYYKLR |
| 920.4625 | 920.4198 | −0.0427 | −46 | 171 | 178 | GSFPWQAK |
| ·1708.850·1 | 1708.8895 | 0.0394 | 23 | 111 | ″!3″! | LRTEGDGVYTLNNEK |
| 1857.9198 | 1857.9403 | 0.0205 | 11 | 137 | 153 | AVGDKLPECEAVCGKPK |
| 185?.9198 | 1851.94tn | 0.0205 | 11 | ″137 | 153 | AVGDKL PECEAVCGKPK |

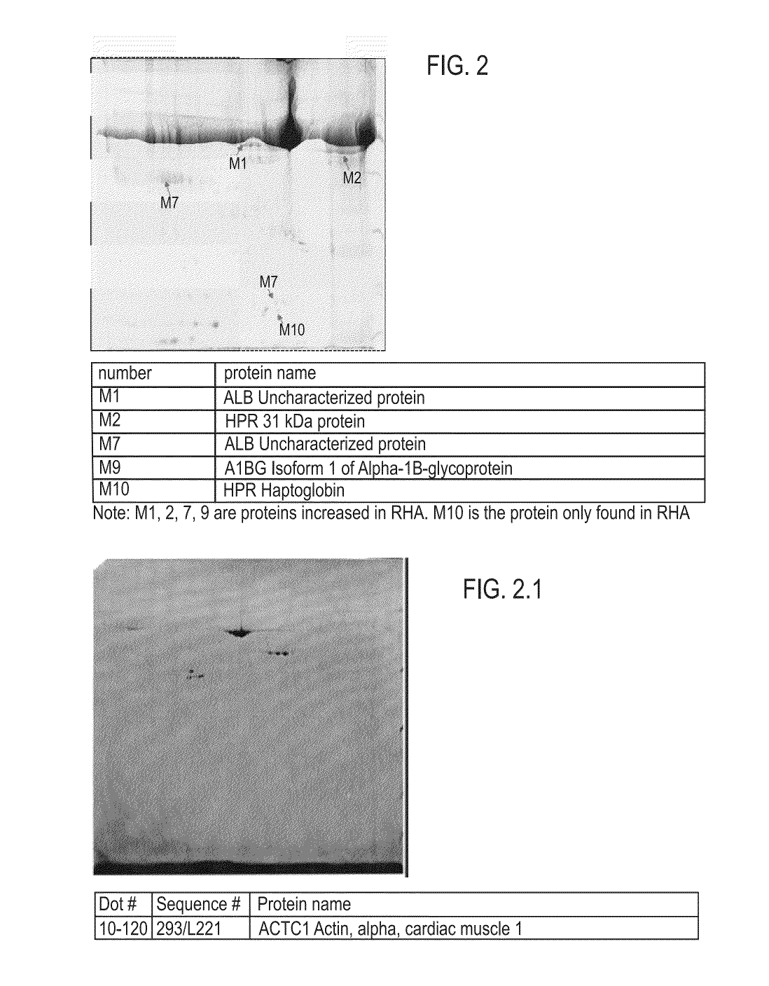

In the final comparison AFOD RAAS 101 product contains a total of six proteins ALB Uncharacterized protein, HPR 31 kDa protein, Albumin Uncharacterized protein, A1BG isoform 1 of Alpha-IB glycoprotein HPR haptoglobin and KH51. In this product it contains HPR Haptoglobulin, ACTCI Actin, alpha cardiac muscle 1 and a newfound protein KH51 both of which are very crucial in the application for cancer and bacteria. These three proteins could not be found in any international imported human albumin.
FIG. 2, 2.1

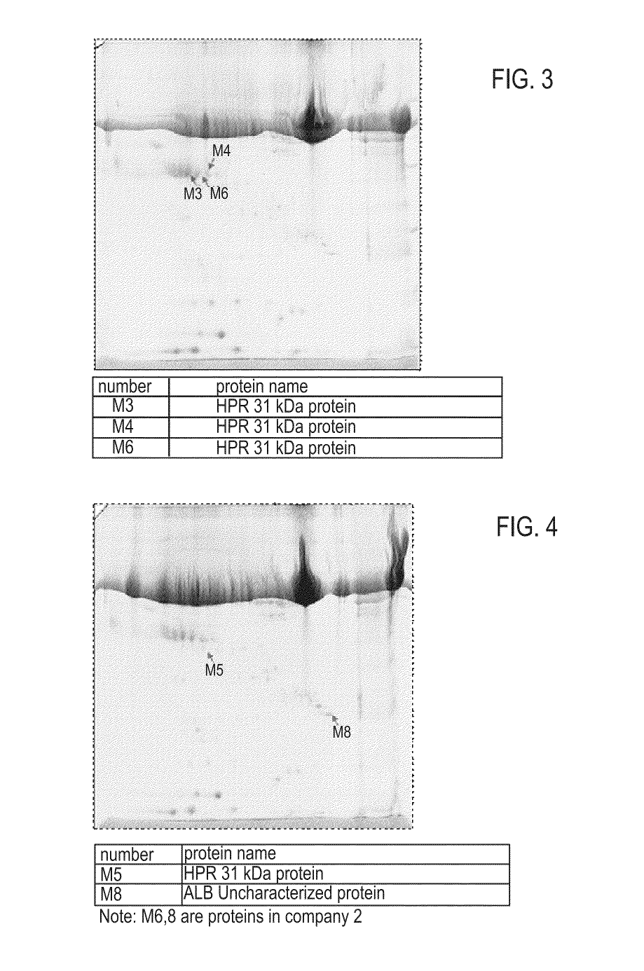

To compare with AFOD RAAS 101 international import company 1 has only one protein HPR 31 kDa Protein vs 7 proteins in AFOD RAAS 101.
FIG. 3

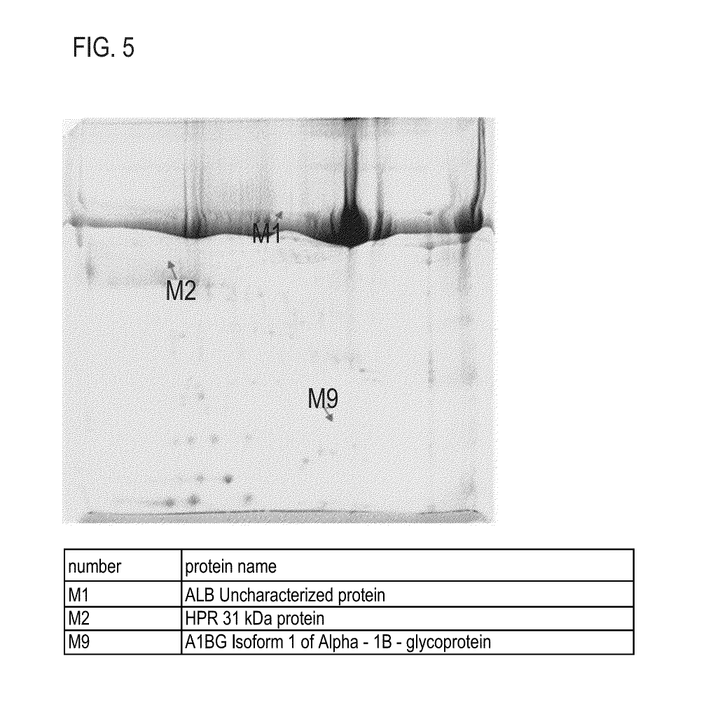

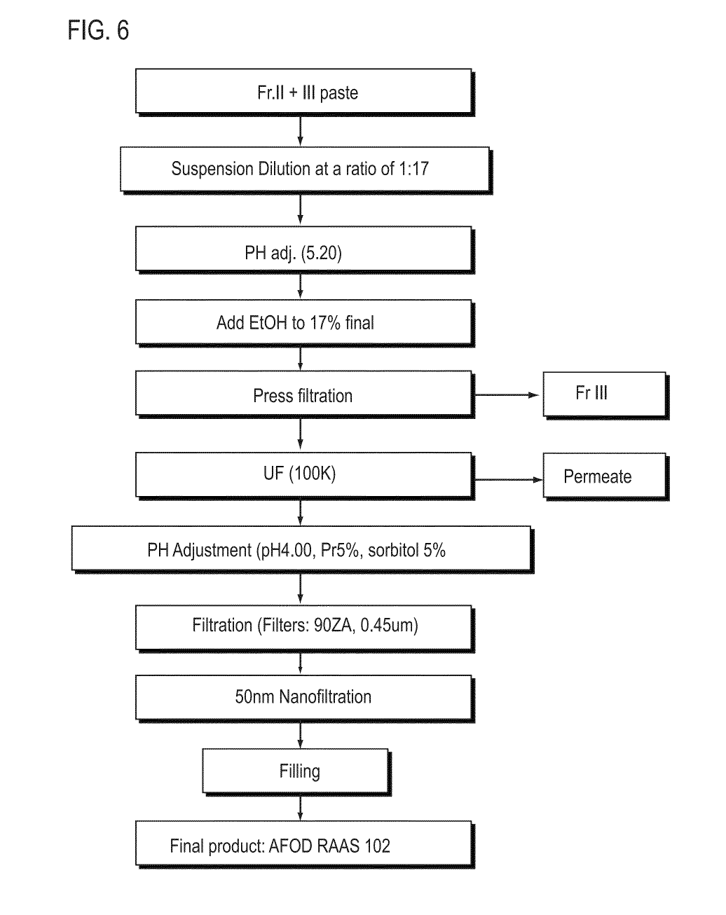

Company 2 has two proteins HPR 31 kDa and Albumin uncharacterized proteins vs 7 proteins in AFOD RAAS 101.
FIG. 4
Company 3 has three proteins Albumin uncharacterized protein, HPR 31 kDa protein and, A1BG isoform
1 of Alpha-1B-glycoprotein vs 7 proteins in AFOD RAAS 101.
FIG. 5
In conclusion the maximum amount of proteins in the international import companies is three or 58% LESS compared to AFOD RAAS 101, and the minimum amount of proteins is one protein or 86% LESS. None of the international import companies contain the existing protein HPR Heptaglobulin, ACTC1
Actin, alpha cardiac muscle 1 and new discovered KH51protein.
   2) AFOD RAAS 1020: Beside the main component of Immunoglobulin AFOD RAAS 102 contains three existing proteins 120/E19 IGHV4-31; IGHG144 kDa protein and 191/H18 IGHV4.31; IGHG1
   32 kDa and IGHV4.31; 1 GHG1Putative uncharacterized protein DKFZp686G11190 proteins including five newly discovered proteins KH33, KH34, KH35, KH36 and KH37. The combination of these five proteins with the concentration at 30% have been found to be very effective against the viruses like H1N1, H5N1, foot and mouth disease and specially changing the protein which causes the Hepatitis B virus to stop the DNA replication and cure the Hepatitis B within the three days in mice and as well as bacteria and solid and blood cancers.
FIG. 6
   Protein sequence

| 120E19 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| IPI00448925 | Tax_!d, %06 Gene_Symboi, | Pi | 44511.3 |
| | IGHV4-31; 1GHG1 44 kDa | 6.55 | |
| | protein | | |

### Peptide Information

| Calc. Mass | Obsrv. | ±da | ±pp, | Start | End | Sequence |
|---|---|---|---|---|---|---|
| 835.4342 | 835.4091 | −0.02 ′\1 | −30 | 132 | 138 | DTLMISR |
| 838.5032 | 838.4759 | −0 0273 | ··33 | 210 | 217 | ALPf\PIEK |
| 838.5032 | 838.4759 | ··0.0273 | −33 | 210 | 217 | ALPAPIEK |
| 851.4291 | 851.4036 | −0.0255 | −30 | 132 | 138 | DTLM!SR |
| 1161.6296 | 1161.6327 | 0.0031 | 3 | 244 | 253 | NQVSLTCLVK |
| 1161.62% | 1161.6327 | o.orn1 | 3 | 244 | 253 | NQVSLTCLVK |
| 1186.6467 | 1186.5533 | −0 0934 | −79 | 5 | 1 t) | GPSVFPLAPSSK |
| 1266.674 | 1286.6965 | 0.0225 | 17 | 228 | 238 | EPQVYTLPPSR |
| 1286.674 | 1286.6965 | 0.0225 | 17 | 228 | 238 | EPQVYTLPPSR |
| 1676.8-125 | 1676.9005 | 0.058 | 35 | 385 | 399 | QT!IPDYRr MIGQGA |
| 1677.802 | ″1677.8694 | 0.0674 | 40 | 158 | 171 | FI′JWYVDGVEVHI′JAK |
| 1677.802 | 1677.8694 | 0.0674 | 40 | 158 | 171 | FtNv′YVDGVEVHt AK |
| 1872.9702 | 18″130851 | 0.1149 | 61 | 228 | 243 | EPQVYTLPPSRDELTK |
| 1872.9702 | 1873.0851 | 0.1149 | 61 | 228 | 243 | EPQVYTLPPSRDELTK |
| 2139.027621 | 2139.0417213 | 0.01410.199 | 7 | 139 | 1571 | TPEVTCVVVDVSHEDPET |
| | | | | | | VK |
| 9.0276 | 9.22″11 | 5 | 93 | 139 | 157 | TPEVTCVVVDVSHEDPE |
| | | | | | | VK |
| 2139.0276 | 2139.22″11 | 0.1995 | B3 | 139 | 15″7 | TPEVTCVvVDVSHEDPE |
| | | | | | | VK |
| 2544.1313 | 2544.37″16 | 0.2403 | 94 | 254 | 275 | GFYPSDIAVEWESNGQP |
| | | | | | | ENNYK |
| 2801.2671 | 2801.4607 | 0.1936 | 69 | 00 | 22 | WQQGI′JVFSCS\Irv1HEAL |
| | | | | | | HNHYTQK |
| 2817. 622 | 2817.5144 | 0.2522 | 90 | 300 | 3 2 | WQQGNVFSCSVMHEAL |

| 191H18 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| IPI00892671 | Tax ld, 9606 Gene_Symboi = | Pi | 32476.2 |
| | IGHV4-31; IGHG1 32 kDa | 8.3 | |
| | protein | | |

### Peptide Information

| | Obsrv. | | | Start | End | |
|---|---|---|---|---|---|---|
| Calc. Mass | Mass | ±da | ±pp, | Seq. | Seq | Sequence |
| −J9W.9318 | 1910.9406 | 0.0088 | s | SG9 | 524 | RPCFSAL.EVDETYVPK |
| 1910.9318 | 1910.9406 | 0.0088 | 5 | 509 | 524 | RPCFSALEVDETYVPK |
| 1996.9294 | 1996.942 | 0.0″126 | 6 | ″!23 | 138 | NECFLQHKDDNPNLPR |
| 2045.0955 | 2045{1938 | −0{1017 | −1 | 397 | 413 | VFDEFKPLVEEPQNLIK |
| 2045.0955 | 2045.0938 | −0.001? | −1 | : 91 | 413 | VFDEFKPLVEEPQNLIK |
| 2124.9875 | 2124.9539 | −0.0336 | −16 | 187 | 205 | A/>.FTECCQ,<\ADKAACLLP |
| | | | | | | K |
| 2260.0227 | 22130{1466 | (1.(J239 | 11 | 525 | 543 | EFNAETFTFHAD!CTLSEK |
| 2545:1665 | 2545.1492 | −0.01?3 | −1 | 525 | 545 | EFNAETFn=HJLDiCrL.SEK |
| | | | | | | ER |
| 2585.1111 | 2585.0925 | −0.0252 | −W | 265 | 286 | VHrECCHGDLLECADDR |
| | | | | | | ADLAK |
| 2585.″!11′1′ | 25850925 | −0.0252 | −10 | 265 | 286 | VHT ECCHGDLLECADDR |
| | | | | | | ADLAK |
| 2599.2914 | 2599.1685 | −0.1289 | −50 | 4″14 | 434 | QNCELFEQLGEYKFQNA |
| | | | | | | LLVR |
| 2650.2642 | 2650.1511 | −0.1131 | −4: | 1: 9 | 160 | LVRPEVDVMCTAFHDNE |
| | | | | | | ETFLI< |
| 2666.259 | 2666.1682 | −0.0908 | −34 | ″!39 | 160 | LVRPEVDVMCrAFHDNE |
| | | | | | | ETFLK |
| 2794.354 | 2794.2439 | −0.1101 | <9 | 1: 9 | 161 | LVRPEVDVMCTAFHDNE |
| | | | | | | ETFLI<K |
| 2?94.: 54 | 2194.2439 | −0.1″10″1 | −39 | ″!39 | 161 | LVRPEVDVMCrAFHDNE |
| | | | | | | ETFLKI< |
| 1161.6296 | 1161.6295 | ··0.0001 | 0 | 209 | 218 | NQVSLTCLVK |
| 1161.6296 | ″1161.6295 | −0.0001 | 0 | 209 | 218 | NQVSLTCLVK |
| 1286674 | 1286.6779 | 0.0039 | 3 | 193 | 203 | EPQVYTLFPSR |
| 1286.674 | 1286.6779 | 0.0039 | 3 | 193 | 203 | EPQVYTLPPSR |
| 18n.9″?02 | 1872.993″1 | 0.0 35 | 13 | 193 | 208 | EPQVYTLPPSRDELTK |
| 1872.9702 | 1872.9937 | 0.0235 | 13 | 193 | 208 | EPQVYTLPPSRDELTK |
| 18″?3.9219 | 1873.9736 | 0.0517 | 28 | 241 | 257 | TTPPVLDSDGSFFLYSK |
| 2544.1313 | 2544.1079 | −0.0234 | −9 | 219 | 240 | GFYPSDIAVEWESI′JGQP EI′JI′JYK |
| 2544.B13 | 2544.10″?9 | −0.0234 | −9 | 219 | 240 | GFYPSDIAVEWESI′JGQP |
| | | | | | | ENNYK |
| 2801.2671 | 2801.2739 | 0.0068 | 2 | 26 ) | 28? | WOQGI′JVFSCSVMHEAL HNHYTQK |
| 2801.2671 | 2801.2739 | 0.0068 | 2 | 265 | 287 | WOQGNVESCSVII\J1 HEAL |
| 2801.2739 | | | | | | |
| | | | | | | HNHYTQK |
| 2817.2622 | 2817.2522 | −0.01 | −4 | 265 | 287 | WQQGr VFSCSVMHEAL Hr HYTQK |

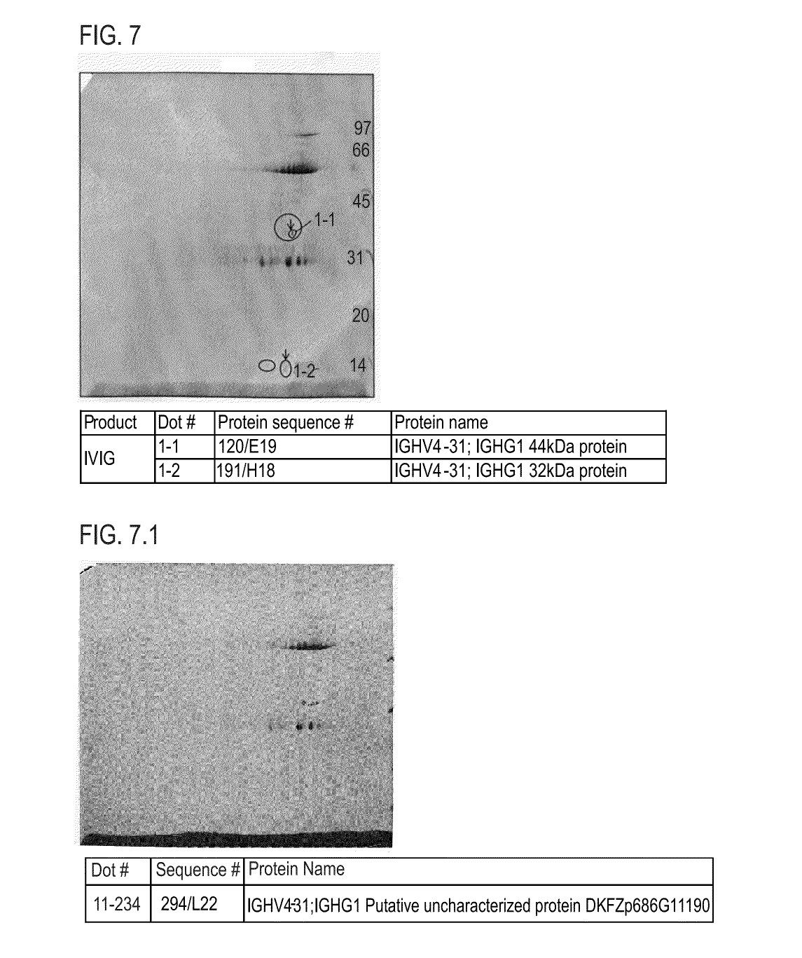

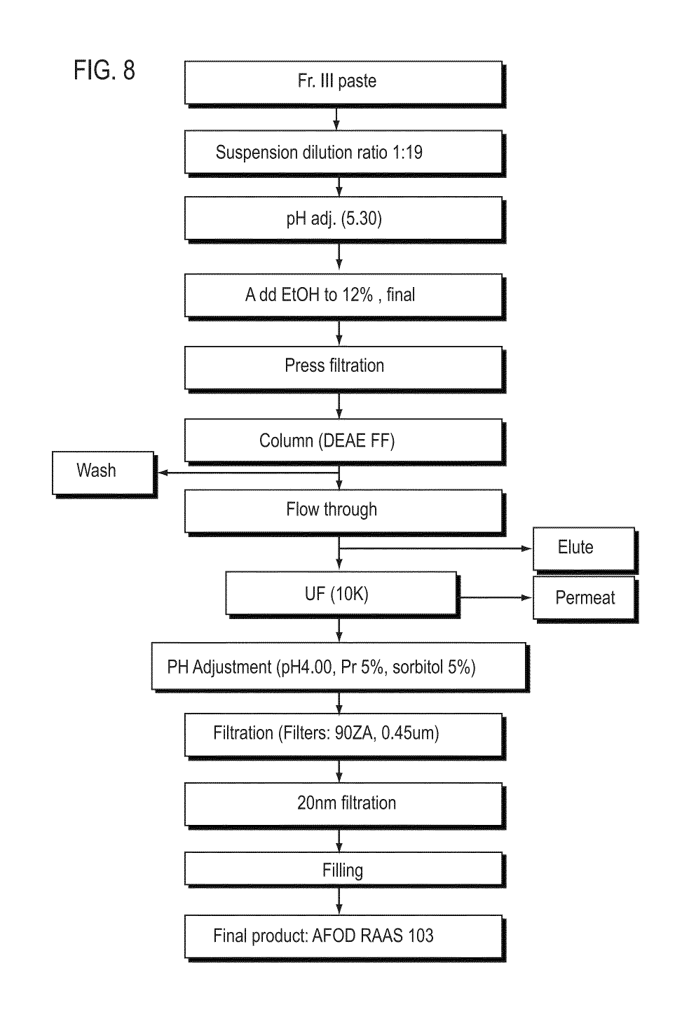

FIG. 7, 7.1
3) AFOD RAAS 103® Contains the four existing discovered proteins 193/H20 TF serotransferrin,
   194/H21APOH beta2-glycoproteln 1, 195/H22 cDNA FU5165, moderately similar to beta-2-glycoprotein, 196/H23FCN3 isoform 1 of Ficolin-3. In addition it may contain KH3, KH4, KHS, KH6, KH7, KH8, KH9, KH10, KH41, KH42 and KH43 proteins. This AFOD RAAS 103 has proven to change the bad protein of the HCV RNA virus into the good protein to cure Hepatitis C.
FIG. 8

### Protein Sequence

| 193/H20 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| IPI00022463 | Tax id = 9606 Gene Symboi = TF | Pi | 79294.5 |
| | Serotransferrin | 6.81 | |
| | protein | | |

### Peptide Information

| | Obsrv. | | | Start | End | |
|---|---|---|---|---|---|---|
| Calc. Mass | Mass | ±da | ±pp, | Seq. | Seq | Sequence |
| 827.4046 | 827.4172 | 0.0′126 | 15 | 565 | 57′i | r PDPWAK |
| 8″?4.4417 | 874.446B | 0.0052 | 6 | 31 t) | 323 | DSAHGFLK |
| 887.4152 | 887.4246 | O.OOB4 | 11 | 468 | 475 | SCHTAVGR |
| 964.5323 | 964.5367 | 0.0044 | 5 | 601 | 609 | APNHAV\ITR |
| 997.4771 | 997.4792 | 0.0021 | 2 | 6L″ | 69 | ASYLDCIR |
| 1000.4985 | 1000.4951 | −0 0034 | −3 | 669 | 676 | YLGEEYVK |
| 1015.5101 | 1015.5131 | 0.003 | 3 | 467 | 475 | KSCHT.AVGR |
| 1125.5721 | 1125.5751 | 0.003 | | 61 | 69 | KASYLDCIR |
| 1166.5913 | 1166.5861 | −0.0052 | −4 | 554 | 564 | HQTVPQt TGGK |
| 1195.542t) | 1195.5465 | 0.0039 | 3 | 363 | 3′71 | WCALSHHER |
| 1195.5525 | 1195.5465 | −0.006 | −5 | 123 | 132 | DSGFQMNQLR |
| 1211.5474 | 1211.5527 | 0.0053 | 4 | 123 | 132 | DSGFQIVINQLR |
| 1249.606 | ″1249.6086 | 0.0026 | .″:. | −154 | −164 | SASDLTWDNLK |
| 1249.606 | 1249.6086 | 0.0026 | 2 | 454 | 464 | SASDLTWDDLK |
| 1273.65% | 1273.6465 | −0.0071 | −6 | 226 | 236 | HSTiFENLANK |
| 12″ Lactate | 1 76.6421 | 0.01 | 8 | 300 | 310 | EFQLFSSPHGK |
| dehydrogenase | | | | | | |
| 6321 | | | | | | |
| 1283.5692 | 1283.5695 | 0.0003 | 0 | 531 | 541 | EGYYGYTGAFR |
| 1283.5692 | 1283.5695 | 0.0003 | 0 | 531 | 541 | EGY′r″GYTGAFR |
| 1317.5892 | 1317.5931 | 0.0039 | 3 | 27 | 37 | WCAVSEHEATK |
| 1323.6475 | ″1323.6637 | 0.0162 | 1.″: | 122 | 132 | KDSGFQMNQLR |
| 13 9.6423 | 1339.6395 | −0.0028 | −2 | 122 | 132 | KDSGFQMPJQLR |
| 1354.6307 | 1354.6305 | −0.0002 | 0 | 577 | 587 | DYELLCLDGTR |
| 13″?1.7009 | 1377.699 | −0.0017 | −1 | 453 | 464 | KSASDLWVDN1.K |
| 1415.72 | 1415.7227 | 0.0027 | 2 | 47 | 60 | SVIPSDGPSVACVK |
| 1478.73-19 | ″1478.7483 | 0.0134 | g | 332 | 343 | MYLGYEYVTAIR |
| 1491″159 | 1491.7654 | 0.0064 | 4 | 298 | 3′10 | SKEFQLFSSPHGK |
| 1491.759 | 1491.7654 | 0.0064 | 4 | 298 | 10 | SKEFQLFSSPHGK |
| 1494.7297 | 1494.7448 | 0.0151 | 10 | 332 | 343 | MYLGYE′\″VTAIR |
| 1521.7367 | 1521.7344 | ··0.0023 | ·2 | 372 | 384 | LKCDEWSVNSVGK |
| 1531.688 | 1531.7039 | 0.0159 | 10 | 684 | 696 | CSTSSLLEACTFR |
| 1531.688 | 1531.7039 | 0.0159 | 10 | 684 | 696 | CSTSSLLEACTFR |
| 1539.7″108 | ″1539.7297 | 0.0189 | 1.″: | 240 | 251 | DQYELLCLDI′JTR |
| 1565.7992 | 1565.8019 | 0.0027 | 2 | 647 | 659 | DLLFRDDTVCL!-\K |
| 1565.7992 | 1565.8019 | 0.0027 | 2 | 647 | 659 | DLLFRDDTVCLAK |
| 1577.6577 | 1577.699 | 0.0413 | 26 | 495 | 508 | FDEFFSEGCAPGSK |
| 1586.7744 | 1586.787 | 0.0126 | 8 | 588 | 600 | KPVEEYANCHLAR |
| 1 ′\86.?744 | 1 ′186.187 | 0.0126 | 8 | 588 | 600 | KPVEEYANGHLAR |
| 1593.8094 | 1593.7748 | −0.0346 | 22 | 47″t) | 489 | TAGWNIPMGLLYNK |
| 1615.8187 | 1615.8096 | −·0.0091 | −6 | 226 | 239 | HSTIFENL!-\NKADR |
| 162S1.8159 | 162S1.799 | −0.0169 | −10 | 108 | 121 | EDPOTFYYAVAVVK |
| 1659.783 | 1659.7869 | 0.0039 | 2 | 683 | 6S16 | KCSTSSLLEACTFR |
| 1689.849 | 1689.8651 | 0.0161 | 10 | 259 | 27,′J, | DCHLAQVPSHTVVAR |
| 1705.7″527 | 1705.7793 | 0.0 66 | 16 | 4% | 509 | FDEFFSEGCAPGSi\K |
| 1?06.7659 | 1706.7622 | −0.003′7 | 2 | 516 | 530 | LCMGSGLNLCEPNNi\ |
| 1725.767 | 1725.7515 | −00155 | −9 | 385 | 399 | IEGVSAETTEDGIAK |
| 1817.8044 | 1817.7971 | −·0.0073 | −4 | 347 | 362 | EGTCPEAPTDECKPVK |
| 1881.876 | ″1881.88″12 | 0.0052 | 3 | 237 | 251 | ADRDQYELLCLDI′JTR |
| ·:sa·:.876 | 1881.8812 | 0.0052 | 3 | 237 | 251 | ADRDQYELLCLDt TR |
| 1952.9382 | 1952.9524 | 0.0142 | 7 | 572 | 587 | NLNEKDYELLCLDGTR |
| 2549 293 | 2549.3508 | 0.0578 | 3 | 252 | 273 | KPVDEYi\DCHL.AQVPSH |
| | | | | | | TVVAR |
| 19W.9318 | 1910.9406 | 0.0088 | s | SG9 | 524 | RPCFSAL.EVDETYVPK |
| 1910.9318 | 1910.9406 | 0.0088 | 5 | 509 | 524 | RPCFSALEVDETYVPK |
| 1996.9294 | 1996.942 | 0.0″126 | 6 | ″!23 | 138 | NECFLQHKDDNPNLPR |
| 2045.0955 | 2045{1938 | −0{1017 | −1 | 397 | 413 | VFDEFKPLVEEPQNLIK |
| 2045.0955 | 2045.0938 | −0.001? | −1 | :91 | 413 | VFDEFKPLVEEPQNLIK |
| 2124.9875 | 2124.9539 | −0.0336 | −16 | 187 | 205 | A/>.FTECCQ,<\ADKAACLL |
| | | | | | | P K |
| 2260.0227 | 22130{1466 | (1.(J239 | 11 | 525 | 543 | EFNAETFTFHAD!CTLSEK |
| 2545:1665 | 2545.1492 | −0.0173 | −1 | 525 | 545 | EFNAETFn=HJI.DiCrL.SEK |
| | | | | | | ER |
| 2585.1111 | 2585.0925 | −0.0252 | −W | 265 | 286 | VHrECCHGDLLECADDR |
| | | | | | | ADLAK |
| 2585.″!11′1′ | 25850925 | −0.0252 | −10 | 265 | 286 | VHT ECCHGDLLECADDR |
| | | | | | | ADLAK |
| 2599.2914 | 2599.1685 | −0.1289 | −50 | 4″14 | 434 | QNCELFEQLGEYKFQNA |
| | | | | | | LLVR |
| 2650.2642 | 2650.1511 | −0.1131 | −4: | 1: 9 | 160 | LVRPEVDVMCTAFHDNE |
| | | | | | | ETFLI< |
| 2666.259 | 2666.1682 | −0.0908 | −34 | ″!39 | 160 | LVRPEVDVMCrAFHDNE |
| | | | | | | ETFLK |
| 2794.354 | 2794.2439 | −0.1101 | <9 | 1: 9 | 161 | LVRPEVDVMCTAFHDNE |
| | | | | | | ETFLI<K |
| 2794.: 54 | 2194.2439 | −0.1″10″1 | −39 | ″!39 | 161 | LVRPEVDVMCrAFHDNE |
| | | | | | | ETFLKI< |

Instr./Gel Origin

| 194H21 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| IPI00298828 | Tax_id 9606 Gene_Symboi, APOH | Pi | 39584.1 |
| | Beta-2-giycoprolein | 8.34 | |
| | protein | | |

### Peptide Information

| | Obsrv. | | | Start | End | |
|---|---|---|---|---|---|---|
| Calc. Mass | Mass | ±da | ±pp, | Seq. | Seq | Sequence |
| 1022.5266 | 1022.5289 | 0.0023 | 2 | Seq.271 | Seq.279 | ATv′VYQGER |
| 1022.5266 | ″1022.528S1 | 0.0023 | ″- | 271 | 279 | ATVVYQGER |
| 1104.5472 | 1104.5469 | −O.OOO i | 0 | 328 | 3% | EHSSLAFWK |
| 1104.5472 | 1104.5469 | −0.0003 | 0 | i28 | 36 | EHSSLAFWK |
| 1150.6216 | 1150.61″?6 | −0.004 | −3 | 2′70 | 2′79 | KATVVYQGER |
| 1502.7784 | 1502.7891 | 0.0107 | 7 | 83 | 96 | VCPFAGILENGAVR |
| 1502.7784 | 1502.7891 | 0.0107 | 7 | 83 | 96 | VCPFAGILENGAVR |
| 1914.0042 | 1913.9966 | −0.0076 | −4 | 2L″ | 38 | TCPKPDDLPFSTVVPLK |
| 1914.0042 | ″1S113.9966 | −0.0076 | −4 | 22 | 38 | TCPKPDDLPFSTVVPLK |
| 2085.9104 | 2085.8286 | −0.0818 | −39 | 307 | 324 | CSYTEDAQCIDGTiEVPK |
| 2383.0911 | 2383.1409 | 0.0498 | 21 | 39 | 58 | TFYEPGEEITYSGKPGYV SR |
| 2383.0911 | 2383.1409 | 0.0498 | 2′1 | 39 | 58 | TFYEPGEEITYSCKPGYV |
| | | | | | | SR |
| 2385.9963 | 2386.1001 | 0.1038 | 44 | 230 | 250 | ATFGCHDGYSLDGPEEiE |
| | | | | | | CTK |
| 2731.3337 | 2731.426 | 0.0923 | 34 | 205 | 227 | GPFPSRPDNGFVNYPAK |
| | | | | | | PTLYYK |

| 195/H22 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| PI00910625 | Tax id = 9606 Gene Symbol = - | Pi | i1402.2 |
| | eDNA FLJ51265, | 8.19 | |
| | moderately s;milar to-Beta-2- - | | |
| | glycoprotein | | |

### Peptide Information

| Calc. Mass | Obsrv. | ±da | ±pp, | Start | End | Sequence |
|---|---|---|---|---|---|---|
| 1022.5266 | 1022.5208 | −·0.0058 | −6 | 200 | 208 | ATVVYQGER |
| 1022.5266 | 1022.5208 | −0 0058 | −6 | 200 | 208 | ATV\IYQGER |
| 1104.5472 | 1104.5475 | 0.0003 | 0 | 257 | 265 | EHSSLAFWK |
| 1104.5472 | 1104.5475 | 0.0003 | 0 | 257 | 265 | EHSSLAFWK |
| 1150.6216 | 1150.6241 | 0.0025 | 2 | 199 | 208 | KATVVYQGER |
| 1 ′\02.″1784 | 1502.8273 | 0.0489 | 33 | 83 | 96 | VCPFAGILENGAVR |
| 1502.7784 | 1502.8273 | 0.0489 | 33 | 83 | 96 | \ICPFAGILENGAVR |
| 1914.0042 | 1914075 | 0.0708 | 37 | 22 | 38 | TCPKPDDLPFSTV\IPLK |
| 1914.0042 | 1914.075 | 0.0708 | 37 | 22 | 38 | TCPKPDDLPFST\IVPLK |
| 2085.S1104 | 2085.9956 | 0.0852 | 4″1 | 236 | 253 | CSYTEDAQCIDGTiEVPK |
| 2383.0911 | 2383.2917 | 0.2006 | 84 | 39 | 58 | TFYEPGEEITYSCKPGY\1 |
| | | | | | | SR |
| 2383.0911 | 2383.2917 | 0.2006 | 84 | 39 | 58 | TFYEPGEEITYSGKPGYV |
| | | | | | | SR |

Ficoiin-3

| 196/H23 | Instr./Gel Origin |
|---|---|
| [1] Sample Project | Instrument Sample Name |
| 20120614 | |

| Accession | | | |
|---|---|---|---|
| No. | Protein Name | Protein | Protein MW |
| IPI00293925 | Tax_id 9606 Gene_Symboi, FCN3 | Pi | 33395.2 |
| | Isoform 1 of Ficoiin-3 | 6.2 | |

### Peptide Information

| | Obsrv. | | | Start | End | |
|---|---|---|---|---|---|---|
| Calc. Mass | Mass | ±da | ±ppm, | Seq. | Seq | Sequence |
| 941.5064 | SI41.4953 | −0.0111 | −12 | 286 | 293 | GVGHPYRR |
| 1024.4846 | 1024.4824 | −0.0022 | −2 | 27? | 28 ) | YGIDWASGR |
| 1024.4846 | 1024.4824 | 0.0022 | −2 | 277 | 285 | YGIDWASGR |
| 1046.5265 | 1046.5337 | 00072 | 7 | 267 | 276 | Y!-\VSE!-\1-\AHK |
| 1070.4902 | 1070.486 | −0.0042 | −4 | ″137 | ″145 | QDGSVDFFR |
| 1070.4902 | 1070.486 | −0.00-12 | −−1 | 137 | 145 | QDGSVDFFR |
| 1113.5-176 | 1111 1.54 i6 | −0.004 | −4 | 191 | 199 | TFAHYATFR |
| 1113.5476 | 1113.5436 | −0.004 | −4 | 1B1 | 1B9 | TFAHYATFR |
| 1166.6165 | 1166.5963 | −0.0 02 | −17 | (′″) | gr [symbol] | GEPGDPVNLLR |
| 1226.5913 | 1226.5856 | 0.0057 | ··5 | 136 | 145 | RQDGSVDFFR |
| 1226.5913 | 1226.5856 | −0 0057 | −5 | 136 | 145 | RQDGSVDFFR |
| 1555.8-79 | 1555.8181 | −0.0298 | −19 | 200 | 213 | LLGEVDHYQLALGK |
| 1555.8479 | 1555.8181 | −0.0298 | −·:S1 | 200 | 213 | LLGEVDHYQLALGK |
| 15B5.821 | 1595.7993 | −0.0217 | −14 | 71 | 85 | MGPKGEPGDPVNLLR |

[symbol]

indicates data missing or illegible when filed

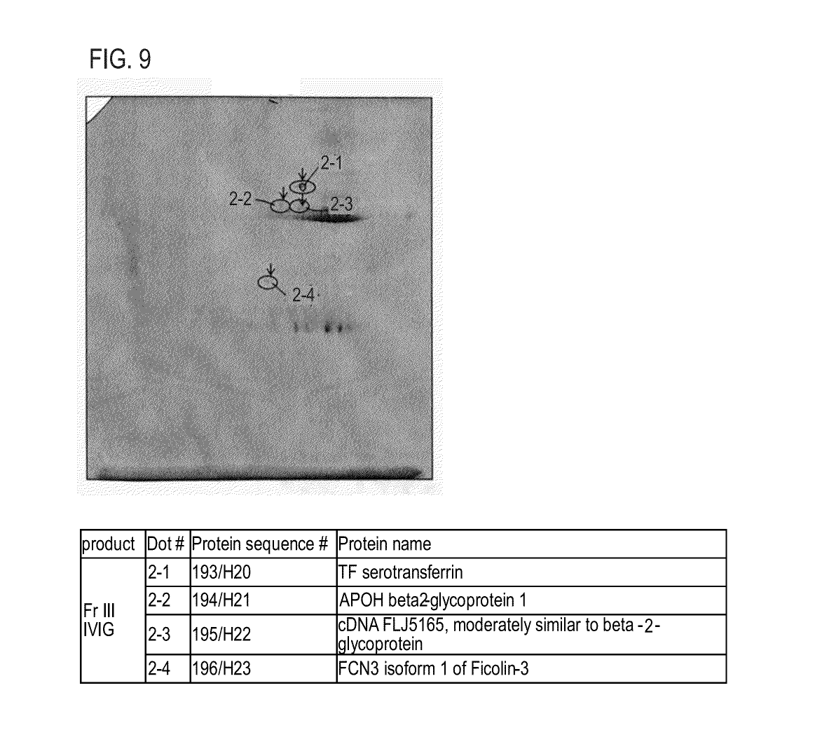

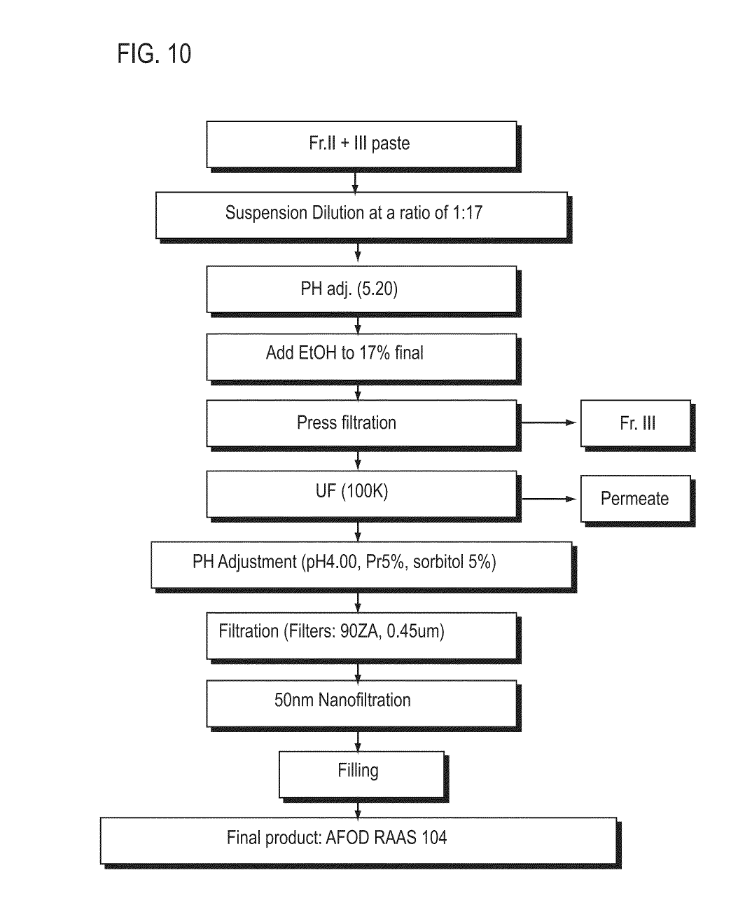

FIG. 9
4) AFOD RAAS 104 g. contains HEPATITIS B IMMUNEGLOBULIN with high titer of Hepatitis B antibody, in addition it contains TF protein sequence#197/H24 TF serotransferrin and may contain newly discovered proteins KH33, KH34, KH35, KH36 and KH37. The Hepatitis B antibody has been known to prevent the infection of the Hepatitis B virus in the health care worker, who
may accidentally stick the contaminated needle from the Hepatitis B patient. In the product HepaRAAS® Hepatitis B lrmnunoglobulin used to prevent the reoccurrence of the Hepatitis B virus in the liver transplant patient. In addition with the combination of one or many of these newly discovered proteins KH33, KH34, KH35, KH36 and KH37 the AFOD RAAS 104 can immunediately stop the replication of the Hepatitis B virus in mice models and completely transform the Hepatitis B virus cell, which produces the sick protein that causes the Hepatitis B, into a good protein to eliminate the Hepatitis B virus in the mice within 4 days of 1dose a day administration.
FIG. 10

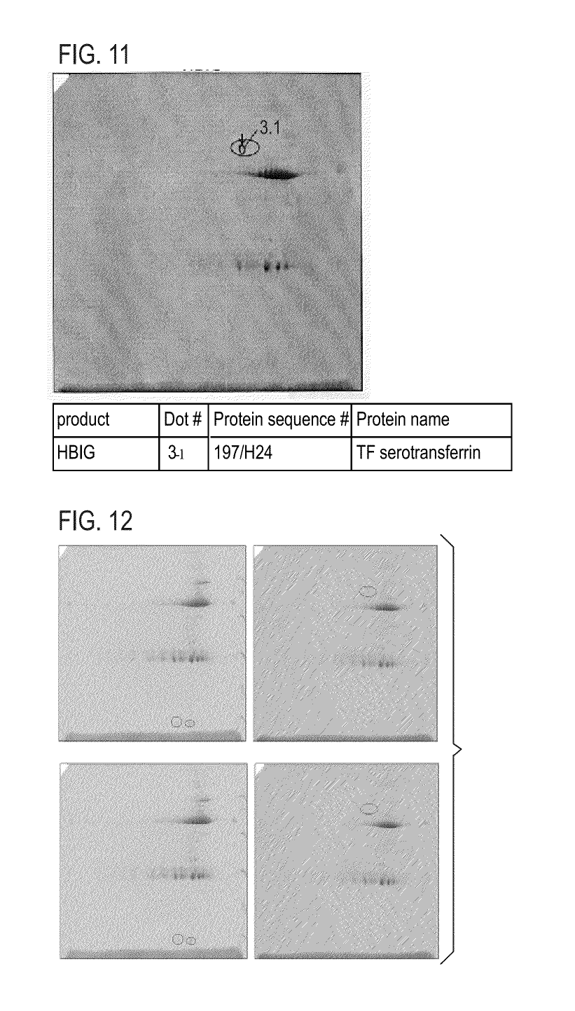

Beside the main component of the Immunoglobulin in each of the three processes namely AFOD RAAS 102, AFOD RAAS 103 and AFOD RAAS 104 each product also has an additional proteins that differ from one another.
FIG. 11, 12.

Finally in the AFOD RAAS 102. we found the following proteins: IGHV4-3I.; IGHG:I. 44 kDa protein, IGHV4-31; IGHC1 32.kDa protein, IGHV4-31; 1GHG1. Putative uncharacterized protein DKFZp686G11190.

In AFOD RAAS 103 we found the following proteins: TF serotransferrin, APOH beta2-glycoprotein 1, cDNA FU5165, moderately similar to beta-2-glycoprotein, FCN3 isoform 1 of Ficolin-3.

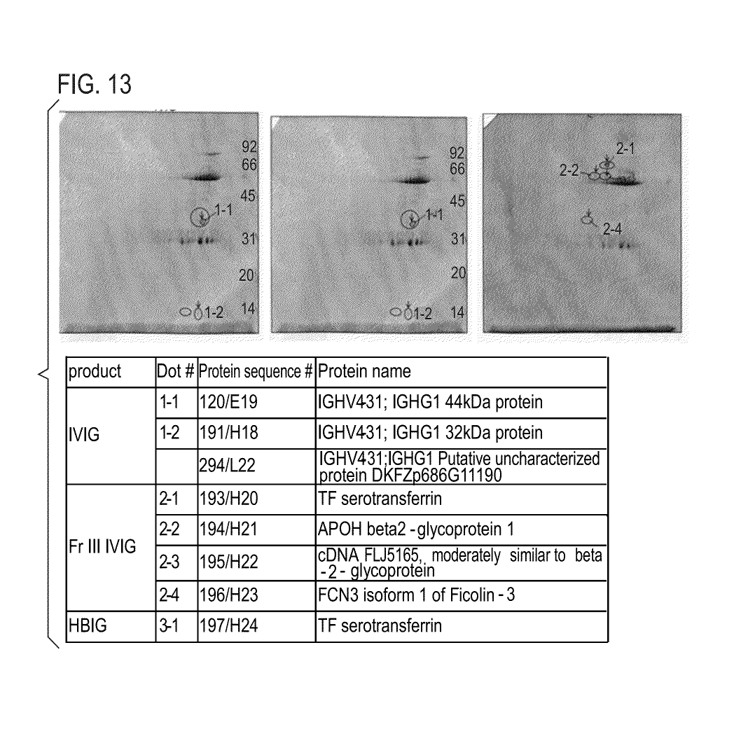

In AFOD RAAS 104 we found the following protein: TF serotransferrin.
FIG. 13
5) AFOD RAAS 1050 is formulated due to the scarcity of Hepatitis B antibody while the treatment for the Hepatitis B virus demands rnore of the product. AFOD RAAS 105 is the combination of 80% AFOD RAAS 102 and 20% AFOD RAAS 104. Both when combined will give more products not only for Hepatitis B but also for the treatment of cancers, especially liver cancers or liver diseases, and other neurological diseases. Both of the products must have a concentration by ultra filtration up to 30%. This combination will provide the product of AFOD RAAS 105 with five newly discovered proteins KH33, KH34, KH35, KH36, KH37 and KH51 which may contain newly discovered GOOD HEALTHY CELLS which synthesize the new good proteins.

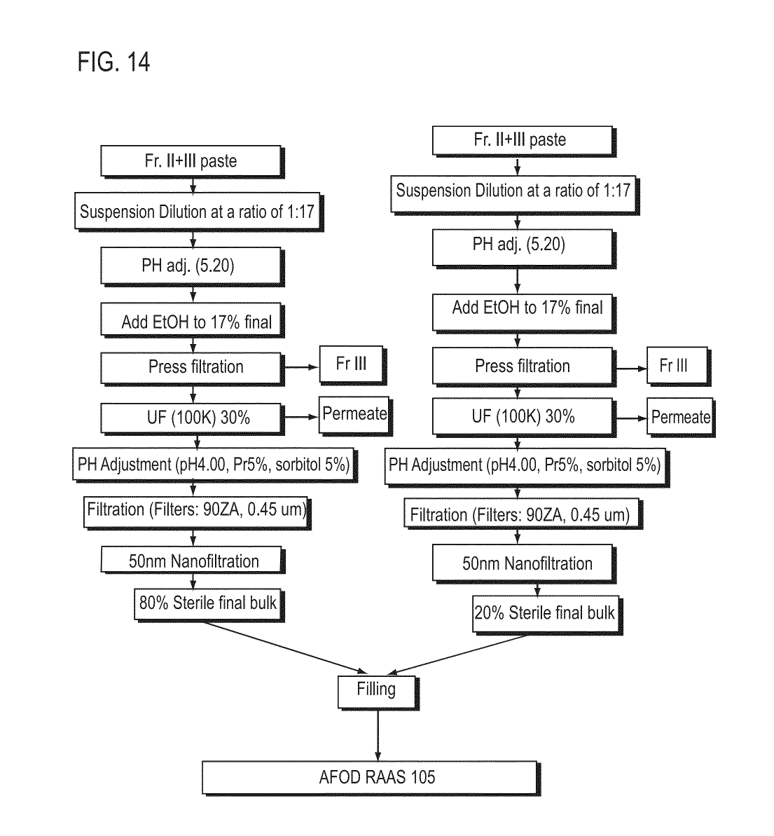

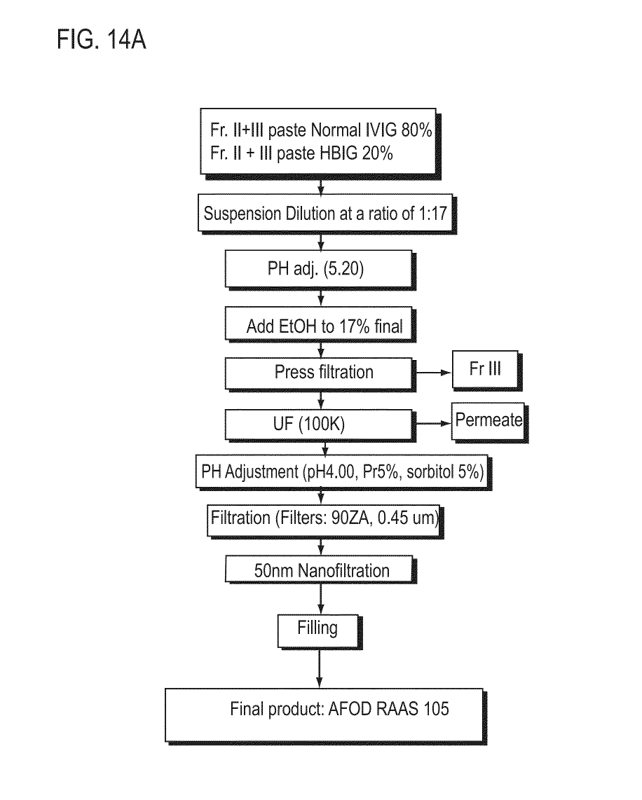

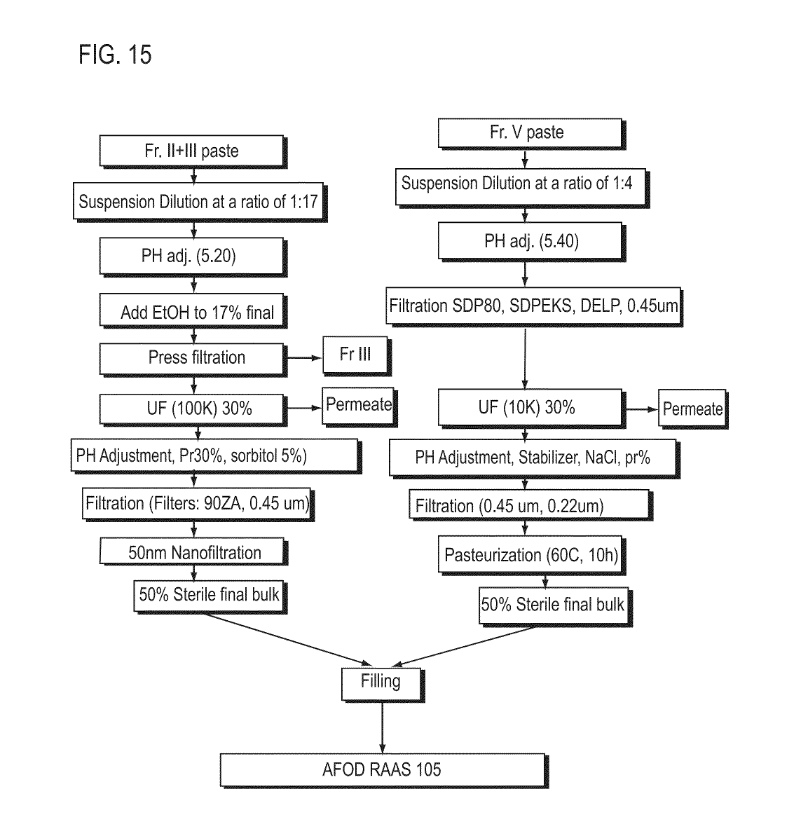

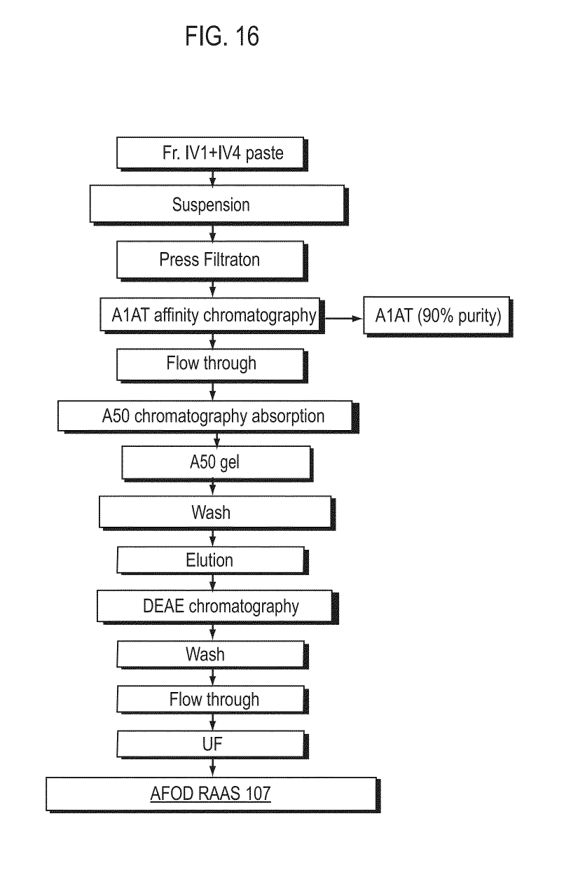

There are two methods of manufacturing AFOD RAAS 105®:
Method 1: Follow manufacturing protocol to separately manufacture normal Immunoglobulin and Hepatitis B antibody until the step of non-sterile final bulk for both products come, take 80% of the normal Immunoglobulin non-sterile final bulk and mix with 20% of Hepatitis B antibody non-sterile final bulk. Perform sterile filtration for filling for AFOD RAAS 105®
Method 2: Take 80% of normal immunoglobulin fraction II+III and 20% of Hepatitis B antibody fraction II+III then dissolve together in the process tank for production of the normal Immunoglobulin until the filling for AFOD RAAS 105@.
FIG. 14, 14a
6) AFOD RAAS 106@ is the combination of AFOD RAA5 101 with seven discovered proteins plus newly discovered KH51 and i\FOD RAA5 102 with a total of 8 proteins, including newly discovered protein KH33, KH34, Kh35, KH36 and KH37 has become a very potent combination of all this newly discovered proteins in Human Albumin and Immunoglobulin which enables this combination to work effectively against all cancers, bacteria, specially staphylococcus aureus which is resistant to the current antibiotics.
FIG. 15
7) AFOD RAA5 107® contains mainly the protein 1 CP 98 kDa and possibly a lot more new proteins that are under investigation. Protein 1 CP 98 kDa contain Nup98 and Nup96 play a role in the bidirectional transport. The 98 KD nucleoporin is generated through a biogenesis pathway that involves synthesis and proteolytic cleavage of a 186 KD precursor protein. The human gene has been shown to fuse to several genes follmNing chromosome translocatons in acute myelogenous leukemia (AML) and T-cell acute lymphocytic leukemia (T-ALL). This gene is of the several genes located in the imprinted gene domain of 11p15.5, an important tumor-suppressor gene region. Alterations in this region have been associated with the Beckwith-VJiedemann syndrome, Wilms tumor, rhabdomyosarcoma, adrenocortical carcinoma, and lung, ovarian and breast cancer. This protein along with a lot more new proteins under investigation have proven efficacy against the breast cancer and other cancers as described above.
FIG. 16

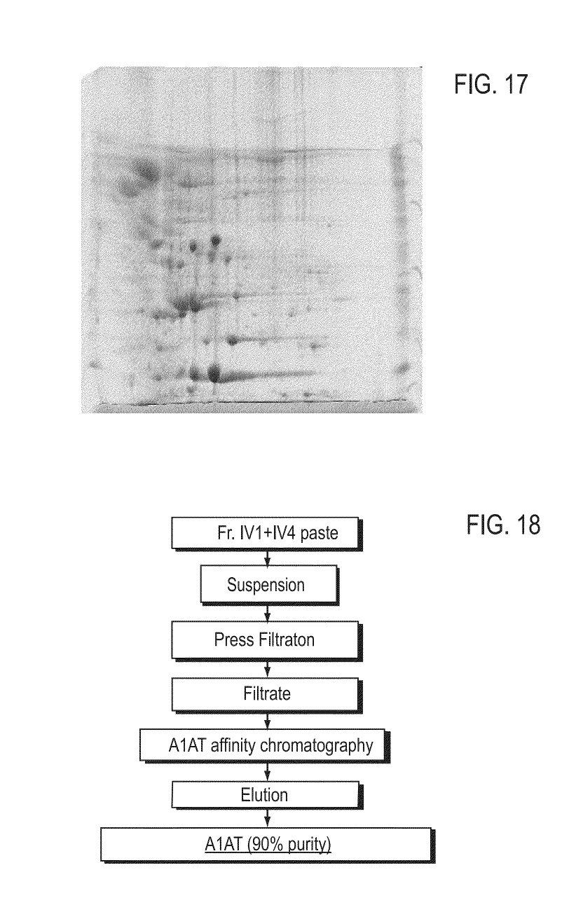

20 electropherosis of plasma derived protein CP98 kOa shows numerous newly discovered KH proteins, rnore new proteins under investigation or already discovered proteins.
FIG. 17
8) AFOO RAAS 108 g. contains mainly Alpha 1 antitrypsin protein which has been used in the treatment of the Alpha 1 Antitrypsin deficiency and also for the treatment of emphysema. Currently it is also being used under trial for Diabetic patients. With the complex of the new found proteins like KH21, KH22, KH23, KH24, KH25, KH26, KH27, KH48, KH49 and KHSO the efficacy of AFOD RAAS 108 will be more effective in the treatment of cancers, diabetic and many other diseases or deficiencies.
FIG. 18

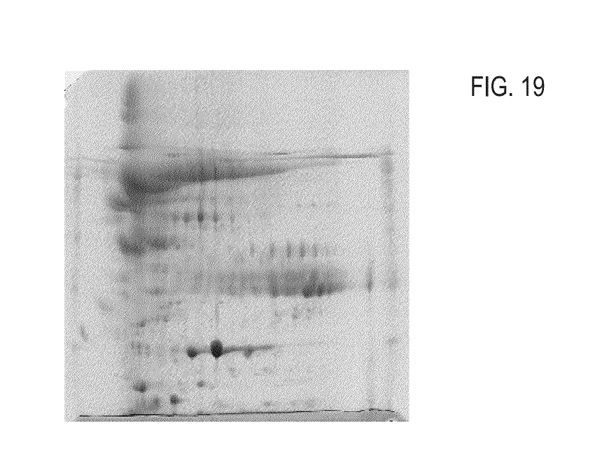

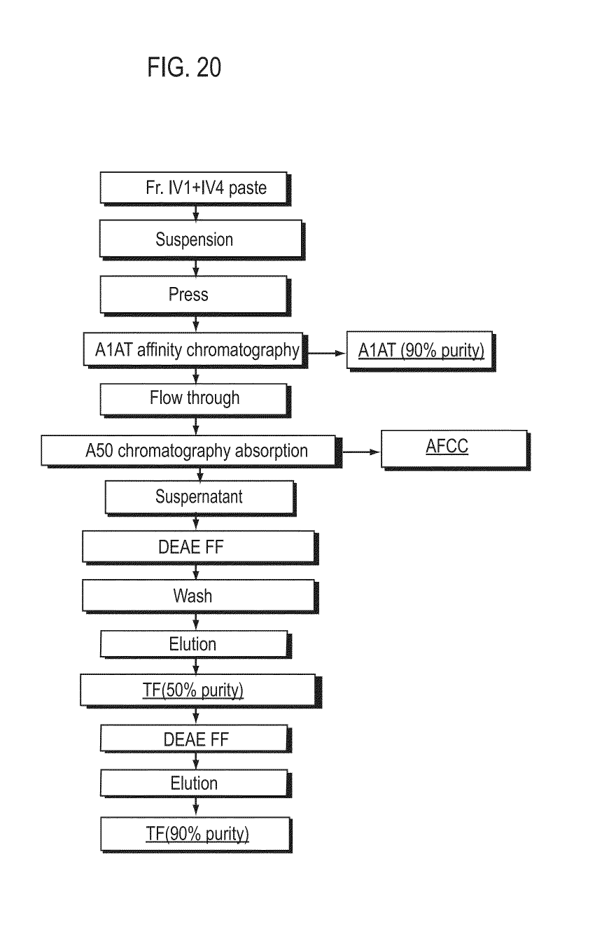

20 electropherosis of plasma derived protein A1AT shows numerous ne\Niy discovered KH proteins, more new proteins under investigation or already discovered proteins.
FIG. 19
9) AFOO RAAS 109® contains mainly Transferrin protein which has not been used for any clinical application however used for diagnostic purpose. With the complex of the new found proteins like KH2J, KH2.2, KH2.3, KH2.4, KH25, KH26, KH27, KH48, KH49 and KHSO the efficacy of AFOD RAAS 109 will be more effective in the treatment of cancers, diabetic, cardiovascular and many other diseases or deficiencies. The inventor believes that with enough dosage of AFOD RAAS 109 it will provide enough good healthy cells to synthesize the protein which produces insulin in the patient to certain point that the patient will no longer need to inject the insulin anymore.
FIG. 20

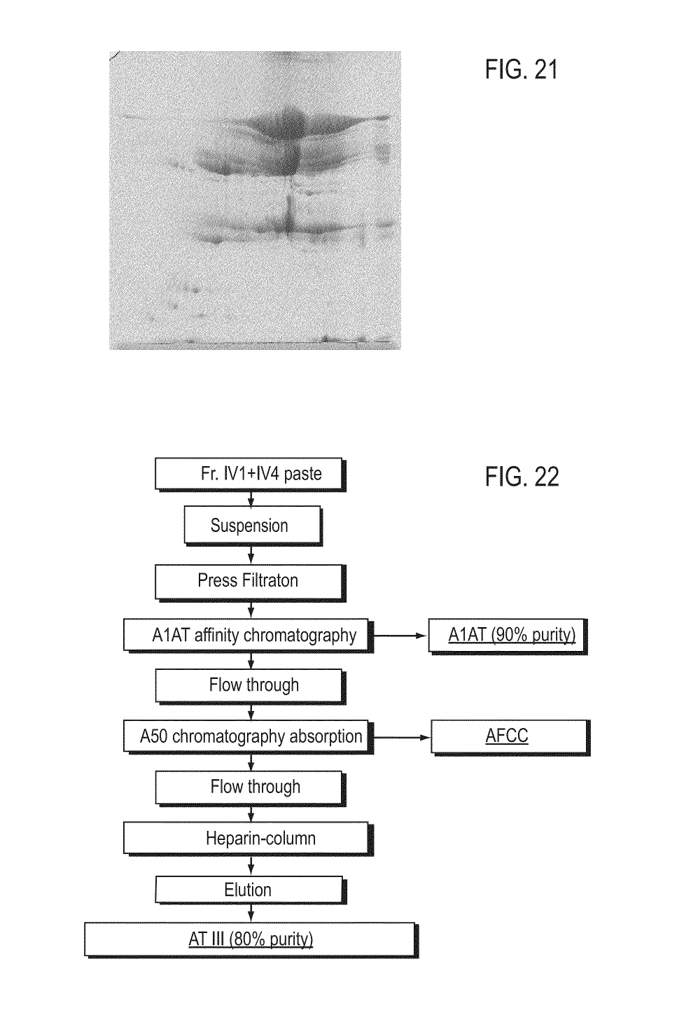

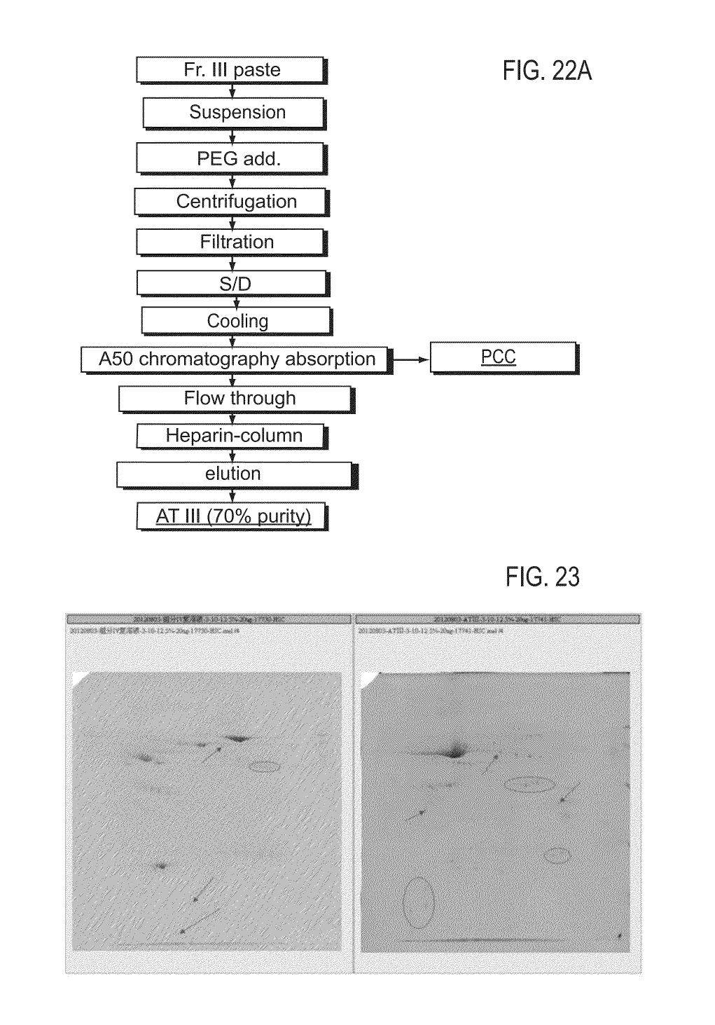

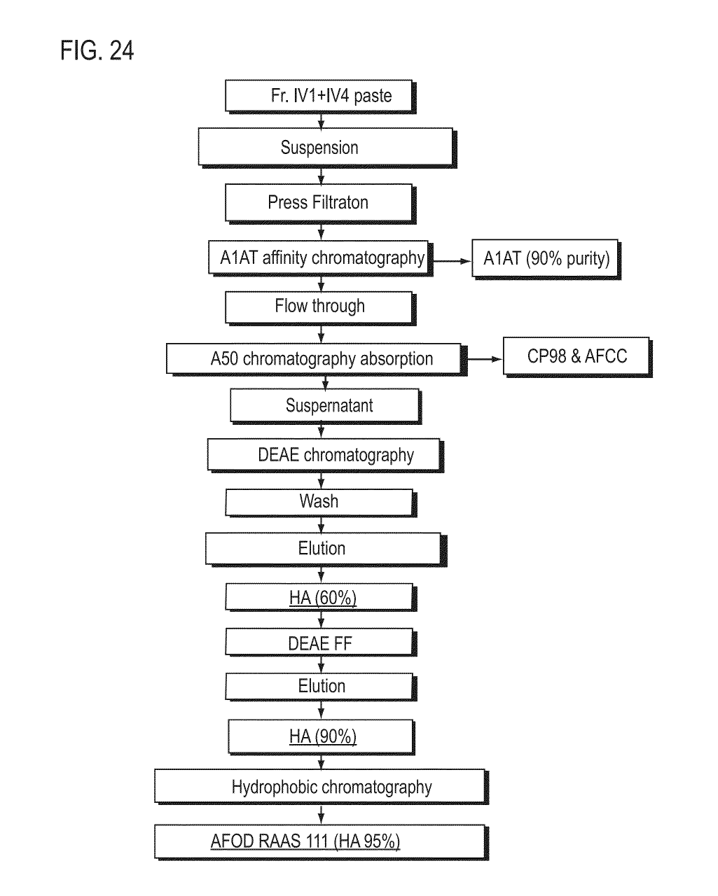

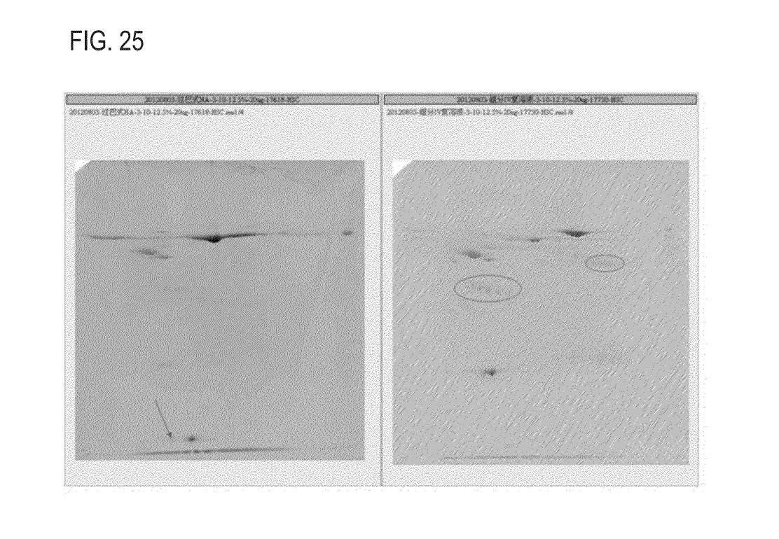

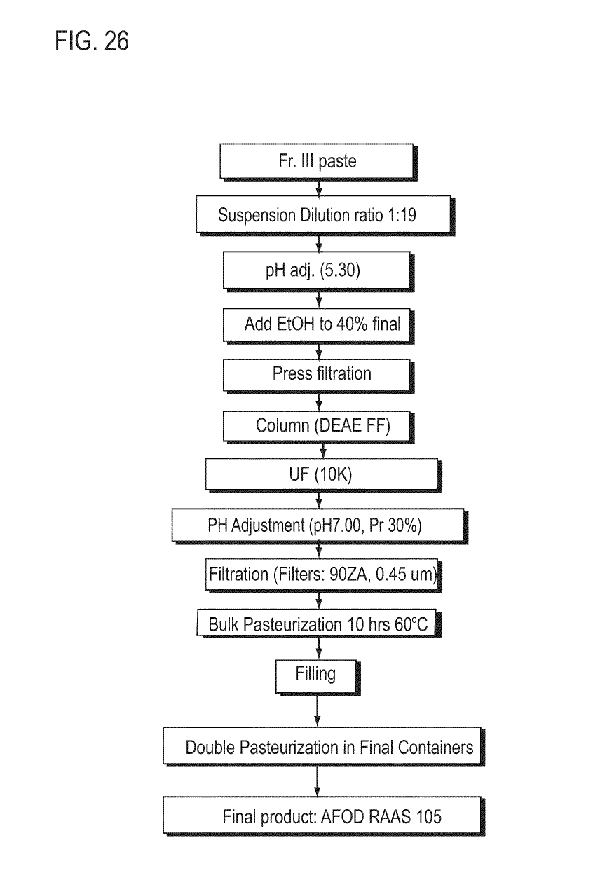

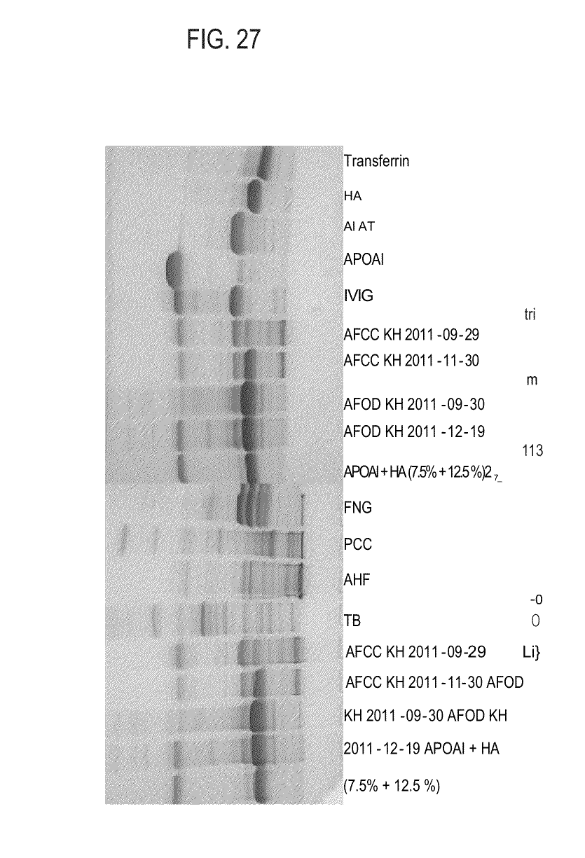

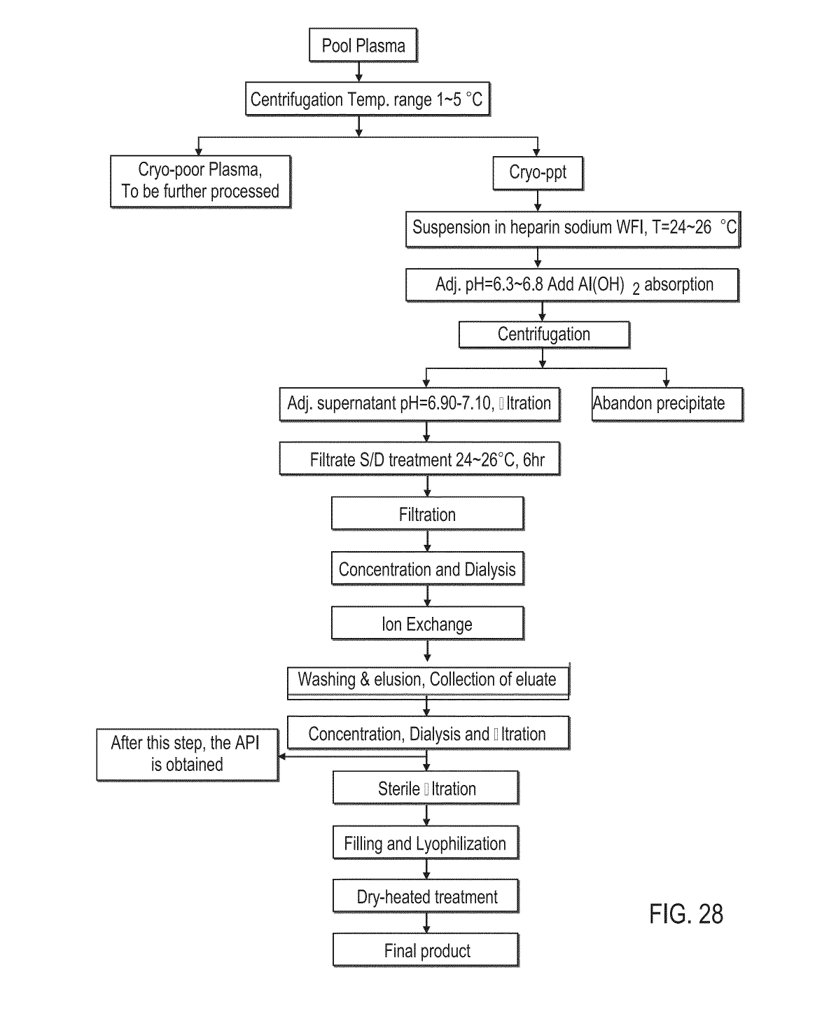

20 electropherosis of plasma derived protein Transferrin shows numerous newly discovered KH proteins, rnore new proteins under investigation or already discovered proteins.
FIG. 21
10) AFOD RAAS 110 g. contains mainly AntiThrombin III protein commercially available but with no significant efficacy has been proven. With the complex of the new found proteins like KH21, KH22, KH23, KH24, KH25, KH26, KH27, KH48, KH49 and KHSO the efficacy of AFOD RAAS 110 will be more effective in the treatment of thrombosis, stroke patients and cardia vascular diseases in cornbination with AFOD RAAS 1 (APOAI)
FIG. 22, 22a
11) AFOD RAAS 111 mainly beside Human Albumin, it also contains ne\Niy discovered proteins like KH21, KH22, KH23, KH24, KH25, KH26, KH27, KH48, KH49 and KHSO. The efficacy of AFOD RAAS 111\Nill be more effective. The inventor believes that with enough dosage of AFOD RAAS 111 it will provide enough good healthy cells to synthesize the protein which produces insulin in the patient to certain point that the patient will no longer need to inject the insulin anymore.
FIG. 24
12) AFOD RAAS 112® contains a small amount of the Human Albumin protein, however this Human Albumin together with the ne\Niy discovered protein KH3, KH4, KH5, KH6, KH7, KH8, KH9, KI-UO, KH19, KH20, KH38 KH39, KH40, KH41, KH42 and KH43 have been known through our animal studies, to prevent the death caused by H1N1 virus in the mice. It also has shown in vitro studies to eliminate the HIV virus. rv1ore proteins from AFOD RAAS 112 are under investigation. The inventor believes that with enough dosage of AFOD RAAS 112 it will provide enough good healthy cells to synthesize the protein which produces insulin in the patient to certain point that the patient will no longer need to inject the insulin anymore.
FIG. 26
13) AFCC Ri\AS 101® contains mainly protein Human Coagulation Factor VIII mainly for use in the stop of the bleeding in patients with Hemophilia A. However AFCC RAAS 101 not only contains Coagulant Factor VIII but it also contains newly discovered proteins KH1, KH2, KH2.8 and KH29. With the addition of these newly found proteins which has shown in in-vitro studies to reduce the tumor growth of solid cancers. The inventor believes that with enough dosage of AFCC RAAS 101 it will provide enough good healthy cells to synthesize the Factor VIII protein in the patient to certain point that the patient will no longer need to inject coagulant factor VIII anymore.
FIG. 28

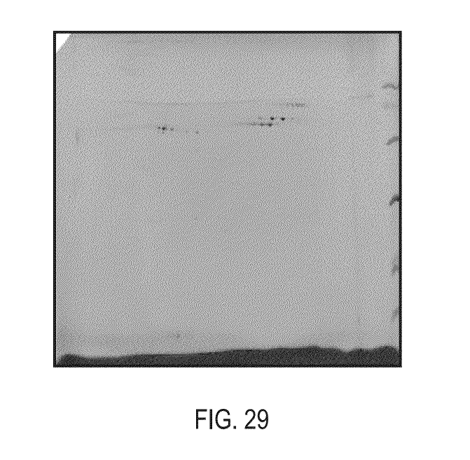

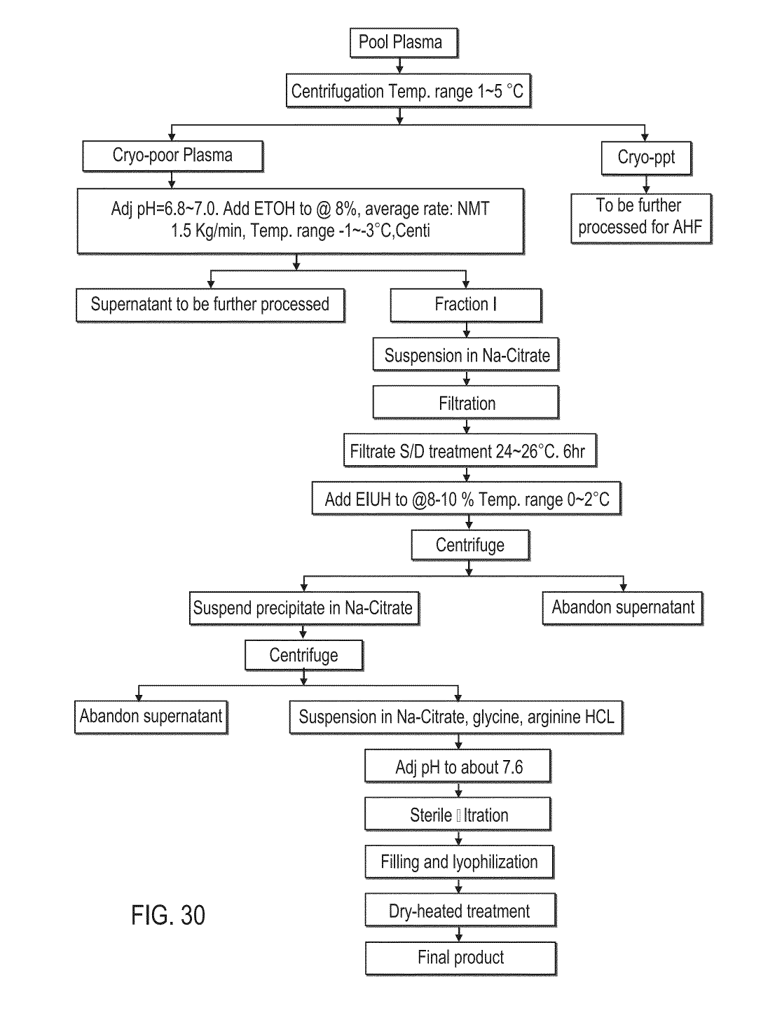

20 electropherosis of plasma derived protein Human Coagulation Factor VIII shows numerous newly discovered KH proteins, more new proteins under investigation or already discovered proteins.
FIG. 29
14) AFCC RAAS 102® contains mainly Human Fibrinogen protein which is used mainly for the treatment of liver diseases and trauma. With the addition with our five newly discovered proteins KH1, KH2, KH30, KH31 and KH32 has shown in in-vitro studies to reduce the growth of solid tumors.
FIG. 30

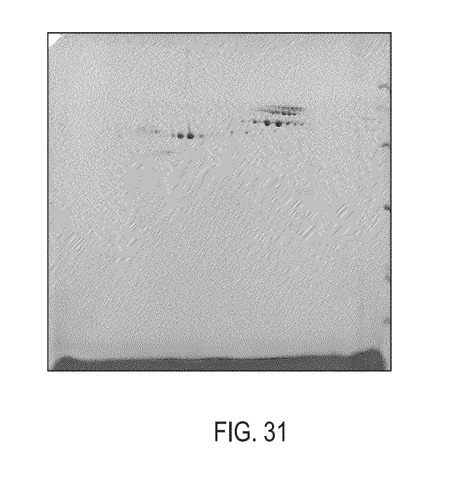

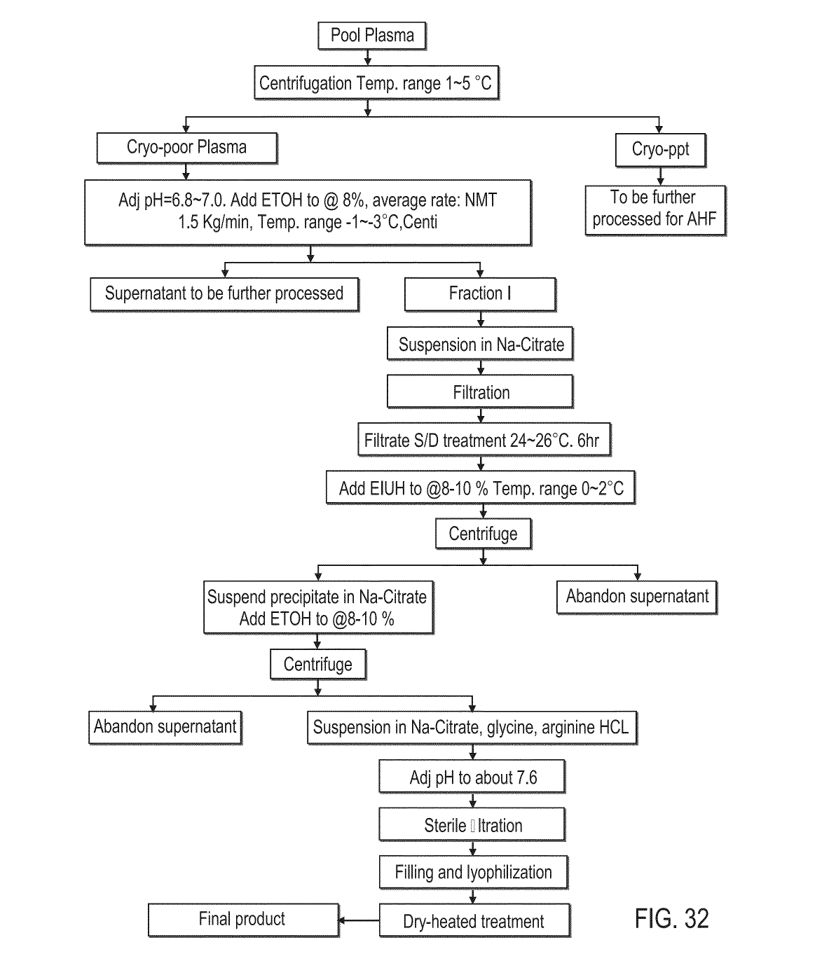

20 electropherosis of plasma derived protein Human Fibrinogen shows numerous newly discovered KH proteins, more new proteins under investigation or already discovered proteins.
FIG. 31
15) AFCC RAAS 103® contains mainly High Concentrate Human Fibrinogen protein which is used in combination with Thrombin to create a Fibrin Glue membrane (as in FibringluRAAS®) in order to stop the bleeding during the surgical operations. With the addition of newly discovered proteins KHI, KH2, KH30, KH31, KH32 and specially KH52 AFCC RAAS 103® has been proven to be very effective in stopping the tumor growth in liver cancer, colon cancer and lung cancers in animal studies which are used for the submission of the application for licensing.
FIG. 32.

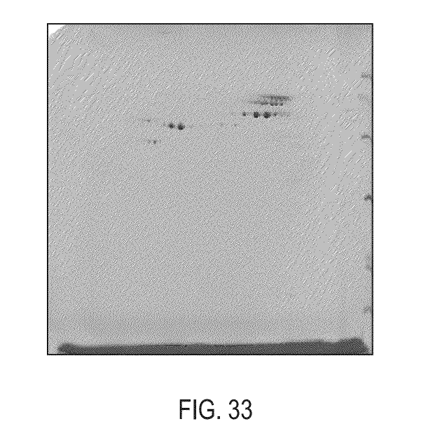

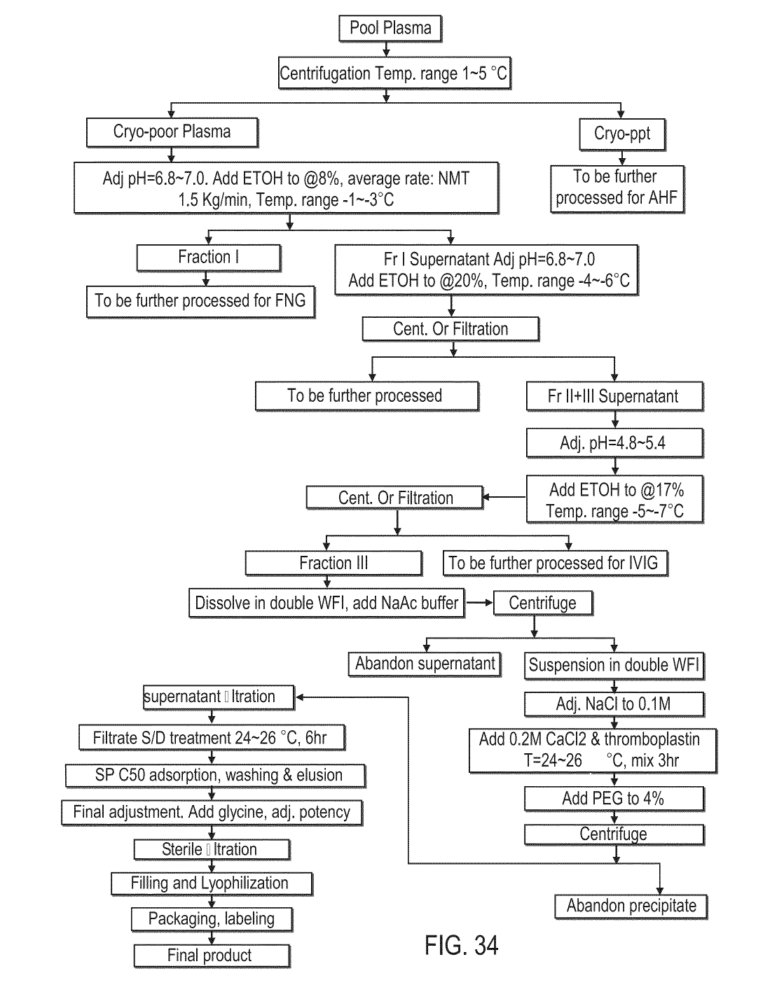

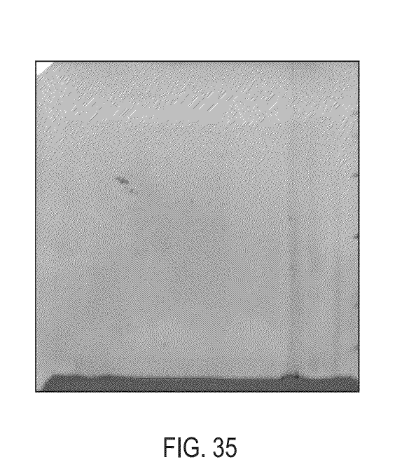

20 electropherosis of plasma derived protein High Concentrate Human Fibrinogen shows numerous newly discovered KH proteins, more new proteins under investigation or already discovered proteins.
FIG. 33
16) AFCC RAAS 104® contains mainly Human Thrombin protein which is used in combination with High concentrate Human Fibrinogen protein to create a Fibrin Glue membrane (as in FibringluRAAS®) in order to stop the bleeding during the surgical operations. With the addition of newly discovered proteins KH44, KH45, KH46 and KH47 in our AFCC RAAS 104® has been proven to be very effective in stopping the tumor growth in liver cancer colon cancer and lung cancers in animal studies which are used for the submission of the application for licensing.
FIG. 34

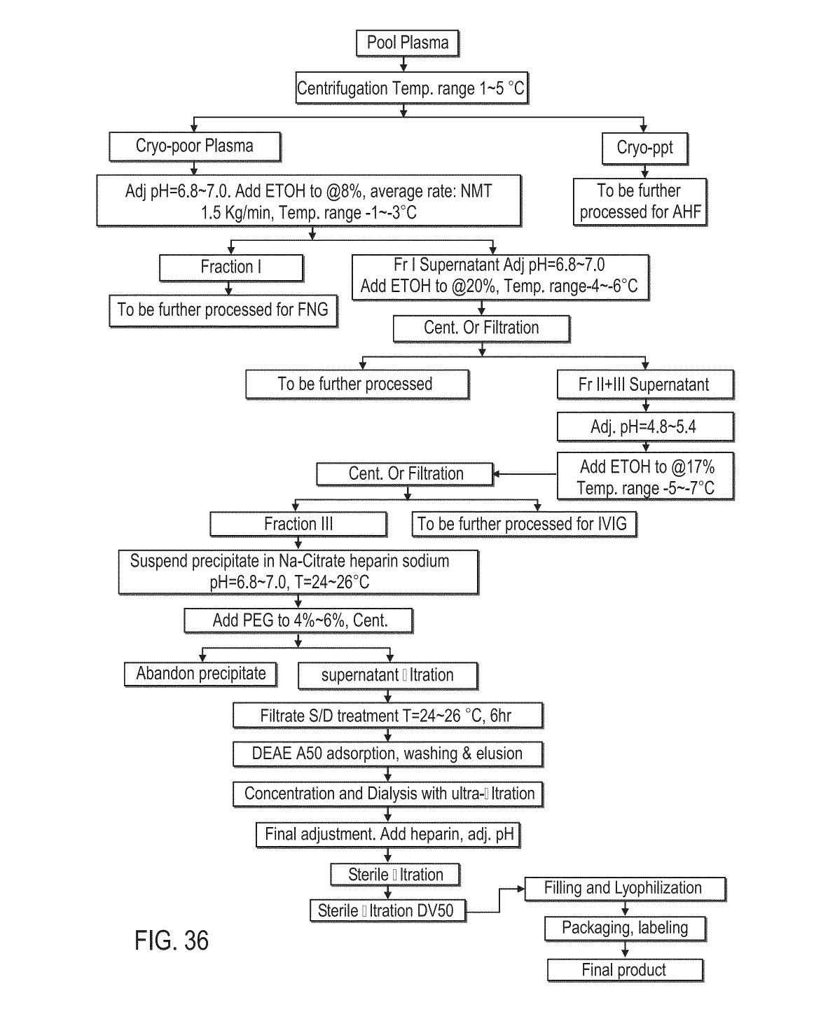

2D electropherosis of plasma derived protein Human Thrombin shows numerous newly discovered KH proteins, rnore new proteins under investigation or already discovered proteins.
FIG. 3.5
17) AFCC RAAS 105® contains mainly Human Prothrombin Complex protein \Nhich include Factor II, Factor VII, Factor IX and Factor X. In the world it is mainly used for the treatment of Hemophilia Bas a Factor IX or it can be used for Hemophilia A treatment with inhibitor. In China Prothrombin Complex is used mainly in the treatment of the liver disease. AFCC RAAS 105@ contains eight newly discovered proteins: Kf-111, Kf-112, KHB, Kf-114, KH15, KH16, KH17 and KH18. The inventor has found that the HIV virus cannot be killed in PCC by solvent detergent method using TNBP and TWIN80, that led to the in-vitro testing of the original AFCC RAAS 105 (formerly AFCC RAAS 1) and has found that the HIV virus has been eliminated in enzyme also the viral load has become negative in the PCR testing. Confirmation of the HIV replication and the animal study is being done with the help of the National AIDS research center at Tsing Hua University in Beijing. This formulation can only be used for the Hemophilia A or B with HIV, but for non hemophilia patients the dosage and prescription must be highly controlled from the physician, because if too much product is given then the patients could be fatal.
FIG. 36

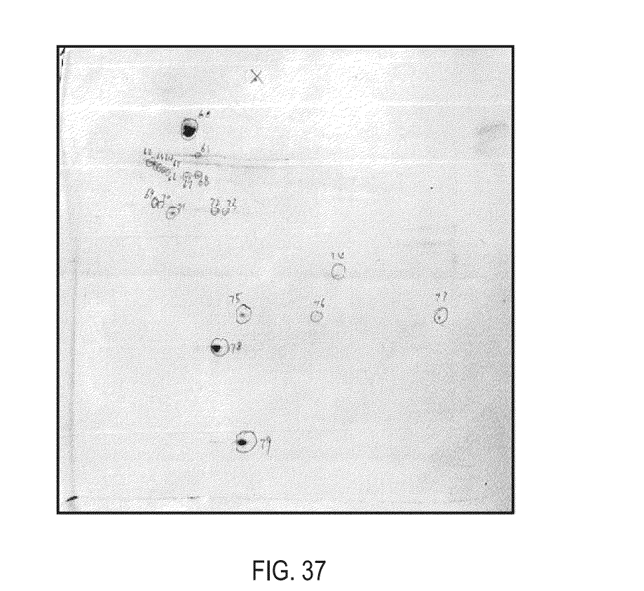

2D electropherosis of plasma derived protein Human Prothrombin Complex shows numerous newly discovered KH proteins more new proteins under investigation or already discovered proteins.
FIG. 37
18) AFCC Ri\AS 106® mainly contains all newly discovered proteins KH2J, KH2.2, KH2.3, KH2.4, KH25, KH26, KH27, KH48, KH49 and KH.SO in fraction IV. The color of which is blue from pile, so we assume that it is PCC. But when we tested for the content of Factor IX, we were not able to find any factor IX. The Inventor see the problem associated with AFCC RAAS 10.5® as they are from fraction III and this is the most complicated complex of proteins which include Prothrombin and Thrombin therefore the inventor wants to have the same product of AFCC RAi\S 1.05® which can kill the HIV virus or others but will not cause harm to the NON hemophilia patients, therefore this formulation was created.

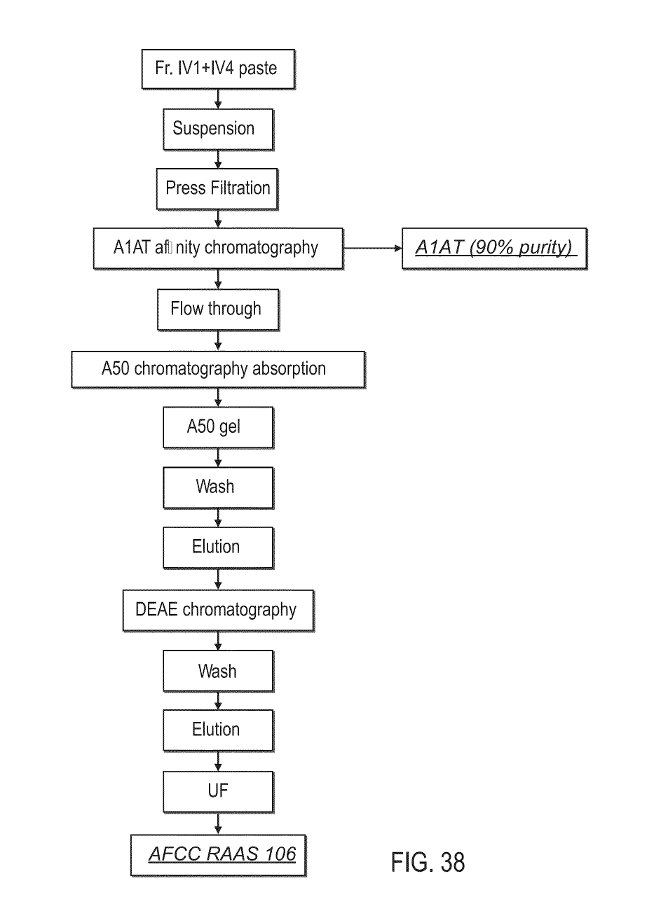

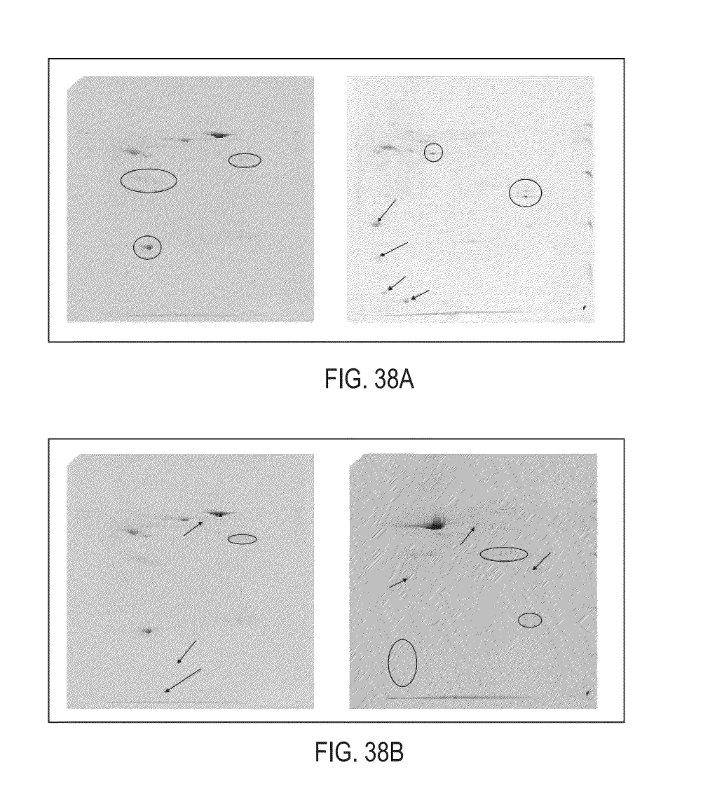

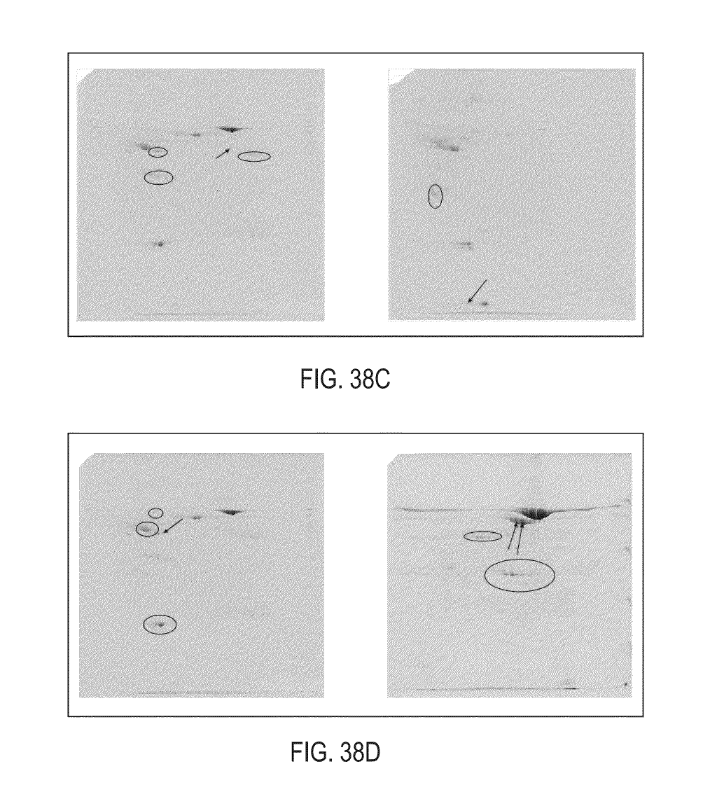

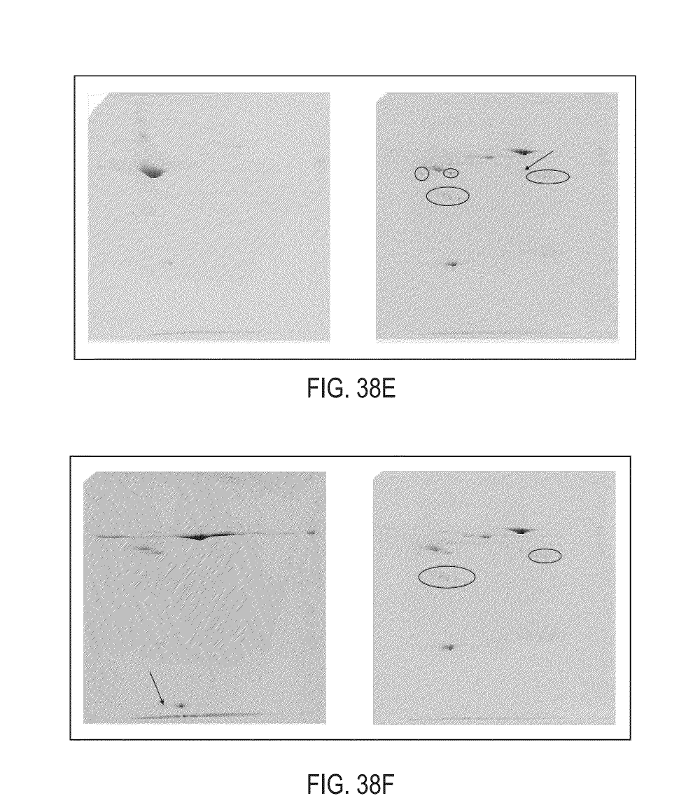

2D electrophoresis of plasma derived proteins in i\FCC from fraction IV in the red circles and red arrows shows numerous newly discovered KH proteins, more new proteins under investigation or already discovered proteins.
FIG. 38a

20 electrophoresis of plasma derived protein Anti Thrornbin III from fraction IV in the red circles and red arrows shows numerous newly discovered KH proteins, more new proteins under investigation or already discovered proteins.
FIG. 38b

2D electrophoresis of plasma derived protein CP98 from fraction IV in the red circles and red arrows shmNs numerous newly discovered KH proteins, more new proteins under investigation or already discovered proteins.
FIG. 38c

2D electrophoresis of plasma derived protein Transferrin from fraction IV in the red circles and red arrows shows numerous newly discovered KH proteins, more new proteins under investigation or already discovered proteins.
FIG. 38d

20 electrophoresis of plasma derived protein Alpha 1 Antitrypsin from fraction IV in the red circles and red arrows shows numerous newly discovered KH proteins, more new proteins under investigation or already discovered proteins.
FIG. 38e

2D electrophoresis of plasma derived containing only pure protein Alpha 1 Antitrypsin from fraction IV.
FIG. 38f

### Animal Blood Plasma

In the animal study we have found the prevention of influenza H1N1 which can also affect the birds, therefore the inventor has discovered using the same process of AFOO RAAS 101 through AFOO RAAS also utilized in the blood plasma of healthy animals to fractionate and further process into the product like Human Albumin and Immunoglobulin, and others for the prevention of the infection of the virus like HINI, SARS, H5N1, foot and mouth disease, mad cow disease and other epidemic unknown diseases.

FDA has recently forbidden the use of antibiotic in the cow as the antibiotic are resistant and It could get to the population.

In our study of the H1N1 for the prevention of the H1N1virus after one week of injection, the mice has survived as the product has injected the good healthy cells that send the signal to the DNA to transform the RNA of these infected mice to produce a good protein against the H1N1 virus. The long term study of how long this protection will last is still ongoing, so far the study has been going for 6 weeks. H1N1 is not as so important as the foot, hand and mouth disease that affects over 1 million people in China right now.

In addition to that we can test for mad cow disease but so far we have neither vaccine, nor product to take care of mad cow disease which has caused England not to allow their population to donate plasma and to import plasma from the United States of America.

In the USA we randomly check the cows and recently it was discovered some cases of mad cow disease. In Vietnam there are cases of Pigs with blue ear disease and in China H5N1 influenza has been found.

In brief there are still a lot of animals that are in as much danger as the human being for the virus infections and at any moment there could be an outbreak, if the animals are not vaccinated or treated with these products.

These products are not only for the prevention but to cure the diseases and to stop the disease from spreading, therefore meat eaters can feel safe about consuming any type of meat, since there is no use of hormones, antibiotic or chemical drugs in their bodies that can affect the consumer health.

AHC: RAAS 1 through AHC: RAAS 10 are under development to cure or prevent the any disease or outbreak in cows, pigs, chicken, lamb, goat sheep.

This product can also prevent the death of animals such as Panda. When they are sick and there is no product to protect and treat them. Also the strongest and fierce animal such as the Tiger could be saved as in the incident in October 2004 in Thailand, the inventor has found that ninety tigers from That Zoo had died after eating the carcass of the bird flu chicken.

The investigation is undergoing for different kind of animals and of course we will discover more cells and proteins, like the case in human that we are doing.

With the good healthy cells of any animal to send the signal to the DNA to transform the RNA in order to synthesize the good healthy proteins to fight the disease and infections in any animal.

### Recombinant DNA Proteins

Due to the shortage of plasma worldwide for the production of plasma derived products we have come up with also recombinant DNA proteins using the existing sequences of those existing proteins and specially the inventor has discovered 52 newly found proteins with their sequences and he has come up with different process following the process of making recombinant factor VIII. The plasmid construction for both mammalian yeast has been constructed, following the sequence of our newly found 52 proteins KH1, KH2, KH3, KH4 KH5, KH6, KH7, KH8, KH9, KH10 KH11 KH12, KH13, Kf-114, KH15, KH1KH17, KH1KH1KH2KH2L KH2KH23, KH2KH25, KH26, KH27, KH28, KH2 KH30, KH31, KH32, KH33, KH34, KH35 KH36, KH37, Kf-138, Kf-139, Kf-140, Kf-141, Kf-142, KH43, KH44, KH45, KH46, Kf-147, Kf-148, Kf-149, KH50, KH51 and Kf-152.

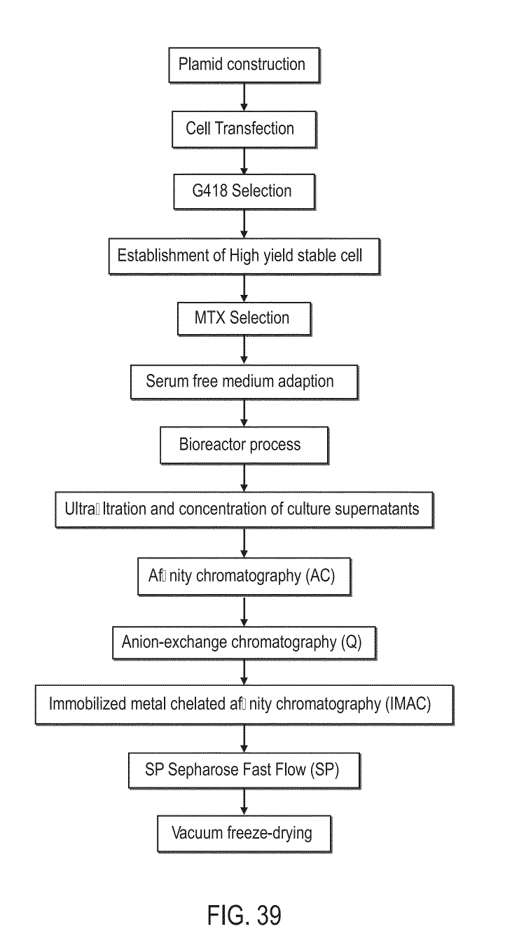

In addition to this new found proteins we have created a recombinant factor VIII which contain this new sequences. This recombinant factor VIII, factor VII or Von Willebrand can cure the Hemophilia patient with Hepatitis B, Hepatitis C, HIV and eventually build enough coagulant for the Hemophilia A or Hemophilia B.
FIG. 39

### Monoclonal Antibodies

In certain products like Hepatitis B antibody AFOD RAAS 104® with the new found proteins KH made from the high titer Hepatitis antibody from the human healthy donor are very short in supply. Monoclonal Antibodies can be created for such a major product, as they can cure Hepatitis B virus and liver cancer or any disease that is associated with the liver. In addition to this Hepatitis B monoclonal antibody the plasmid construction of the following sequences of our newly found 52 proteins KH1, KH2, KH3, KH4, KHS, KH6, KH7, KH8, KH9, KI.110, KH11, KH12, KH13 KH14 KH15, KH 1 KHIKHIKH19, KH2KH21, KH22, KH23, KH2KH25, KH2KH2 KH28, KH2KH3 KH31, KH32, KH33, KH34, KH35, KH36, KH37, KH38, KH39, KH40, KH41, KH42, KH43, KH44, KH45, KH46, KH47, KH48, KH49, KH50, KH51 and KH52 to make the rnonoclonal antibodies with good proteins synthesized by the good healthy cells.

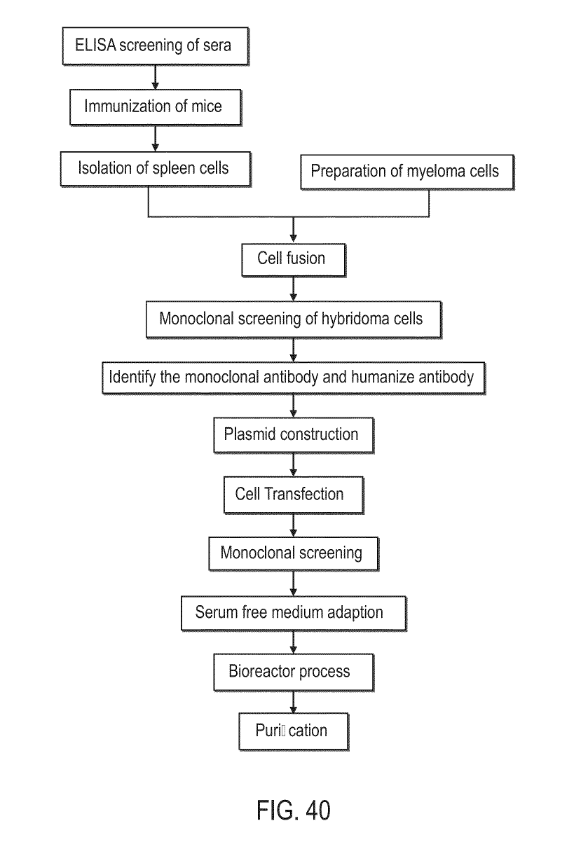

To cure diseases, viruses infections, bacteria infections, auto immune disease, neurological disorder, all type of solid and blood cancer, coagulation, diabetic, inhibitor, immune deficiency, muscle and nerve repair and restoration from Human or animal.
FIG. 40

The use of cultured cell from a product to express in order to obtain the desired proteins.

The inventor has discovered a number of new cells under different patent. The discovery led to the use of existing products like AIbuRAAS®, GammaRAAS®, HemoRAAS®, ProthoRAAS®, FibroRAAS®, ThrombiRAAS®, FibringluRAAS® and HepaRAAS® to culture to obtain the desired cell for expression, in addition to the newly discovered cells.

The desired cells can be obtained through culture of the plasma or the fraction or the final products including the AFOD RAAS and AFCC RAAS products.

After harvesting the desired cells for a certain protein, the cell expression to increase the cell population to produce enough desired proteins for further process in the final product.

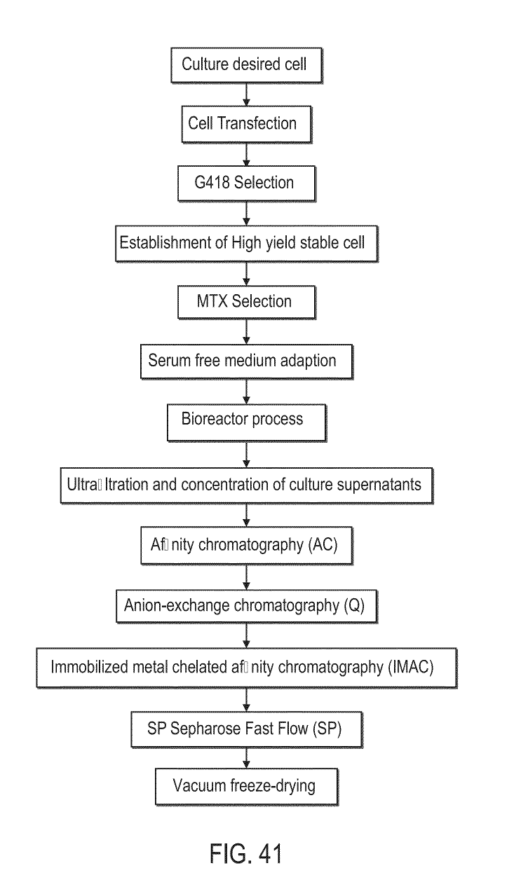

Such a method include the selection of various mediums or amino acids to help grow the cells.
FIG. 41

The manufacture of AFOD RAAS and AFCC RAAS products by using the direct cell from cell culture for expression to synthesize the desired already discovered or newly found proteins.

In this study we also found a lot of cells from different mediums of plants, fruits, vegetables, rice, Oatmeal or any source of meat or seafood, it was amazing that we have found a lot of cells in these mediums which can generate the cells within seconds to get up to 20-30 million cells, while the CHO cell for our recombinant factor VIII it will take a week to grow up to 10 million cells.

We also use 50 g of rice to produce 5 liters of medium and instantly this medium has 2.0 million cells, using this medium to mix with our products of Human Albumin and Immunoglobulin to observe the growth of cells for expression.

The same process can apply for the existing products as stated above and the newly discovered proteins KHI, KH2, KH3, KH4, KH5, KH6, KH7, KH8, Kf-19, KI-IIO, KH11, KH12, KH13 KH14 KH15, KH16, KH17, KHIKH19, KH2KH2L KH22, KH2.3, KH2.KH2.5, KH2.6, KH2.7, KH28, KH29, KH3 Kf-131, KH32, KH33, KH34, KH35, KH36, KH37 KH38, KH39, KH40, Kf-141, Kf-142, Kf-143, KH44, KH45, KH46, KH47, KH48, KH49, Kf-150, Kf-151 and KH52.

Thrombin which contains good protein, synthesized by good healthy cells can be delivered by microscopy.

In order to have products for oral applications by metabolism the enzymes of all these products can be extracted formulated in powder form and put in a capsule.

In conclusion all these processes can provide all products for the following routes of applications

1. In liquid form for injection.

2. In powder form for topical applications

3. Enzyme in powder in capsule for oral application

### Mechanism

KH 1-through KH-52, and more KH proteins are being discovered in GOOD HEALTHY CELLs-named KH CELLS. KH CELLS are GOOD HEALTHY CELLS in which the RNA synthesizes good proteins that:

1—Send signal to the DAMAGED, SICK, AND BAD CELLS that triggers that synthesis of good proteins that transform these cells to become GOOD healthy cells.

2—Send signal to the other currently undamaged cells to synthesis of good proteins to protect them from being DAMAGED, INFECTED and PRONE to DNA and other cellular alterations.

3—Send signal to the body to produce ne\N cells that are healthy and forbid them from being affected by intra- and extracellular damaging signals.

The mechanisms that govern these processes is the KH good healthy cells provide innate good signals that make good proteins to boost the immune system in order to CURE, TO PROTECT, and TO PREVENT diseases, viruses infections, bacteria infections, auto immune disease, neurological disorder, all type of solid and blood cancer, coagulation, diabetic, inhibitor, immune deficiency, muscle and nerve repair and restoration from Human, animal or substances by the method of fractionation, purification, recombinant DNA, monoclonal antibody, transgenic and expression of cells from the cultured GOOD HEALTHY CELLS.

The following studies have been performed to provide critical evidence for the three mentioned above mechanisms:
1) The study of APOA1 protein in preventing atherosclerosis and related cardiovascular diseases
2) The lipid profile results and quantification of atherosclerosis plaque in 18 ApoE mice fix 4 weeks stduy.
3) RAAS AFOD RAAS 1(APOA1) in ApoE mice for 8 weeks.
4) RAAS AFOD RAAS 1(APOA1) in ApoE mice for 16 1 veeks.
5) Efficacy study of RAAS antibodies on Type 2 diabetic mouse model in db/db mice
6) In Vivo Efficacy Testing of eight RAAS compounds in 4T1-LUC Breast Cancer Cell Orthotopic Model
7) In Vivo Efficacy Testing of eight RAAS compounds in 4T1-LUC Breast Cancer Ceil Orthotopic Model
8) Anti-tumor efficacy of high concentrated fibrinogen enriched alat thrombin and Afod (FS) in combination with Afod RAJ\.S 2 or Afod RA.AS 4 in patient-derived tumor xenograft (PDX) models in nude mice.
9) Characterization of lymphoid tissues and peripheral blood in nude mouse treated with and without AFCC.
10) Antiviral efficacy of AFOD RAAS-2 in an influenza H1N1-infected mouse model
11) ″″″″″″″″′<?of AFOD on 6-OHDA rat model of Parkinson's disease
12) Antiviral efficacy of AFCC in an influenza EI1N1-infected mouse model
13) Antiviral efficacy of AFOD and AFCC in an influenza H1N1-infected mouse model
14) Efficacy of AFOD RAAS 104C:8:) (f(umerly AFOD RAAS 8) in the EIBV Mouse Hydrodynamic Injection :1\.t!odel.

The recent tsunami and earthquake in Japan in March of 2011, caused panic and economy loss not only in Tokyo but around the world as people tried to escape from Tokyo due to the radiation caused by leaks in the country nuclear power plants. Such a fear of radiation that would spread into the ocean, plants, humans and animals which caused a great economic loss. The fear of radiation exposure continues to haunt the people of Japan and around the world. In addition there was no protection for the workers in the plant to stop the radiation leaks in time to minimize the damage and economic loss. With this invention the workers now can be protected and can do their job under hardiest conditions as they will not develop any type of cancer.

In addition with this invention it is possible that the nuclear power industry with hundreds of billions at stake could be saved if the workers are protected then can operate the power plant. Not only the human beings can be protected from the radiation exposure, but also food and animals can be protected as well. (Under another patent application, internal number RAA025)

In vitro Studies have been performed
for: Plasma Products
Animal derived
products
Recombinant
Products
Monoclonal
Products Cell
Expression products

### Plasma Products

### In Vitro Studies For HIV Virus 1 & 2

### HIV Study Report

PROJECT ID: RAAS<201110178

STUDY TITLE: In vitro Anti HIV Activity of Human Plasma Derived Proteins on HIV RT Enzyme

STUDY PERIOD: Nov. 16-Nov. 21, 2011

REPORTING DATE: Nov. 24, 2011

The research service was conducted in accordance with sound scientific principles. This report accurately reflects the raw data from the assay.

### I. Study Objective:

To analyze human plasma derived proteins for anti HIV activity on HIV RT enzyme

### II. Study Protocols:

1. Materials:

1.1 Samples information: RMS provided the test articles in the form of dry powder or liquid (Table

1). Wuxi provided reference compound in Drv1SO solution.

TABLE 1
Sample information
AFCC RONA 0.00001% Lyophilized AFOD KH 10 ml

| Name | Protein Conc. | Formulation | Dilutents |
|---|---|---|---|
| AFOD KH | 10% | Liquid | |
| AFCC KH | 3.50% | Liquid | |
| AFC RASS1 | 4% | Lyophilized | AFOD KH 10 mL |
| AFCC RASS4 | 0.0020% | Lyophilized | AFOD KH 10 mL |
| AFCC RONA | 0.00001% | AFOD KH 10 ml | AFCC RONA |
| | Lyophilized | | |

1.2 Reagents:

TABLE 2
List of reagents

| Reagents/Plates | Vendor | Cat. # |
|---|---|---|
| HIV-1 Reverse Transciptase | Merck | 38129-SOOU |
| Wild type enzyme | | |
| Avidin standard plates | | MSD-L15AA-6 |
| RNA template t500 syntheic | IB/GMBH | Cat. #89142N/S |
| piece of RNB | | |
| CHAPS | Pierce | Pirece-28300 |
| EGTA | Sigma | Sigma-E3889-10G |
| DTT | Sigma | Sigma-D43815-SG |
| d-ATP | Sigma | Sigma-D6500-10MG |
| d-GTP | Sigma | D4010-10MG |
| d-CTP-Na2 | Sigma | D4635-10MG |
| Water (DEPC treated) | Invitrogen | Invitrogen-750023 |
| dry bipD500 primer | Shanghai | |
| | Shenggong | |
| BSA | Sigma | Sigma-A3294 |
| 4-Read buffer T | MSD | MSD-R92TD-1 |
| Ru - d- UTP | MSD | Lot: DG2005245071 |
| 96-well round bottom | Costar | Costar-3365 |
| polypropylene plates | | |
| PCR tubes | AXYGEN | AXYGEN-PCR-0208-C |
| PCR tube covers | AXYGEN | AXYGEN-PCR-2CP-RT-C |

1.3 Instrument
Sector Imager S6000 (MesoScale Discovery MSD)
Eprnotoin (Eppendorf)
Janus (perkinelrner)
Orbital shaker

2. Methods

2.1 !C50 measurement
2.2.1 Drug treatment: Human plasma derived protein dilutions are made by using EpMotion with 2-fold serial dilutions for 10 concentrations, each in duplicate.
a) Add 30 !JL of enzyme solution per well of the Costar 96 well plates.
b) Add 5 !JL of test article or PBS or DMSO.
c) Seal plate and shake for 2 minutes on an orbital shaker
d) Incubate for 30 minutes on an orbital shaker at room temperature.
e) Add 15!JL of the Master Mix to initiate the reaction.
f) Seal plate and shake for 5-10 minutes.
g) Incubate at 37 degree for 90 minutes.
h) While this is incubating, add 100 iJL of 5% BSA in PBS to the wells of the avidin plates.
i) Seal the avidin plates and incubate for 1 hour at room temperature.
j) After the 90 minute incubation, add 60 μl of quenching buffer to the reaction wells.
k) Seal the plates and incubate for 5 minutes on the plate shaker.
l) Transfer 50 iJL of the well contents to MSD blocked plates (the blocking buffer is simply dumped off. No wash is needed).
m) Incubate MSD plates at RT for 60 minutes.
n) Freshly dilute the 4× read buffer T to 1× using distilled water (not DEPC-treated)
o) Wash rv1SD plates 3 times with 150 μl of PBS per well per wash.
p) Add 150 iJL of 1× read buffer T to tile wells.
q) Read on the Sector Imager Instrument.

2.2.2 Sample or Compound Addition

Test samples were diluted in PBS as 3.5×10⁴ pg/ml stocks. Sample dilutions are made by using Epmotion with 2-fold serial dilutions for 10 concentrations plus PBS (see below for final compound concentrations in the HIV-RT enzyme assay). Reference compound were dissolved in DMSO as 10 mM stocks and dilutions are made by using Eprnotion with 3-fold serial dilutions for 10 concentrations plus Drv1SO (see below for final compound concentrations).

TABLE 3
Sample or compound concentrations for !C50 measurement
Name

| | Concentration (ug/ml) | | | | | | | | | |
|---|---|---|---|---|---|---|---|---|---|---|
| AFOD KH | 400 | 2.00 | 100 | 50 | 2.5 | 12.5 | 6.25 | 3.1 | 1.6 | 0.8 |
| AFCC KH | 400 | 2.00 | 100 | 50 | 2.5 | 12.5 | 6.25 | 3.1 | 1.6 | 0.8 |
| AFCC RAASI | 400 | 2.00 | 1.00 | 50 | 2.5 | 12.5 | 6.25 | 3.1 | 1.6 | 0.8 |
| AFCC RAAS 4 | 400 | 2.00 | 1.00 | 50 | 2.5 | 12.5 | 6.25 | 3.1 | 1.6 | 0.8 |
| AFCC RDNA | 400 | 2.00 | 1.00 | 50 | 2.5 | 12.5 | 6.25 | 3.1 | 1.6 | 0.8 |
| | Concentration (nM) | | | | | | | | | |
| Reference | 100 | 33.3 | 11.1 | 3.7 | 1.2 | 0.4 | 0.1 | 0.05 | 0.02 | 0.01 |
| Compound | | | | | | | | | | |

2.2.3 Data analysis:
Percent of HIV-RT inhibition by protein or compound is calculated using the following equation:

% lnh.=[1−(Signal of sample−Signal of control)/(Signal of DMSO or PBS control−Signal of control) [symbol] *100.
Dose-response curves are plotted using Prism

### III. Assay Results:

3.1 Raw data from the HIV-RT enzyme assay.

3.1.1 HIV-RT enzyme assay Plate Map*:

### Plate 1

| column | column | column | colurnn | column | colurnn | coiurnn | column | column | colurnn | column | colurnn |
|---|---|---|---|---|---|---|---|---|---|---|---|
| 1 | 2 | 3 | 4 | S | 6 | 7 | 8 | 9 | 10 | 11 | 12 |

| | | | |
|---|---|---|---|
| raw A | p | AFOD KH | B |
| raw B | B | | G |
| raw C | S | AFCC KH | |
| raw D | | | |
| raw E | p | AFCC RAAS 1 | p |
| raw F | B | | B |
| raw G | S | Reference Compound | S |
| raw H | | | |

* BG: background

### Plate 2

| column | column | column | colurnn | column | colurnn | coiurnn | column | column | colurnn | column | colurnn |
|---|---|---|---|---|---|---|---|---|---|---|---|
| 1 | 2 | 3 | 4 | S | 6 | 7 | 8 | 9 | 10 | 11 | 12 |

| | | | | |
|---|---|---|---|---|
| *BG: | raw A | p | AFCC RAAS 4 | B |
| background | raw B | B | | G |
| | raw C | S | AFCC BONA | |
| | raw D | | | |
| | raw E | p | Reference Compound | p |
| | raw F | B | | B |
| 3. | raw G | S | Dtv1SO | S |
| 1. | raw H | | | |
| 2. | Raw data | | | |

### Plate 1:

| column | coiumn | coiumn | column | column | column | column | column | column | column | column | coiumn |
|---|---|---|---|---|---|---|---|---|---|---|---|
| 1 | 2 | 3 | 4 | 5 | 6 | 7 | 8 | 9 | 10 | 11 | 12 |
| 2439 | 1596 | 2113 | 2160 | 2304 | 2448 | 2214 | 2152 | 2307 | 2360 | 2357 | 60 |
| 2569 | 1866 | 2154 | 2343 | 2351 | 2371 | 2397 | 2317 | 2310 | 2454 | 2245 | 64 |
| 2571 | 281 | 329 | 393 | 563 | 805 | 1157 | 1683 | 2011 | 2304 | 2384 | 60 |
| 2361 | 267 | 306 | 376 | 518 | 762 | 1156 | 1600 | 1912 | 2158 | 2185 | 58 |
| 59 | 1238 | 1782 | 2097 | 2230 | 2299 | 2326 | 2374 | 2368 | 2329 | 2449 | 2267 |
| 52 | 1248 | 1812 | 2166 | 2300 | 2406 | 2462 | 2398 | 2369 | 2346 | 2353 | 2366 |
| 54 | 87 | 142 | 246 | 469 | 850 | 1241 | 1629 | 1791 | 1873 | 1851 | 2263 |
| 53 | 85 | 132 | 241 | 474 | 833 | 1349 | 1651 | 18B | 1924 | 1907 | 2438 |

### Plate 2:

| column | coiumn | coiurnn | column | column | column | column | column | column | column | column | coiurnn |
|---|---|---|---|---|---|---|---|---|---|---|---|
| 1 | 2 | 3 | 4 | 5 | 6 | 7 | 8 | 9 | 10 | 11 | 12 |
| 2491 | 1713 | 1940 | 2168 | 2411 | 2358 | 2378 | 2459 | 2289 | 2262 | 2038 | 43 |
| 2596 | 1674 | 2220 | 2344 | 2547 | 2491 | 2418 | 2541 | 2443 | 2476 | 2104 | 45 |
| 2539 | 1147 | 2176 | 2381 | 2522 | 2388 | 2433 | 2314 | 2459 | 2358 | 2369 | 44 |
| 2544 | 1689 | 2123 | 2305 | 2453 | 2385 | 2400 | 2426 | 2204 | 2049 | 2168 | 39 |
| 44 | 91 | 146 | 270 | 514 | 957 | 1429 | 1801 | 1807 | 1895 | 1880 | 2142 |
| 38 | 85 | 139 | 263 | 472 | 946 | 1377 | 1614 | 1708 | 1850 | 1853 | 2292 |
| 45 | 2119 | 2160 | 2084 | 2046 | 2069 | 1963 | 1975 | 2002 | 1961 | 1912 | 2343 |
| 43 | 2052 | 2038 | 2039 | 1975 | 1954 | 1860 | 1968 | 1972 | 1875 | 2042 | 2405 |

3.2 Activity of the Samples or compounds. IC50 values are summarized in Table 4. GraphPad

Prism files containing dose-dependent curves are presented in this report, as shown in FIG. 1. Table 4. !C50 Summary of the human plasma derived proteins and the reference compounds.

| Name | IC50 (ug/ml) |
|---|---|
| AFOD KH | >400 |
| AFCC KH | 9.89 |
| AFC RASS1 | 49% inhibition at 400 ug/ml |
| AFCC RASS4 | >400 |
| AFCC RDNA | >400 |
| | IC50 (nM) |
| Reference | 0.9 1.2 |

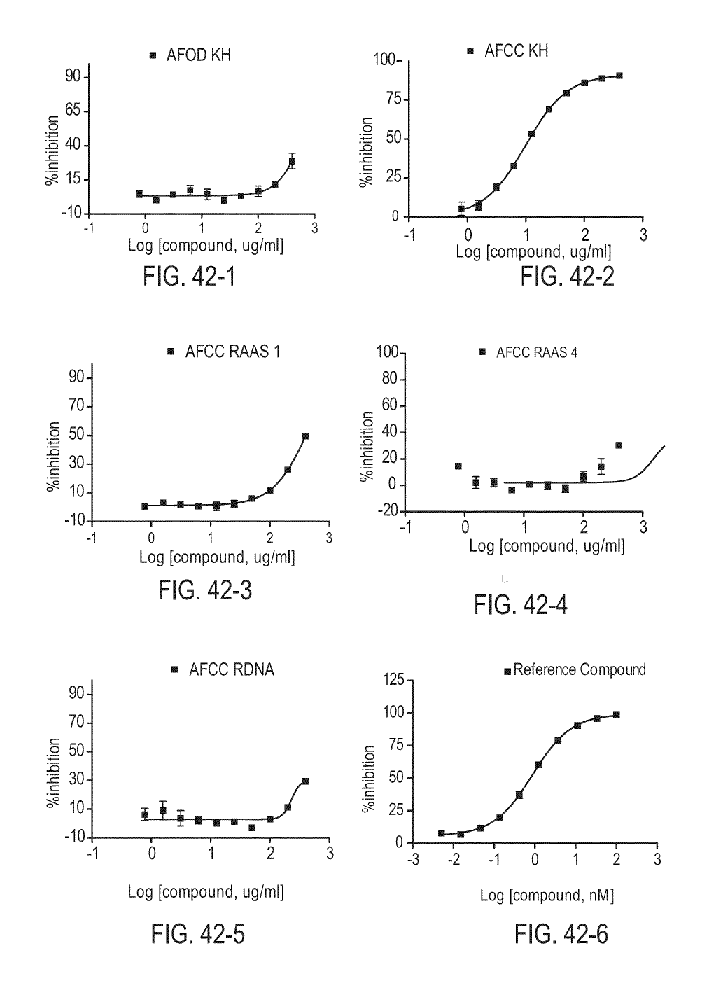

FIG. 42. Dose-Dependent Curves (by GraphPad Prism}

4. Conclusions

The Z factors of the two plate were 0.84 (plate 1), 0.80 (plate 2), which were much better than QC standard of OS Therefore, the assay data rnet our QC qualification.
The IC50s of positive control in this study were 0.9 nM (plate 1), 1.2 nl\!1 (plate 2) and these results are consistent with our previous data.

### In Vitro Studies of Hepatitis B Virus

### HBV Study Report

### PROJECT CODE: RAAS 20110815C

STUDY TITLE: To analyze human plasma derived proteins for anti HBV activity in HepG2.2.15 cells

### STUDY PERIOD: Nov. 24-Dec. 6, 2011

### REPORTING DATE: Dec. 23, 2011

L Study Objective: To test human plasma derived proteins for anti-HBV potency and cytotoxicity in a stable HBV cell line

### II. Study Protocols:

1. Materials:

Cell line: HepG2.2.15

1.2 Samples:

RAAS provided the test articles in the form of dry powder or liquid {Table 1 pi::st samples were diluted in PBS as 3.5×1041 Jglml stocks. Sample dilutions are made by Janus with 2-fold serial dilutions for 8 concentrations plus PBS. Lamivudine is diluted with 3-fold for 9 concentrations.

TABLE 1
Sample information

| | Protein | | |
|---|---|---|---|
| Name | cone. | Formulation | Diluents |
| AFOD KH | 10% | _{9- -} | |
| AFCC KH | −5.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

1.3 ECso and CCso measurement Test human plasma derived proteins in the stable HBV cell line HepG2.2.15 for anti-HBV potency.
i) Cell culture medium: RPM 1640-4% FBS-1% PeniStrep-1% Glutamine
ii) HepG2.2.15 cell culture: Grow the cells in T75 flask. Incubated at 3TC, 950 ft, humidity, 5% CO2. Perform 1:3 split every 2-3 days. iii) EC5o measurement:

1) Drug Treatment
a) Human plasma derived protein dilutions are made by using Janus with 2-fold serial dilutions for 9 concentrations, each in duplicate.
b) Check cells under microscope.
c) Prepare cell suspension and count cell number. d) Seed the HepG2.2.15 cells into 96-well plates.
e) Treat the cells with cell culture medium containing individual human plasma derived protein 24 hours after cell seeding, the final concentrations of the samples are
shown in Table 2.

Name

| | Concentration (ug/ml) | | | | | | | | | |
|---|---|---|---|---|---|---|---|---|---|---|
| AFOD KH | 400 | 2.00 | 100 | 50 | 25 | 12.5 | 6.25 | 3.1 | 1.6 | 0.8 |
| AFCC KH | 400 | 2.00 | 100 | 50 | 25 | 12.5 | 6.25 | 3.1 | 1.6 | 0.8 |
| AFCC RAAS [symbol] | 400 | 2.00 | 100 | 50 | 25 | 12.5 | 6.25 | 3.1 | 1.6 | 0.8 |
| AFCC RAAS 4 | 400 | 2.00 | 100 | 50 | 25 | 12.5 | 6.25 | 3.1 | 1.6 | 0.8 |
| !\FCC RDNA | 400 | 200 | 100 | 50 | 25 | 12.5 | 6.25 | 3.1 | 1.6 | 0.8 |
| | Concentration (uM) | | | | | | | | | |
| Lamivudine | 2 | o.6667 1 | o.nn 1 | o.o741 | 0.0247 | o.oo82 1 | 0000n | o.ooog l | o.oo03 l | o.oom i |

[symbol]

indicates data missing or illegible when filed

f) Refresh protein-containing medium on day 3 of drug treatment g) Collect culture media from the HepG2.2.15 plates on day 6 followed by HBV DNA extraction using QIAamp 96
DNA Blood Kit (QIAGEN #51161).

2) Real time PCR for HBV DNA quantification. a) Dilute HBV plasmid standard by 10-fold from 0.1 ngiul to 0.000001 ng/ul. b) Prepare realtime PCR mix as shown blow.

| | | |
|---|---|---|
| | | Volume for 100 |
| PCR reagents | Volume | Reactions |

| | | | |
|---|---|---|---|
| DEPC Water | 1.i ul | •11O | ul |
| Taqman Universal Master Mix(2X) | 12.5 ul | 1250 | ul |
| HBV Primer ForNard(50 uM) | 0.2 ul | 20 | ul |
| HBV Primer Reverse(50 uM) | 0.2 ul | 20 | u1 |
| HBV Probe(5 uM) | 1 ul | ″IOO | ul |
| Total | 15 ul | i50 | ul |

c) Add i5 ul/well PCR mix to 96-well optical reaction plates.
d) Add 10 ul of the diluted plasmid standard to C12-H12. The amount of HBV DNA in each standard well is: 1 ng, 0.1 ng, 0.01 ng, 0.001 ng, 0.0001 ng, and 0.00001 ng, respectively.
e) Transfer 10 ul of the extracted DNA to the other wells (from Row A-H to the corresponding wells in the optical reaction plates). f) Seal the plates with optical adhesive film. g) Mix and centrifuge. h) Place the plates into realtime PCR system and set up the program according to the table blow.

| | | |
|---|---|---|
| 50′C | 2 rnin | 1 cycle |
| 95′C | 10 min | 1 cycle |
| | 15 s | |
| 60′C | 60 s | 40 cycle |

3) Data analysis: A standard curve is generated by plotting Ct value vs. the amount of the HBV plasmid standard, and the quantity of each sample is estimated based on the Ct value projection on the standard curve; percent of HBV inhibition by protein or compound is calculated using the following equation: % lnh.=[1−(HBV quantity of sample−HBV quantity of HepG2 control)/(HBV quantity of 0% Inhibition control−HBV quantity of HepG2 control)]*100.

Test human plasma derived proteins in the stable HBV cell line HepG2.2.15 for cytotoxicity
i) Cell culture medium: RPM 1640-4% FBS-1% Pen/Strep-1% Glutamine
ii) HepG2.2.15 cell culture: Grow the cells in T75 flask. Incubated at 3TC, 95% humidity, 5% 002. Perform 1:3 split every 2-3 days.
iii) CC50 measurement
   a) Human plasma derived protein dilutions are made by using Janus with 2-fold serial dilutions for 9 concentrations, each in duplicate.
   b) Check cells under microscope.
   c) Prepare cell suspension and count cell number.
   d) Seed the HepG2.2.15 cells into 96-well plates.
a) Treat the cells with cell culture medium containing individual human plasma derived protein 24 hours after cell seeding, the final concentrations of the samples are shown in Table 2.
   e)
   f) Refresh protein-containing rnediurn on day 3 of drug treatment.
   g) Test cell cytotoxicity on day 6 using CeiiTiter-Biue Cell Viability Assay KIT.

iII. Assaresults:

TABLE 3
ECso raw data (Plate 1, DNA quantity, ng)

| | Sample final dose (ug/ml) | | | | | | | | | | |
|---|---|---|---|---|---|---|---|---|---|---|---|
| | 400 | 200 | 100 | 50 | 255 | 12.5 | 6.25 | 3.13 | 1.56 | 0% | |
| AFOD KH | 0. GOG | 0. 005 | 0. 005 | 0. 006 | 0. 007 | 0. 006 | 0. 006 | 0. 007 | 0. 007 | 0. 007 | |
| AFCC KH | 0. 006 | 0. 008 | 0. 007 | 0. 007 | 0. 007 | 0. OOG | 0. OOG | 0. 002 | 0. 007 | 0. 002 | |
| AFCC 1H | 0. 009 | 0.009 | n. OO′i | n. OO′i | 0. 006 | 0. 006 | 0. 006 | o. out. | (I UOi) | o. out. | |
| AFCC RAAS 1 | U. 006 | 0. OOb | 0. OOb | 0. OOb | 0. OOti | 0. (II)i> | 0 00;′ | (I UOG | 0. 007 | (I UOG | |
| AFCC RAAS 1 | 0. 00′3 | 0. OOG | 0. oo:: | 0. OOG | 0. (11)9 | 0. (IIk | 0 (IQ(i | (I uo:; | 0. 008 | (I uo:; | |
| | | | | | | | | 10 | II | [symbol] | \| |

[symbol]

indicates data missing or illegible when filed

TABLE 4
EC5o raw data (Plate 2, DNA quantity, ng)

| | Sample final dose (ug/ml) | | | | | | | | | |
|---|---|---|---|---|---|---|---|---|---|---|
| | 400 | 200 | 100 | 50 | 255 | 12.5 | 6.25 | 3.13 | 1.56 | 0% |
| AFOD KH | 0. 00!3 | 0. G08 | 0. G07 | 0. G07 | 0. G09 | 0. G09 | 0. G09 | 0. 012 | 0. 00!3 | 0. G08 |
| AFCC KH | 0. 007 | 0. 00′1 | 0. 00′1 | 0. 00′1 | 0. 00′1 | 0. 008 | 0. 00′1 | 0. 008 | 0 | 0. 007 ! |
| | | | | | | | | | | 0. 006 |
| | | | | | | | | | | i 0. |
| | | | | | | | | | | 006 |
| AFCC 1H | . 007 | 0. 007 | 0. GOG | 0. G07 | 0. G07 | 0. GOG | 0. G07 | 0. GOG | 0. G07 | . 007 |
| AFCC RAAS 1 | 0. 001 | 0.0( )1 | 0. G01 | 0. G02 | 0. G03 | 0. G05 | 0. G07 | 0. G11 | 0. G10 | 0. 001 |
| AFCC RAAS 1 | 0. 001 | 0, 001_{—} | 0. 00\ | 0. 002 | U, 004 | U, OO′I | U, 010 | 0. 012 | U, 014 | 0. 001 |

TABLE 5
CCso raw data (Plate 1)

| | 5,a:;;-lp le 1- LnoLclose- (ug/ml) | | | | | | | | | | | |
|---|---|---|---|---|---|---|---|---|---|---|---|---|
| | Λ | 400 | 200 | J00 | fi0 | 2S | L2. 50 | 6. 25 | 3. t,) | 1. SG | | DMEM |
| .″>FOD KH | B | 5580:\ | 64r{9′l | ? l230 | rf2l, 9 | rnl39 | ″/8!39 | ?Wt04 | ?9161 | ″!9i!•1 | 81561 | llil8 |
| AFDD 1\H | \ | 56.S2:\ | 6(; :33 | ?0631 | nl31 | r{f,( )(;8 | ″/30 ll | Tf9!J6 | ?i!•120 | ?!J1Eo2 | 8168″1 | llil3 |
| ,L>,FCC KH | | 82ns | EA496 | g::S96 | 8m:n | 193.:J4 | ,s l008 | 809 · ? | E\089Eo | Tf356 | ?90:34 | ll 93 |
| AFCC KH | E | 815013 | 1′7561 | ″{t1728 | 30•lOJ | 73910 | 82101 | 8:35f17 | 1′601′7 | ″lr!99l | 32662 | 1168 |
| AFCC RAr′\S 1 | F | 66408 | 74141 | 78364 | 78223 | 76486 | 77972 | 75031 | 78457 | 66609 | 70886 | llGl |
| AFCC R1\AS −j | 6T?46 | 17(!)\) | ?4032 | rfS l93 | “(8[”!9 | ″/66′l | 803130 | 19r{!)′l | 694?:3 | TI56.:J | ll″/(l | AFCC |
| | H | | | | | | | | | | | |

Note:
DrvIEl\!1-′100 · ;; inhibition control

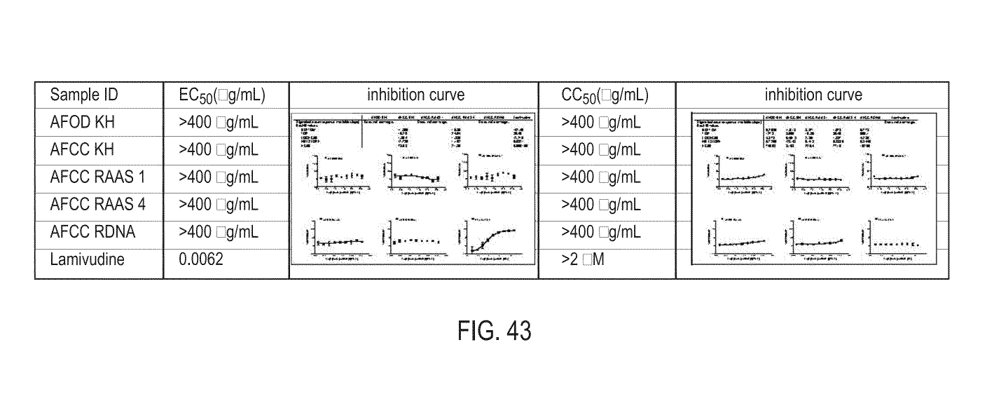

FIG. 43: Table 7. EC5o and CC5o Summary

### IV. Conclusions

The EC5D of the positive control larnivudine in this study is 0.0062 ul\!1, which is consistent with our previous data.

### In Vitro Studies of Hepatitis C Virus

### HCV Study

### Report

### PROJECT CODE: RASSD20111017A

STUDY TITLE: Test human plasma derived proteins against HCV genotype 1a, 1b and 2a replicons for antiviral activity (EC₅₀)

### STUDY PERIOD: Nov. 16-Nov. 21, 2011

### REPORTING DATE: Nov. 24, 2011

The research service was conducted in accordance with sound scientific principles. This report accurately reflects the raw data from the assay.

### I. Study Objective:

To analyze human plasma derived proteins for anti HCV activity (EC₅₀) and cytotoxicity (CC₅₀) using HCV is b and 2a rep!icon culture systems

### II. Study Protocols:

3. Materials:

1.1 Ce!!Une:

Replicon cell lines 1a and 2a were established following published methods (1,2) using Huh? by G4″18 selection. The replicons were assembled using synthetic gene fragments. The GT 1a line is derived frorn H77 and contains PVIRES-Luciferase-Ubi-Neo, and two adaptive mutations: P1496L, 822041. The 2a line contains no adaptive mutations and encodes a Luciferase reporter. The 1b replicon plasmid is also assembled using synthetic gene fragments. The replicon genome contains PVIRE8-Luciferase Ubi-Neo gene segments and harbors 1 adaptive mutation (822041), and the backbone is Con1.

1.2 Compounds:

The test articles are supplied in the form of dry powder or 10 mM solution, and Ribavirin as control, in duplicate.

1.3 Reagents:

[symbol]

TABLE 1
List of reagents

| | VENDOR Ca [symbol] | |
|---|---|---|
| REAGENT | tal [symbol] | REAGENT |
| ! Dimethyl sulfoxide (mv1SO) | Sigma | Cat#34869 |
| 1---o fEM-------------------------------------------------- | | ---cai#T1-96o o-ii r------ |
| ! Fetal Bovine Serum (FBS) | Gibco | Cat#16140 |
| ! MEM non-essential amino acids | Invitrogen | cat#11140-050 |
| [---c=8iLiTa_m_iile-------------------------------------- | | ---caw25o3o o-sT |
| ! Trypsin/EDTA | Invitrogen | Cat#25200-072 |
| ! 96 well cell plate | Greiner | Cat#655090 |
| h eiiTTter-¥fLi_o_;:-------------------------------------- | | ---caw-i3-6os T----------- |
| ! Bright-Gio | Promega | Cat#E2650 |

[symbol]

indicates data missing or illegible when filed

1.4 Instrument

to Envision(Perkinelmer)

to Multidrop(Thermo)

to Janus (Perkinelmer)

4. Methods

2.1 Cell Addition

T150 flask containing 1a, 1b and 2a replicons cell monolayer is rinsed with 10 ml pre-warmed PBS. Add 3 ml of pre-warmed Trypsin 0.25% and incubate at 5% CO₂, 37 cC for 3 minutes. Nine milliliters of DMEM complete media are added, and the cells are blown for 30s by pipetting. The cells are counted using hemocytometer.

1a, 1b and 2a replicons cells are resuspended in medium containing 10% FBS to reach a cell density of 64,000 cells/ml (to obtain a final cell plating density of 8000 cells/125 ul/well). Plate cells in Greiner 96 black plate using Multidrop. Incubate plate at 5% CO₂, 37t for 4 hours.

2.2 Compound Addition

RAAS provided the test articles in the form of dry powder or liquid (Table 2).Test samples were diluted in PBS as 3.5×10\Jg/rnl stocks. Sample dilutions are made by Janus with 2-fold serial
dilutions for 10 concentrations plus PBS. Ribavirin is also diluted by Janus with 2-fold for 10 concentrations. The final sample concentrations of tile HCV replicon assay are described in Table 3.

TABLE 2
Sample information

| Name | Protein cone. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 ml |
| AFCC RAAS 4 | 0.0020% | Lyophilized | AFOD KH 10 ml |
| AFCC RONA | 0.00001% | Lyophilized | AFOD KH 10 ml |

TABLE 3
Sample or compound concentrations for EC₅₀ and CC₅₀ measurement

| Name | HCV Genotype | | | | | | | | | | |
|---|---|---|---|---|---|---|---|---|---|---|---|
| | | Concentration (pg/ml) | | | | | | | | | |
| AFOD KH | 1a/1b/2a | 400 | 200 | 100 | 50 | 25 | 12.5 | 6.3 | 3.1 | 1.6 | 0.8 |
| AFCC KH | | 400 | 200 | 100 | 50 | 25 | 12.5 | 6.3 | 3.1 | 1.6 | 0.8 |
| AFCC RAAS 1 | | 400 | 200 | 100 | 50 | 25 | 12.5 | 6.3 | 3.1 | 1.6 | 0.8 |
| AFCC RAAS 4 | | 400 | 200 | 100 | 50 | 25 | 12.5 | 6.3 | 3.1 | 1.6 | 0.8 |
| AFCC RONA | | 400 | 200 | 100 | 50 | 25 | 12.5 | 6.3 | 3.1 | 1.6 | 0.8 |
| | | Concentration (IJM) | | | | | | | | | |
| Ribavirin | | 320 | 160 | 80 | 40 | 20 | 10 | 5 | 2.5 | 1.3 | 0.6 |

2.3 Detection (after 72 Hours of Incubation)
Bright-Gio Luiferase and C:ei!Titer-Fluor′M are prepared and stored in dark while allowing to equilibrate to room temperature. Plates are removed from incubator to allow equilibration to room temperature. Multidrop is used to add 40 ul C:eliTiter-Fluor′″ to each well of compound-treated cells.

The plates are incubated for 0.5 hour, and then read on an Envision reader for cytotoxicity calculation. The cytotoxicity is calculates using the equation below.

$$\text{?} = 1 - \begin{matrix}Cmpd - Background \\ D 1 \backslash fSO - Background\end{matrix} \times 100$$

$$\text{?} \text{indicates text missing or illegible when filed}$$

100 ul of Bright-Gio are added to each well, incubated for 2 minutes at room temperature, and chemi-luminescence (an indicator of HCV replication) is measured for EC₅₀ calculation.
The anti-replicon activity(% inhibition) is calculated using the equation below

$$\text{?}$$

$$D \text{?} {vfS}^{′} O - \\ back , ground$$

$$\text{?} \text{indicates text missing or illegible when filed}$$

Dose-response curves are plotted using Prism.

### III. Assay: Results:

1 Assay Plate Map

plate 1

| | | |
|---|---|---|
| C | AFOD KH | P |
| T | AFCC KH | B |
| L | AFCC RAAS 1 | S |

plate 2

| | | |
|---|---|---|
| C | AFCC RAAS 4 | P |
| T | AFCC RONA | B |
| L | Ribavirin | S |

2 Raw data

2.1 Raw Data of Cytotoxicity Assay

| | | | | | | | | | | | |
|---|---|---|---|---|---|---|---|---|---|---|---|
| 11788 | 3?82D | 7G241 | ?9783 | 8I′I094 | 89352 | 8G4?5 | 84132 | 79122 | 8231? | 78529 | 84888 |
| 10513 | 38733 | 73718 | 79841 | 90368 | 82949 | 84058 | 85256 | 86834 | 85378 | 81751 | 78143 |
| 11907 | 71545 | 83521 | 89′104 | 9183′1 | 87528 | 88304 | 89908 | 89782 | 81452 | 87404 | 80906 |
| 10873 | 82130 | 82349 | 86032 | 91782 | 13224 | 90052 | 88416 | 8502P | 87835 | 82113 | 80·121 |
| 1201; | G1801 | 825?4 | 7 i31G | 91001 | i01 iD | 94232 | 932D3 | i04W | 91IG4 | 85286 | 7 ′i43 i |
| 10586 | 51803 | 75949 | 84140 | 89954 | 84298 | 85969 | 87016 | 87714 | 84577 | 81008 | 81025 |
| 12214 | 59805 | 68928 | 67259 | 68991 | 70963 | 70986 | 72721 | 80578 | 72648 | 86545 | 75138 |
| 10586 | 55271 | 62901 | 59758 | 63586 | 63753 | 510′14 | 64486 | 70755 | 74224 | 8488′1 | 74471 |
| 121f37 | 75390 | 86019 | 93902 | 94512 | 84075 | 78058 | 81G19 | 7841P | 813′11 | 8′1G04 | 83′171 |
| 10838 | 79348 | 85248 | 88417 | 90128 | i098I | 8 1205 | 87054 | 8037P | 82′154 | ?9328 | 84·191 |
| 1200G | 42127 | ′i6fr16 | 5S340 | 70tFG | ′133 -m | 84894 | 85941 | 8?58′1 | 9W10 | 91748 | 7D542 |
| 10398 | 52814 | 54925 | 59760 | 72108 | 85112 | 88015 | 84100 | 88429 | 87978 | 88712 | 79154 |

| | | | | | | | | | | | | |
|---|---|---|---|---|---|---|---|---|---|---|---|---|
| 11859 | 51104 | 57291 | 50533 | 71572 | 7·1590 | 7·1590 | 72696 | 63905 | 67′104 | 54951 | 63293 | 68405 |
| 10705 | 46415 | 52869 | 63478 | 66044 | 76232 | 76232 | 75102 | 64′:101 | 70704 | f34733 | 73663 | 6586 1 |
| 11915 | 48782 | 62222 | 70988 | 7006·! | 72337 | 72337 | 70822 | 62570 | 61489 | f3′:1424 | 67863 | 62024 |
| | | | | | | | | | | f38 | | |
| 10fj98 | 54?87 | f$′7780 | 7′4332 | ′77817 | ?fj2( )f$ | ?fj2( )f$ | 71439 | 69920 | tm2oD | ′i73 | ll0 ′i5 | 71)183 |
| 11617 | 56776 | 72151 | 78099 | 73707 | 80133 | 80133 | 77881 | 71345 | 74569 | 75191 | 72729 | 67333 |
| ′10389 | 55289 | 73692 | 79149 | 720fJ8 | 79174 | 79174 | 80854 | 75314 | 7fJ363 | 74574 | 59452 | 70933 |

| | | | | | | | | | | | |
|---|---|---|---|---|---|---|---|---|---|---|---|
| | | | | | | | 59277 | | | | |
| 1F81 | 46220 | 70386 | 71631 | 74038 | 70501 | 65402 | f349f38 | 577′14 | 59415 | 60015 | 55776 |
| | | | | l00P·! | | | f3294B | | | | |
| 10f359 | 50913 | 63077 | 71054 | ?0043 | 6627? | | 63481 | 68110 | 7H346 | 58898 | 58925 |
| | | | | fj5994 | | | | | | | |
| 11S 10 | | 37580 | 66840 | 4859, | l)6523 | | 62875 | | 67B81 | l)9418 | |
| 10463 | 59788 | 35505 | 38330 | 43076 | 75550 | G02f33 | 65543 | 64S91 | 64326 | 61607 | |
| | | | | | | | 59277 | | | | |
| 112.15 | 31282 | 70386 | ,m247 | 74038 | 59252 | 68223 | f349f38 | 63360 | 6681, | 58225 | 64260 |
| | | | | l00P·! | | | f3294B | | | | |
| 10340 | 34855 | 63077 | 71631 | ?0043 | 5620fl | 61155 | 63481 | 64284 | 66557 | 56655 | 60285 |
| | | | f33452 | fj5994 | | | | | | | |

| | | | | | | | | | | | | |
|---|---|---|---|---|---|---|---|---|---|---|---|---|
| 11260 | 62423 | 63994 | 60008 | 66320 | 63246 | 63076 | 62824 | 5422fj | 5422fj | 52388 | 56f;80 | 52388 |
| 10127 | 54433 | 51255 | 51,m7 | 55262 | 59280 | 558fJ0 | 50222 | 55138 | 55138 | 55625 | 575, 2 | 56526 |
| 11453 | 52361 | 58693 | f32869 | 69429 | 56045 | 58716 | 5B284 | f30293 | f30293 | 637?8 | 581 1? | 63778 |
| | | | | | l′l34S1 | | | | | | | |
| 10728 | )f$90B | f3SS47 | f Y7010 | 64930 | 60082 | G4533 | f33630 | | | 64781 | l)4208 | G47B1 |
| | | | | | lB244 | | | | | | | |
| 11424 | 50095 | 64112 | 61153 | 63665 | 63246 | 61140 | 62072 | | | 68446 | 61890 | 58446 |
| 10165 | 52406 | 60200 | 68101 | 64203 | 59280 | 61168 | 64479 | 66478 | 66478 | 64375 | 6130fj | 64375 |
| 12001 | 668fJ8 | 51275 | 50,53 | 63884 | 6·1264 | 60534 | 50138 | 50138 | 5546fl | 62475 | 68167 | 66469 |
| 10936 | 66043 | f30181 | 55?62 | 59218 | 56456 | 64f353 | 56607 | f31353 | 60143 | 60143 | 56251 | 61353 |
| | | | | | l)2106 | | | | | | l)0706 | |
| | | | | | l)9f348 | | | | | | l)69?5 | |
| 117S1 | G0500 | St1343 | Gf3462 | 644?0 | 6017f; | G33f34 | St1872 | 65B81 | 62280 | 62280 | 70185 | G58B1 |
| | G4051) | GH127 | | 60913 | 59597 | G17O1 | | 65950 | 64 i31 | 64 i31 | 5945fl | |
| 10f3 l3 | 37011 | 43034 | 47350 | 54734 | 56456 | 68095 | f3?3S9 | 68319 | 70444 | 70444 | 56251 | |
| | | | | | l)2106 | | | | | | l)0706 | |
| | | | | | l)9f348 | | | | | | l)69?5 | |
| 1177fj | 38973 | 42537 | ,B897 | 5302, | 6017f; | 67739 | 70369 | 65506 | 65H3 | 65H3 | 70185 | 68319 |

2.2 Raw Data of Anti-Rep!icon Activity Assay

1a plate 1

| coiumn | column | column | column | column | coiumn | column | column | column | column | coiumn | column |
|---|---|---|---|---|---|---|---|---|---|---|---|
| 8 | 732 | 3758 | 3795 | 4068 | 4308 | 3768 | 3932 | 3632 | 3,108 | 3540 | 3592 |
| 24 | 10GO | 3388 | 417f3 | 3 104 | 3f372 | 38′:16 | 3340 | 3132 | 3468 | 3248 | 3236 |
| 28 | 3″172 | 39″i6 | 4364 | 415G | 3f3G0 | 3384 | 3312 | 35H3 | 3380 | 3336 | 3G84 |
| 32 | 3731) | 4300 | 4028 | 4428 | 3840 | 3904 | 36f38 | 3828 | 3852 | 3812 | 3804 |
| 20 | 2120 | 4036 | | 4452 | 4276 | 3728 | 3708 | 4092 | 3676 | 3656 | 4148 |
| 28 | 2040 | 4080 | | 4·!56 | ,13″16 | 4084 | 4008 | 3fJ12 | 3992 | | 3844 |

1a plate 2

| coiumn | column | column | column | column | coiumn | column | column | column | column | coiumn | column |
|---|---|---|---|---|---|---|---|---|---|---|---|
| | 3312 | 41G8 | 3624 | 4348 | 3636 | 3592 | 3756 | 3188 | | 3488 | 3396 |
| 28 | 3552 | 4188 | 3480 | 4268 | 3f3 12 | 3580 | 3592 | 3832 | 3748 | 3384 | 33′:16 |
| 28 | 379 | 4396 | 4 7f3 | 4S04 | :f7t18 | 429 | 3688 | 3452 | | 3f300 | 3720 |
| 20 | 4112 | 728 | J508 | 2804 | 3524 | 40.12 | 4076 | | 3760 | 3856 | 4032 |
| 12 | 52 | 6 | | 1088 | 2800 | 3880 | 4000 | 4284 | 4360 | 3912 | 4188 |
| 24 | 341f3 | | 3304 | 3688 | 3620 | 3400 | 3400 | 3348 | 3048 | 309G | 3388 |
| 28 | 3464 | 3236 | 3852 | | 3400 | 3760 | 3316 | 321fj | 3048 | 3020 | 3338 |
| 24 | 2968 | 3176 | 347f; | | 3324 | 3440 | 3196 | 2748 | 2628 | 3108 | 3524 |
| 40 | 3″180 | 2932 | 3408 | 2956 | 3696 | 3264 | 2912 | 3480 | 2768 | 2776 | 3596 |
| 28 | 3″132 | 3760 | 3P32 | 3175 | 3548 | 3452 | 39f38 | 3172 | 319G | 3228 | 3740 |
| 20 | 3248 | 397t) | 3888 | 3724 | 40t10 | 3484 | 3440 | 3328 | 3028 | 309G | 3496 |
| 20 | 3?88 | 38S2 | 3f3t14 | 3728 | 3944 | 84 | 3436 | 3192 | 3348 | | 3 ′>88 |
| 36 | 3548 | 3964 | 341fj | 3352 | 3280 | 3232 | 3188 | 3200 | 3052 | | 3576 |
| 32 | 3856 | 3876 | 4044 | | 3364 | 3876 | 3600 | 3080 | | 3356 | 3524 |
| 24 | 4048 | 4036 | 3980 | 3124 | | 3704 | 3780 | 3388 | 3312 | 3504 | 3880 |
| 24 | 172 | 1180 | | 3318 | 3591 | 3591 | 3820 | 3208 | 3024 | | 4340 |
| 16 | 32 | 232 | 752 | 2216 | 3372 | 3668 | 4032 | 4116 | 3852 | 4208 | 4095 |

row H

[symbol]

2a plate 2

| | | | | | | | | | | | |
|---|---|---|---|---|---|---|---|---|---|---|---|
| 2,1 | 2844 | 2950 | 2856 | 2,112 | 25,14 | 2548 | 2388 | 2388 | 2304 | 2564 | 2352 |
| 32 | 3′172 | 2856 | 2708 | 2652 | 2388 | 2200 | 2428 | 205f3 | 2444 | 2328 | 2224 |
| 32 | 2″136 | 2504 | 2360 | 2268 | 2108 | 2156 | 2248 | 209f3 | 2304 | 2056 | 24′:12 |
| 20 | 2280 | 2720 | 21)84 | 2260 | 2332 | 2244 | !304 | 2572 | 2208 | 1888 | 2S32 |
| 28 | 3068 | 2664 | 2908 | 2524 | 2804 | 3092 | 2484 | 2f;08 | 2380 | 2232 | 241fj |
| 15 | 2820 | 2984 | 3016 | 28fJ2 | 2944 | 2955 | 2804 | 2392 | 2752 | 2628 | 32.15 |

row [symbol]
row H

2a plate 2

| | | | | | | | | | | | |
|---|---|---|---|---|---|---|---|---|---|---|---|
| 20 | 2700 | 2812 | 2628 | 2572 | 2524 | 2504 | 2450 | 2450 | 2,184 | 2456 | 2596 |
| 20 | 2700 | 2812 | 2628 | 2572 | 2524 | 2504 | 2450 | 2450 | 2,184 | 2456 | 2596 |
| 28 | 2752 | 2768 | 24H3 | 2208 | 2804 | 2440 | 2188 | 2884 | 2204 | 2240 | 2548 |
| 24 | 2508 | 30·H.1 | 2S68 | 2S80 | 2′744 | 20 [symbol] .14 | 504 | 2288 | 2084 | 21 ( ) 8 | 2S04 |
| 36 | 2676 | 2740 | 2740 | 2404 | 2536 | 2632 | 2236 | 2016 | 2408 | 2228 | 2232 |
| 28 | 56 | 184 | 548 | 1024 | 1428 | 2435 | [symbol] | 28 | 56 | 184 | 548 |
| 20 | ,18 | 200 | 588 | 13fJ6 | 1856 | 2248 | 2712 | 2532 | 2284 | 2520 | 2820 |

3 Cytotoxicity and anti-replicon activity of the human plasma derived proteins. CC:;o and EC50 values are summarized in Table 4. GraphPad Prism files containing dose-dependent curves are presented in this report. CC₅₀ and EC₅₀ values are shown in FIG. 1 and FIG. 2 respectively.

TABLE 4
CC₅₀ and EC₅₀ Summary of the human plasma derived
proteins Ribavirin

| | 1a | | 1b | | 1c | |
|---|---|---|---|---|---|---|
| | CC50 | EC50 | CC50 | EC50 | CC50 | EC50 |
| Name | (ug/ml) | (ug/ml) | (ug/ml) | (ug/ml) | (ug/ml) | (ug/ml) |
| AFOD KH | 60.7% @ | 76.5% @ | >400 | >400 | >400 | >400 |
| | 400 ug/ml | 400 ug/ml | | | | |
| AFCC KH | >400 | >400 | >400 | >400 | >400 | >400 |
| AFCC | 33.8% @ | 44.5% @ | >400 | >400 | >400 | >400 |
| RAAS1 | 400 ug/ml | 400 ug/ml | | | | |
| AFCC | >400 | >400 | >400 | >400 | >400 | >400 |
| RAAS4 | | | | | | |
| AFCC | >400 | >400 | >400 | >400 | >400 | >400 |
| RDNA | CC50 | EC50 | CC50 | EC50 | CC50 | EC50 |
| | (uM) | (uM) | (uM) | (uM) | (uM) | (uM) |

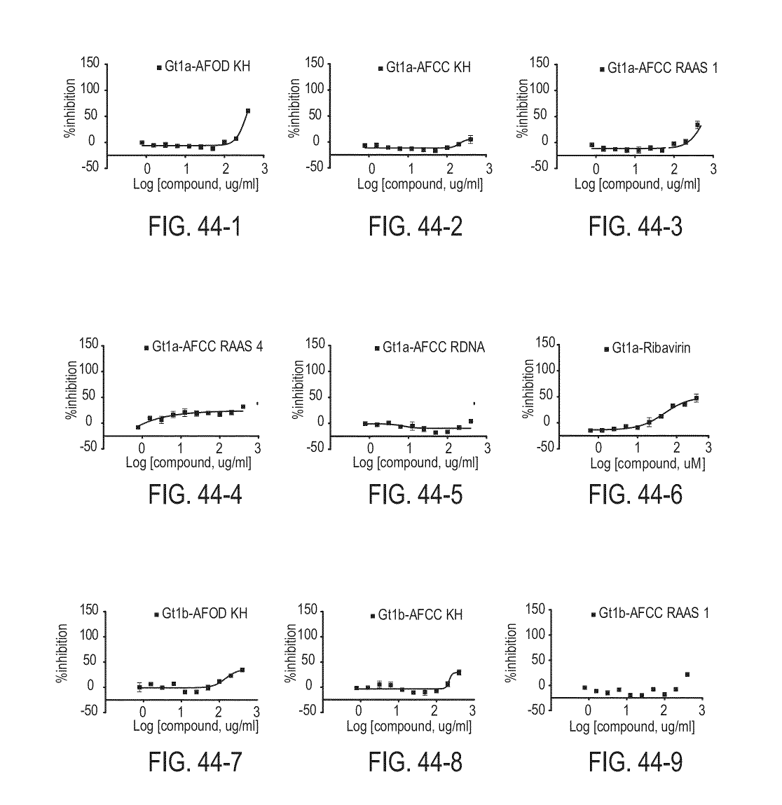

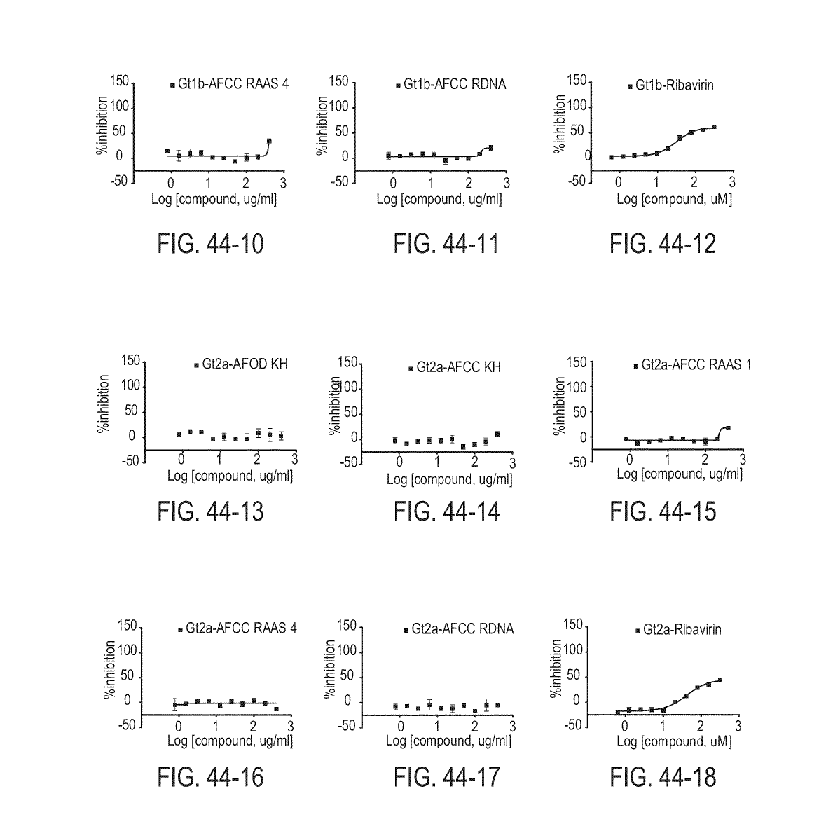

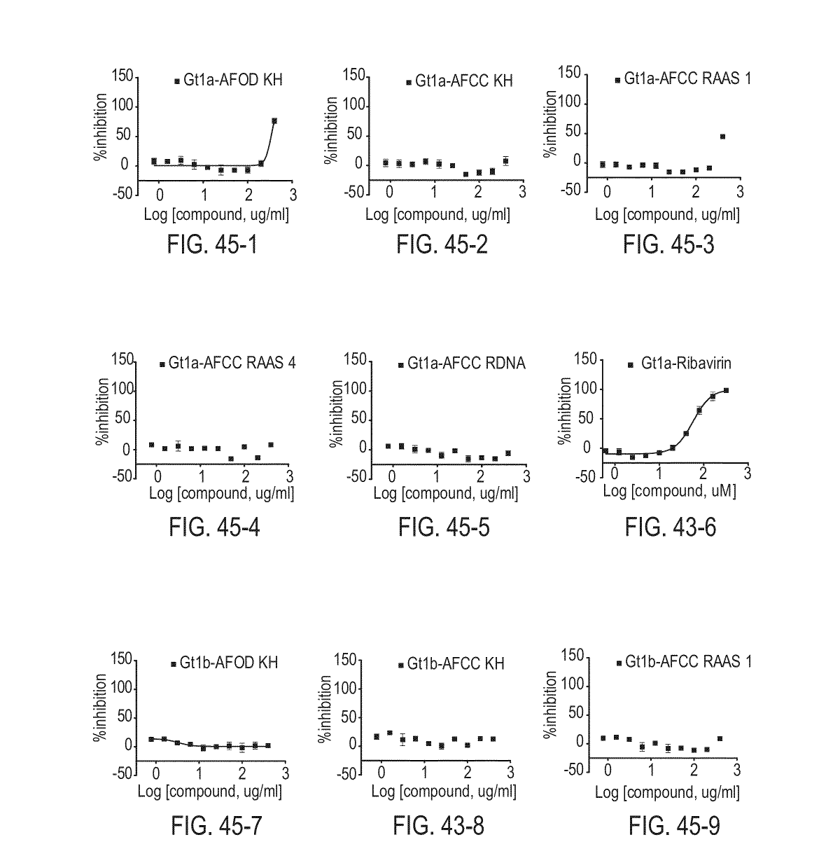

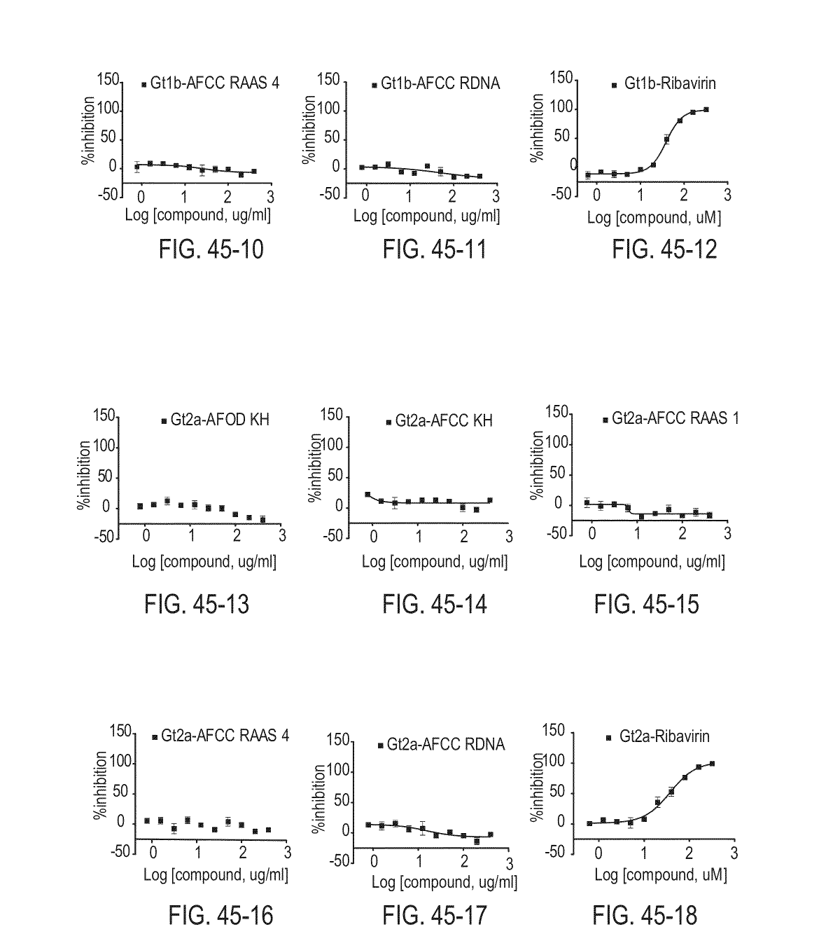

FIG. 44. Dose-Dependent Curves (CC₅₀ Values)
FIG. 45 Dose-Dependent Curves (EC₅₀ Values)

### IV. Conclusions

The Z factors of the cytotoxicity assay plates are 0.83(1a-plate!), 0.79 (1a-plate2), 0.71 (1b-plate1), 0.68 (1b-plate2), 0.65 (2a-plate1) and 0.83(2a-plate2), which are better than our QC standard.
   The Z factors of the anti-replicon assay plates are 0.75(1a-plate1), 0.70 (1a-plate2),
   0.87 (1b-plate1), 0.75 (1b-plate2), 0.58 (2a-plate1) and 0.75(2a-plate2), which are better than our QC standard.
   EC₅₀ of the positive control Ribavirin in this study are 57.58 uM (1a), 39.04 uM (1b), and :37.44 (2a), which are consistent with our previous c!ata.

### V. References

1. Mutations in Hepatitis C Virus RNAs Conferring Cell Culture Adaptation V. Lohmann et al., 2001 J. Virol.

2. Development of a replicon-based phenotypic assay for assessing the drug susceptibilities of HCV NS3 protease genes from clinical isolates. Qi X et al., Antiviral Res. 2009 February; 81(2:)166-73

### IN Vitro Study-PCR Testing for HCV

| | undiluted |
|---|---|
| CT | 20.1 |
| Q | 2.98E+07 |

| Negative plasma dilution | 2.0 fold | 2.000 fold | Drug alone |
|---|---|---|---|
| CT | 2i.6 | 3o.e | N |
| Q | 2.55E+06 | 1.69E+04 | N |
| Drug dilution | 20 fold | 2000 fold | Drug alone |
| CT | 25.B | 3′1 [symbol] | N |
| Q | 5.62E+05 | i.37E+04 | N |

[symbol]

indicates data missing or illegible when filed

Results: after 10 days incubation of samples diluted on 2012.-06-01 at 4 C refrigerators, the test was conducted again. It showed that Ct value was 2 Ct advanced in negative plasma than in drug diluted at
20 fold dilution. There is no difference at 2.000 fold dilution.

### IN Vitro Study-PCR Testing for HIV

| | undiluted |
|---|---|
| CT | |
| | 2.30E+07 |

| | 20 fold | 2000 fold | Drug alone |
|---|---|---|---|
| Negative plasma dilution | | | |
| CT | 23.9 | 30.3 | N |
| O | 2.1.1E+06 | 2.32E+04 | N |
| Drug dilution | | | |
| CT | 2?.B | N | N |
| O | •1.34E+05 | N | N |

Results: after 10 days incubation of HIV samples diluted on Jun. 1, 2012 at 4 C refrigerators, the test was conducted again. It showed that Ct value was 4 Ct advanced in negative plasma than in drug diluted at 20 fold dilution. There is no detection at 2.000 fold dilution of drug dilution.

### IN Vitro Study-PCR Testing for HBV

| | undiluted | |
|---|---|---|
| CT | 27.91 | 27.7 |

| | |
|---|---|
| CT mean | 27.8 |
| Q | 1.21E+0311.38E+03 |
| Qmean | 1.29E+03 |

Results: AFOD RAAS 104® (formerly AFOD RAAS 8) was diluted for 10 fold with normal saline and then the HBV positive plasma (1000) was diluted by this to 500 (2 fold dilution) and 100 (10 fold dilution). Negative plasma was also used as diluents for negative control. The CT value of 2 fold negative plasma diluted sample was 1 CT advanced drug diluted. One of the duplicate in drug 10 fold dilution didn't detect virus. 10 fold dilution of negative plasma was not consistent in duplication.
Samples were kept at 4 C refrigerator for 3 days, Jun. 5, 2012

| | undiluted | |
|---|---|---|
| CT | 28.51 | 28.3 |

| | |
|---|---|
| CT mean | 28.4 |
| Q | 9.46E+0211.10E+03 |
| Qmean | 1.02E+03 |

| Drug dilution | 2 fold | | 10 fold | | Drug alone |
|---|---|---|---|---|---|
| CT | 30. 1 | 31.0 | 3131 | 31.7 | N |

| | | | |
|---|---|---|---|
| CT mean | 30.6 | 31.S | N |
| Q | 3.04E+0211.72E+02 | 1.42E+0211.07E+02 | N |
| Qmean | 2.3SE+02 | 1.24E+02 | N |

| Negative | | | | | Negative |
|---|---|---|---|---|---|
| plasma | | | | | plasma |
| dilution | 2 fold | | 10 fold | | alone |
| CT | 29.91 | 30.7 | 33.21 | 33.1 | N |

| | | | |
|---|---|---|---|
| CT mean | 30.3 | 33.; | N |
| | 3.65E+0212.10E+02 | 3.84E+01 14.04E+01 | N |
| Qmean | 2.B8E+02 | 3.94[+0.1 | N |

Result: after 3 days incubation, there was no difference between negative plasma dilution and drug dilution in CT value at 2 fold dilution. The CT value in negative plasma dilution at 10 fold dilution was 2 CT advanced than drug dilution.

### In Vitro Anti-HBV Efficacy Test

### Method and Materials

1) Cell model: HepG2 cell infected with HBV virus, which is HepG2 2.2.15 cell

2) Cell viability is analyzed by MTT method

3) EIA test to detect the inhibition of HBsAg and HBeAg

4) Positive control drug: Lamivudine

5) RT-PCR detection of HBV-DNA

### Procedure

1) Toxicity of drug to cell

HepG2 2.2.15 cells are seeded in 96-”.vell plate. Fresh medium “.Vith various concentration of drug is added 48 hour later. Cell viability is analyzed 9 days later by MTT method.

2) The inhibition of HBV virus

EiepG2 2.2.15 cells are seeded in 96-\vell plate. Fresh medium With various concentration of drug is added 48 hour later. The HBsAg and HBeAg are detected 5 days, 7 days, and 10 days later. RT-PCR detection of HBV-DNA

### Results

| i\FOD | HBsAg | | HBeAg | |
|---|---|---|---|---|
| (!J.g/rnL) | OD | Inhibition rate % | OO | Inhibition rate % |
| 10 | 0.611 | 47.6 | 1.020 | 17.6 |
| 5 | 0.695 | 40.4 | 1.059 | 14.5 |
| 2..5 | 0.7!5 | 33.5 | 1.115 | 10.0 |
| 1.2.5 | 0.897 | 23.1 | 1.165 | 5.9 |
| Negative control | 1.166 | / | 1.238 | / |

### Quantification Test Results for HBV and HCV

| Sample Name | Quantification Test Results (IU/ml) |
|---|---|
| 10⁵ HCV + AFOD--KH | 2.8E+04 |
| 10⁵ HCV + AFCC-RAAS-2 | 8.1E+05 |
| 10⁵ HCV + AFCC-RAAS--6 | <25.0 |
| 500 HBV + AFOD-KH | 8.18E+l |
| 500 HBV + AFCC-RAAS-2 | <2.00E+1 |
| 500 HBV + AFCC-RAAS-6 | 5.04E+l |
| 500 HBV + AFC:C-RAAS-8 | <2.00E+1 |
| 500 HBV + Negative Plasma | 4.41E+l |

Note: The detection limit for HBV quantification is 2.00E+11 U/mL

| | |
|---|---|
| 10⁵ HCV + AFCC-RAi\S-8 | 2.4E+05 |
| 10⁵ HCV + Negative Plasma | 2.11E+3 |

Note: The detection limit for HBV quantification is 2.5 IU/ml.

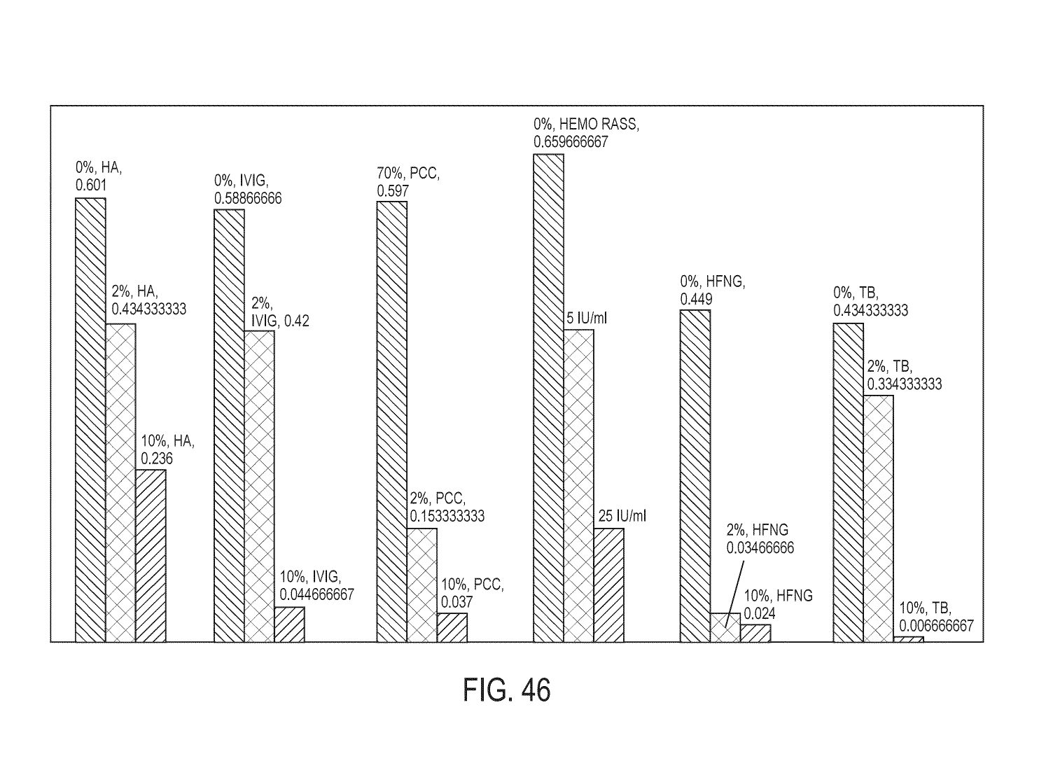

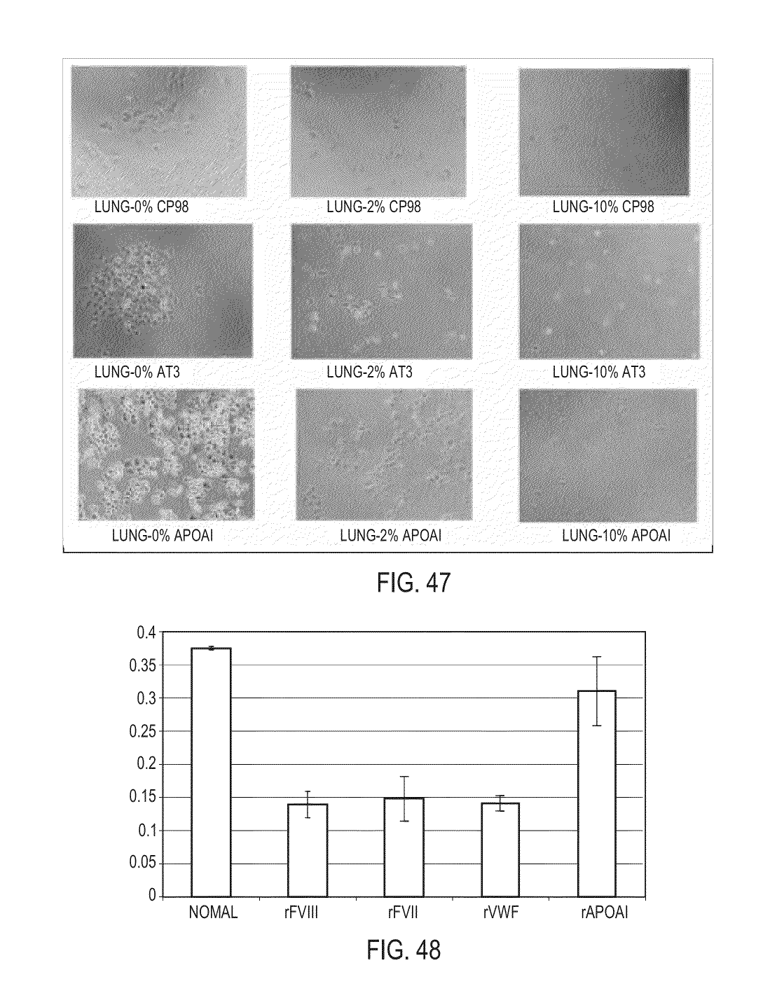

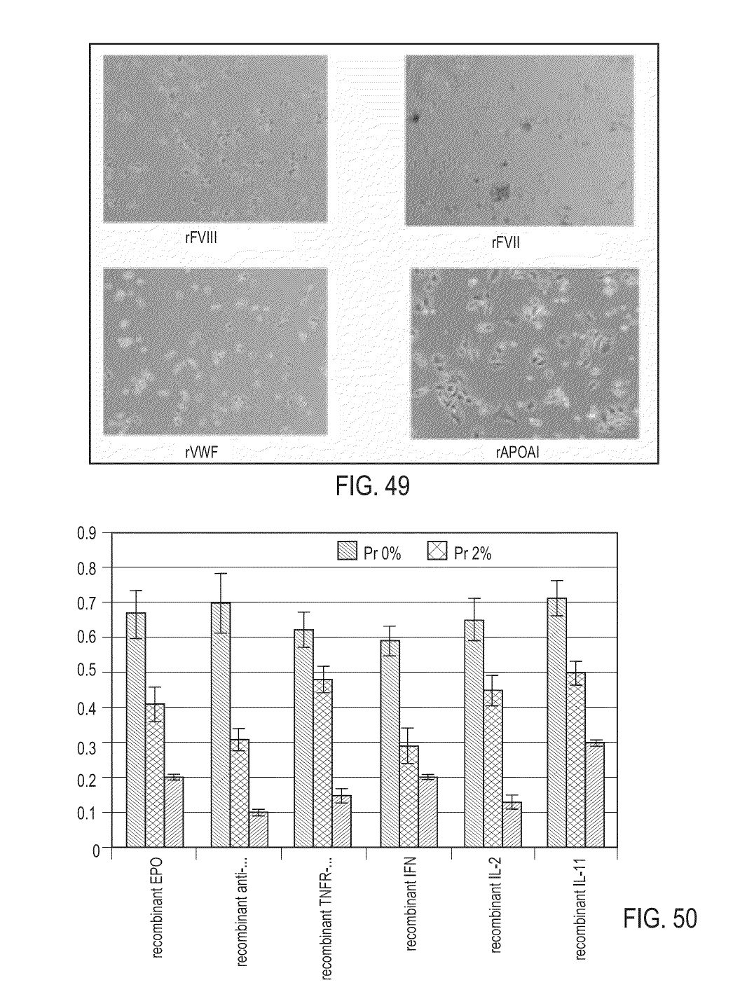

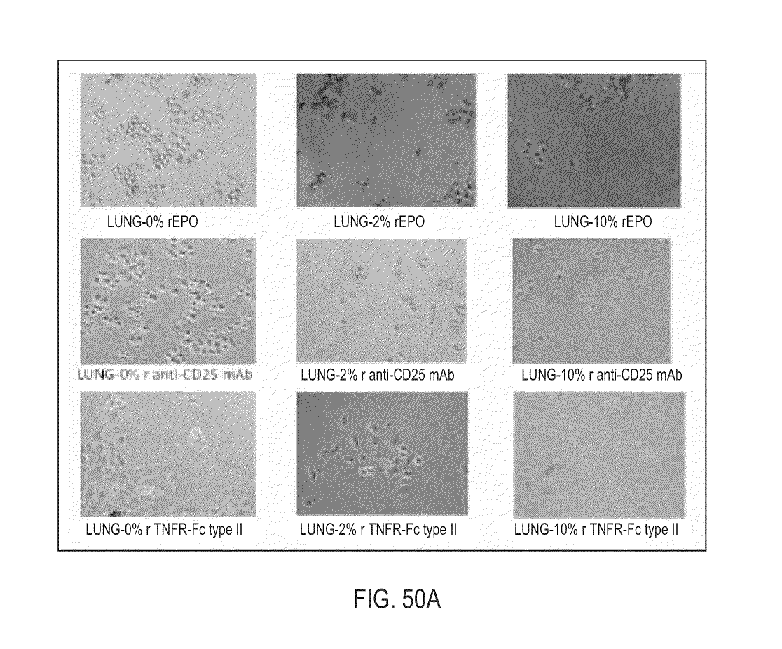

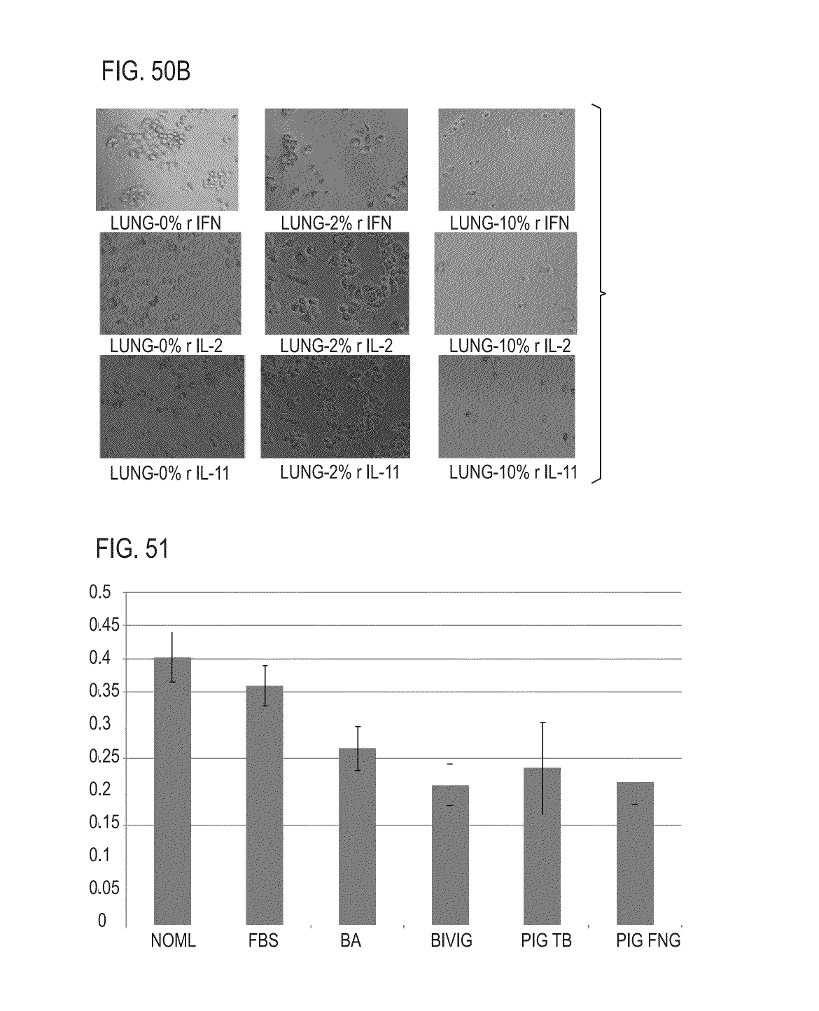

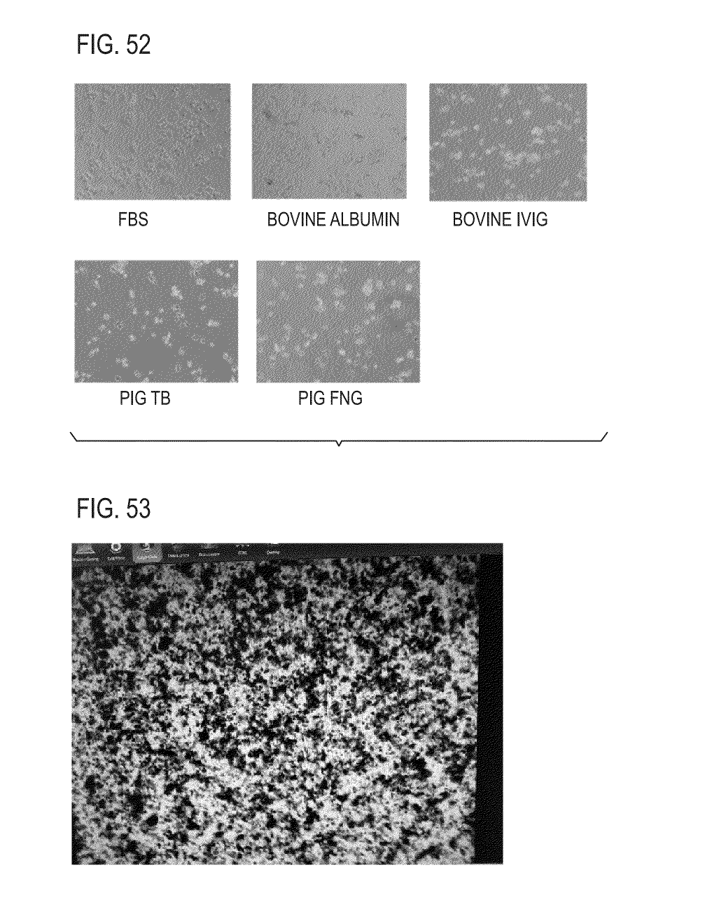

FIG. 46
FIG. 47
FIG. 47a
FIG. 48
FIG. 49
FIG. 50
FIG. 50a
Figure SOb
FIG. 51
FIG. 52.

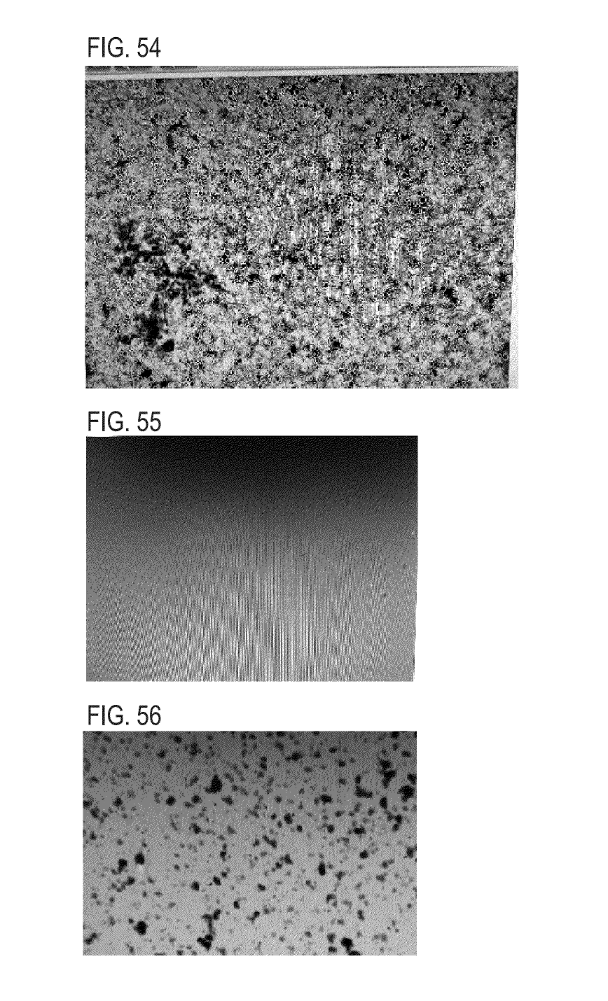

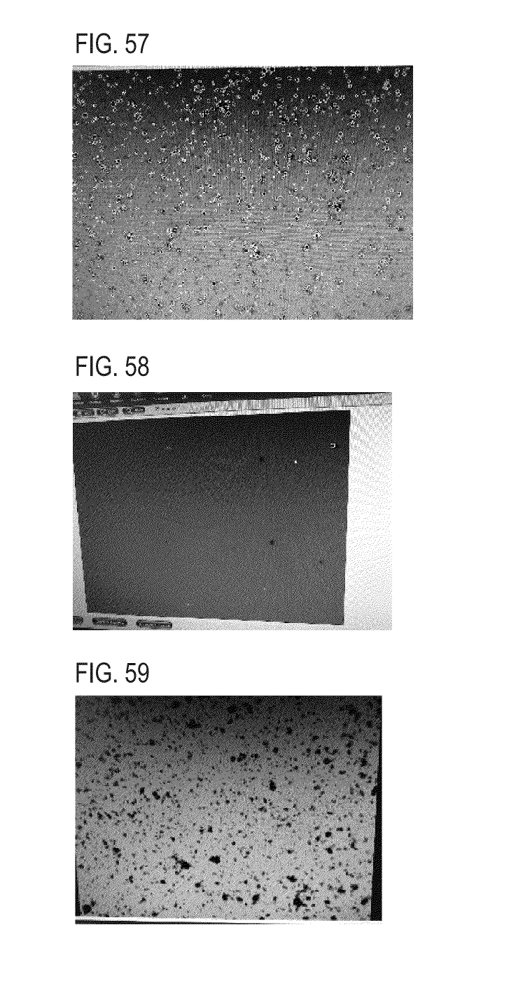

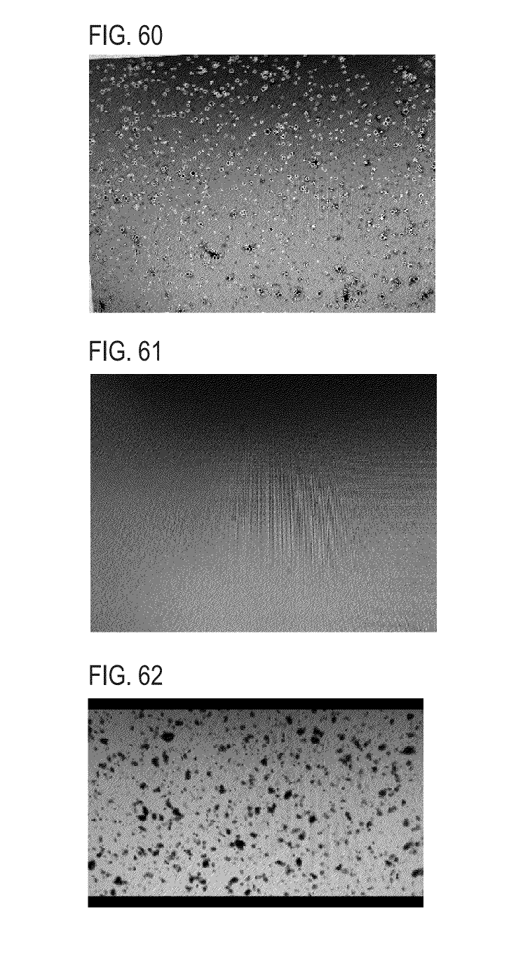

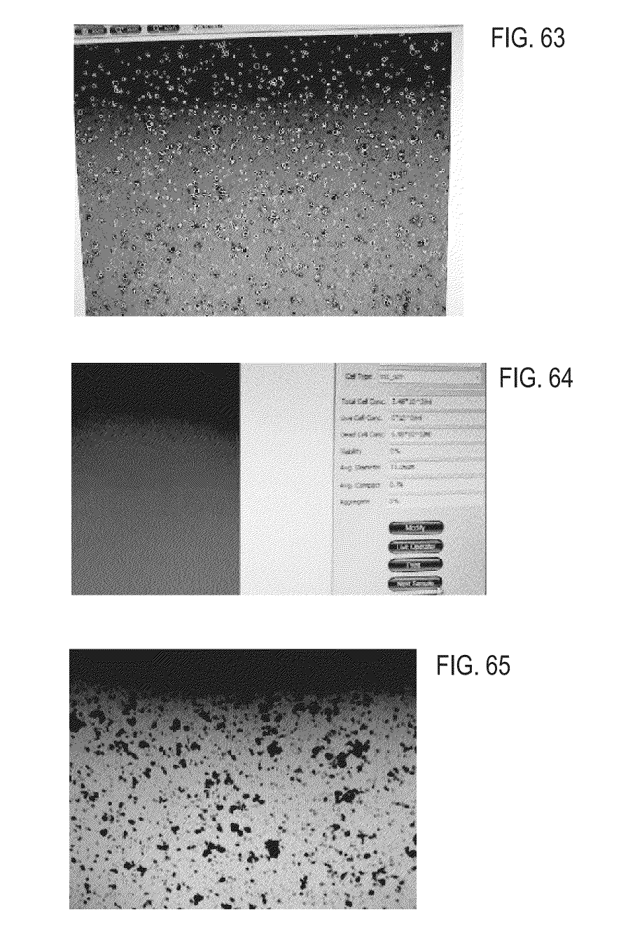

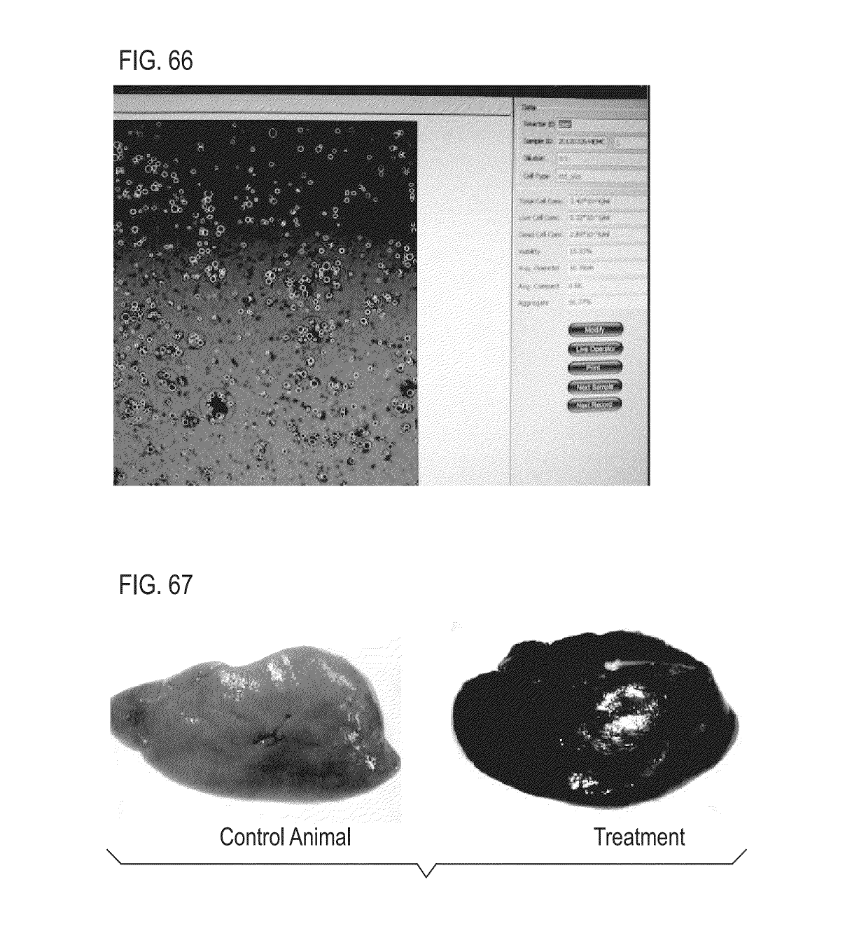

In vitro studies of the KH mediums using to express the cultured cells in order to obtain a desired protein.
KH 101 Medium Alone
KH1011 VIedium alone FIG. 53.
KH101 medium alone—Nearly 20 million cells
FIG. 54
KH 101Medium with AFCC product
AFC:C: alone-8,000 cell count
FIG. 55
AFCC with KH101 medium
FIG. 56
AFCC with KH101 medium after 5 days 4.5 million cell count
FIG. 57
KH 101 Medium with APOA1 product
APOA1alone - - - 20,000 cell count
FIG. 58
APOA11Nith KH101 Medium
FIG. 59
APOA1 with KH101medium after 5 days 4 million cell count
FIG. 60
KH 101 Medium with AFOD Product
AFOD alone-10 000 cell count
FIG. 61
AFOD with KH101 medium
FIG. 62
AFOD with KH101 medium after 5 days - - - 4.6 million cells
FIG. 63
KH 101 Medium with Factor VIII product
Factor VIII alone-5,400 cells
FIG. 64
Factor VIII with KH101medium
FIG. 65
Factor VIII with KH101medium after 5 days-3.4 million
FIG. 66

### In Vivo Studies

The study of APOAI protein in preventing atherosclerosis and related cardiovascular diseases

Study conducted hl: Fudan University, Zhang Jiang cmnpus

### Department: School of Pharmacy, Fudan University

Original data kept in: School of Pharn lacy, Fudan University

The current study was designed to investigate the human serum APOAI protein in preventing the atherosclerosis. New Zealand rabbits were adopted in this animal study and divided into 5

groups. They were high dose, medium dose and low dose of treatment, positive and vehicle control. The treatment groups were given APOAJ via auricular vein once a 1 veek Vehicle controls received normal saline via auricular vein once a week. Positive controls were given Liptor daily by p.o. with a dose of 0.45 mg/kg body weight. The body Weight of animal was determined every week and whole blood was drawn every three weeks. The study duration was 19 weeks. At the end of study, all animals were sacrificed. The important organs like liver, heart, kidney, aorta, and arteria carotis were observed in gross and pathological sections. Lipid content ‘lvas examined in liver and aorta. And liver index was also determined. Results showed that there was no significant change in body weight. The HDL-C was significantly high in ail treatment groups when compared with vehicle control. Although the liver index was lower in treatment group, but there's no statistical difference found. The area of atherosclerosis was significant less in medium group when compared with vehicle control. The pathological examination showed that there was no calcification found in either vehicle control or treatment group. However there was one animal with calcification in positive control group. The pathological change of aorta was better in medium group when considering endothelium swelling, smooth muscle migrating and foam cell formation compared with vehicle control. But there is no significant improvement in low dose group. The cellular swelling and fat degeneration was better in the liver of medium than that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.

### Purpose of the Experiments:

To investigate the human serum APOAI in in preventing atherosclerosis and related cardiovascular diseases and provide experimental basis for clinical application.

Methods and materials

1, Tested Reagent

Product name: human Apoiipoprotein AI, injection Produced

By: Shanghai RAAS Blood Products Co. Ltd. Lot number:

Size: 50 mg/mL

Appearance: colorless liquid

Positive control: Liptor

2, Animal

Strain: New Zealand vvhite rabbit

Vendor: Shanghai JieSiJie Laboratory Animal Co., Ltd

Qualification number:

Sex: male

Body weight: 1.8-LO kg

3 High Fat Diet Recipe

1%) cholesterol +99 normal diet, provide by Shanghai SiLaiKe Laboratory Animal Center

4 Experimental Design

4.1 Model

Male New Zealand white rabbits were used in this study. The body weight was between 1.8-2.0 kg. The animals were quarantined fix 5-10 days With normal diet before study. Blood samples were taken 12 hour after fasting before study to determine the blood lipid parameters.

4.2 Group

Animals were randomly divided into 5 groups including vehicle control, high dose, medium dose, lmv dose and positive control group. Ten to 14 rabbits were in one group. Each rabbit was fed with 30 gram of high fat diet fo llmved by 120 gram of normal diet with free access to lvater.

Housing condition: Ordinary Animal Lab with temperature of 24J-:20C and humidity of 55<%±10%.

4.3 Administration

First dose was given 1 week before high fat diet. The frequency of dosing was once a week Dose was 80, 40, 20 mg/kg body weight respectively. Drug was given by intravenous injection via auricular vein with the volume of 5 mL

Liptor 1 vas given by intragastric administration

5 Parameters Tested:

5.1 body weight: body weight of each rabbit was detem1ined once a Week.

5.2 blood lipid parameters: whole blood was drawn every three weeks. Animals were subject to 12 hour fast bef;xe taking blood. Resulted blood samples were kept still for 2 hours and then spin with 4,000 rpm for 10 min. The upper layer of serum was then separated and examined for total cholesterol (TC), total triglyceride (TG), low density lipoprotein cholesterin (LDL-C), and high density lipoprotein cholesterin(HDL-C). Test reagents were purchased from Shanghai Rong Sheng R10-pharmaceutical Co. Ltd.

5.3 Pathological Examination

A: The atherosclerosis of aorta (plaque area lj)

B: Liver index

C: Aorta, liver, heart, arteria carotis, kidney

### Resutls

1 The Establishment of Animal Model

Animals were f(d with high kd diet and treatment as described above. All blood lipid parameters significantly increased. There was no significant difference between vehicle control and treatment groups (data shown below). After 12 weeks of high fat diet, 1 animal in vehicle control or treatment group was sacrificed respectively. The liver of animal in vehicle control showed cream white in color and there was no atherosclerosis observed in aorta. There was no abnormal change in the liver and aorta of animal in treatment group. After 16 weeks of high fat diet, 1 animal of vehicle control was sacrificed and found about 20% of plaque on the inner surface of aortic arch. Animal continued to be fed with high fat diet and treatment for 3 more weeks. After 19 weeks of high fat diet, all animals were sacrificed.

2 Animal Procedures and Tissue Sampling

All animals were anesthetized by 20 of ethyl carbamate and then sacrificed with air injection. Abdomen cavity was opened. Whole blood was taken from heart. 1-Ieart was harvested along with 7 em of aorta. Then other organs like liver, kidney and arteria carotis were harvested.

Connective tissue was stripped from resulted organs or tissues followed by washing in normal saline fix 3 times. Pictures were taken then.

Aorta was cut from aortic arch, opened longitudinally and taken picture. The aorta \vas dissected for 0.5 em from aortic arch, split longitudinally and then kept in cryo-preservation tube for later lipid analysis. One piece of this sample was fixed in fomlalin for further pathological analysis.

The weight of liver was determined immediately. Two pieces of specimen were cut from hepatic lobe. One was kept in cryo-preservation tube for lipid analysis and another one was fixed in formalin for ftniher pathological analysis.

One piece of kidney sample was taken from renal pelvis and fixed in fomlalin for further pathological analysis.

Arteria carotis was dissected, cleaned and fixed in Formalin for further pathological examination.

The Formalin solution was replaced by fresh one about 4 hours and sent to pathological depmiment for pathological section.

3 Results

3.1 Change of Body Weight

The body weight of each animal was detem1ined before high fat diet and once a week thereafter. The change of body weight in each group lvas shmvn in table 1.

TABLE 1
The change of body weight in different groups

| Group | | | | |
|---|---|---|---|---|
| (animal | \VkO | ′\Vk 19 | [ncrease | lncrease |
| number) | (kg) | (kg) | (kg) | (%) |
| Vehicle | 1.94 ± 0.231 | 3.23 ± 0.284 | 1.29 ± 0.361 | 66.5% |
| (n = 9) | | | | |
| High dose | 1.68 ± 0.078 | 3.49 ± 0.221 | 1.81 ± 0.209 | 107.1%; |
| (n === 8) | | | | |
| Mediumdose | 1.8 ± 0.22 | 2.99 ± 0.52 | 1.18 ± 0.286 | 65.5% |
| (n = 9) | | | | |
| Low dose | 2.L1-AU74 | 3.19-.-i-:( ).278 | 1.09 . + :JL529 | 51.9% |
| (n === 12) | | | | |

3.2 Plasma Lipid Parameters

Animals were fast for 12 hours before taking blood samples via auricular vein. Resulted blood samples were kept stii1f;x 2 hours. The upper layer of serum lvas then separated and examined ±or total cholesterol (TC), total triglycelide (TG), lmv density lipoprotein choleste lin (LDL-C), and high density lipoprotein cholesterin (HDL-C). Test reagents were purchased from Shanghai Rong Sheng Bio-pharmaceutical Co. Ltd.

TABLE 2
Change of total triglyceride (TG)

| Group | | | | |
|---|---|---|---|---|
| (animal | ′\VkO | ′\Vk 19 | Increase | Increase |
| number) | (mmol/L) | (mmol/L) | (mmol/L) | (%) |
| Vehicle | 0.823J:0.294 | 1.864-.-H).871 | 1.04H.-0.933 | 126.5 |
| (n = 9) | | | | |
| Mediumdose | 0.656 ± 0.19j | 2.j44 ± 1.043 | 1.488 ± 0.988 | 226.8% |
| (n = 9) | | | | |
| l,ow dose | 0.786 ± 0.229 | 1.267 ± 0.772 | 0.482 ± 0.839 | 61.3lj |
| (n = 12) | | | | |

TABLE 3
Change of total cholesterol (TC)

| Group | \VkO | ′\Vk 19 | Increase | Increase |
|---|---|---|---|---|
| (anim.al mnnber) | (mmol/L) | (mmol/1,) | (m.moliL) | (%) |
| Control | 1.15 ± 0.23 | 8.049 ± 2.99 | 6.896 ± 3.03 | 598.3% |
| (n = 9) | | | | |
| High dose | 1.59.t-J}.48 | 12.49-t-2.81 | 10.90J:2.66 | 685.5% |
| (n === 8) | | | | |
| Mediumdose | 1.77 ± 0.783 | 10.28 ± 5.82 | 8.505 ± 5.37 | 453.0% |
| (n = 9) | | | | |
| l,ow dose | 1.06.-i-:0.27 | 9.07-. + :4.92 | 8.0Lt-A.87 | 755.6% |
| (n = 12) | | | | |

TABLE 4
Change of high density lipoprotein cholesterin (HDL-C)

| Group | \VkO | \Vk19 | Increase | Increase | |
|---|---|---|---|---|---|
| (animal Iunnber} | (m.moliq | {m.moliq | (m.moliq | C %} | Sig |
| Control(n = 9) | 0.94 ± 0.262 | 3.527 ± 2.007 | 2.588 ± 1.918 | 275.3%) | |
| High dose (n = 8) | 1J83 + 0.149 | 4.993·- + :2.018 | 3.8H2.025 | 322.1-′0 | 0.( )35* |
| Mediumdose(n = 9) | 0.67 ± 0.207 | 4.343 ± 2.439 | 3.674 + 2.413 | 548.4% | o.ol |
| Low dose (n = 12) | 0.705 ± 0.246 | 3.744 ± 2.14 | 3.04 ± 2.019 | 431.2′% | 0.028* |

p < 0.05

TABLE 5
Change ofligh density lipoprotein cholesterin (LDL-C)

| Group | \VkO | Wk 19 | Increase | Increase |
|---|---|---|---|---|
| (anim.al mnnber) | (rnmol/I.) | (rnrnol/L) | (mm.ol/L) | (%) |
| Control(n = 9) | 0.872 ± 0.386 | 5.826 + 2.909 | 4.954 ± 2.953 | 568.1% |
| High dose (n = 8) | 0.92 ± 0.324 | 14.1 ± 4.188 | 13.18 + 4.053 | 1432.6% |
| Mediumdose(n ==== 9) | j.06 ± 0.298 | 6.357 ± 4.475 | 5.297 + 4.373 | 499.7%; |
| Low dose (n = 12) | 0.826 ± 0.279 | 7.298 + 4.60 | 6.472 ± 4.468 | 783.5 · |

TABLE 6
Liver index

| Group | Body weight | Liver weight | Uver index | |
|---|---|---|---|---|
| (animal number) | (kg) | (g) | (%) | Sig |
| Control(n === 9) | 3.083:1:.0.279 | 123.08-+.-22.31 | 3.984:1:.0.579 | |
| High dose (n = 8) | 3.565 ± 0.205 | 151.69 ± 18.49 | 4.257 ± 0.482 | 0.26 |
| Mediumdose(n = 9) | 3.009-.-i-:0.554 | 112.006-- + .-25.79 | 3.708-.-i-:( ).391 | 0.267 |
| Low dose (n = 12) | 3.3 ± 0.329 | 128.096 ± 20.43 | 3.886 ± 0.489 | 0.571 |

3.3 Plaque Area of Aorta

The aorta was dissected and opened for 7.5 em from aortic arch longitudinally. Pictures were taken and atherosclerosis changing was analyzed. The area of atherosclerosis was graded by clinical standard according to its area to whole area of dissected aorta, by which grade I was less than 25 ?-),grade H ‘lvas behveen 25% to 50%, grade HI was behveen 50% to 75% and Grade IV was greater than 75%.

TABLE 7
atherosclerosis change in vehicle control group

| Animal number | Plaque area/amia area | Grade |
|---|---|---|
| 5 | 8.62 | I |
| 6 | 16.67 | I |
| 7 | 37.5 | n |
| 9 | 39.47 | II |
| 11 | 1.67 | |
| 12 | 10 | I |
| 17 | 92.86 | IV |
| 18 | 70.91 | n |
| 19 | 25.17 | II |

Grade I: 4 animals; Grade II: 4 animals; Grade HI: 0 animal; Grade IV: 1 anirnai.

TABLE 8
atherosclerosis change in low dose group

| Animal number | Plaque area/aorta area | Grade |
|---|---|---|
| 31 | 10 | I |
| 32 | 26 | II |
| 36 | 1.92 | I |
| 37 | 76.79 | III |
| 38 | 11.11 | I |
| 39 | 2.88 | I |
| 40 | 6.67 | I |
| 41 | 2 | I |
| 42 | 92 | IV |
| 43 | 6.67 | I |
| 44 | 0.18 | I |
| 48 | 23.36 | I |

Grade I: 9 animals; Grade II: 1 animal; Grade HI: 0 animal; Grade IV: 2 animals.

Statistical analysis of low dose group: Mann-Whitney test

| Grade 0 | | [symbol] | | | | | | I | | I | |
|---|---|---|---|---|---|---|---|---|---|---|---|
| Levell | I | | I | I | [symbol] | [symbol] | I | | I | | [symbol] |
| Theoretic | J | 2 | 3 | 4 | 5 | 6 | 7 | 8 | 9 | 10 | 11 |
| level | | | | | | | | | | | |
| Level | 7 | 7 | 7 | [symbol] | 7 | [symbol] | 7 | 7 | [symbol] | [symbol] | [symbol] |
| Level 0 | | 7 | | | | | | 7 | | 7 | |
| Levell | 7 | | 7 | [symbol] | [symbol] | 7 | 7 | | 7 | | 7 |
| Grade 0 | I | | n | | [symbol] | [symbol] | III | | | IV | |
| Levell | | [symbol] | | [symbol] | | | | m | IV | | |
| Theoretic | 12 | 13 | 14 | 15 | 16 | 17 | 18 | 19 | 20 | 21 | |
| level | | | | | | | | | | | |
| Level | 7 | 7 | 15.5 | 15.5 | 15.5 | 15.5 | 18.5 | 18.5 | 19.5 | 19.5 | |
| Level 0 | 7 | | 15.5 | | 15.5 | 15.5 | [symbol] 8.5 | | | 19.5 | |
| Levell | | [symbol] | | 15.5 | | | | 18.5 | 19.5 | | |

Level sum in Vehicle control 112.8
Level sum in lovv dose group: 116.5
To.os′″71 T > To.os no statistical difference [symbol]
indicates data missing or illegible when filed

[symbol]

TABLE 9
atherosclerosis change in medium dose group

| Animal number | Plaque area/a01ia area | Grade |
|---|---|---|
| 21 | 36.53 | II |
| | 1.69 | |
| 23 | 18.75 | I |
| 25 | 19.17 | I |
| | 11.67 | I |
| 28 | 1.82 | I |
| 29 | 61.67 | II |
| 30 | 1.6 | I |

Grade I: 6 animals; Grade II: 2 anirna!s; Grade III: 0 animal; Grade IV: 0 animaL

Statistical analysis oflovv dose group: Mann-Whitney test

### Level 2 15.5 13

Level sum in Vehicle control: 112.8
Level sum in low dose group: 46
To.os=51 T<To.o.s statistical difference

TABLE 10
atherosclerosis change in high dose group

| Animal number | Plaque area/a01ia area | Grade |
|---|---|---|
| 50 | 62.5 | II |
| 51 | 100 | IV |
| 52 | 56.88 | II |
| 53 | 40.13 | II |
| 54 | 100 | IV |
| 55 | 27.19 | II |
| 60 | 68.03 | II |
| 62 | 95.00 | IV |

Grade I: 0 animal; Grade II: 5 animals; Grade III: 0 animal; Grade IV: 3 animals.

3A Pathological examination
3A.l A01ia

The pathological change was better in medium group when considering endothelium svveiling, smooth muscle migrating and foam cell formation compared with vehicle control. But there is no significant improvement in low dose group

3.4.2 Liver Gross and Pathological Examination

The cellular swelling and fat degeneration was better in the liver of medium than that of vehicle control. Although the cellular swelling was same in low dose group and vehicle control but the fat degeneration ‘lvas better in liver of low dose group than that of vehicle control.

3.4.3 Hemi, Arteria Carotis and Kidney

There was no pathological change found in heart and kidney either in vehicle control or treatment groups. There was no atherosclerosis change found in Arteria carotis.

3.4.3 Lipid Content in Tissues

1) Lipid Content in Liver

| | Control | Lmv dose | Middle | High |
|---|---|---|---|---|
| TC | 3.056 ± 0.775 | 2.95 ± 0.809 | 2,214 ± 0.515 | 2.841 ± 0.298 |
| TG | 1.817 ± 0.446 | 1.369 ± 0.251 | 1.081 ± 0.31 | 1.3 ± 0.171 |
| HDL- | 0.712 ± 0.244 | 0.803 ± 0.236 | 0.815 ± 0.249 | 0.825 ± 0.129 |
| C | | | | |
| LDL- | 2.035 ± 0.328 | [.857 ± 0.559 | 1.407 ± 0.418 | 2.302 ± 0.054 |
| C | | | | |

Statistics analysis of lipid content in liver

| | Lovv dose | Medium | High |
|---|---|---|---|
| TC | 0.775 | 0.(22 | 0.564 |
| TG | 0.022 | 0,,0it | 0.009 |
| HDL-C | 0.81 | 0.74 | 0.684 |
| LDL-C | 0.436 | OJ)]1 | 0.989 |

The lipid content in liver showed that TG in low and high dose group was significantly lower than that in vehicle control. The TC, TG and LDL-C in medium group were significantly lower than those in vehicle control.

2) Lipid Content in Amia

| | Control | Lmvdose | Middle | High |
|---|---|---|---|---|
| TC TG | 0.331 ± 0.097 | 0.28 ± 0.047 | 0.332 ± 0J35 | 0.29 ± 0.098 |
| ElDL-C | 0.406 ± 0.178 | 0.337 ± 0.055 | 0.388 ± 0.124 | 0.402 ± 0.101 |
| LDL-C | 0.065 ± 0.032 | 0.092 ± 0.066 | 0.128 ± 0.064 | 0.111 ± 0.057 |
| | 0.323 ± 0.116 | 0.254 ± 0.078 | 0.307 ± 0.043 | 0.318 ± 0.05 |

Statistics analysis of lipid content in aorta

| | Lovv dose | Medium | High |
|---|---|---|---|
| TC | 0.387 | 0.879 | 0.483 |
| TG | 0.341 | 0.80 | 0.952 |
| HDL-C | 0.416 | 0.065 | 0.171 |
| LDL-C | 0.138 | 0.73 | 0.912 |

The lipid content in aorta was lower in treatment groups than that in vehicle control but there was no statistical significance.

### Summary:

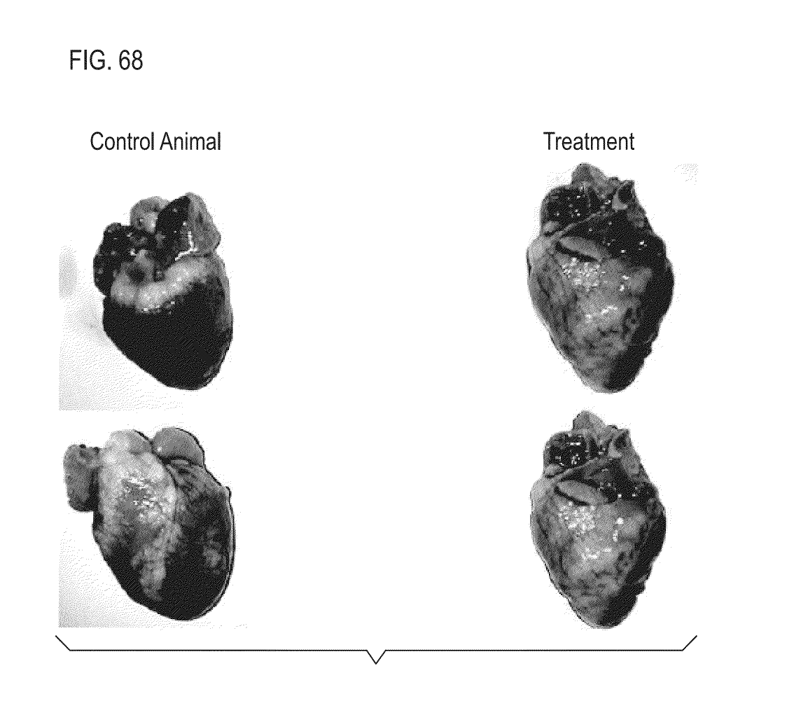

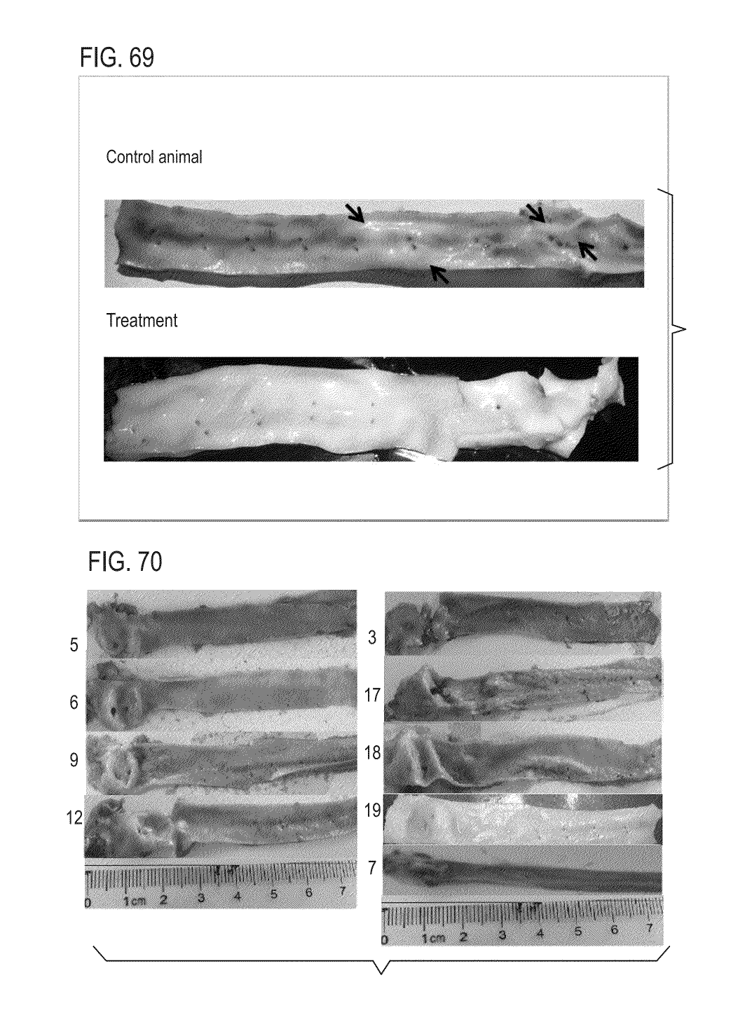

This study vvas designed to investigate the prevention efficacy of APOAi in atherosclerosis. The test article was given along with high fat diet which caused no significant decrease in blood lipid parameters. However the treatment significantly increased the HDL-C level in all treated groups. There was no dose escalation effect found in three treatment groups upon anatomic, pathological and biochemistry examination. It has been showed that the atherosclerosis in medium dose group was significantly less than that in vehicle control. The pathological change was better in medium group when considering endothelium swelling, smooth muscle migrating and foam cell formation in aorta compared \vith vehicle control. But there is no significant improvement in low dose group. The cellular swelling and fat degeneration was better in the liver of medium than that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.
FIG. 67
FIG. 68
FIG. 69

From vehicle and treated two rabbits, sacrificed and operated to determine the fat build up during the first 8 lveeks of the study.

Appendix 1: pictures of amia
Vehicle control
Low dose group FIG. 70 [symbol]
Medium dose group

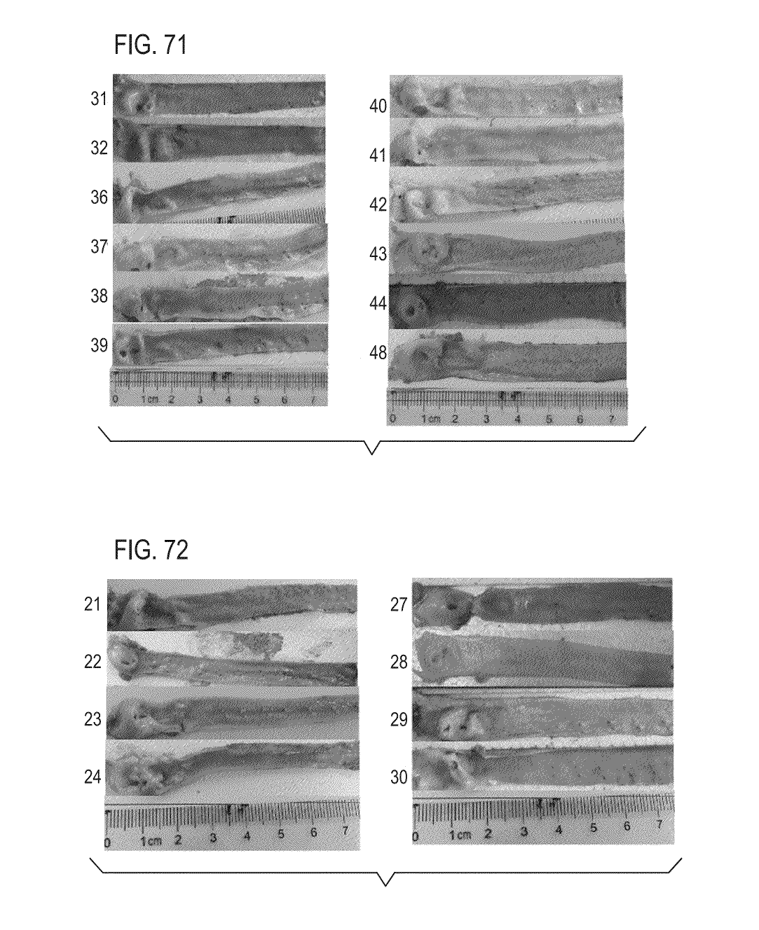

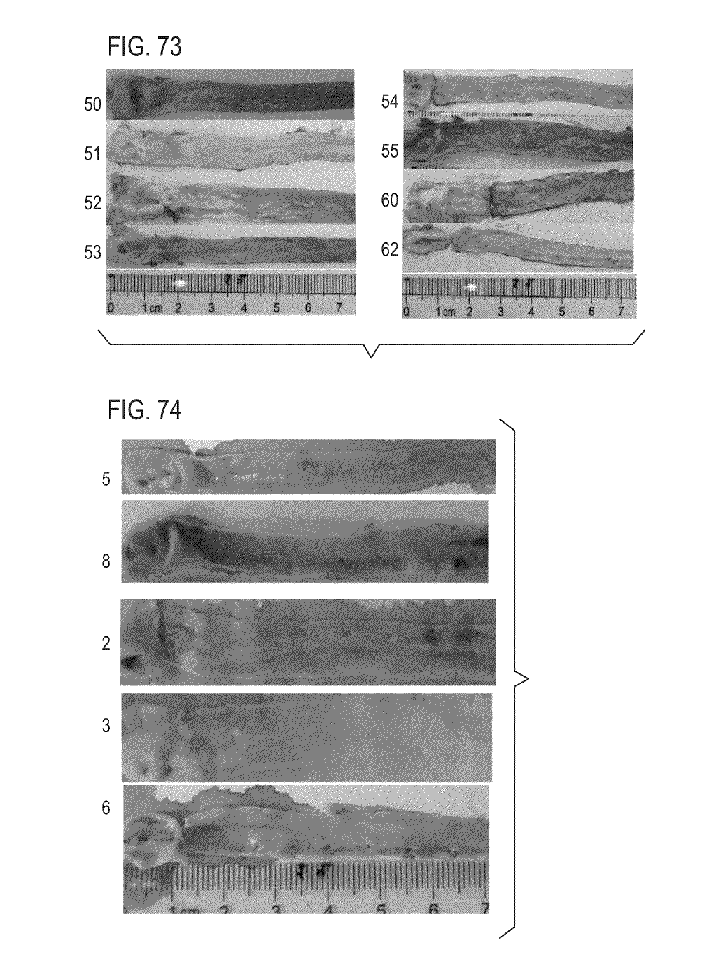

### FIG. 71

FIG.
72
   High dose group
FIG.
73 [symbol] [symbol] [symbol]
   Positive control (Lipitor)
FIG.
74

The lipid profile results and quantification of atherosclerosis pla(JUe in 18 i\poE tnice for 4 ‘veeks Study®

18 male Apo E (−/−) were fed with HFD/High cholesterol diet starting on .hn.11, 2012

′″18 Apo E{−/−) mice were assigned to 4 groups based on the BW,TC, HDL level after fed with HFD for 4 weeks and all mice were treated with test articles starting nn
Fdd3, 2012.
Vehicle
ApoA1 0.2 ml iv/ip n=5
AFOD 0.2 ml iv/ip n=4
AFCC 0.2 ml iv/ip n=4

Collected 300 ul of blood for lipid profile measurement on 13 Mar. 2012 after 14dose (S wks) treatment. AH the mice were sacrificed on March 16 and all AORTA were dissected for atherosclerotic plaque analysis by oil red staining later.

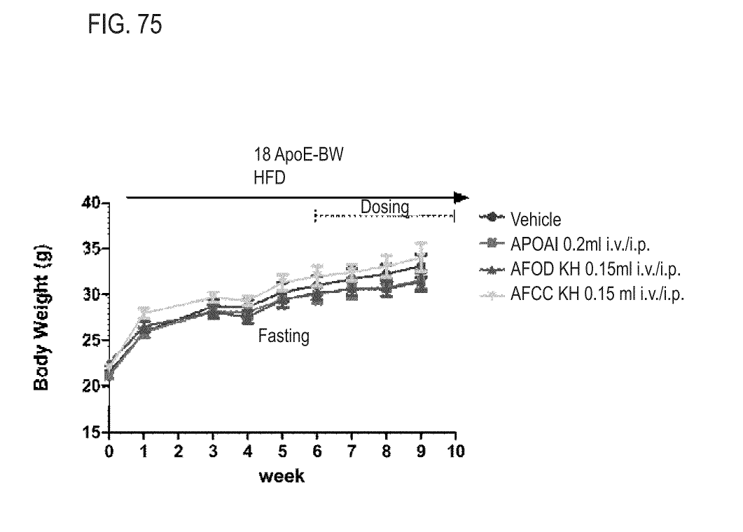

Body weight in 18 ApoE mice
FIG. 75
′″t ooks Hk 2:thn$ ni: bods d-dn't dL:sturt3 th:3 ner 3.: 3E3 Gf bt>dy ‘N·3j 1ht;n tho: 3=mk:3 aftr 6 . . . |E3 k tr trn·n

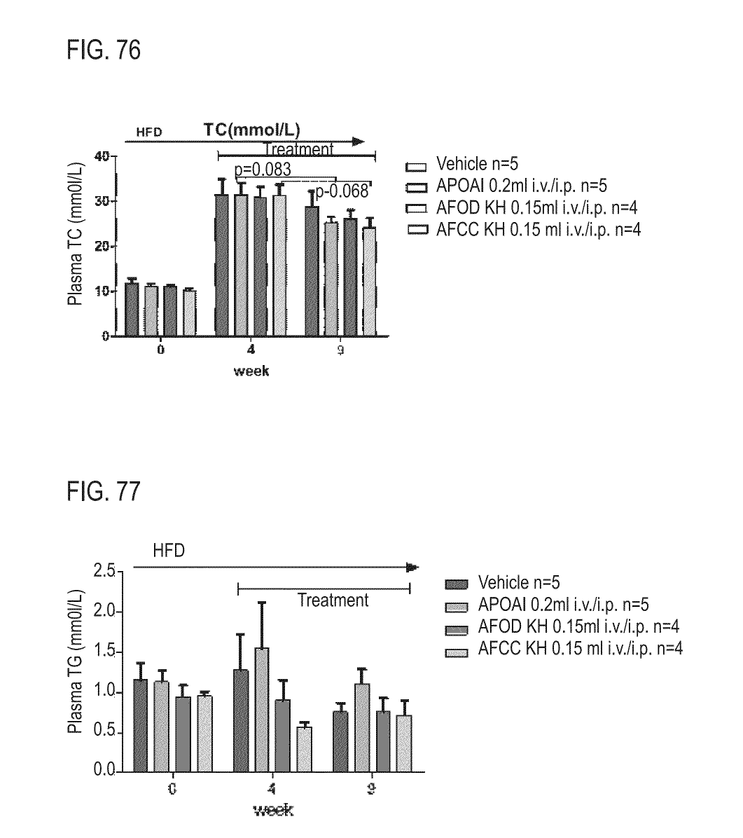

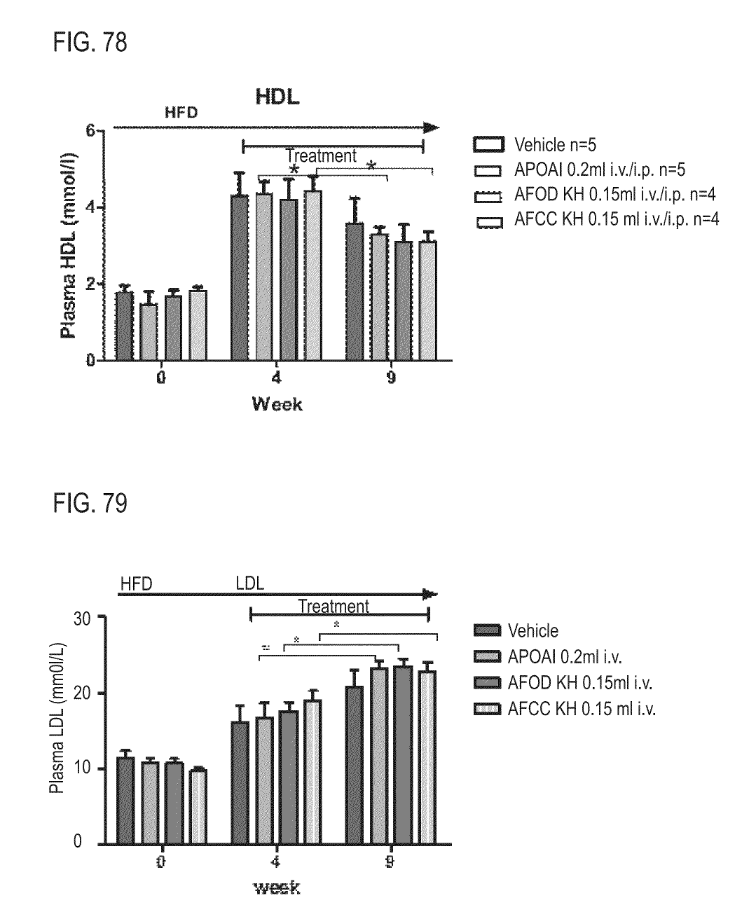

Blood plasma lipid profile at three time points in 18 Apo E(−/−} mice
FIG. 76
FIG. 77
FIG. 78
FIG. 79

18 Apo E(−/−) mice at 8 weeks old were fed with HFD/High Cholesterol diet for 4 weeks. Then were treated with AFCC, APOAh nd AFOD for 5 weeks. It looks like three antibodies didn't improve the lipid profile in those mice after 5 weeks treatment.

Three time points: 0 week: Before HFD; 4 week: Fed with HFD for 4 week; 8 week; After 4 weeks treatment

### Illustration of AORTA

Sites of predilection for lesion development are indicated in black:

(1) aortic root, at the base of the valves;

(2) lesser curvature of the aortic arch;

(3) principal branches of the thoracic aorta;

(4) carotid artery;

(5) principal branches of the abdominal aorta;

(6) aortic bifurcation;

(7) iliac artery; and

(8) pulmonary arteries.

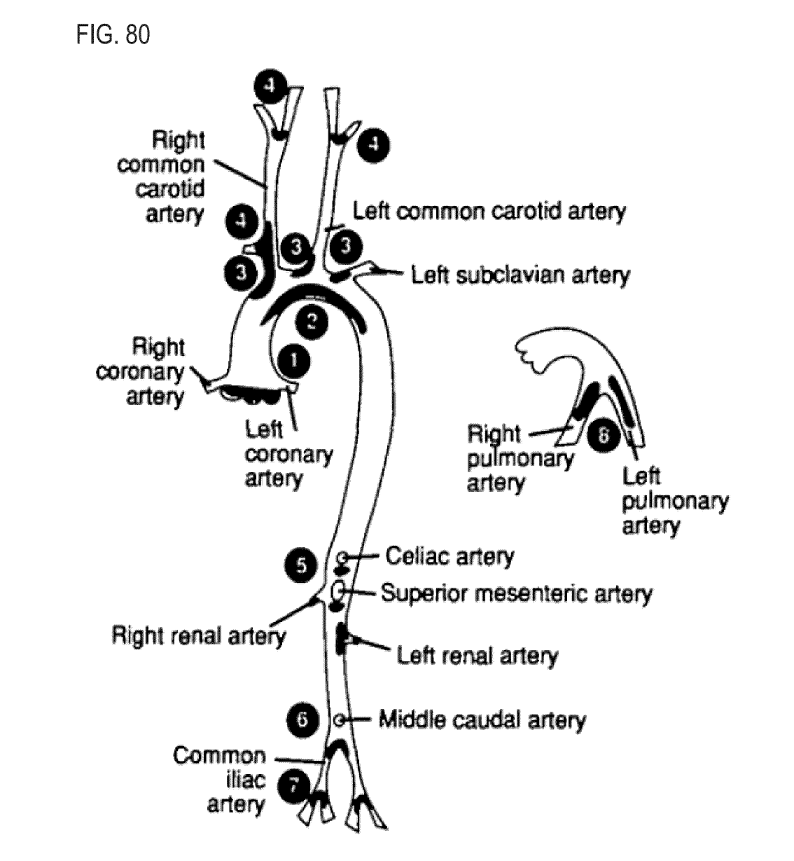

Quote frorn Y Nakashirna, 1994
FIG. 80

Oil Red staining procedure:
Sacrificed the mice and heart, aorta, and arteries were dissected under the dissecting microscope. Briefly wash with PBS and fixed in 4% paraformaldehyde (PFA) overnight at 4° C.
Rinse with 60% isopropanol
Stain with freshly prepared Oil Red 0 working solution 10 mins.
   Oil red 0 stock stain:0.5% powder in isopropanol
      Working solution: dilute with distilled water (3:2) and filter with membrane
Rinse with 60% isopropanollO second.

Dispel the adherent bit fat outside of the aorta under the dissecting microscope.
Cut the vascular wall softly and keep the integrated arteries using the microscissors.

Unfold the vascular inner wall with the cover and slides glass and fix it by water sealing tablet.

### Image Analysis Procedure:

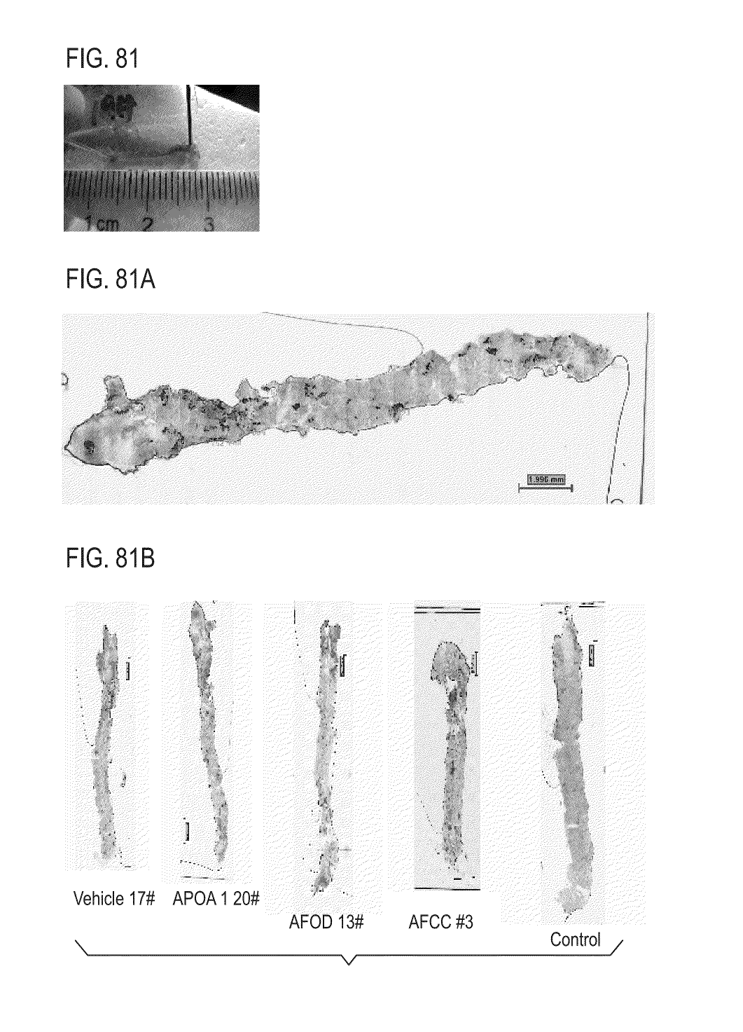

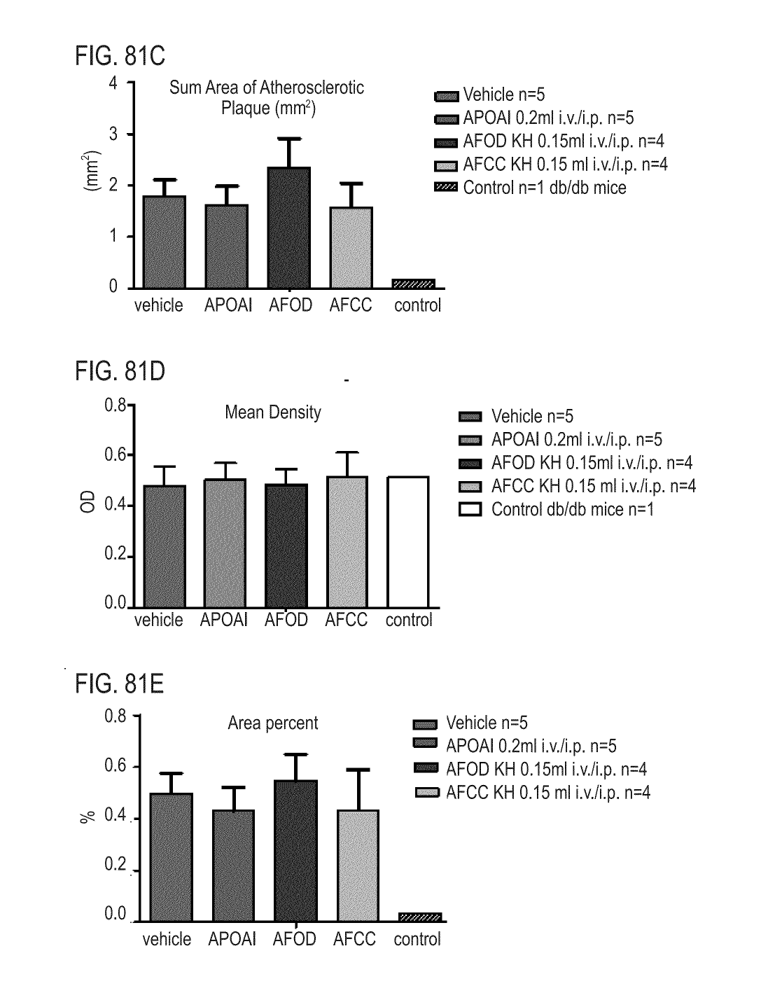

The unfolded vascular inner wall “I.Vere scanned with Aperio ScanScope system and the area of atherosclerotic plaque was measured by Image-Proplus software after oil red O staining as follow picture shmvn.
FIG. 81a.

### Photos:

FIG. 81b.

### Results:

\Ve measured the sum lesion areas and mean density using ipp software and calculated atherosclerotic percent.

Area percent(%)′″ Sum area of atherosclerotic plaque (mm²)/whole area of vascular inner wall (mm²)
FIG. 81c
FIG. 81d
FIG. 81e

### Summary:

The atherosclerotic plaques/lesions were obviously labeled in the luminal surface area of the aorta compared with the control. This results is consistent with the published literatures. The atherosclerotic animal model was established in Apo E(−/−) mice fed with the high fat diet for 9 weeks.
ApoAl shmved a trend on reducing the atherosclerotic plaques/lesions compared to the vehicle group after 14 dosing.
Reference:
Y Nakashima et al. ApoE-deficient mice develop lesions of all phases of atherosclerosis throughout the a lierial tree. Arteriosclerosis and Thrombosis Vol 14, No 1 Janumy 1994
1 inal Report of Efficacy Study on
   RAAS AFOD RAAS 1 (APOA1) in ApoE mice for 8 weeks

Study Title: Efficacy study of 4F( )D RAAS 1 (AP( ) 41) on atherosclerosis model in ApoE nlice

Study Number: CPB-P1′-2504-RAAS

Date: Jun. 29, 2012

1. Abbreviations and Definitions

[symbol]

| | |
|---|---|
| kg | kilogram g |
| | gram |
| Mg | milligram |
| ng | Nanogram ml |
| | Milliliter |
| !JL | microliter |
| h | hours min |
| | minutes Cpd |
| | Compound |
| BW | Body Weight |
| BG | Blood Glucose |
| FBG | Fasting Blood Glucose |
| DOB | Date of Birth |
| TC | Total Cholesterol |
| TG | Triglyceride |
| LDL | Low Density Lipoprotein |
| HDL | High Density Lipoprotein |
| FBW | Fasting Blood Glucose |
| SO | Standard Deviation |
| SE | Standard error |
| i.p | Intraperitoneal injection |
| PFA | paraformaldehyde |

2. Introduction

The study described in this report evaluated in vivo efficacy of RAAS antibody APOA 1 in atherosclerotic nlodel.

3. Purpose

To evaluate the efficacy effect of RAAS antibody APOA I on plasma lipid profile, lesion plaque of inner aorta and related parameters in atherosderotic model.

4. Materials

4.1. Test artide: RAAS APOA I; Atorvastatin (Reference Compound)

4.2. Animal: ApoE Knock Out (ko) Mouse
Sex: male
Strain: C57BLKS
Vender: Beijing Vitol River
Age: 8 weeks (arrived on 23 Dec. 2011)
Number: 60

4.3. Upid Profile Test: Shanghai DaAn Medical Laboratory, Roche Modular Automatic Biochemistry Analyzer

4.4. Heparin Sodium Salt: TCL, H0393

4.5. Capillary: 80 mm, 0.9-1.1 mm

4.6. Ophthalmic Tweezers and Scissors: 66 Vision-Tech Co,. LTD, Suzhou, China. Cat#53324A, 54264TM

4.7. High Fat Diet:TestDiet, Cat#58v8(35% kcal Fat 1% chol)

4.8. Glycerol Jelly Mounting Medium: Beyotime, Cat# C0187.

4.9. Glucose Test Strips: ACCU-CHEK Performa: ROCHE (Lot#470396)

4.10. !mage Analyse: Aperio ScanScope System; Image-Proplus 6.0 Software; Aperio Image Scope Version 11.0.2.725 Software.

4.11. Aorta Staining: Oil Red 0 (A!fa Aesar) Isopropanol (Lab Partner)

5. Experiment Method

5.1. Grouping Mice:

10 ApoE ko mice were fed with regular chow diet and used as negative control group. 50 ApoE ko mice were fed with high fat diet (35% kcal fat, 1% cholesterol) for 8 weeks, and then the plasma samples were collected for lipid profile measurement before the treatment. 50 ApoE ko mice were assigned into 5 groups based on the fasting overnight plasma TC and HDL leveL The group information is shown in the table below.
Table 1 Information of groups

5.2. Study Timeline:

23 Dec. 2011: 60 ApoE mice arrived at chempartner and were housed in the animal facility in the building#3 for the acclirnation.

6 Jan. 2012: Measured the body weight for each mouse” 50 mice were fed with high fat diet and 10 mice were fed with normal chow diet”
2 Mar. 2012: Ail mice were fasted over night and plasma samples (about 300 ul whole blood) were collected for lipid profile measurement before treatment with RAAS antibody,
19 Mar. 2012 to 6 Apr. 2012: Group the mice based on the TC and HDL level and start the treatment with 3 doses of antibody APOA1 by i.p daily on the weekday (The first dose was administered by iv injection

through the tail vein. The reference compound atorvastatin was administered by oral dosing every day.

7 Apr. 2012 to 12 Apr. 2012: Stop dosing for 5 days. After 15 doses treatment with the antibody, several mice died in the treatment groups. The dient asked for stopping treatment for a while.

13 Apr. 2012-14 May 2012: The treatment with antibody APOA1was changed to i.p injection every two days (Monday, Wednesday, and Friday) per client's instruction.

17 Apr. 2012: All mice were fasted over night and plasma sample for each mouse (about 300 ul whole blood) was collected for lipid profile measurement after 4 weeks treatment.

14 May 2012: Ali mice were fasted over night and plasma sample for each mouse (about 300 ul whole blood) was collected for lipid profile measurement after 8 weeks treatment. Blood glucose was also measured for each mouse.

17 May 2012: The study was terminated after 8 weeks treatment. Measure BW, sacrificed each mouse dissected the aorta, heart, liver and kidney and fixed them in 4% PFA.

5.3. Route of Compound Administration:

Antibody products were administrated by intraperitoneal injection every two days (Monday, Wednesday, and Friday).and the positive compound was administered by p.o every day.

5.4. Body weight and blood glucose measurement: The body weight was weighed weekly during the period of treatment. The fasting overnight blood glucose was measured at the end of study by Roche glucometer.

5.5 24 h food intake measurement: 24 hours food intake for each cage was measured weekly

5.6. Plasma lipid profile measurement: About 300 ul of blood sample was collected from the orbital vein for each mouse and centrifuged at 7000 rpm for 5 min at 4° C. and the plasma lipid profile was measured by Roche Modular automatic biochemistry analyzer in DaAn Medical Laboratory

5.7. Study Taken Down:

After RAAS antibody products treatment for 8 weeks, all mice were sacrificed. Measured body weight and collected blood sample for each mouse. Weighed liver weight and saved a tiny piece of liver into 4% paraformaldehyde (PFA) fixation solution for further analysis. At same time, take the photos with heart, lung, aortas and two kidneys.

5.8. Oil Red Staining Procedure:

1. Sacrificed the mice and dissected the heart, aorta, and arteries under dissecting microscope.

2. Briefly wash with PBS and fixed in 4% paraformaldehyde (PFA) overnight at 40 C.

3. Rinse with 60% isopropanol

4. Stain with freshly prepared Oil Red 0 working solution 10 min.
1). Oil red O stock stain: 0.5% powder in isopropanol
2).Working solution: dilute with distilled water (3:2) and filter with membrane(0.22 um)

5. Rinse with 60% isopropanol 10 second.

6. Dispel the adherent bit fat outside of the aorta under the dissecting microscope.

7. Cut the vascular wall gently and keep the integrated arteries using the micro scissors.

8. Unfold the vascular inner wall with the cover slides and fix it by water seaHng tablet.

5.9. Image Scanning and Analysis:

Scanning the glasses slides with the Aperio ScanScope system and analyze with the image proplus software to measure the area of atherosclerotic plaque iession. The results were expressed as the percentage of the total aortic surface area covered by lesions. The operation procedure of software was briefly described as follow: Converted the sys version photos into JPG version, then calibrated it and subsequently selected the red regions and then calculate the total area automatically by image proplus software.

5.10. Clinic Observation:

Atorvastatin significantly reduced the body weight after 5 weeks treatment. APOA1 showed a trend on reducing body weight but didn't reach statistic difference compared to the vehicle group. Total 5 mice from different groups died during the 5 months study period due to kidney infection or Lv injection or the accident while performing blood collection. The information of dead animals was shown in the table below and the more detail information about dead mice was listed on the sheet of clinic observation of raw data file.

TABLE 2
The information of dead and wounded mice

| Group | Dead | Reason | Wounded | Reason |
|---|---|---|---|---|
| Negative control | 1 | No reason disappeared | 0 | fighting |
| Vehicle Saline | 1 | Died and the unclear | 2 | each |
| | | reason | | o [symbol] er |
| APOA 1 high dose | 2 | Kidney infection & | 1 | |
| | | i.v injection | | |
| APOA 1 mid dose | 1 | Blood collection | 1 | |
| APOA 1 low dose | 0 | | 3 | |
| Positive control | 0 | | 1 | |

[symbol]

indicates data missing or illegible when filed

6. Data Analysis

The results were expressed as the Mean±SEM and statistically evaluated by student's t-test. Differences were considered statistically significant if the P value was <0.05 or <0.01.

7. Results

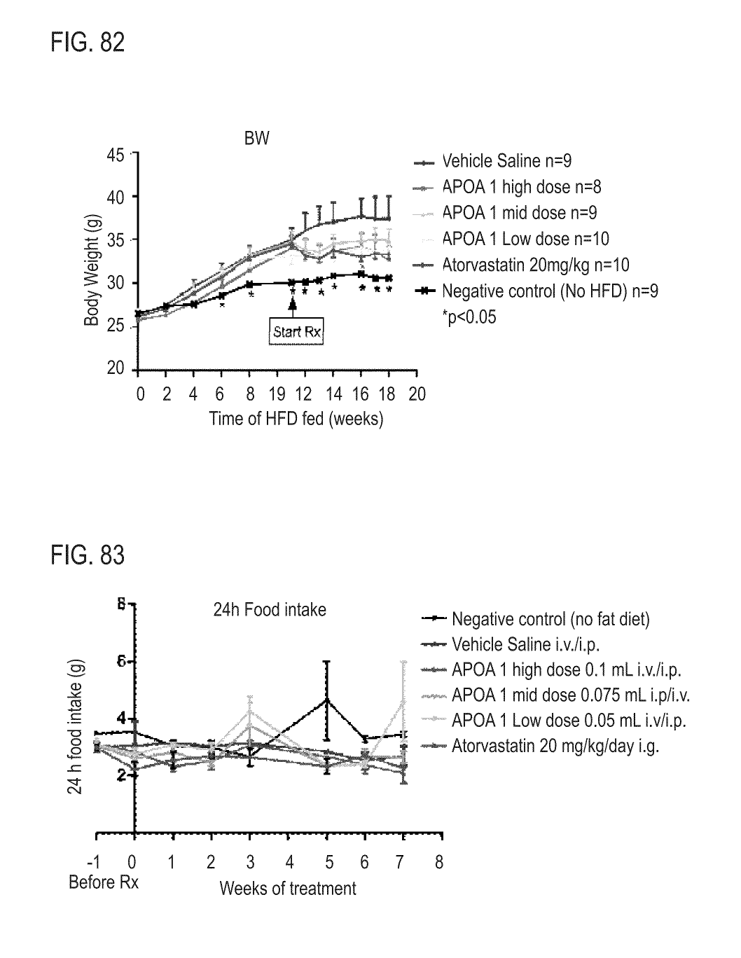

7.1. Effect of APOA Ion Body Weight
FIG. 82. Body weight

The body weight in Apo E knockout mice fed with HFD significantly increased after 6 weeks treatment compared with the mice in negative control group that were fed with normal diet. Atorvastatin significantly reduced the body weight after 5 weeks treatment. APOA1 showed a trend on reducing body weight but didn't reach statistic difference compared to the vehicle group.

7.2. Effect of 24 Food Intake.
FIG. 83. 24 h food intake

As shown in FIG. 2, mice in the negative control group eat a little bit more than the mice fed with HFD but no statistic difference.

7.3. Effect of HFD on Lipid Profile in ApoE ko Mice

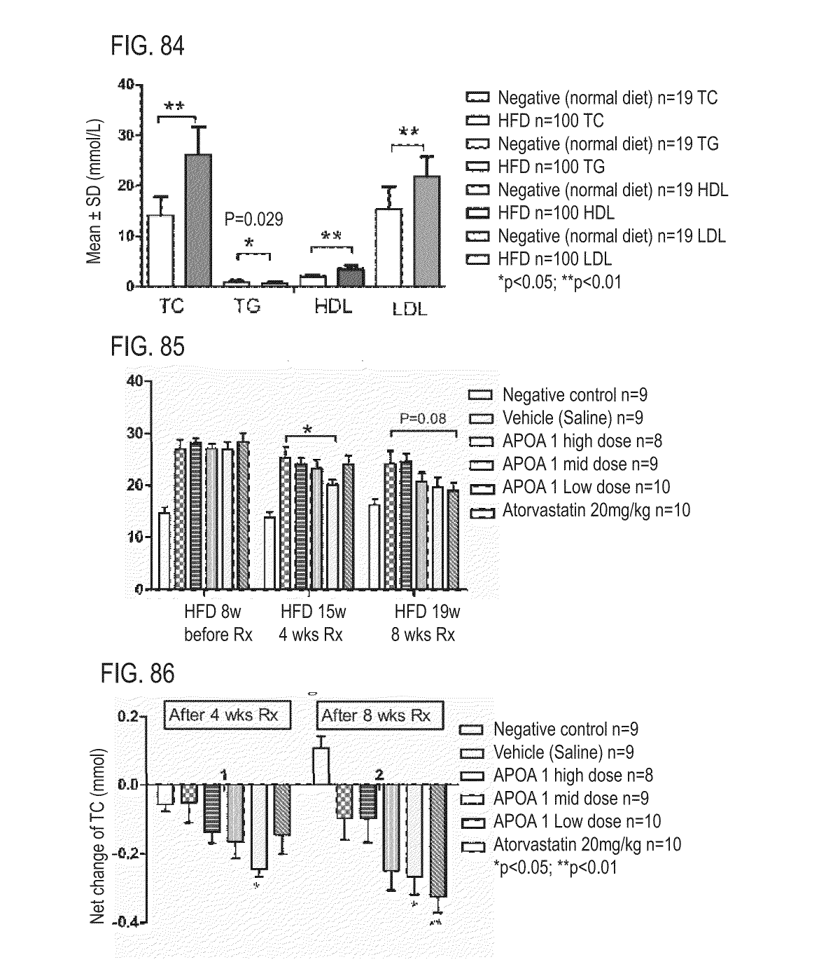

FIG. 84. Compare the lipid profile of ApoE mice fed with common diet and high fat diet.

The lipid profile was measured in Apo E ko mice fed with high fat diet for 8 weeks. As shown above, plasma TC, TG, LDL as well as HDL in Apo E ko mice fed with high fat/high cholesterol for 8 weeks were significantly increased compared to Apo E KO mice fed with normal chow diet.

7.4. Effect of RAAS Antibody on Total Cholesterol (TC)
FIG. 85, Plasma TC
FIG. 86. Net change of plasma TC

As shown in the figure above, positive control atorvastatin and low dose of APOA1 can significantly lower total cholesterol level after 8 week treatment in ApoE ko mice after 8 week treatment.

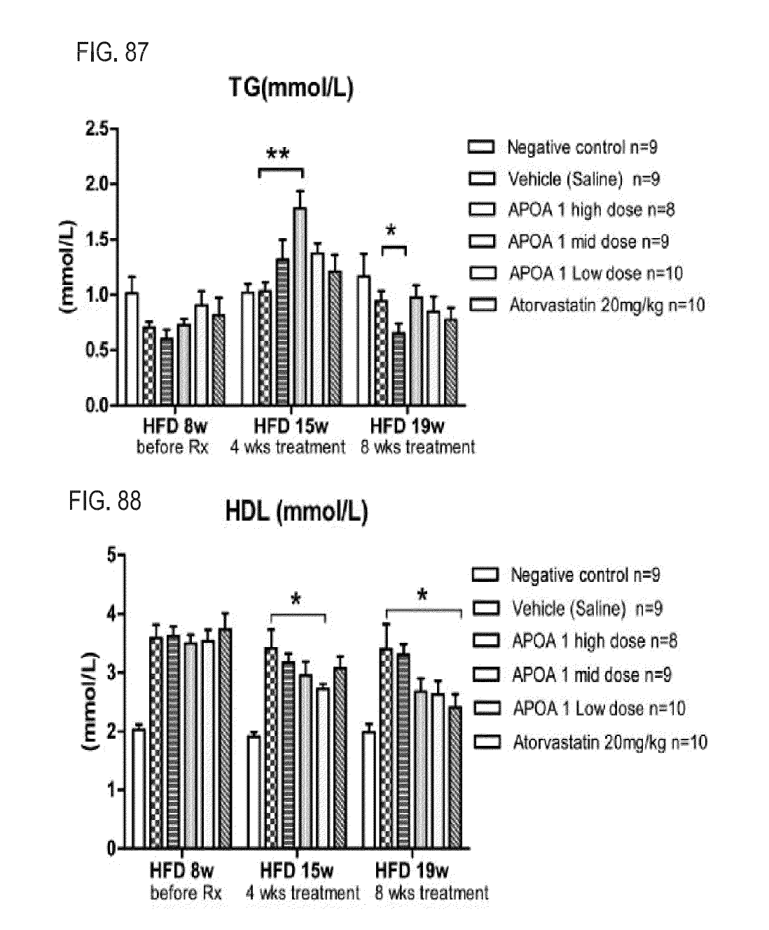

7.5. The effect of RAAS antibody on Triglyceride (TG}
FIG. 87. Plasma TG

As shown in figure above, positive control atorvastatin and RAAS antibody had no effect on plasma TG level in Apo E ko mice fed with HFD after 8 weeks treatment.

7.6. The Effect of RAAS Antibody on High Density Lipoprotein (HDI)
FIG. 88. Plasma HDL

As shown in FIG. 6, positive control atorvastatin can significantly lower high density lipoprotein in Apo E ko mice fed with HFD after 8 week treatment and RAAS antibody at low dose significantly decrease the HDL !eve! in ApoE ko mice after 4 weeks treatment.

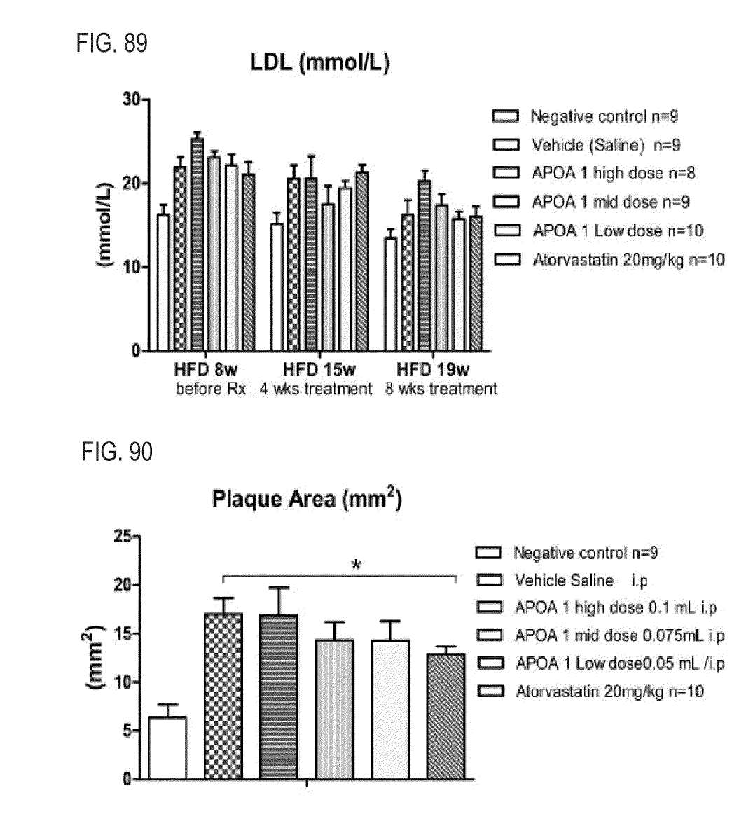

7.7. The Effect of RAAS Antibody on Low Density Lipoprotein (IDI)
FIG. 89. Plasma LDL level

There is no significant difference on plasma LDL between groups.

7.8. The Effect of RAAS Antibody on Atherosclerosis Plaque Lesion Area
FIG. 90. Atherosclerosis plaque area
FIG. 91. Percent of plaque area

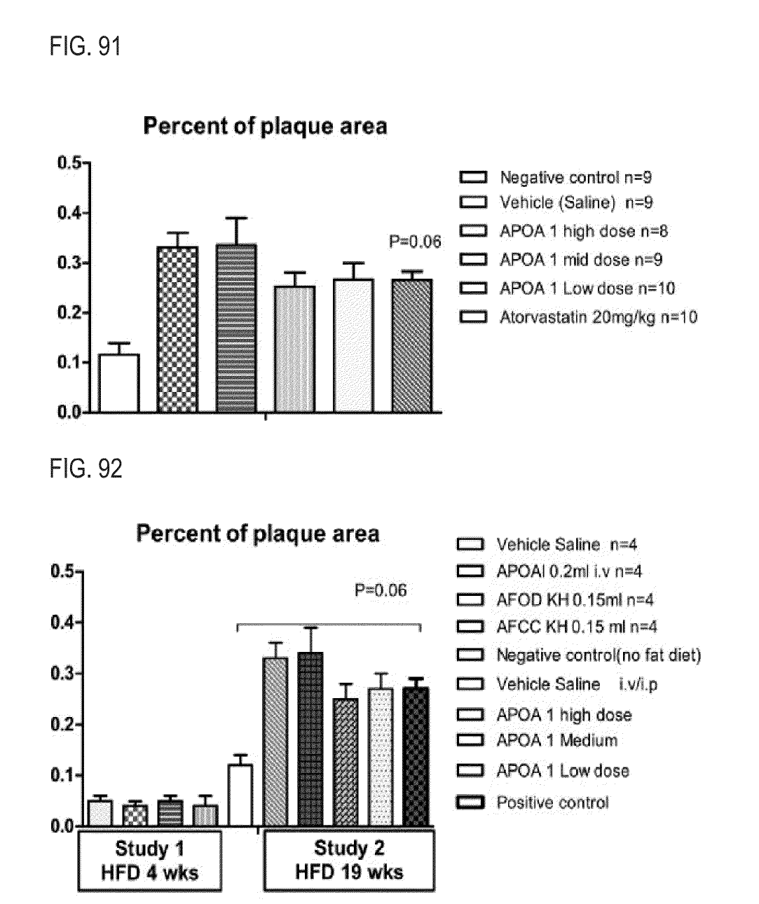

As shown in figures above, Atorvastatin significant reduced the plaque lesion area in ApoE knockout mice after 8 weeks treatment. RAAS antibody APOA1 !ow dose showed a trend on reducing the plaque lesion area of aorta in ApoE knout mice after 8 weeks treatment.
FIG. 92. Comparison percent of plaque area in study 1 and study 2.

We also compared percent of plaque area in the study 1 and study 2. In study 1, all ApoE ko mice were fed with HFD for 4 weeks and mice were sacrificed at 14 weeks of age. In study 2, ail ApoE ko mice were fed with HFD for 19 weeks except the mice in negative control group and all mice were sacrificed at 29 weeks of age. Obviously the percentage of plaque lesion area in all groups of mice in study 2 significantly increased than the one in study 1. The model of atherosclerosis in aorta was established successfully.

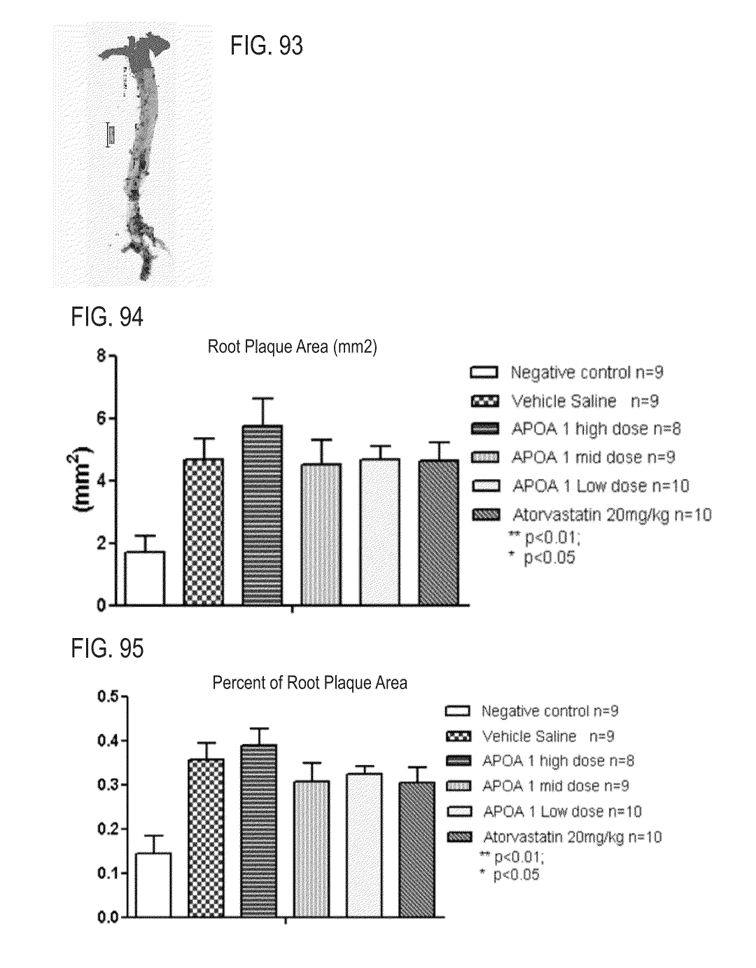

We analyzed the aortic plaque in different regions as shown in below:
FIG. 93, illustration of analyzing artery regions

Because the total lumen area in arterial arch is very difficult to identify in en face vessel, we measured the total area at the length of about 2 mm frorn aortic root down to the thoracic artery.
FIG. 94, Root plaque area

FIG. 95, Percent of root plaque area

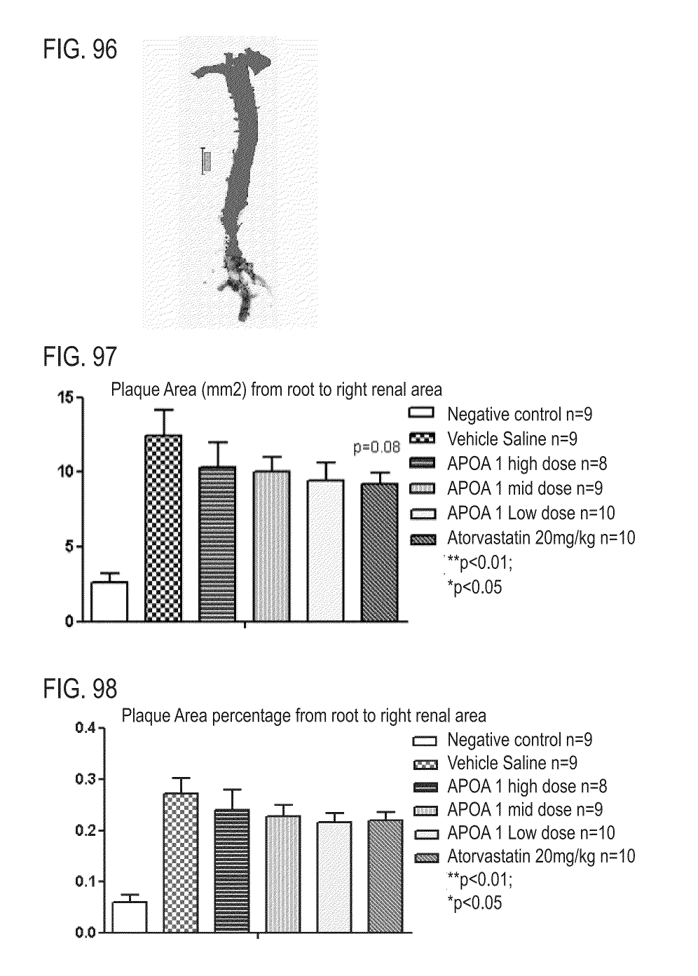

Atorvastatin and APOA1 mid dose and low dose showed a trend of reducing the arteriosclerosis plaque lesion in the region of thoracic aorta but didn't reach significant difference compared to the vehicle group
FIG. 96, illustration of artery analyzing regions

As shown in the above panel, the total area from the aortic root to the right renal artery was measured.
FIG. 97, results of plaque area from root to right renal
FIG. 98, percent results of plaque area from root to right renal

As shown in the figure above, Atorvastatin showed a trend of reducing the atherosderosis plaque lesion in the region from the aortic root to right renal artery but didn't reach the significant difference (p=0.08). RAAS antibody APOA1 also showed a trend of reducing the atherosclerosis plaque lesion in a dose dependent manner in this region.

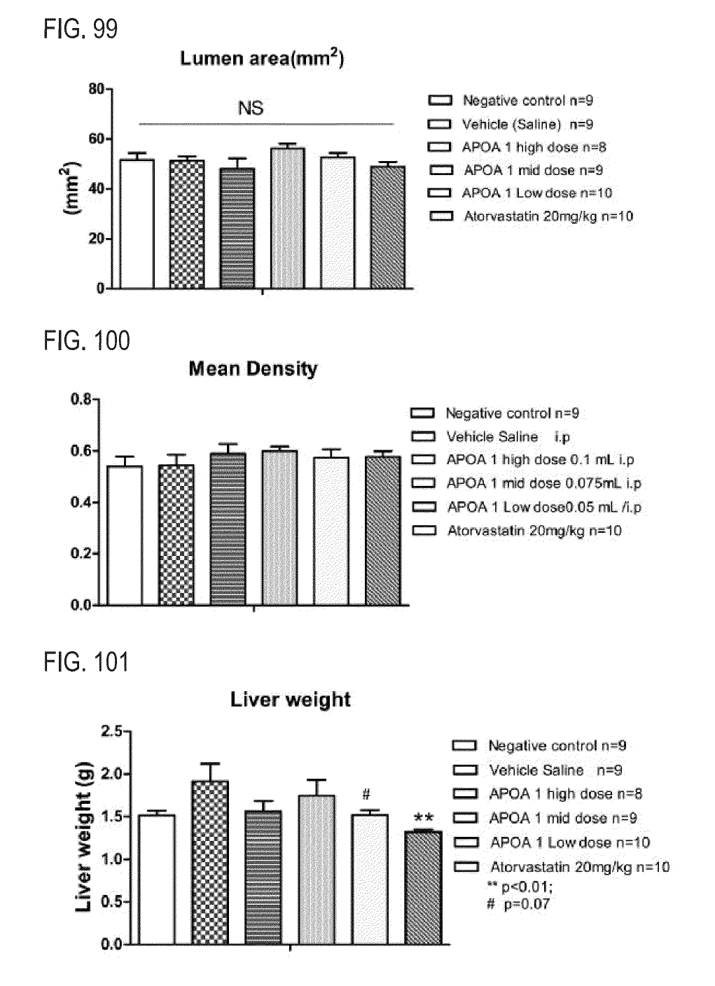

7.9. The Effect of Aortic Inner Lumen Area and Mean Density
FIG. 99. Aortic inner lumen area
FIG. 100. Mean density

There is no significant difference on aortic inner lumen area and mean density between the groups.

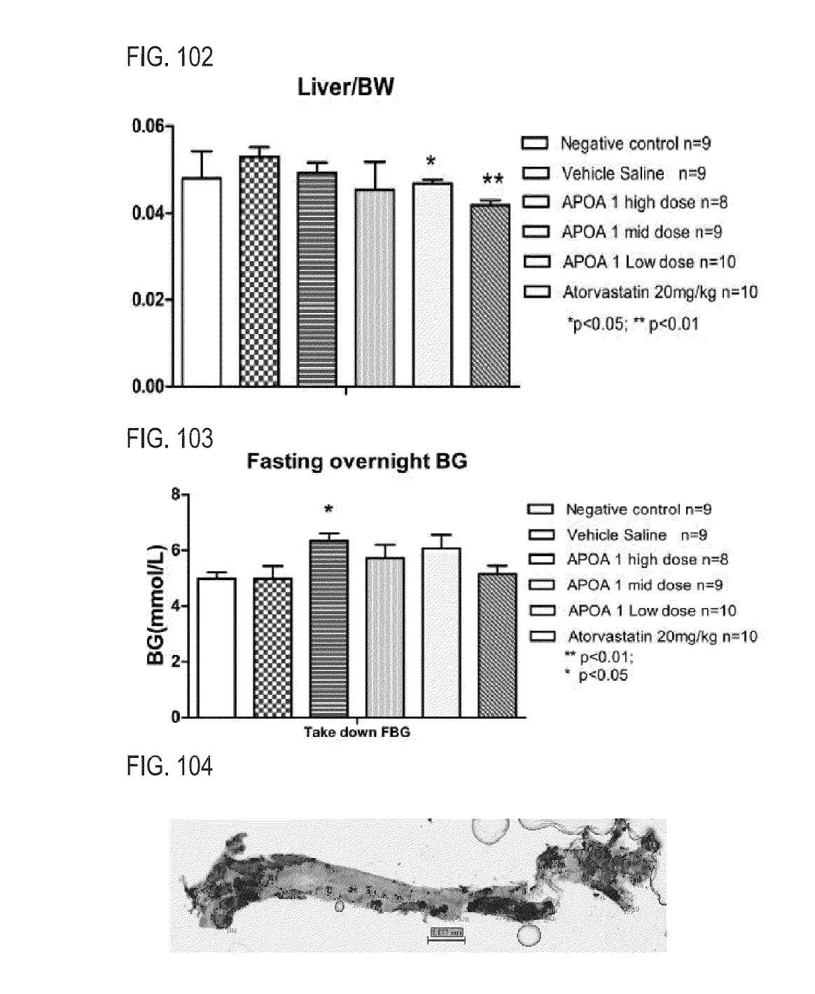

7.10. The Effect of RAAS Antibody on Liver Weight
FIG. 101. Liver weight
FIG. 102. liver weight index

RAAS antibody at the low dose reduced the ratio of liver weight/body weight significantly in ApoE ko mice after 8 weeks treatment compared to the vehicle group. Atorvastatin at 20 mg/kg reduced liver weight and the ratio of liver/body weight significantly in ApoE ko mice after 8 weeks treatment compared to the vehicle group

7.11. The Effect of RAAS Antibody on Fasting Overnight Blood Glucose
FIG. 103. Fasting overnight blood glucose

Atorvastatin and RAAS antibody had no effect on fasting overnight blood glucose after 8 weeks treatment compared to the vehide group.

7.12. Image of Aorta Red Oil Staining

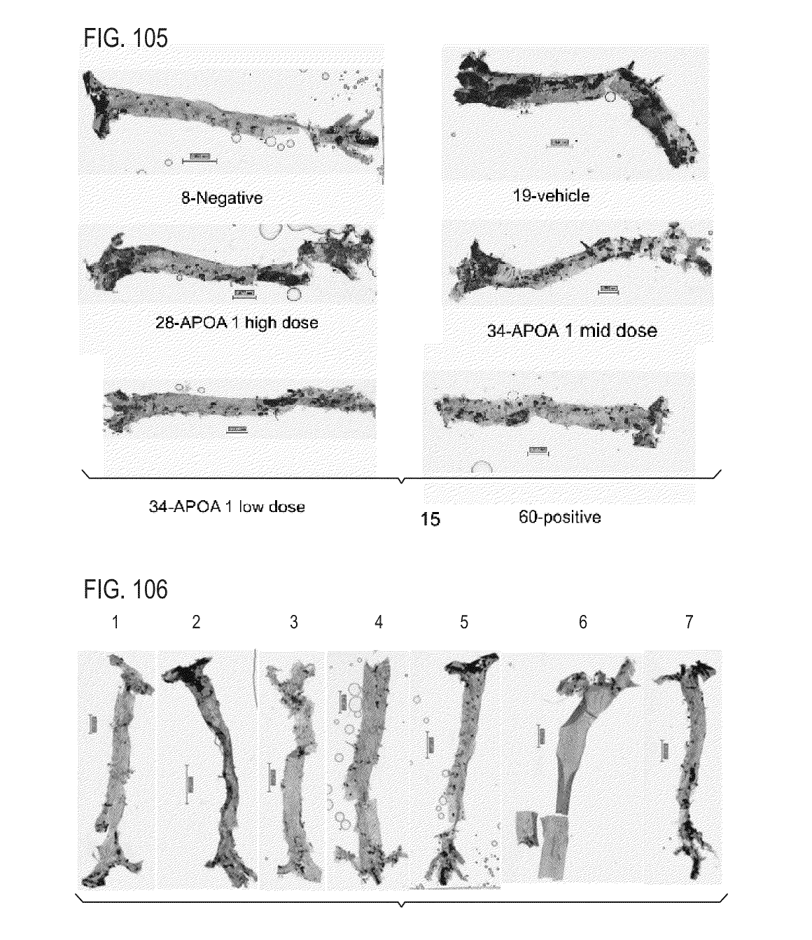

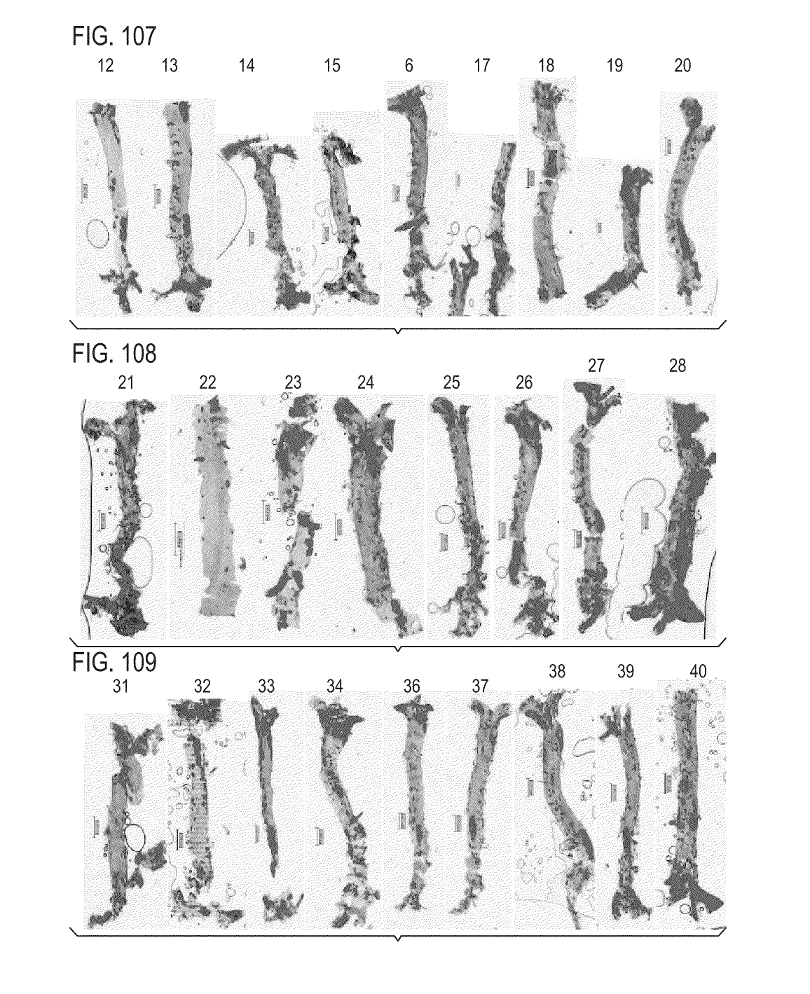

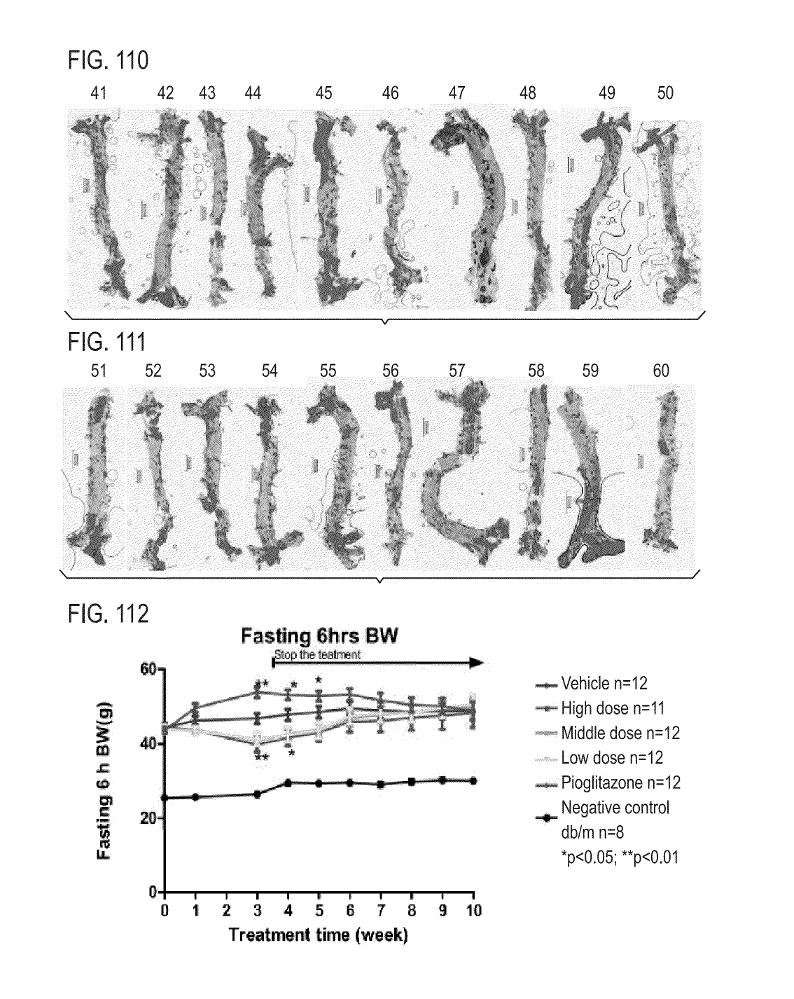

We selected some image of aorta stained by oil red and presented as below. The branches of artery and the lipid plaques could be observed clearly and the plaques mainly distribute in the aortic root and principal branches of the abdominal aorta. It is consistent with the reference literatures.
FIG. 104, Aorta stained by oil red
FIG. 105, Aorta stained by oil red in different groups
   Negative control
FIG. 106
   Vehicle control
FIG. 107
   APOAI high dose
FIG. 108
   APOAI medium dose
FIG. 109
   APOAI low dose
FIG. 110
   Positive control
FIG. 111

8. Conclusion

1) Atorvastatin at 20 mg/kg significantly reduced body weight, plasma TC, liver weight and the ratio of !iver/BW, the plaque lesion area of aorta in ApoE ko mice after 8 weeks treatment.

2) RAAS antibody APOA11ow dose significantly reduced plasma TC and the ratio of !iver/BW in ApoE ko mice after 8 weeks treatment.

3) RAAS antibody APOAll ow dose showed a trend of reducing body weight, plasma TC level, liver weight, the plaque lesion area of aorta in ApoE ko mice fed with HFD continuously for 18 weeks after 8 weeks treatment.

### Conclusion of 3 Studies on Lipid Panel:

We have performed the above 3 studies for 4 weeks, 8 weeks and 16 weeks. According to all the previous published studies on ApoE knockout mice the HDL (good cholesterol) and LDL (bad cholesterol) have shown a very disturbing result in the vehicle group, which has higher HDL and lower LDL to compare with the treated groups. When the vehicle which have been fed a HIGH FAT DIET AND CHOLESTEROL for 8 weeks befixe the injection of the tested AFOD RAAS (APOAI), and continue to be fed for another 4 weeks, and another 8 weeks and another 16 weeks.

However in comparison with the vehicle control it has shovvn a decrease in total cholesterol and triglycerides in tested groups.

Final Report of Efficacy Study on AFOD KH in db/db
mice

Study Title: Efficacy study of RL\i\S antibodies on ‘“fype 2 diabetic nlouse nlodelin db/db mice

Study Nunlber: CPB-P11-2504-RAAS

Date: Mar. 28, 2012

1. Abbreviations and Definitions

[symbol]

| | |
|---|---|
| kg | kilogram |
| g | gram |
| Mg | milligram |
| ng | Nanogram |
| ml | Milliliter |
| !JL | microliter |
| h | hours n1in |
| | minutes |
| Cpd | Compound |
| BW | Body \1Veight |
| BG | Blood Glucose |
| FBG | Fasting Blood Glucose |
| DOB | Date of Birth |
| TC | Total Cholesterol |
| TG | Triglyceride |
| LDL | Low Density Lipoprotein |
| HDL | High Density Lipoprotein |
| FB\N | Fasting Blood Glucose |

Standard Deviation Standard error Intraperitoneal injection paraformaldehyde

2. Introduction

The study described in this report evaluated in vivo effica.cy of RAAS antibody

AFOD, AFCC and APOA 1 in db/db mouse model

3. Purpose

To evaluate the efficacy effect of RAAS antibodies 0.1\FOD.' AFCC and APOi\ ! on blood glucose and related parameters in dbldb mouse model.

4. Materials

4.1 Compound: AFOD, AFCC, APOA

4.2 Animal: db/db and db/+C57 BLKS

Sex: male

Strain: C57BLKS

Vender: CP in house breeding

Age: 10 weeks (DOB:26 Aug. 2011) Number:

60 db/db mice and 8 db/m mice

4.3. Glucose test strips: ACCU-CHEK Performa: ROCHE (Lot#470396 2012-06-30)

4.4. CRYSTAL Mouse Insulin ELISA Kit (Cat#90080 Lot#
10NOUM1148,11NOUM!200)

4.5. Microplate Reader: Spectra Max PLUS384 Molecular Devices

5 Experiment Method

5.1. Original Group:

Fasting 6 hours and overnight blood glucose were measured. 60 db/db mice were assigned into 5 grouped based on the fasting 6 h blood glucose and body weight. Two mice with very low body weight were excluded from group. 8 db/rn lean mice was used as negative control group

TABLE 1
the information of groups

| | |
|---|---|
| Vehicles | 12 db/db mice |
| High Dose: | 12 db/db mice |
| Mid Dose: | 12 db/db mice |
| Low Dose: | 12 db/db mice |
| Negativity Control (db/m lean mice) | 8 db/db mice |

5.2. Study Duration: This Study was Conducted in Two Periods:

Period 1: Oct. 13, 2011-Feb. 10, 2012: Test 3 doses of AFOD Period

2: Feb. 13-Mar. 16, 2012: Test 3 antibody products

TABLE 2
The introduction of 2 periods

| | Period 1 | | Period 2 | |
|---|---|---|---|---|
| Antibody | AFOD | | AFOD, AFCC, APOA I | |
| Duration | Nov. 18, 2011-Feb. 10, 2012 | | Feb. 13-Mar. 16, 2012 | |
| | (0-10 wks) | | (10-15 wks) | |
| Group | Vehicles | 12 db/db | Vehicles | 12 db/db |
| | Positive | 12 db/db | Positive | 12 db/db |
| | (Piogiltazone 30 | | (Piogiltazone 30 | |
| | mg/kd/day) | | mg/kd/day) | |
| | High Dose: | 12 db/db | High Dose: | 12 db/db |
| | AFOD | | AFOD | |
| | 1.2 ml I.p | | 0.2 ml I.p | |
| | Mid Dose: AFOD | 12 db/db | Mid Dose: AFOD | 12 db/db |
| | 1.0 ml I.p | | 0.2 ml I.p | |
| | Low Dose: | 12 db/db | Low Dose: | 12 db/db |
| | AFOD | | AFOD | |
| | 0.8 ml I.p | | 0.2 ml I.p | |
| | Negative Control | 8 db/+ | Negative Control | 8 db/+ |
| | (db/m lean mice) | | (db/m lean mice) | |

Treatment 8 dose
Note:
5 mice died during the 11 weeks study period and their BW decrease significantly after AFOD injection

### Timeline

Period 1: Oct. 13, 2011-Feb. 10, 2012;

Nov. 18, 2011: Measure fasting overnight blood glucose and body weight

Nov. 21, 2011: Measure fasting 6 h blood glucose and body weight.

Nov. 23, 2011: Fasted overnight and co!lect the blood plasma for insulin test before the treatment.

Nov. 28, 2011: Group the mice based on the fasting 6 h blood glucose and fasting body weight and start the treatment with 3 doses of antibody AFOD by i.p every two days (Monday, Wednesday, and Friday).

Dec. 16, 2011-Feb. 10, 2012: Stop all the treatment including the positive control group.

Nov. 28, 2011-Feb. 10, 2012: Measure body weight and blood glucose weekly.

Jan. 13, 2012& Feb. 9, 2012: Weigh the body weight and collect blood p!asrna for insulin measurement (fasted overnight).

Period 2: Feb. 13-Mar. 16, 2012:

Feb. 13, 2012: Start the treatment with 3 antibodies by i.p every two days (Monday, Wednesday, Friday) after 8 weeks washout from previous treatment.

Feb. 13-Mar. 16, 2012: Measure body weight and blood glucose weekly.

Mar. 13, 2012: Weigh body weight and collect the fasting overnight blood plasma for insulin measurement.

Mar. 16, 2012: Sacrific the mice and collect the plasma for lipid profile measurement, measure the body and liver weight, and collected pancreas by fixing in the 4% paraformaldehyde.

5.3. Route of Compound Administration:

Antibody products were administrated by intraperitoneal injection and the positive compound was mixed into food at the dose 30 mg/kg/day.

5.4. Body weight and blood glucose measurement: Fasting 6 hours body weight and blood glucose concentration were measured by Roche glucometer weekly.

5.5. Plasma insulin measurement: About 30 ul of blood sample was collected from the orbital vein for each mouse and centrifuged 7000 rpm at 4° C. for 5 min. Plasma samples were saved in −70 l-::. The plasma insulin level was measured with EUSA kit (CRYSTAL, cat#90080),

5.6. Plasma lipid profile measurement: The plasma lipid profile were measured by the DaAn Clinic central lab.

5.7. Study taken down: After 14 dose antibody products treatment, all mice were sacrificed. Measure body weight and collect blood sample for each mouse. Measure liver weight and save one piece for pathology study and freeze one piece in liquid nitrogen for further analysis in the future. Save pancreas into 4% paraformaldehyde (PFA) fixation solution for future analysis.

5.7. Clinic observation: Several mice lost body weight significantly after AFOD treatment as shown in the results. Total 7 mice from different groups died during the 4 months study period due to kidney infection or skin ulcer or skin abscess. The information of dead animals was shown in the table below and the more detail information about dead mice was listed on the sheet of dinic observation of raw data file.

TABLE 3
The information of dead mice

| | Part 1 | | | | | | | |
|---|---|---|---|---|---|---|---|---|
| | low | | | | Part 2 | | | |
| | blood | kidney | lung | No | kidney | intestinal | kidney | |
| | glucose | infection | bleeding | reason | infection | bump | bleeding | Total |
| Group | | | | | | | | 11 |
| Vehicle | | | | | | | | 0 |
| high dose | 1 | | | 1 | 2 | | | 4 |
| mid dose | | | | | 2 | | | 2 |
| low dose | | 1 | 1 | | | 1 | | 3 |
| Positive | | 1 | | | | | 1 | 2 |
| group | | | | | | | | |
| Negative | | | | | | | | 0 |
| control | | | | | | | | |

6. Data Analysis

The results were expressed as the Mean±SEM and statistically evaluated by student's t-test. Differences were considered statistically significant if the P value was <0.05 or <0.01.

7. Results

PART 1: Nov. 18, 2011-Feb. 10, 2012 (0-10 weeks)

7.1.1. Effect of AFOD on body weight
FIG. 112, Body
weight

AFOD at 3 doses significantly reduced body weight in db/db mice after 3 weeks treatment compared with vehicle group but the difference disappeared after the treatment stopped from week 4. The Positive control Pioglitazone significantly increased body weight in db/db mice after 2 weeks treatment but lost difference after the treatment stopped.

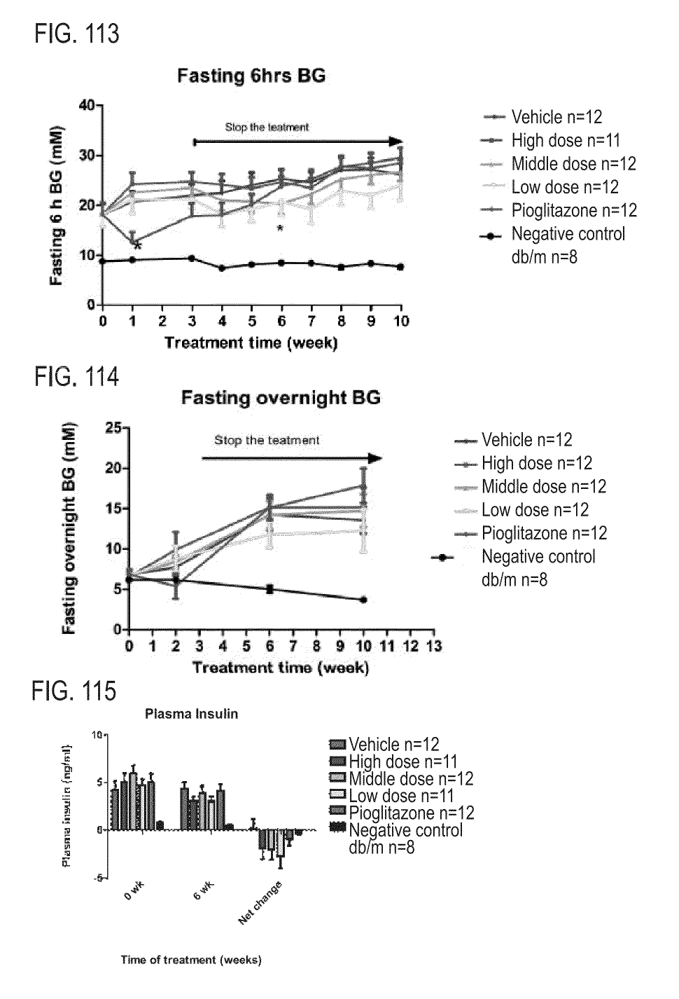

7.1.2. Effect of Products on Blood Glucose (Fasting 6 h).
FIG. 113. Blood glucose (Fasting 6 h)

As shown in FIG. 2, positive control Piog!itazone significantly reduced blood glucose in db/db mice after 1 week treatment and blood glucose level was back to vehicle group levels 10 days after treatment stop. AFOD at low dose showed the effect on lowering blood glucose after 8 doses treatment.

7.1.3. Effect of Products on Fasting Overnight BG
FIG. 1.14. Fasting overnight BG

AFOD has no effect on fasting overnight BG in db/db mice but the positive control Pioglitazone can significantly lower blood glucose after 1 week treatment and blood glucose level back to the vehide control levels gradually after the treatment stopped.

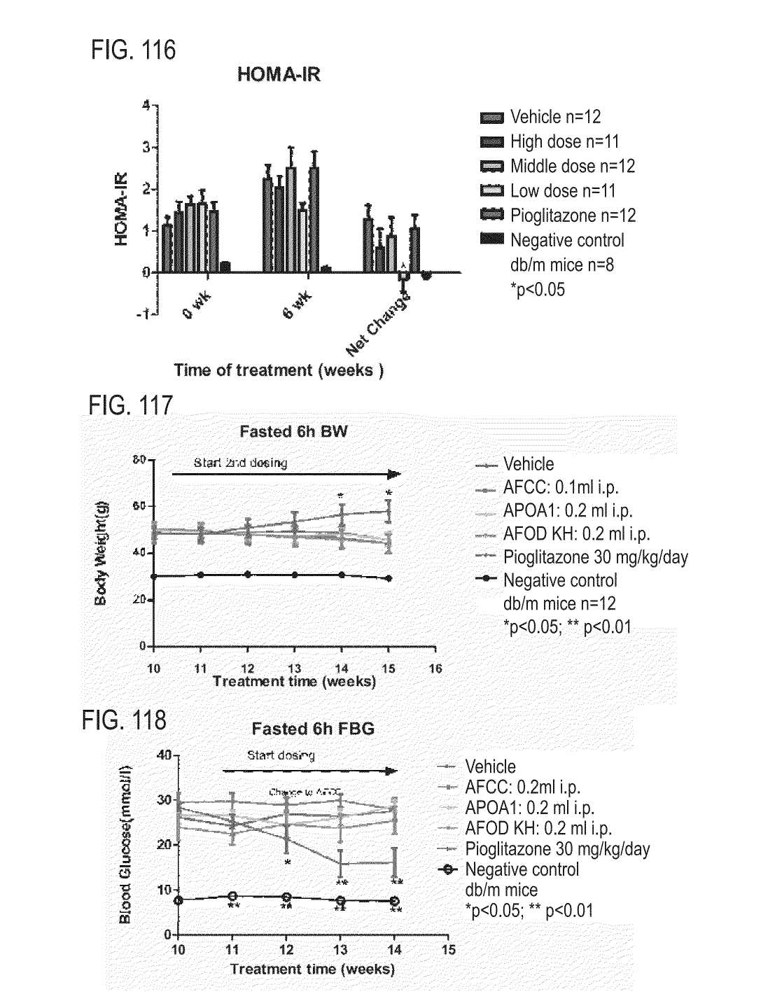

7.1.4. The Effect of AFOD on Plasma Insulin and HOMA-IR
FIG. 115. Plasma insulin
FIG. 116 HOMA-IR

As shown in FIGS. 4A and 4B, AFOD at low dose showed a trend on reducing plasma insulin level and improving insulin resistance in db/db mice after 8 doses treatment.

PART 2: Feb. 13-Mar. 16, 2012

7.2.1. The Effect of AFODAFCCAPOA Ion Body Weight
FIG. 117. The effect of AFOD, AFCC, APOA I on body weight

Three products have no effect on body weight in db/db mice compared to vehicle group but the positive control pioglitazone showed an effect on increasing body weight.

7.2.2. The Effect of AFOD,AFCC,APOA I on Fasted 6 h Blood Glucose
FIG. 118. Blood glucose (fasted 6 h)

There is significant difference on blood glucose between the pioglitazone group and vehide group but the three test articles” showed no effect on fasting 6 h blood glucose.

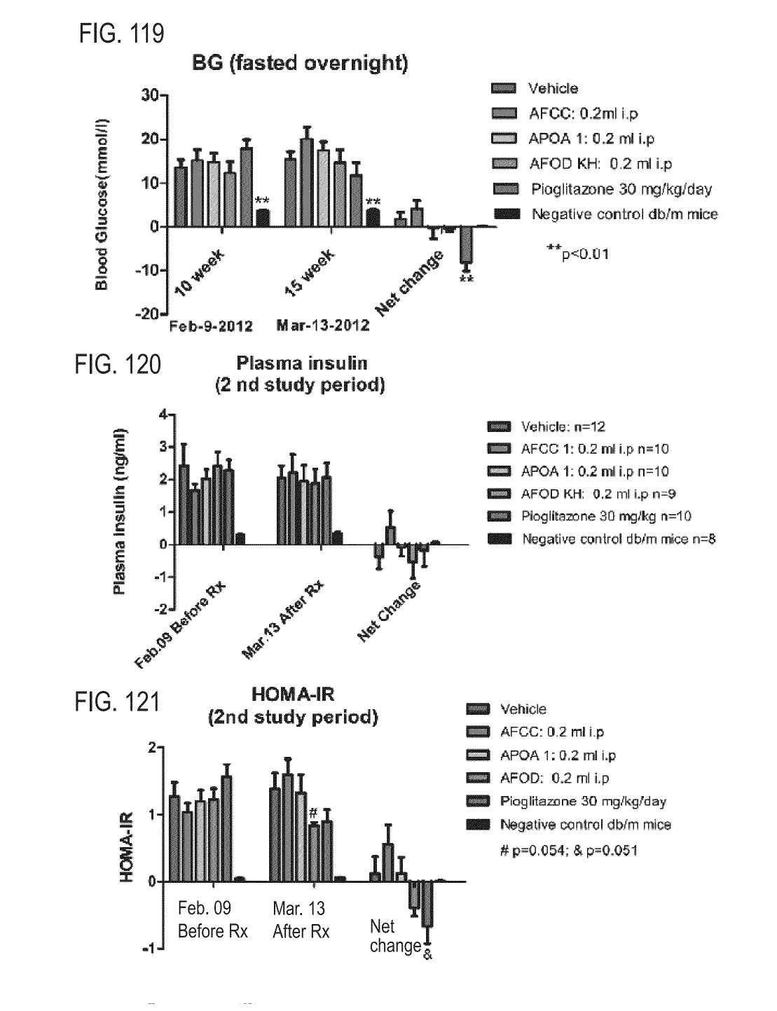

7.2.3. The Effect of Three Products on Overnight Fasting Blood Glucose
FIG. 119. Blood glucose (fasted overnight}

Three antibody products had no effects on overnight fasting blood glucose in db/db mice compared to the vehicle group, but positive control piog!itazone significantly reduced the fasting overnight blood glucose level after 4 weeks treatment in db/db mice.

7.2.4. The Effect of Three Products on Plasma Insulin and HOMA-IR
FIG. 120. Plasma insulin
FIG. 121. HOMA-IR

AFOD showed a trend on improving plasma insulin resistance in db/db mice after 14 doses treatment (p=0.054), the pioglitazone also showed an trend on improving insulin resistance after 5 weeks treatment in aging db/db mice at 6 months old (p=0.051).

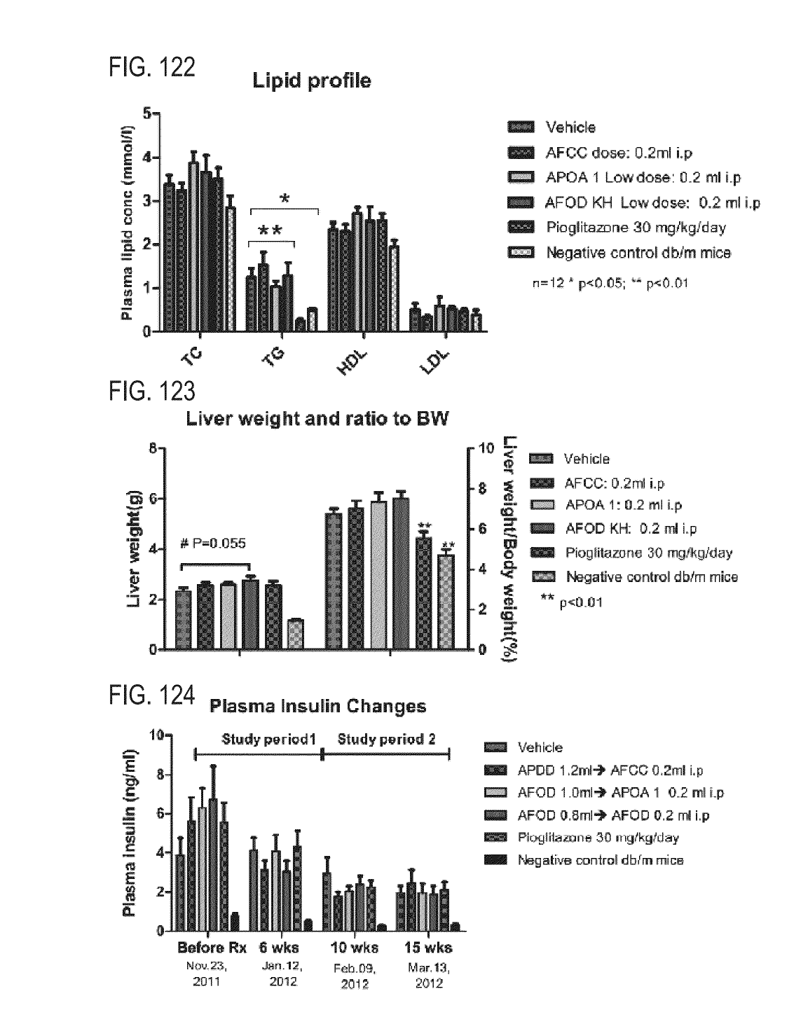

7.2.5. The Effect of AFOD,AFCC,APOA Ion Plasma Lipid
FIG. 122. Plasma lipid profile

Three antibody products have no effects on plasma lipid profile in db/db mice after 14 doses treatment compared to the vehicle group; but positive control pioglitazone significantly lowered the plasma triglyceride !evel in db/db mice after 5 weeks treatment.

7.2.6. The Effect of AFOD,AFCC,APOA I on Liver Weight
FIG. 123. Liver weight

Three antibody products have no effect on liver weight and the ratio of liver/body weight compared to the vehicle group. The positive control pioglitazone showed the effect on reducing the ratio of !iver weight to body weight due to the increase of body weight.

7.2.7. Plasma Insulin Level in Db/Db Mice During Two Periods of Study
FIG. 124. Four measurements of plasma insulin

The plasma insulin level in db/db mice were gradually declined when mice are getting older.

8. Conclusion

Study Period 1:

[symbol] Positive control piog!itazone significantly reduced the blood glucose !eve! and increased body weight after 1 week treatment in db/db mice compared to the vehicle group. Both b!ood glucose and body weight in this group of mice gradually went back to baseline after the treatment stopped.

Y AFOD at three doses reduced the body weight significantly after 3 weeks treatment in db/db mice compared to the vehicle group. AFOD at low dose (0.8 ml i.p injection, q.o.d) showed a trend on lowering blood glucose and improving insulin resistance compared to the vehicle.

Study Period 2:

? The positive control pioglitazone has follow effects in db/db mice after 4 weeks treatment:
../ lower blood glucose (Fasted 6 h and overnight)
../ increase body weight
./. reduce plasma triglyceride level
../ improve the insulin resistance

? RAAS product AFOD at low dose showed a trend on improving insulin resistance in db/db mice after 4 weeks treatment (14 doses i.p. injection) but didn't reach the statistic difference (p=0.054) compared to the vehicle group.

### In Vivo Efficacy Testing of Eight RAAS Compounds in 411-IUC Breast Cancer Cell Orthotopic Model

Apr. 25, 2012-Jun. 28, 2012

### Executive Summary

Effects of AFOD RAAS 1/8, AFOD RAAS 2, AFOD RAAS 3, AFOD RAAS 4, AFOD RAAS 5, AFOD RAAS 6, AFOD KH and AFCC KH on tumor growth in Balb/c nude mouse orthotopic model from 4T1-LUC cell line were investigated in this study. Toxicity was evaluated by body weight monitoring as well as daily observation. Bioluminescence was measured with !VIS Lumina !! machine. Mice treated with AFOD RAAS 1/8, AFOD RAAS 2, AFOD RAAS 3, AFOD RAAS 4, AFOD RAAS 5, AFOD RAAS 6, AFOD KH and AFCC KH exhibited a significant reduction of Relative ROI 6 and 9 days after compounds administration, as cornpared to vehicle control.

During the first 16 days post administration (Daylto Day 16L body weight of all of the testing article and gemcitabine treated mice, got increased stably, which indicated that both the testing compounds and control agent gemcitabine were well tolerated at this stage by current dosing schedule. However, significant body weight loss was found in testing article treated mice since Day 17 and the situation got even worse on Day 22 probably because dosing volume changed from 0.4 ml/mouse to 0.6 ml/mouse on that day. As the dosing schedule was changed to 1.0 ml/mouse BID on Day 23, dramatic body weight loss was continuously observed. Macroscopically, all the mice in the testing article treated groups suffered from serious abdomen swelling, so administration was halted for 4 days (Day 25 to Day 28L and the remaining mice were monitored closely. During the experimental period (Day 1 to Day 28) totally 42 mice died, significant body weight loss was found before death. On Day 29, the recovered mice in AFOD RAAS 3 and AFOD RAAS 5 treated groups were IP treated with 0.4 ml/mouse, while the other mice in AFOD RAAS 4, AFOD KH and AFCC KH groups were kept untreated due to bad status. In addition, mice in gemcitabine group were monitored by IVIS after stop dosing. The results indicated that although the testing compounds might have potential anti-tumor effect, dose, schedule and route of administration were also Important for validation of such effect.

1. Objective

Determine the effects of AFOD RAAS 1/8, AFOD RAAS 2, AFOD RAAS 3, AFOD RAAS 4, AFOD RAAS 5, AFOD RAAS 6, AFOD KH and AFCC KH on primary tumor growth and metastasis in Balb/c nude rnouse orthotopic model established frorn 4T1-luc breast cancer cells.

2. Materials and Method

2.1. Animals, reagents and instruments

2.1.1 Animal Specifkations

Species: Mus Musculus

Strain: Balb/c nude mouse

Age: 6-8 weeks

Sex: female

Body weight: 18.20 g

Number of animals: 80 mice plus spare

2J 0.2 Animal Husbandry

The mice were kept in laminar flow rooms at constant temperature and humidity with 3 or 4 animals in each cage.
Temperature: 20 2.5 ′C.
Humidity: 40-70%.

[symbol] Light cycle: 12 hours light and 12 hours dark.

Cages: Made of polycarbonate. The size is 29 em×17.5 ern×12crn (L×W×H). The bedding material is wood debris, which is changed once per week.

Diet: Animals had free access to irradiation sterilized dry granule food during the entire study period.

Water: Animals had free access to sterile drinking water.

Cage identification: the identification labels for each cage contained the following information: number of animals, sex, strain, date received, treatment, study number, group number, and the starting date of the treatment.

Animal identification: i\nimals were marked by ear punch.

2.1.3 Animal Procedure

i\11 the procedures related to animal handling, care, and the treatment in this study were performed according to guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were checked and recorded for any effects of tumor growth on normal behavior such as mobility, food and water consumption (by looking only), body weight gain/loss, eye/hair matting and any other abnormal effect.

2.L4 Reagents and Instruments

4T1-LUC cell line (Caliper, USA); RPIVII 1640 medium (Invitrogen, USA); FBS (Invitrogen, Australia); DPBS (Fisher, USA); PBS (Gibco, USA); Sodium-Heparin (Sigma, USA); I′ v1C (Sigma, USA); Formaldehyde (Sinopharm, China); Twelve-hydrated isodium hydrogen phosphate (Sinopharm, China); Sodium dihydrogenphosphate (Sinopharm, China);

CO₂ Incubator (Thermo Scientific, USA); Biological Safety Cabinet (BSC-A₂, Shanghai, China); Centrifuge (Eppendorf, USA); Centrifuge (Thermo Scientific, USA); Pipettor (Thermo Scientific, USA); Finnpipettor (Eppendorf Research, USA); Pipette (Corning, USA); Plastic Cell Culture Flask (Corning, USA); Tube (Greiner Bio-one, Germany); Microscope (Nikon, Japan); Parafilm (Parafilm M, USA); Electronic Analytical Balance (Sartorius, Germany); Barnstead Nanopure (Thermo Scientific, USA); Cryopreservation of refrigerator (Haier, China).

2.2. Prm Edure and Method

2.2.L14T1-LUC Cell Thaw

2.1 4T1-LUC Cell Culture

One tube of 4T1-I.UC (from Caliper) cells were thawed according to the following procedure:

1. Cells were thawed by gentle agitation of vial in a 37″C water bath. To reduce the possibility of contamination, the O-ring and cap were kept out of the water. The whole process should be rapid (approximately 2 minutes);

2. Vials were removed from the water bath as soon as the contents were thawed, and was decontaminated by spraying with 7.5% ethanol. All the operations from this point on should be carried out under strict aseptic conditions;

3. The content of the vials was transferred into a centrifuge tube containing 10 ml of complete culture medium (RPMI1640+10% FBS) and was spin at 1000 rpm for 3 minutes. Supernatant was discarded;

4. Cell pellet was resuspended with the 5 ml of medium. The suspension was transferred into a 17.5 cm² flask, 2.5 ml of complete culture medium was added and mixed;

5. Cells were incubated at 37° (, 5% CO 2.

2.2.1.2 Subculture of the 4T1-luc Cells

4T1-luc cells were split according to the following procedure:

1. Cells were aspirated by gently pipetting;

2. 1 ml of the cell suspension was added into a new 175 en} flask, 30 ml of complete culture medium was added and the flask was gently shaked to spread the suspension throughout the bottom. The subculture ratio was 1:10;

3. Cells \Nere observed under an inverted microscope and were incubated at 3FC, 5% CO₂.

2.2.1.3 Harvest of 4T1-luc Cells

4TI-luc cells were harvested according to the following procedure:

1. Cells were harvested in 90% confluence and viability was no less than 90%. 4TI.luc cells were transferred into a conical tube and centrifuged at 1000 rpm for 6 min, supernatant was discarded;

2. Cells were rinsed with 50 ml of PBS twice, the viable cells were counted on a counter, 14×10⁷ cells were obtained;

3. 14 ml of PBS was added to make a cell suspension of 10×10⁶ cells/ml and mixed.

2.2.2 Animal Model Establishment

A total number of 92 female Balb/c nude mice were purchased. These mice were allowed 3 days of acclimatization period before experiments start.

The cell suspension was carried to the animal room in an ice box. 100 fiL of 1×10⁶ 4TI-luc cells was implanted orthotopiclly into the right rear mammary fat pad lobe of each mouse. Totally 80 mice were selected and divided into 10 groups. All mice were monitored daily.

2.2.3 Mea:sun.'nH.'nts

Tumor growth status was monitored by both IVIS Lurnina II and a digital caliper twice weekly since the day after cell implantation.

2.2.3.1R01 (Region of Interest) Measurement.

For IVIS Lumina II measurement, bioluminescence intensity of primary tumor and metastatic tumor was obtained according to the following procedure:

1. Tumor-bearing mice were \Neighted and intra peritoneally administered luciferin at a dose of 150 mg/kg (10 rnI/kg);

2. After 10 min, mice were pre-anesthetized with the mixture of oxygen and isoflurane. When the animals were in complete anesthetic state, move them into the imaging chamber and obtain bioluminescence images with IVIS machine (Lumina II);

3. ROI data was calculated with IVIS Lumina II software and relative ROI was calculated to express the tumor growth status.

Relative ROI :::ROit/ROI₁, where

ROI, - - - R01 value at day t ROl₁ . . . ROI

value at day 1

2.2.3.2 Tumor Volume Measurement

Tumor size was measured twice a week in two dimensions using a caliper and the tumor volume (V) was expressed in mm³ using the formula: V=0.5 ax b² where a and bare the long and short diameters of the tumor, respectively.

2.2.4.1Compounds Preparation:

2.2.4 Formulation Preparation

(1) AFOD RAAS 1/8, AFOD RAAS 2, AFOD RAAS 3, AFOD RAAS 4, AFOD RAAS 5, AFOD RAAS 6, AFCC KH solutions were provided by client and stored at 4° C.

2.2.4.2 AFOD KH Solutions were Filtered with Millipore Membrane Filters Before Dosing.

2.2.4.3 Gemcitabine Solution Preparation:

200 mg gemcitabine was dissolved in 33.3 m1 0.9% NACL. and vortexed to obtain 60 mg/ml gemcitabine solution.

2.2.5 Animal Experiment

2.2.5.1Random Assignment of Treatment Groups

8 days post 4Tinoculation, when tumors reached an average volume of 79 mm³, 80 out of the 88 mice were selected based on relative ROI and tumor volume. These animals were randomly assigned to 10 groups (n=8).

2.2.5.2 Administration of the Animals

1. 1 Vlice were treated with AFOD RAAS 1/8, AFOD RAAS 2, i\FOD RAAS 3, AFOD RAAS 4, AFOD RAAS 5, AFOD RM\S 6, AFOD KH, AFCC KH and gemcitabine since Random assignment according to Table I The first administration day was denoted as Day 1.

TABLE 1
Experimental design

| | Dosage | | Dosing | Animal |
|---|---|---|---|---|
| Treatment | (ml/mouse) | Dosing Route | Schedule | Number |
| Control | n/a | n/a | n/a | 8 |
| Gerncita bine | 60 mg/kg | IP | 2X/WK | 8 |
| AFOD RAAS 1 | 0.2/0.4 | 1V/IP | OD | |
| AFOD RAAS 2 | 0.2/0.4 | 1V/IP | OD | |
| AFOD RAAS 3 | 0.2/0.4 | 1V/IP | OD | |
| AFOD RAAS 4 | 0.2/0.4 | IV/IP | QD | |
| AFOD RAAS 5 | 0.2/0.4 | IV/IP | QD | |
| AFOD RAAS 6 | 0.2/0.4 | IV/IP | QD | |
| AFOD KH | 0.2/0.4 | IV/IP | QD | |
| AFCC KH | 0.2/0.4 | IV/IP | OD | |

Note:
1. Animals in vehicle group did not receive any treatment.

2. For every administration group, detailed dosing information could be found in Exhibit 3.

2. Mice were observed daily to identify any overt signs of adverse, treatrnent-related side effects of compounds, any upset and uncomfortable of mice were recorded. Body weights were measured and recorded twice weekly.

2.2.6 Experimental Endpoint

1. On Day 31(39 days post inoculation), all anirnals in vehicle group died.

2. On Day 35 (43 days post inoculation), all AFOD RAAS :1./8, AFOD R1\AS 2, AFOD RAAS 3, i\FOD RAAS 4, AFOD RAAS 5, AFOD RAAS 6, AFOD KH, AFCC KH treated animals died.

3. Animals in gemcitabine group are monitored by IVIS after stop dosing.

2.3 Statistical Analysis

2.3.1 TGI (Tumor Growth Inhibition, in Percentage)

TGI (tumor growth inhibition, in percent) was calculated according to the following equation:

TGI(%)={1−(TI−TO)/(C1 . . . CQ)}, where
C1-median tumor volume of control mice at timet T:I.-median tumor volume of treatment mice at time t CO-median tumor volume of control mice at time 0 TO-median tumor volume of treatment mice at time 0

2.3.2 T/C (%). Calculation

T/C (%) was calculated based on the tumor volume data collected on Day 27.

2.3.3 ANOVA Analysis

The difference between the mean values of tumor volume in treatment and vehicle groups was analyzed for significance using one way ANOVA test at each time point after log transformation.

3. Results and Discussion

3.1 Tumor Growth Curve Based on Relative ROJ

FIG. 1 showed the relative ROI changes after administration of vehicle, gemcita bine and AFOD RAAS 1/8, AFOD RAAS 2, AFOD RAAS 3 AFOD RAAS 4, AFOD RAAS 5, AFOD RAAS 6, AFOD KH, AFCC KH. As shown in Table 2 no significant changes in relative ROI were found in all AFOD RAAS 1/8, AFOD RAAS 2, AFOD RAAS 3, AFOD RAAS 4 AFOD RAAS 5, AFOD RAAS 6, AFOD KH, AFCC KH treated groups as compared to vehicle group.

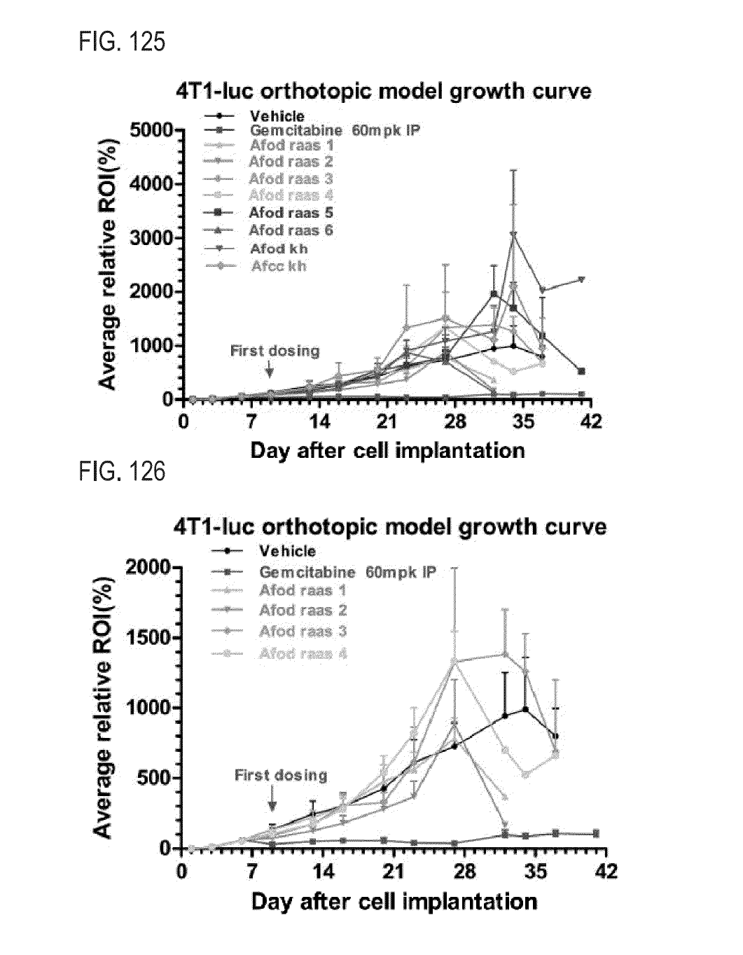

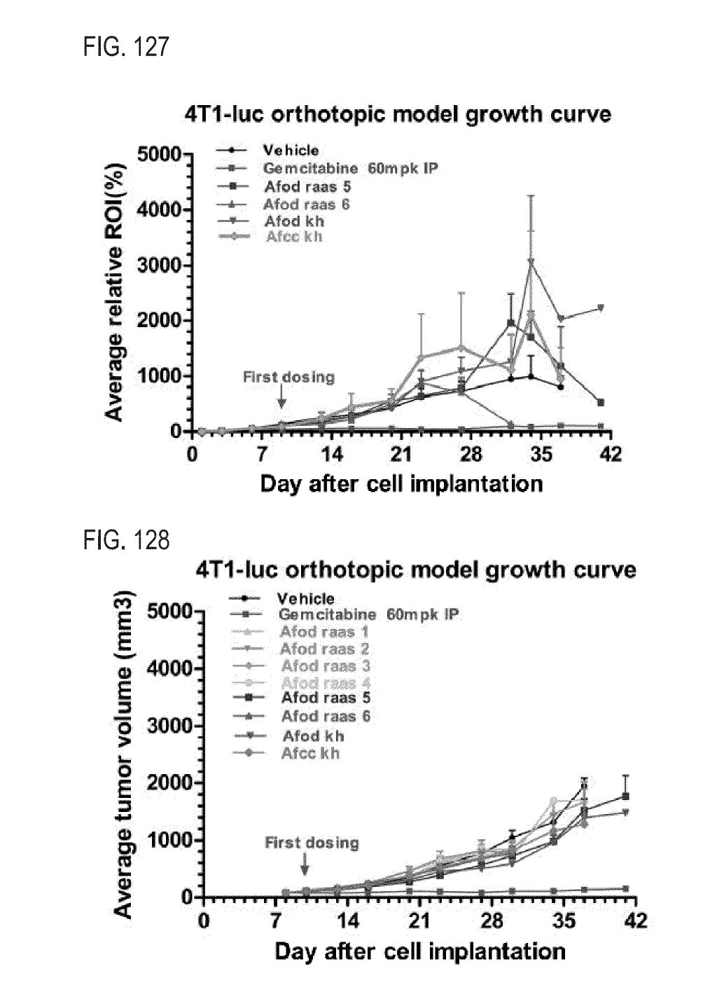

The bioluminescence graphs and the relative ROI values were displayed in Exhibit 1 and Exhibit 2.
FIG. 125
FIG. 126
FIG. 127

FIG. 1 Relative ROI changes of 4T1-LUC-bearing BALB/C nude mice after administration of vehicle, AFOD RAAS 1/8, AFOD RAAS 2, AFOD RAAS 3, AFOD RAAS 4, AFOD RAAS 5, AFOD RAAS 6, AFOD KH, AFCC KH, and Gemcitabine. Data were shown as mean±SEM. Mean value and SEM was calculated based on survived animals.
Table 2 Summary of one-way ANOVA analysis on relative ROI changes

3.2 Tumor Growlb Curve Based on Tumor Volume

FIG. 2 showed the tumor volume changes of 4T1-LUC-bearing Balb/c nude mice after administration of vehicle, AFOD RAAS 1i8, AFOD RAAS 2, AFOD RAAS 3, AFOD RAAS 4, AFOD RAAS 5, AFOD RAAS 6, AFOD KH, AFCC KH, and gerncitabine.

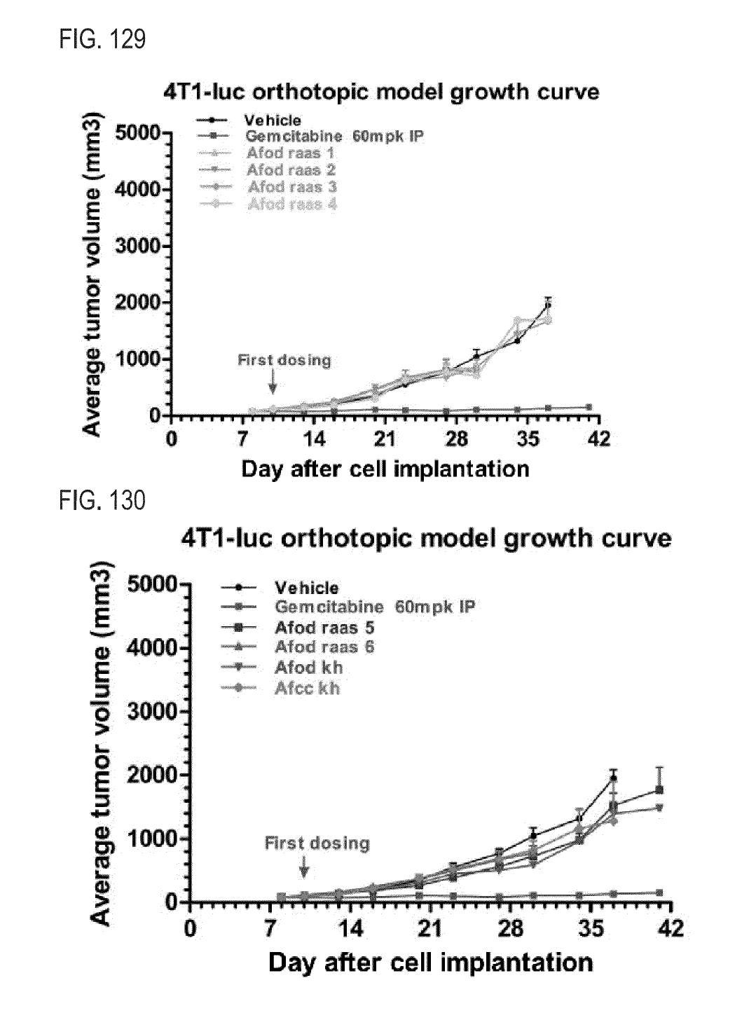

No significant tumor volurne reduction was observed in all AFOD RAAS 1/8, AFOD RAAS 2, AFOD RAAS 3, AFOD RAAS 4, AFOD RAAS 5, AFOD RAAS 6, AFOD KH, AFCC KH treated groups when compared to vehicle group, while gerncitabine exhibited significant tumor volume reduction role since day 13 after administration as compared to vehicle control. (Table 3).
FIG. 12.8
FIG. 129
FIG. 130

FIG. 2 Tumor volume changes of 411-LUC-bearing Ba!b/c nude mice after administration of vehide, AFOD RAAS 1/8, AFOD RAAS 2, AFOD RAAS 3, AFOD RAAS 4, AFOD RAAS 5, AFOD RAAS 6, AFOD KH, AFCC KH, and Gemdtabine. Data were shown as mean±SEM. Mean value and SEM was calculated based on survived animals.

TABLE 3
Summary of one-way ANOVA analysis on tumor volume changes

| | Gemcitabine | AFOD | AFOD | AFOD | AFOD | AFOD | AFOD | AFOD | AFCC |
|---|---|---|---|---|---|---|---|---|---|
| | 60 mpk ip | R1\AS | Rr\AS 2 | Riv\S 3 | RAAS4 | Rr\i\S 5 | RAAS 6 | KH | KH |
| Day | 2X/WK | 1/8 QD | QD | QD | QD | QD | QD | QD | QD |
| 10 | NS | NS | NS | NS | NS | NS | NS | NS | NS |
| 13 | ** | NS | NS | NS | NS | NS | NS | NS | NS |
| ]6 | *** | NS | NS | NS | NS | NS | NS | NS | NS |
| 20 | *′** | NS | NS | NS | NS | NS | NS | NS | NS |
| [symbol] | *** | NS | NS | NS | NS | NS | NS | NS | NS |
| 27 | *** | NS | NS | NS | NS | NS | NS | NS | NS |

[symbol]

indicates data missing or illegible when filed

3.3 Toxidty Evaluation by Body Weight Change (′;) Monitoring and Daily Observation of 4T1-LUC-Bearing Balb/c Nude Mice

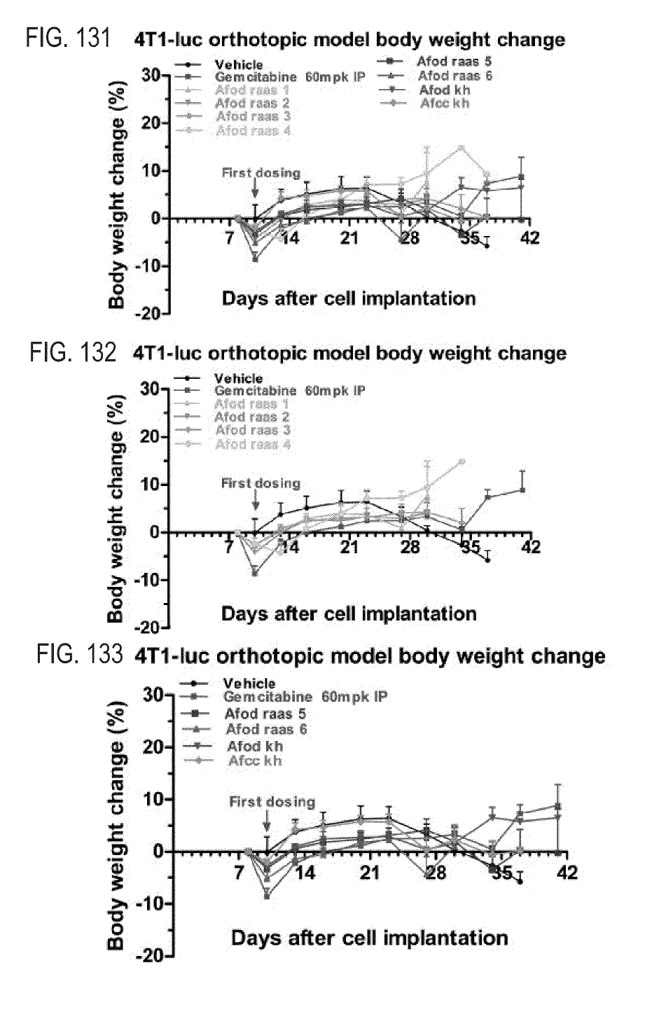

Body weight change (%) is one of the important indicators to exhibit the toxicity of the testing materials. FIG. 3 showed the body weight change (%) during the whole study period (Exhibit 2.). During the first 16 days post administration (Daylto Day 16), body weight of mice in all of the testing article and gerncitabine treated groups increased normally, implying that the compounds were well tolerated via current dosing schedule. However, the body weight loss was found since Day 17 and the situation got even worse on Day 22 by changing dosing volume from 0.4 mlimouse to 0.6 ml/mouse and then to 1.0 ml/mouse BID on Day 23,. Macroscopically, all the mice in the testing article treated groups suffered from serious abdomen swelling, so administration was halted for 4 days (Day 25 to Day 28), and the remaining mice were monitored closely. During the experimental period totally 42 mice died, significant body weight losses were found before mouse death. On Day 29, the recovered mice in AFOD RAAS 3, AFOD RAAS 5 were IP treated with dosing volume of OAml/mouse, while the other mice In AFOD RAAS 4₁ AFOD KH and AFCC KH groups were kept untreated due to bad status. Furthermore, mice in gemcitabine group were monitored by IVIS after stop dosing. It seemed that both the dosing concentration and volume of AFOD RAAS 1/8, i\FOD RAAS2, AFOD RAAS 3, AFOD RAAS 4, AFOD RAAS 5, AFOD RAAS 6, AFOD KH, AFCC KH contributed to the deaths. All of the primary tumors of dead mice were removed and weighed.
FIG. 131; FIG. 132; FIG. 133

FIG. 3 Body weight change(%) of 4T1-LUC-bearing Baib/c nude mice following administration of vehicle, gemcitabine and AFOD RAAS 1/8, AFOD RAAS 2 AFOD RAAS 3, AFOD RAAS 4, AFOD RAAS S, AFOD RAAS 6, AFOD KH, AFCC KH. Data were shown as mean±SEM. Mean value and SEM was calculated based on survived animals.

3.4 TGI (%) Calculation

Table 5 showed the tumor grmNth inhibition (TGI) ratio of treatment groups.

TABLE 5
Tumor growth inhibition of four treatment groups

| | Day | Day | Day | Day | Day | Day |
|---|---|---|---|---|---|---|
| TGI (%) | 10 | 13 | 16 | 20 | 23 | 27 |
| Ger eitabine | 1.09 | 1.14 | 0.95 | 0.88 | 0,96 | 0.99 |
| 60 mpk | | | | | | |
| ip vs Vehicle | | | | | | |
| AFOD RAAS | Vs | −0.52 | −0.35 | − ,39 | −0.20 | −0,08 |
| 1/8 ip | Vehicle, | | | | | |
| AFOD RAAS 2 | 0.23 | −0.38 | −0,35 | −0.45 | −0.26 | −0.06 |
| ip Vs Vehicle | | | | | | |
| AFOD RAAS 3 | −0.59 | −0.72 | −0.36 | −0.07 | −0.11 | 0.12 |
| ip Vs Vehicle | | | | | | |
| AFOD RAAS 4 | −0.04 | −0.08 | 0.11 | −0.03 | −0.16 | 0.00 |
| ip Vs Vehicle | | | | | | |
| AFOD RAAS 5 | 0.45 | 0.02 | 0.16 | 0.29 | 0.35 | 0.30 |
| ip Vs Vehicle | | | | | | |
| AFOD RAAS 6 | −0.22 | −0.39 | −0.34 | −0.09 | 0.11 | 0.14 |
| ip Vs Vehicle | | | | | | |
| AFOD kh ip | 0.05 | 0.27 | −0.07 | 0.15 | 0.21 | 0.38 |
| Vs ′Vehicle | | | | | | |
| AFCC kh ip | | | | | | |

3.5(%) Calculation

T/C (%) was calculated based on the tumor volume data collected on Day 27.

AFOD RAAS 1/8 IP, QD group: T=824.09 mm³, C=768A7 mm³. T/C (%)=1.07

AFOD RAAS 2. IP, QD group: T=8U.:I.:I. mm³, C::: 768.47 mm³. T/C (%)=1.06

AFOD RAAS 3 IP, QD group: T::: 686.52 mm³, C=768.47 mm³. T/C (%)::: 0.89

AFOD RAAS 4 IP, QD group: T=770.20 mm³, C=768.47 mm³. T/C (%)::: 1.00

AFOD RAAS 5 IP, QD group: T=564.66 mm³, C::: 768.47 mm³. T/C (%)=0.73

AFOD RAAS 6 IP, QD group: T=672.66 mm³, C=768.47 mm³. T/C (%)=0.88

AFOD KH IP, QD group: T 506.57 mm³, C::: 768A7 mm³. T/C (%) 0.66

AFC:C: KH IP, QD group: T=690.57 mm³, C::: 768.47 mm³. TIC: (%)=0.90

4. Conclusion

Effects of AFOD RAAS 1/8, AFOD RAAS 2, AFOD RAAS 3, AFOD RAAS 4, AFOD RAAS 5, AFOD RAAS 5, AFOD KH, AFCC KH on tumor growth in Balb/c nude mouse orthotopic model from 411-LUC cell line were investigated in this study. Toxicity was evaluated by body weight monitoring as well as daily observation. Bioluminescence was measured with IVIS Lumina II machine. The results indicated that no significant change in relative ROI as well as in tumor volume was found in all test treated groups as compared with vehicle group.

In this study, we found out that continuous administration of all of the testing articles, including AFOD RAAS :1/8, AFOD RAAS 2, AFOD RAAS 3, AFOD RAAS 4, AFOD RAAS 5, AFOD RAAS 6, AFOD KH and AFCC KH could render dramatic weight loss, although this is not obvious during the first 16 days post treatment, Notably, all the testing article treated mice suffered from serious abdomen swelling. Take together, the results indicated that although the testing compounds might have potential anti-tumor effect, dose, schedule and route of administration were also important for validation of such effect.

### APPENDICES

### Exhibit 1: Fluorescence Images of the Whole Body

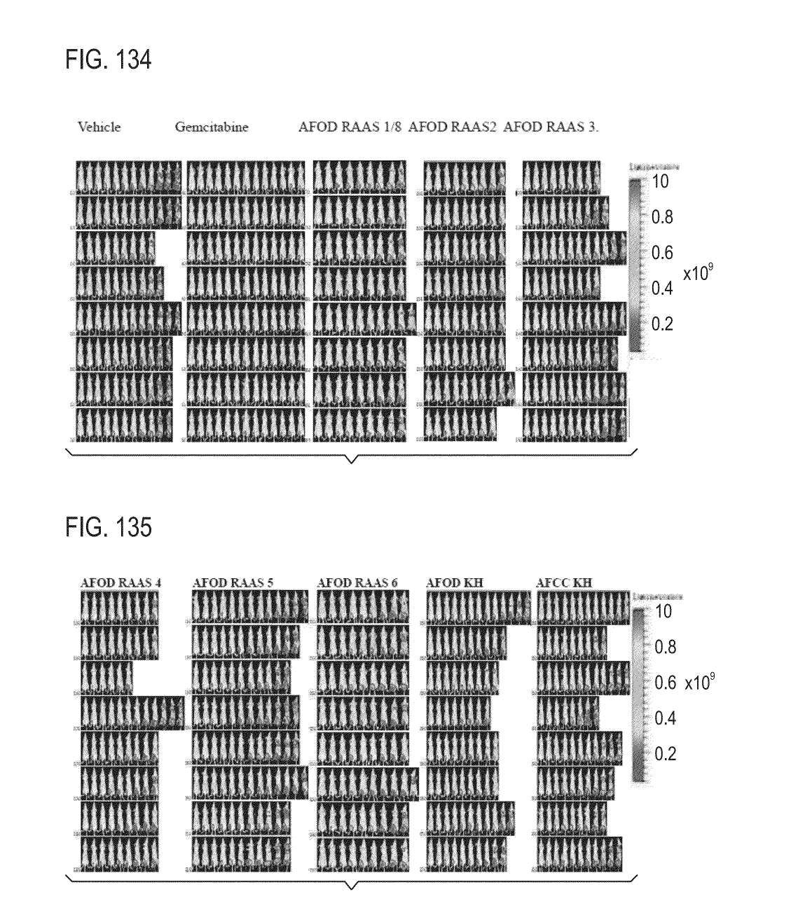

FIG. 134
FIG. 135

RAAS

Title: Anti-tumor efficacy of high concentrated fibrinogen enriched al at thrombin and Afod (FS) in combination with Afod RAAS 2 or Afod RAAS 4 in patient-derived tumor xenograft (PDX) models in nude mice.

Description: Patient-derived liver tumor xenograft (PDX) partial removal model was used to evaluate the anti-cancer efficacy of high concentrated fibrinogen enriched al at thrombin and Afod (FS) in combination with Afod RAAS 2 at different 3 doses or with RAAS 4 at one dose. The results showed FS in combination with Afod RAAS 2 at all dosed or with RAAS 4 significantly inhibited the growth of remaining tumor at the beginning of treatment, but the duration was not long. On day 24 after dosing, the tumor sizes and tumor weights in FS in combination with Afod RAAS 2 groups or with RAAS 4 group were not significantly inhibited compared with sham-operated control group. In summary, FS in combination with Afod RAAS 2 or RAAS 4 inhibited the liver PDX tumor growth temporarily.

Subject high concentrated fibrinogen enriched al at thrombin and Afod (FS), Afod RAAS, patient-derived tumor xenograft model, liver cancer

Summary

Patient-derived liver tumor xenograft (PDX) partial removal model was used to evaluate the anti-tumor efficacy of high concentrated fibrinogen enriched al at thrombin (FS) in combination with RAAS 2 at 3 doses or with Afod RAAS 4 at one dose. The mice were implanted subcutaneously with LI-03-0117 P6 tumors fragments of about 30 mm³. When xenograft tumors reached 200 mm³, a portion of tumor was removed by surgery, and a portion of tumor of 20 mm³ in size was left, and FS or a control agent was applied to wound surfaces of both sides after tumor removal. Injection of Afod RAAS 2 or Afod RAAS 4 was conducted 2 days after the surgery, and lasted for 24 days. Tumor size and body weight were measured once per week. 24 days after injection of test agents, the mice were sacrificed and tumors were dissected and weighed. The tumor volumes and final tumor weights for all groups were statistically analyzed by one-way ANOVA with the significance level set at 0.05. The data showed that FS in combination with Afod RAAS 2 at all doses or with RAAS 4 significantly inhibited the growth of remaining tumor, but anti-tumor efficacy lasted less than 3 weeks. On day 24 after dosing, the tumor sizes and tumor weights in FS in cmnbination with Afod RAAS 2 at all dosed or with RAAS4 group were not significantly inhibited compared with sham-operated control group. In summary, FS in combination with Afod RAAS 2 or RAAS 4 inhibited the liver PDX tumor growth temporarily.

1. Details of Facility, Personnel and Data Location

Location of Raw Data, Original Protocols, Experimental Details and Report

The studies described in this report were carried out on behalf of RAAS at external laboratories:

2. Introduction

The aim of the study was to test anti-tumor efficacy of FS in combination with Afod RAAS 2 or Afod RAAS 4 in patient-derived liver tumor xenograft (PDX) partial removal model in nude mice.

The model used in the study was derived from surgically resected, fresh patient tumor tissues. The first generation of the xenograft tumors in mice was termed passage 0 (PO), and so on during continual implantation in mice. The passage of xenograft tumors at P7 (LI-03-0117) were used in this study.

All the experiments were conducted in the AAALAC-accrediated animal facility in compliance with the protocol approved by the Institutional Animal Care and Use Committee (IACUC).

3. METHODS

3.1. Experimental Preparations

3.LL Animal Preparation

Female Balb/c nude mice, with a body weight of approximately 20 grams, were obtained from an approved vendor (Sin θ-British SIPPR/BK Lab. Animal Co. Ltd., Shanghai, China).

Acclimation/Quarantine: Upon arrival, animals were assessed as to their general health by a member of a veterinary staff or authorized personnel. Animals were acclimated for at least 3 days (upon arrival at the experiment room) before being used for the study.

Animal Husbandry:

Animals \Nere housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sin θ-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcmne of the study.

3.L2. Tumor Tissue Preparation

The liver xenograft tumor models were established from surgically resected clinical tumor samples. The first generation of the xenograft tumors in mice is termed passage 0 (PO), and so on during continual implantation in mice. The tumor tissues at passage 7 (L1-03-0117) were used in this study.

3J.3. Formulation

High concentrated fibrinogen enriched a1 at thrornbin and Afod were provide by RAAS and prepared by RAAS scientist during experiment before use. Matrigel (BD Biosciences; cat. #356234).

3.2. Experimental

Protocol

3.2.1. Establishment of Xenograft Model and Treatment

Grouping and Treatment

Nude mice were assigned to 6 different groups with ·15 or 25 mice/group and each group received different treatment as shown in Table i.

TABLE 1
Grouping and the treatment

| Group | Treatment | N | | Surgery |
|---|---|---|---|---|
| | Sham-operated | 15 | • | Remove 90% of tumor to keep 20 mm³, and close by |
| | control: | | | suturing (no treatment). |
| 2. | Positive control | 15 | • | Remove 90% of tumor to keep 20 mm³, treat the wound |
| | | | | surfaces with Matrigel, and close by suturing. |
| 3. | AFOD RAAS 2 − | 25 | 6 | Remove 90% of tumor to keep 20 mm³, treated the |
| | FFS--- high | | | wound surfaces of both sides with Afod RAAS 2 × 10 |
| | | | | (once every 1 minute for 10 times), and then with 3 times of |
| | | | | FS (about 0.4 m1), and close by suturing. |
| | | | 6 | After 2 days, treat with Afod RAAS 2 (400 ul, QD × 30, iv). |
| 4. | AFOD RAAS 2 + | 15 | • | Remove 90% of tumor to keep 20 mm³, treated the wound |
| | FS- moderate | | | surfaces of both sides with Afod RAAS 2 × 8 (once every 1 |
| | | | | minute for 8 times), and then with 2 times of FS (about 0.3 |
| | | | | ml), and close by suturing. |
| | | | * | After 2 days, treat with Afod RAAS 2 (300 ul, QD × 30, iv). |
| | AFOD RAAS 2 + | 15 | • | Remove 90% of tumor to keep 20 mm³, treated the |
| | FS- low | | | wound surfaces of both sides with Afod RAAS 2 × 6 (once |
| | | | | every 1 minute for 6 times), and then with 1 times of FS |
| | | | | (about 0.2 ml), and close by suturing. |
| | | | 0 | After 2 days, treat with Afod RAAS 2 (200 ul, QD × 30, iv). |
| 6. | AFOD RAAS2 + | 15 | • | Remove 90% of tumor to keep 20 mm³, treated the |
| | FS + RAAS 4 | | | wound surfaces of both sides with Afod RAAS 2 × 10 |
| | | | | (once every 1 minute for 10 times), and then with 3 times |
| | | | | of FS (about 0.431), and close by suturing. |
| | | 100 | • | After 2 days, treat with Afod RAAS 4 (400 ul, QD × 30, iv). |

Experiment Procedures

A Xenograft tumors were collected and cut into pieces of 30 mm³ and implanted into 120 mice subcutaneously (with 30%) extra).

B. When xenograft tumors reach 200 mm³, the animal was anesthetized by i.p. injection of sodium pentobarbital at 60-70 mgikg. The animal skin was sterilized with ethanol solution. Skin was opened.

C. A portion of tumor was removed by surgery, and a portion of tumor of 20 mm³ in size was left for further growth.

D. Apply test agents or positive control agent locally following the study design. OB gel shouldn't be used to avoid potential side effects. E. The skin was closed and sutured.

F. Pictures were taken in representative animals in each group, before and after surgical removal of tumor, and after completion of surgery.

G. Postoperative care was conducted by following SOP-BE0-0016-1.0.

H. Injection of AFOD RAAS 2 or AFOD RAAS 4 was conducted 2 days after the surgery, and lasted for 24 days.

I. During the period of the experiment, health conditions of mice were observed daily. Body weight of mice was monitored once per week.

J. Turnor sizes were rneasured once per week. Turnor volumes (mm³) were obtained by using the following formula: volume=(W2 xL)/2 (W, width; L, length in mm of the tumor).

K. Mice, which showed a significant loss of body weight (>20%), or which were unable to eat or drink, or exhibit ulceration on the skin/tumor, or the tumor size reached 2,000 mm³, were euthanized immediately to minimize the pain and distress. Such actions need to notify the sponsor within 24 hrs (48 hrs during the weekends).

L. Mice were scarified at the end point (24 dafter injection of test agents).
a) Dissemination of cancer was identified macroscopically. The tissue surrounding tumor was also checked for the invasion of cancers.
b) Tumors were collected and their weights will be measured.
c) Pictures of collected tumors were taken.

3.2.2. Evaluation of the Anti-Tumor Activity

Health conditions of mice were observed daily. Body weights were measured once a week during the treatment. Tumor sizes were measured weekly. Tumor volumes (mm³ were obtained by using the following formula: volume [symbol] (W²×L)/2 (W, width; L, length in mm of the tumor). On day 14 after treatment, one mouse in Afod RAAS 2+FS - - - high group was sacrificed due to tumor size reached more than 2,000 mm. On day 20 after dosing, one mouse in Afod RAAS 2+FS-moderate group died. On day 24 after treatment, all mice were sacrificed. Routine necropsy was performed to detect any abnormal signs of each internal organ with specific attention to metastases. Each tumor was removed and weighted.

3.3. Drugs and Materials

High concentrated fibrinogen enriched al at thrombin and Afod (FS), Afod RAAS2 and Afod RAAS 4 were provided by RAAS; Matrigel was from BD Biosciences (San Jose, Calif., cat. #356234).

Digital caliper was from Sylvac, Switzerland.

3.4. Data Analysis

3.4.1. Relative Chage of Body Weight (RCBW)

Relative change of body weight (RCBW) was calculated based on the following formula: RCBW (%)=(BWi−BWO)/BWO×100%; BWi was the body weight on the day of weighing and BWO was the body weight before surgery.

3.4.2. Tumor Weight

Tumors weighed after sacrificing mice.

3.4.3. Statistical Analysis

Data were expressed as mean±SEM; the difference between the groups was analyzed for significance using one-way ANOVA and Dunnett's test

4. Results

4.1. Tumor Growth Inhibition

On 14 days after treatment, the tumor volume in vehicle group reached 1070 nHn³ on average, while tumor volurne on average in Afod RAAS 2+FS-high, Afod RAAS 2+FS-moderate, Afod RAAS 2+FS-low and, Afod RAAS 4+FS groups was 663 mm³, 596 mm³, 640 mm³ and 531 mm³ respectively. On day 24 after dosing, the tumor size and tumor weight in FS combination with Afod RAAS 2 at all dosed or RAAS 4 groups was not significantly inhibited compared with sham-operated control group.

The inhibition on tumor growth were shown in FIG. 1-3.

4.2. Effect on Body Weight

Loss body weight, a sign of toxicity, was not seen in FS in combination with Afod RAAS 2 groups or vvith RAAS 4 groupindicatinq tht:test a t: nt has no/Htt!e side eh\\: cts.

The effect on body weight was shown in FIG. 4 and table 2.

5. Discussion

Patient-derived liver tumor xenograft (PDX) partial removal model was used to evaluate the anti-cancer efficacy of FS in combination with Afod RAAS 2 at 3 doses or with Afod RAAS 4 at one dose. When xenograft tumors reached 200 mm:³ a portion of tumor was removed by surger and a p01iion of tumor of 20 mm³ in size was left for fU!iher growth, and FS or a control agent was applied to wound surfaces of both sides after tumor removal.

The mice were treated 2 days after the surgery, and lasted for 24 days. On 14 days after treatment, the tumor volume in vehicle group reached 1070 mm³ on average, while tumor volume on average in AFOD RAAS 2+FS-high, AFOD RAAS 2+FS-moderate, AFOD RAAS 2+FS-low and, AFOD RAAS 4+FS groups was 663 mm:\ 596 mm:\ 640 mm³ and 531 mm³ respectively, which demonstrated Afod RAAS 2+FS or Afod RAAS 4+FS significantly inhibited the tumor growth. But anti-tumor efficacy did not last long, after about a week (on day 24 after dosing) the tumor size and tumor weight in FS combination with Afod RAAS 2 at all dosed or RAAS 4 groups reached more than 2000 mm³ and exhibited no significant difference with sham-operated control group, indicating no significant inhibitory effects on tumor growth.

In summary, high concentrated fibrinogen enriched al at thrombin (FS) in combination with Afod RAAS 2 or RAAS 4 inhibited the liver PDX tumor growth temporarily.

6. References

N/A

7. Figures

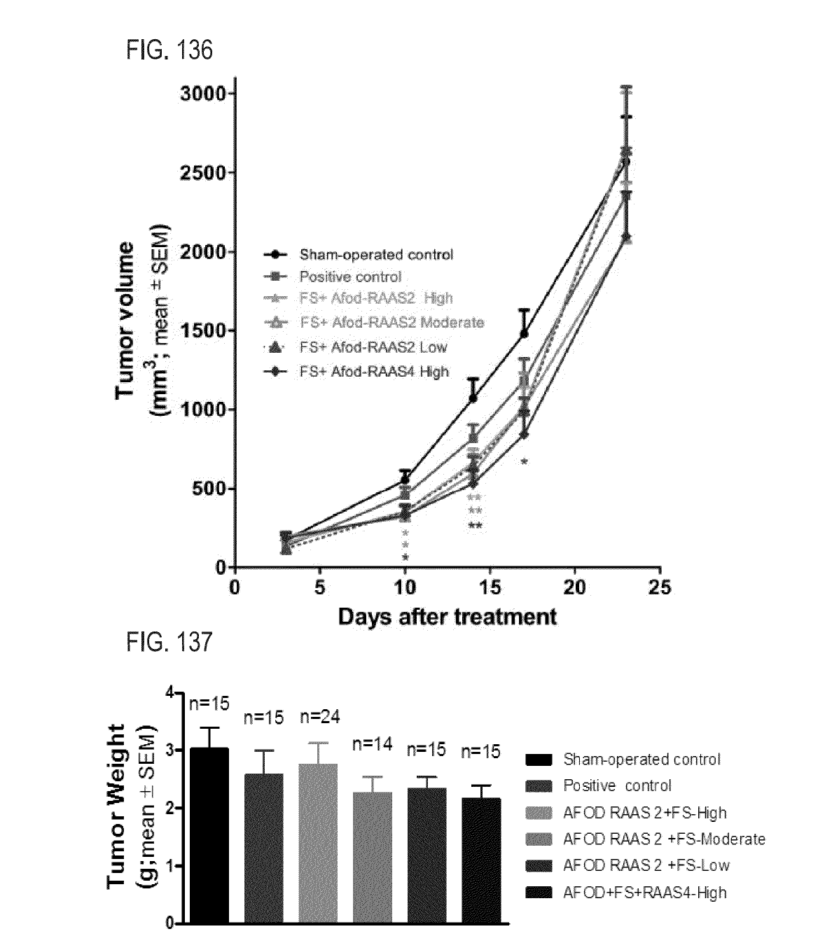

FIG. 136

Data are expressed as mean±SEM. *<0.05, **<0.0.1 vs sham group (one-way ANOVA and Dunnett's test).

FIG. 137

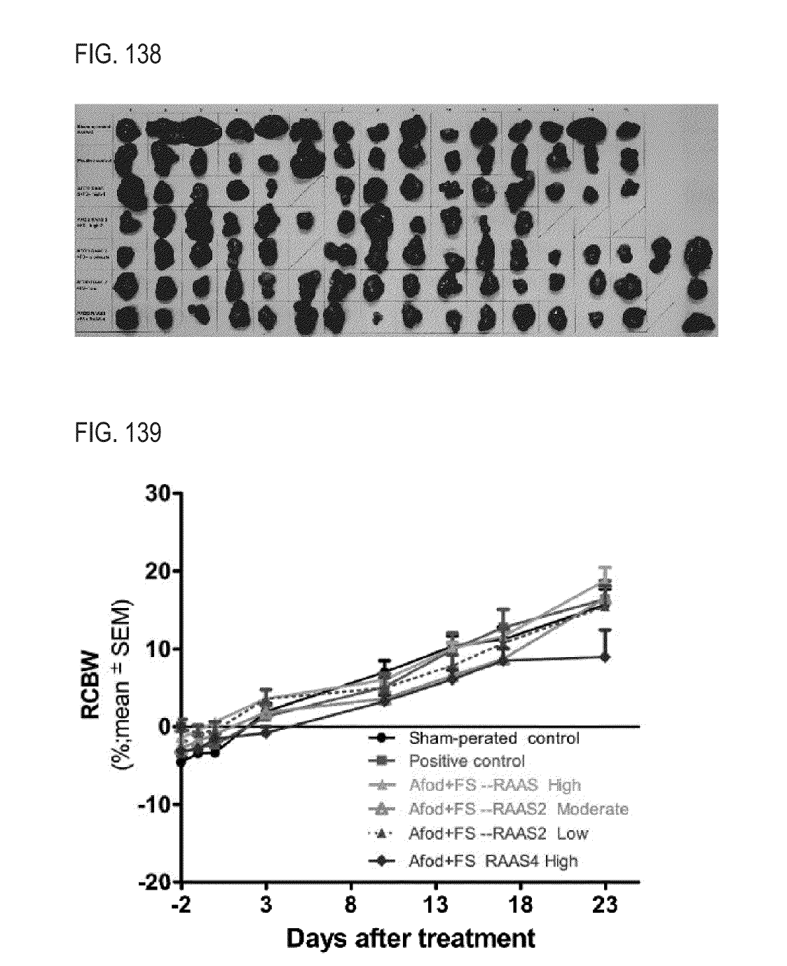

FIG. 138

Tumor was from each mouse of model LI-03-0117 and weighed. Scale bar, 1 em.

FIG. 139

Data are expressed as mean±SEM. Relative change of body weight (RCBW) was calculated based on the following formula: RCBW (%)=(BWi−BWO)iBWO×100%; BWi was the body weight on the day of weighing and BWO was the body weight before surgery.

8. Tables

Final Report

### Characterization of Lymphoid Tissues and Peripheral Blood in Nude Mouse Treated with and “\′vithout A FCC

### Executive Stnnmary

The purpose of this study was to investigate the effect of AFCC on curing tumor through characterizing distinct cell lineage in lymphoid tissues and peripheral blood in nude mouse treated with and without AFCC. Distinct cell lineage was differentiated by cell surface marker proteins. T cells, B cells, activated B cells, myeloid dendritic cell (mDC), plasmacytoid dendritic cell (pDC), granulocytes, and monocytes/macrophages were characterized.

In spleen and lymph nodes except in peripheral blood, AFCC treatment resulted in increased CD3+T cell population compared with that in nude mouse with tumor (FIG. 3, 9, 15). In spleen, lymph nodes and peripheral blood, with AFCC treatment, B cell population together with activated B cells also increased compared with those in nude mouse with tumor (FIGS. 4, 10, 16, 5, 10, and 20). In spite of the increased cell number of B cells and T cells after AFCC treatment, granulocytes decreased (FIG. 7, 14, 18). Macrophages were found to decrease after AFCC treatment In peripheral blood and spleen but not in draining lymph nodes (FIG. 6, 13, 19). mDC and pDC percentages were not greatly affected in nude mouse in the presence of AFCC (FIG. 8, 11, 17).

### LIST OF ABBREVIATIONS

| | |
|---|---|
| FACS | Flow Cytometry mDC |
| | Myeloid dendritic cell |
| pDC | Plasmacytoid dendritic cell |

Materials and Methods

Materials

Reagents

FITC, Rat Anti-Mouse CD4, BD, Cat: 557307

FITC, Rat Anti-MouseCD3 molecular complex, BD, Cat: 561798

PerCP-Cy5.5, Rat Anti-Mouse CD4, BD, Cat: 550954

PE, Rat Anti-MouseB220/CD45R, BD, Cat: 553089

APC, Rat Anti-MouseCD:Ub, BD, Cat: 553312

APC, Ar Ham Anti-MouseCD11c, BD, Cat: 550261

PE, Rat Anti-MouseGR-1(Ly-6G and Ly-6C), BD, Cat: 553128

Purified, Rat Anti-MouseFc blocker CD16/32, BD, Cat: 553141

APC, Ar Ham Rat Anti—MouseCD69, BD, Cat: 560689

7-AAD, BD. Cat: 559925

ACK Lysing buffer, Invitrogen, Cat: A10492-01

PBS, Dycent Biotech (Shanghai) CO., Ltd. Cat: BJ141.

FBS, Invitrogen Gibco, Cat: 10099141

1′VIaterials

Cell strainer (70 flm), BD, Cat: 352350

BD Falcon tubes (12×75 mm, 5 ml), BD, Cat: 352054

Equipments

Vi-CELL Cell Viability Analyzer, Beckman Coulter, Cat: 731050

FACSCalibur flow cytometer, BD, Cat: TY1218

Methods

Cell Isolation and Staining

Peripheral blood was collected through cardiac puncture. After removing red blood cells with lysis buffer followed by two rounds of washing using 1×PBS, mononuclear cells (monocytes, macrophages, dendritic cells, and lymphocytes) and granulocytes were obtained. Spleen and lymph nodes cell suspension were also obtained after filtering through 70 flrn cell strainer. Cell viability and number were analyzed by Vi-CELL Cell Viability Analyzer. Cell surface labeling was performed after that. Blocked with Fe blocker CD16/CD32 at 49 C for 15 min, cells were centrifuged and resuspended in staining buffer (0.08% NaN3/PBS+1% FBS). Fluorescent-conjugated antibodies were then added into the suspension at the indicated dilution according to the antibody usage protocol from the company. After 30 min incubation at 4 Q (for 30 min in the dark, cells were washed twice with 0.08% NaN3/PBS (200 fll per sample}, and resuspended with 400 fll 0.08% NaNjPBS in BD Falcon tubes (12×75 mm, 5 ml) followed by FAGS analysis.

Data Analysis

FACS data were analyzed by flowjo softvvare.

Study Summary

Study initiation date and completion date

The study was initiated and finished on Apr. 13, 2012.

Study Purpose

The purpose of this study was to investigate the effect of AFCC on curing tumor through characterizing distinct cell lineage in lymphoid tissues and peripheral blood in nude mouse treated vvitb and \vithout AFCC.

Study Results

1′Vlice information

All the mice were transferred from oncology team from vVuxi Apptec. FIG. 1 and FIG. 2 contained the treatment and age information of the mice.

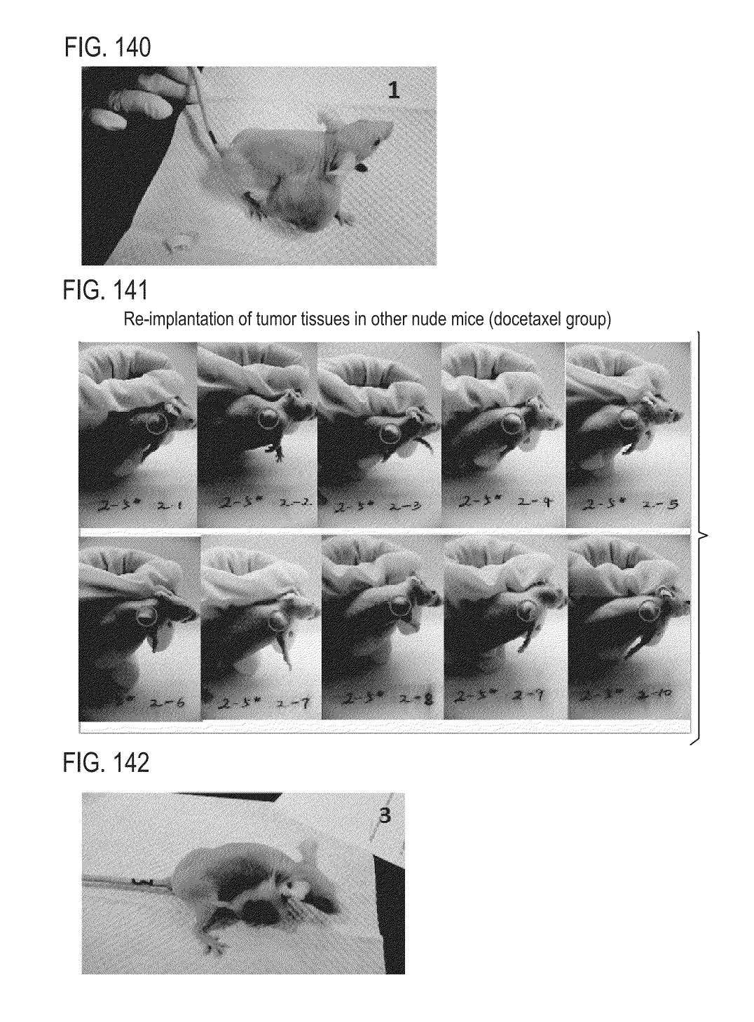

1: Nude m.ice with tumor: nude mice grafted vvith MDA-MB-231-Luc tmnor cells as vehicle for the study.
FIG. 140

10 nude mice from group 2-5 which have been implanted with tumor cells from the 2-5 mice positive control group using Docetaxel in another study done at another CRO lab.
FIG. 141

3: One of the 10 nude mice with MDA-MB-231-Luc tumor cells transferred from 2-5 positive control group using Docetaxel and it is used as positive control for the re-implantation study,
FIG. 142

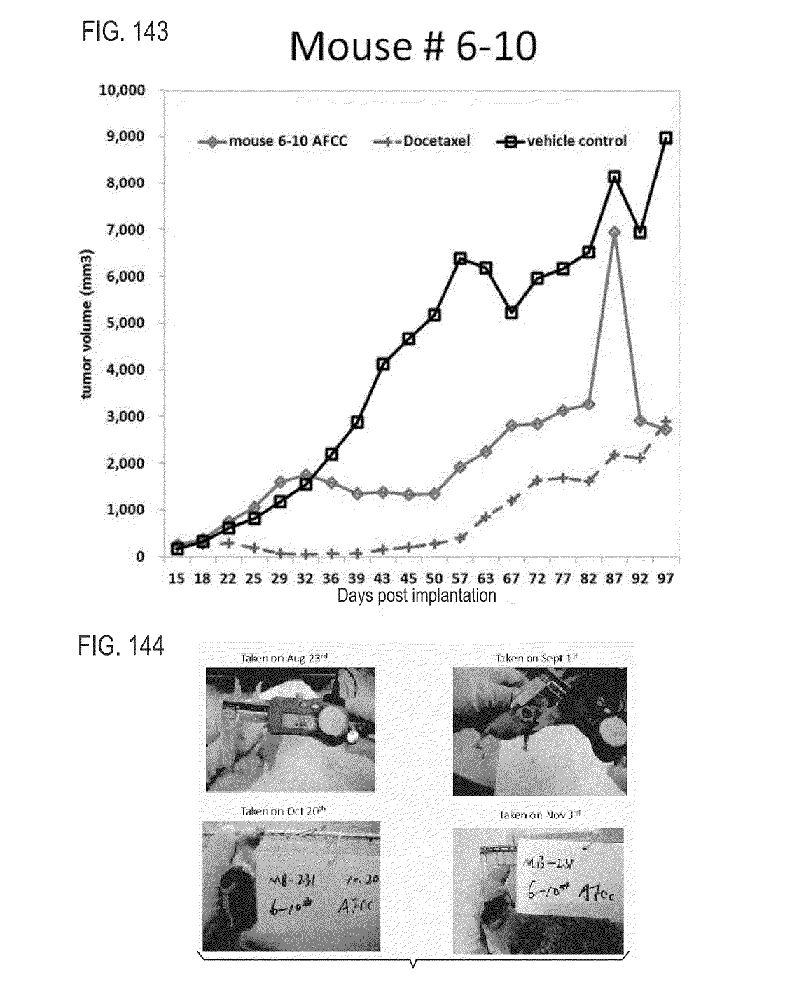

Graph showing the tumor volume of Mice #6-10 from the study done from July until Nov. 11, 2011 when the dead body of mouse #6-10 was removed from one CRO lab to another one for further study.
FIG. 143

Mouse #6-10 taken from Aug. 23, 2011 to November 3n,¹ 2011 showing the growth of the tumor which had been detached from the body was under recovery from breast cancer using AFCC proteins for treatment.
FIG. 144

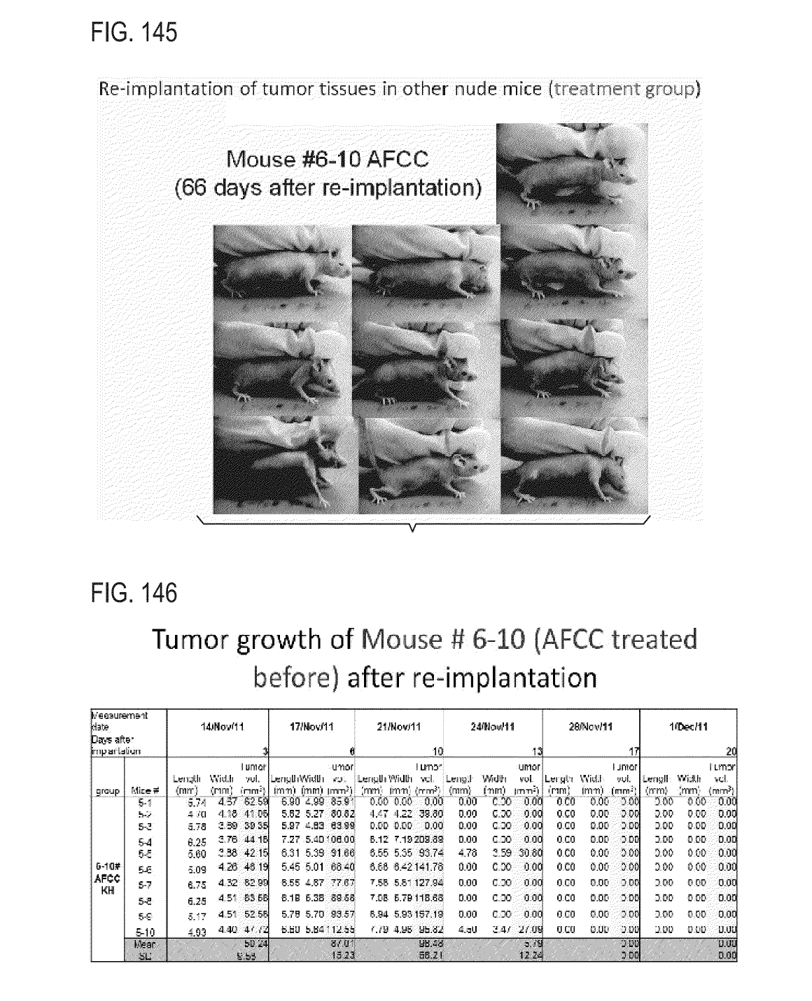

The tissue from the area of mouse #6-1 0 vvhere the tumor had been detached \vas used to implant in the 10 nude mice 66 days after re-implantations show no tumor growth.
FIG. 145

After 66 days 1 with no growth, then we implanted the cancer tumor for a second time. The growth of the tumor in mice 6-10 which had been treated prior with AFCC at another CRO lab after re-implantation on Nov. 11, 2011.
FIG. 146

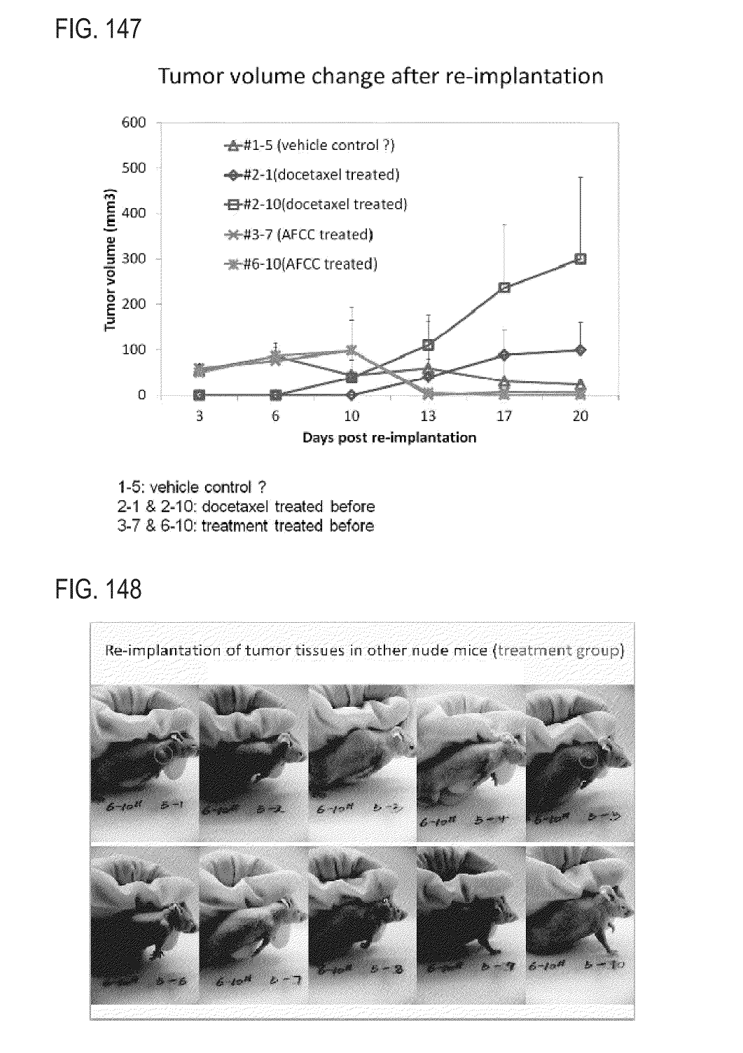

Graph showing 5 groups of nude mice after tumor volume change atler the second re-implantation with the breast tumor cancer, including mice #6-10 and mice #2-10 treated with Docetaxel.
FIG. 147

The picture of the 10 mice in group #6-10 showing mice #5-1 and mice #5-3 growing the tumor after second re-implantation both had been treated with AFCC on Feb. 29, 2012.
FIG. 148

2: Nude Mice with AFCC Treatment:

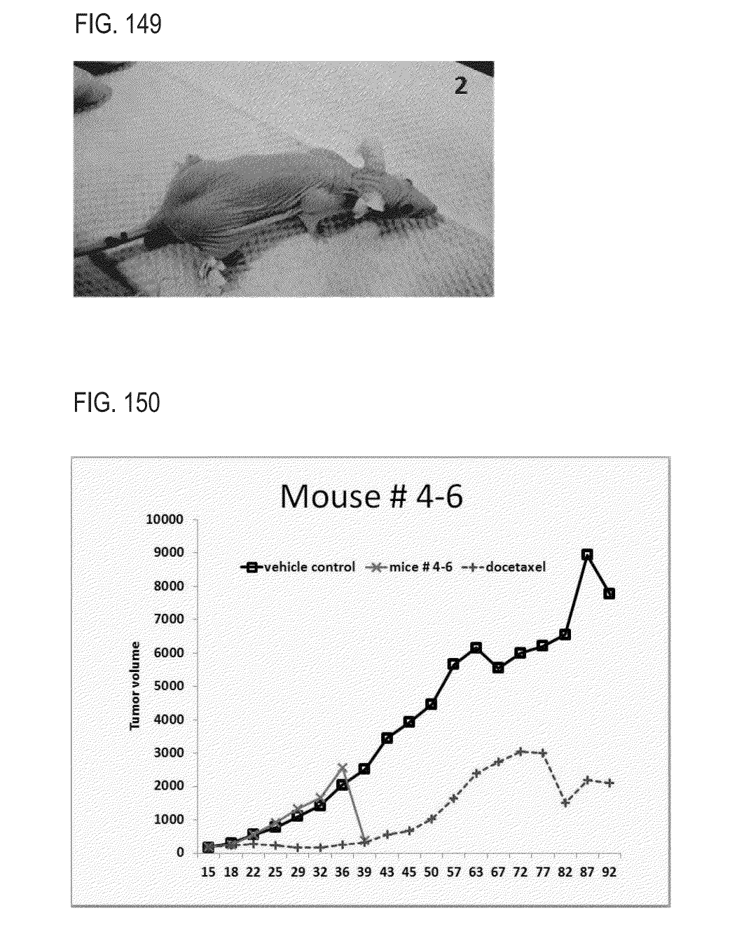

Grafted with tumor cells numbered #6-10 starting at 11-11-2011; received With AFCC provided by RAAS though I.V. or J.P. injection from 2-29-2012. In April mice #6-10 with the second re-implantation has been completely recovered due to the AFCC proteins ‘lvhich contain good healthy cells which sent signal to the DNA of the infected mice with breast cancer tumor, to transform the RNA to synthesize good proteins against the breast cancer eel L
FIG. 149.

Among the groups in the study for breast cancer from mid-July to Nov. 11, 2011 nude mouse #4-6 has shown the quickest recovery period within 24 days. From day 15 when the tumor started to grow to day 39 when the tumor detached from the body.
FIG. 150

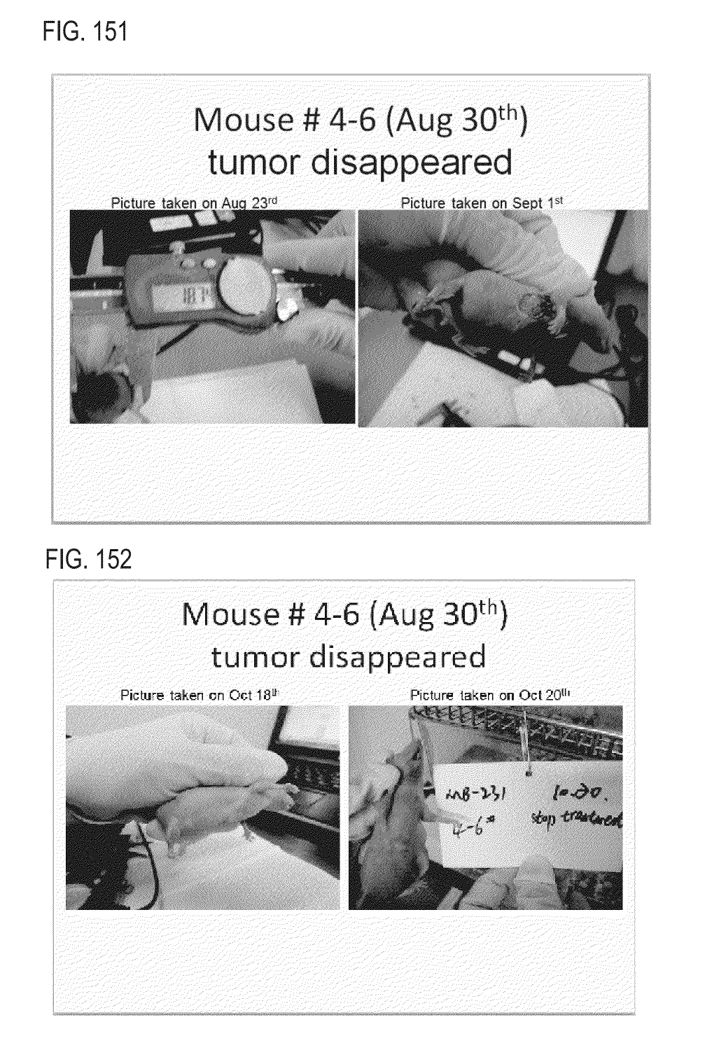

Mouse #4-6 grew the tumor on August 23rd and self-detached from the body September 1⁸\2011.
FIG. 151

Mouse #4-6 on October 18th completely recovered from breast cancer due to the i\FCC KH protein which contains good healthy cells which sent signal to the DNA of the infected mice with breast cancer tumor, to transform the RNA to synthesize good proteins against the breast cancer celL
FIG. 152

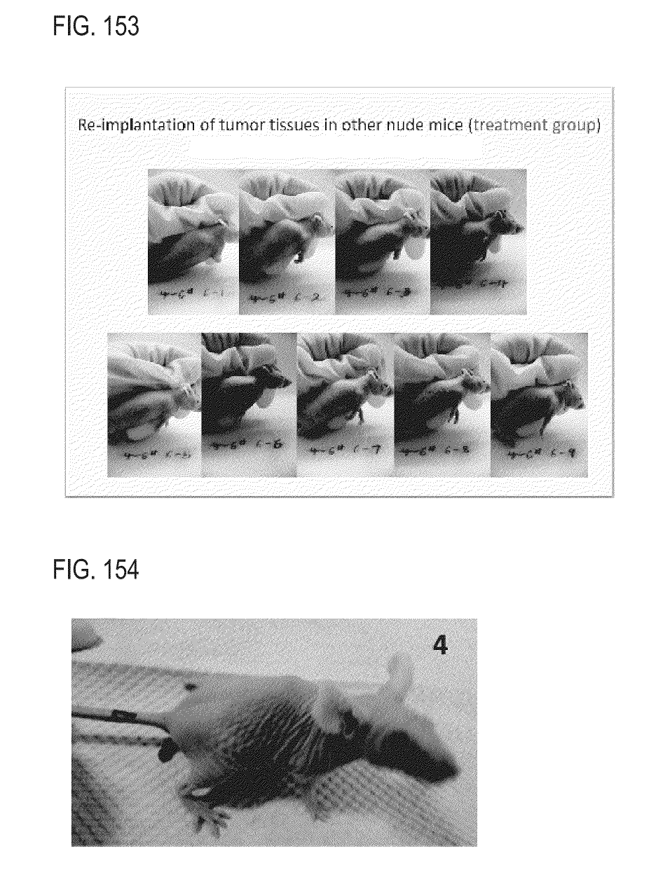

The 9 mice from the #4-6 group first re-implantation of the tumor which had never grown and one of these mice #4 was used in this study for analysis of the cells.
FIG. 153

4: Nude mouse with no tumor: grafted with tumor cells numbered #4-6 starting at Nov. 18, 2011, no further treatment needed due to failure of the tumor grmvth because good healthy cells fi.orn the AFCC treated, which contains good healthy cells which sent signal to the DNA of the infected mice with breast cancer tumor, to transform the RNA to synthesize good proteins against the breast cancer cell.
FIG. 154

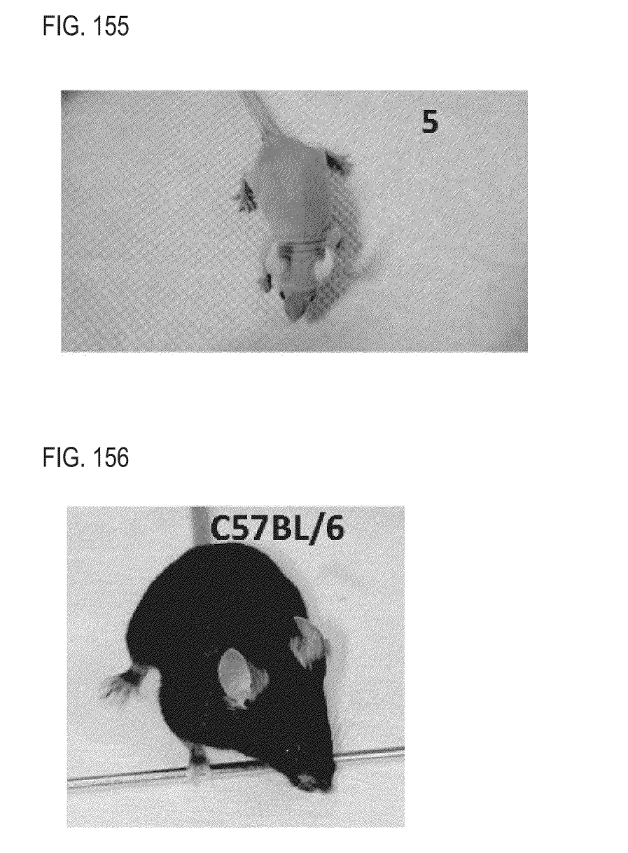

5: Nude na″ive mouse at 8 weeks old was used as a negative normal control to determine the normal nude mice cells.
FIG. 155

6: C57BL/6 mouse at 8 weeks old was used as a negative normal control to determine the normal nude mice cells.
FIG. 156

Cell Population in Peripheral Blood

After whole blood withdrawal, distinct cell lineage was differentiated by cell surface marker proteins. T cells, B cells, activated B cells, mDC, pDC, granulocytes, and monocytes/macrophages were characterized (FIG. 3 to FIG. 8).

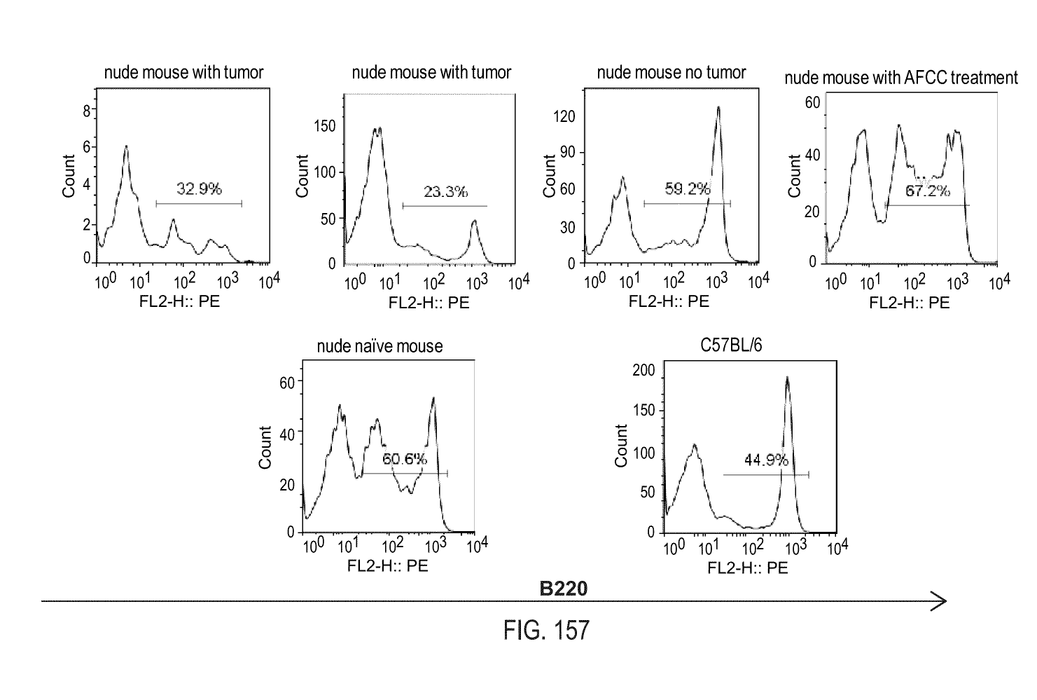

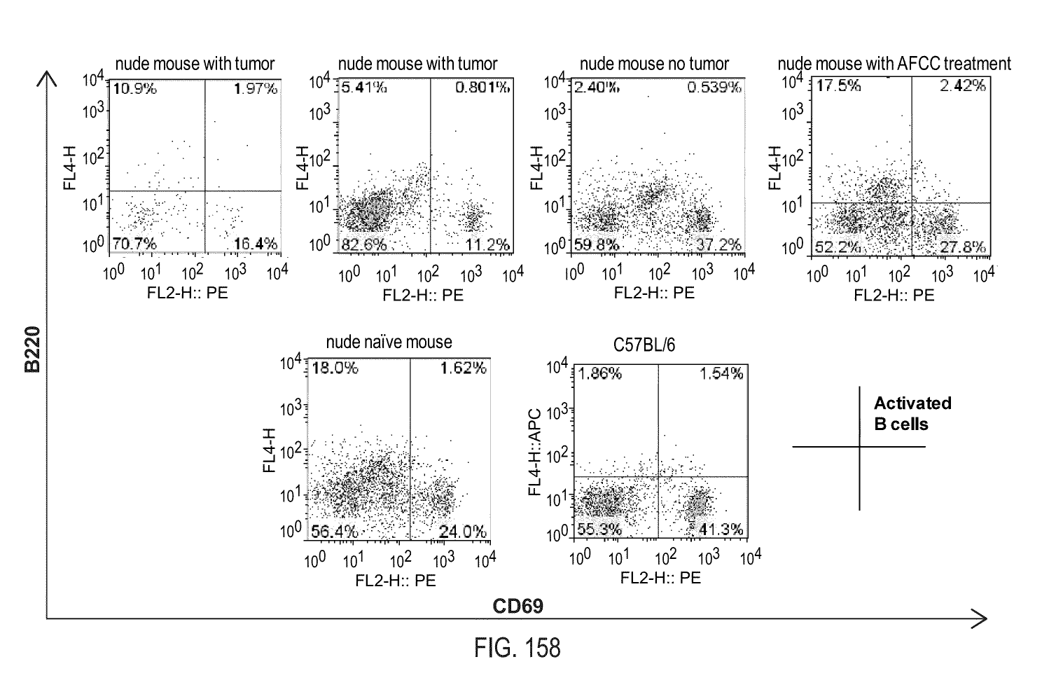

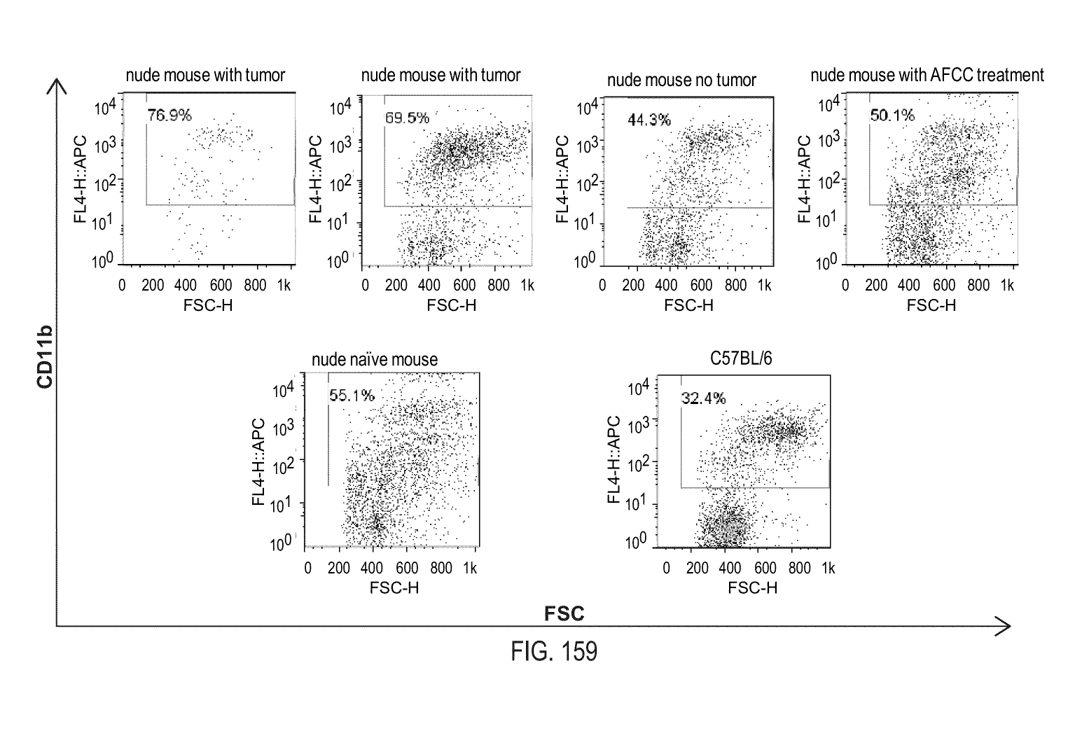

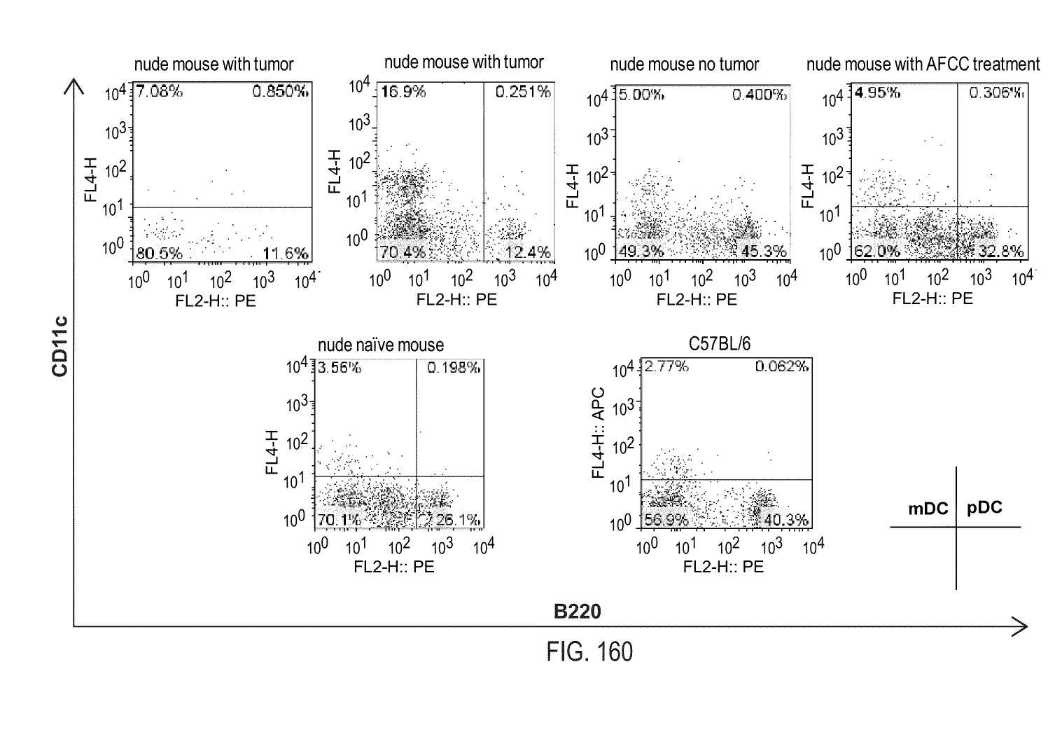

As shown by FIG. 3,AFCC treatment didn't affect CD3+T cell population compared with that In nude mouse with tumor and without tumor. After AFCC treatment, B cell population, on the other hand, increased to the similar percentage as seen in nude mouse no tumor and nude na′ive mouse, suggesting the potential effect of AFCC on B cell lineage (FIG. 4). Activated B cells also increased with AFCC treatment, which was illustrated in FIG. 5. Macrophages and granulocytes decreased after AFCC treatment compared with those in nude mouse with tumor (FIG. 6 and FIG. 7). Nude mouse no tumor and nude mouse with AFCC treatment had similar mDC and pDC percentage shown in FIG. 8.
FIG. 157
FIG. 158
FIG. 159
FIG. 160

Cell Population in Spleen

Distinct cell lineage in spleen cell suspension was further characterized by cell surface marker proteins. T cells, B cells, activated B cells, mDC, pDC, granulocytes, and monocytes/macrophages were included (FIG. 9 to FIG. 14).

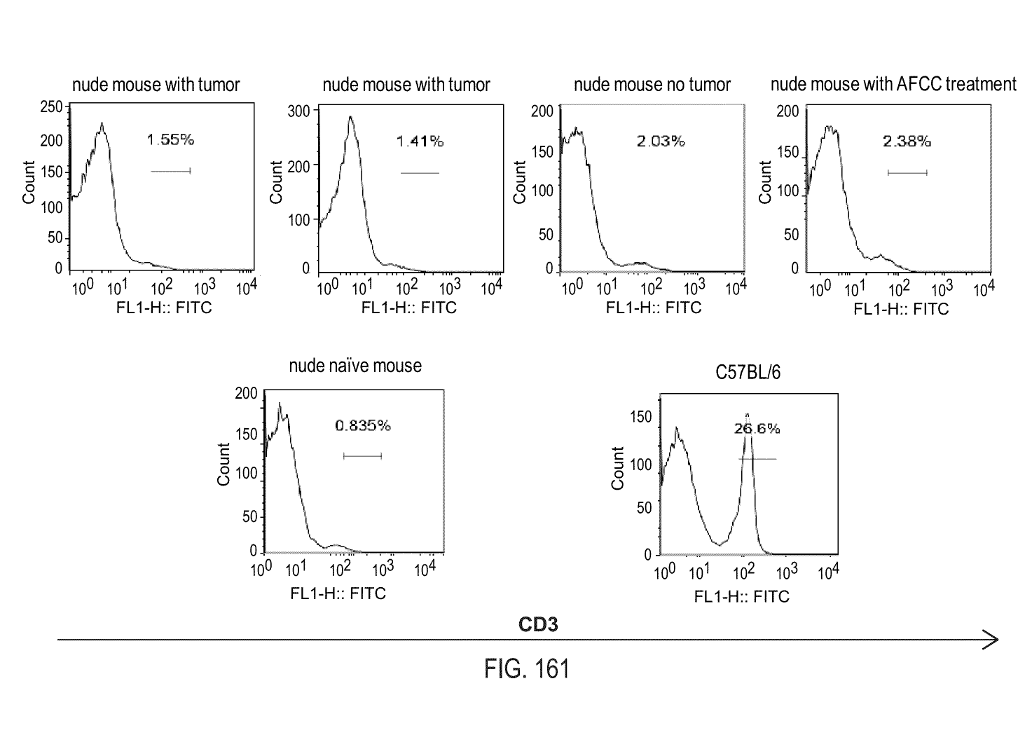

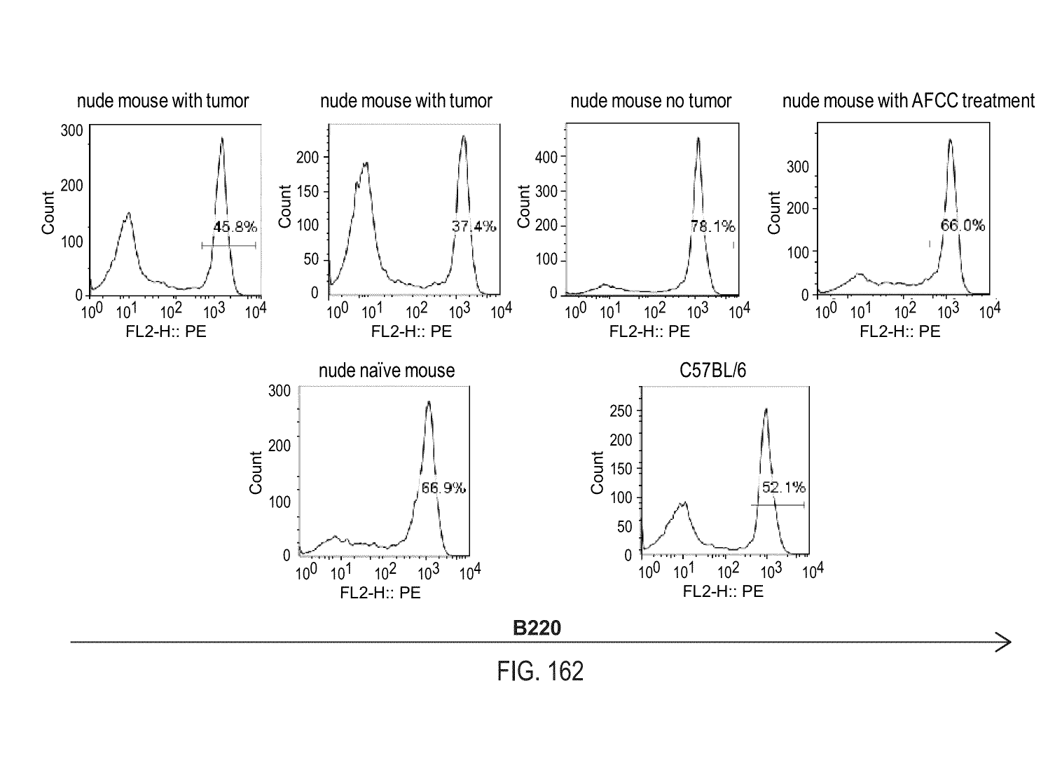

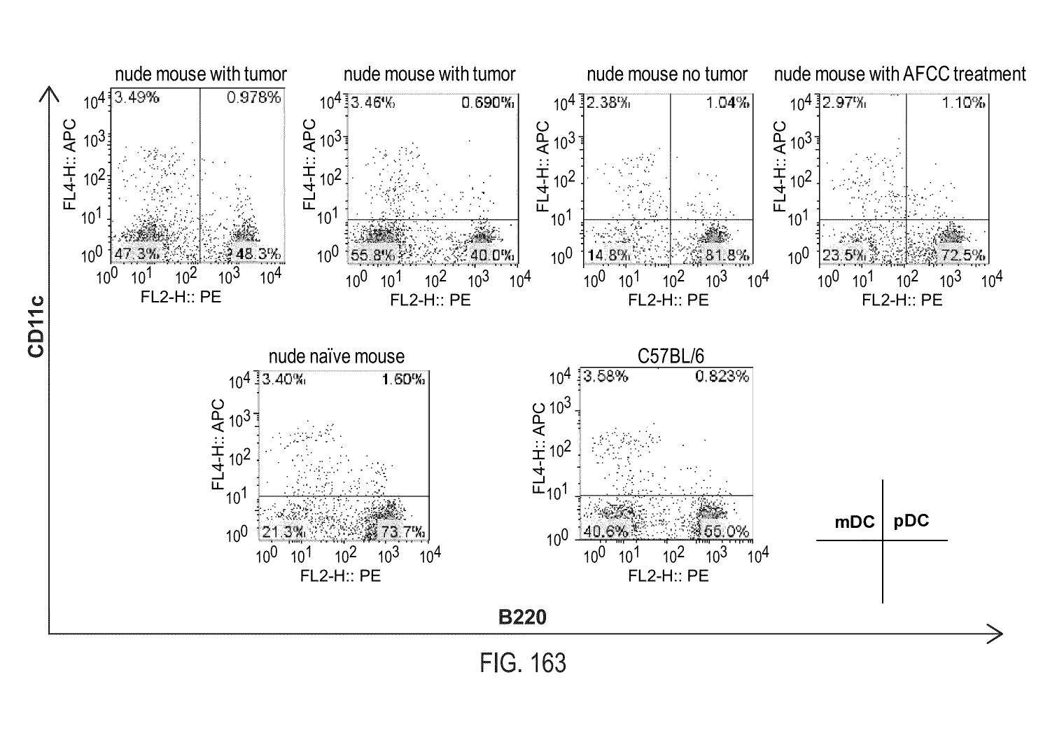

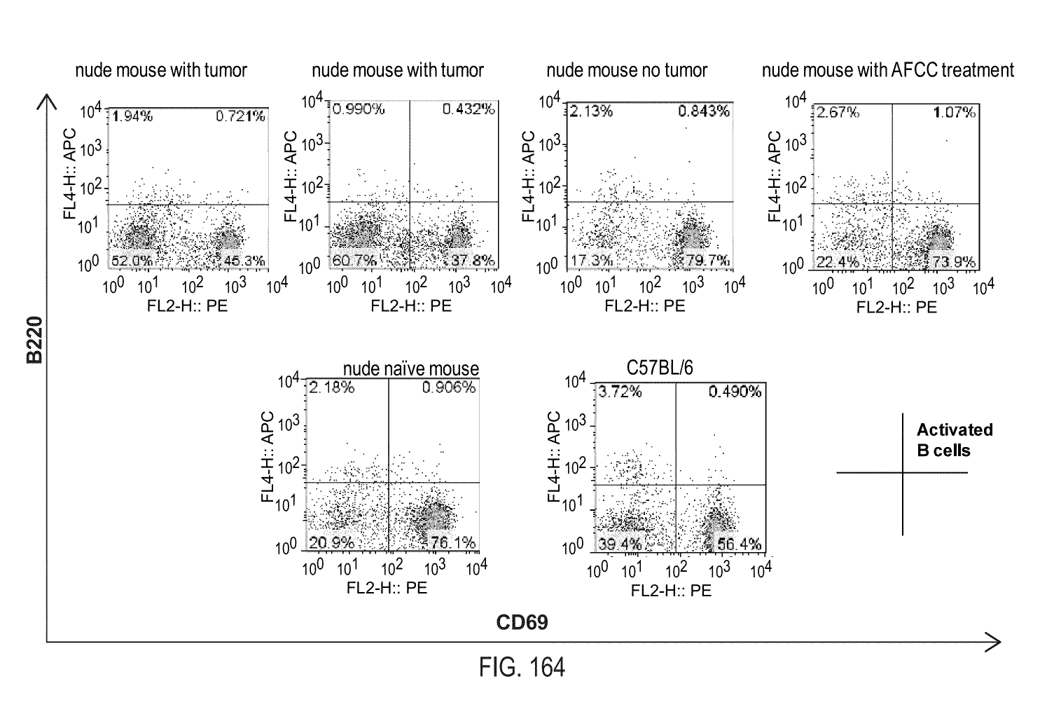

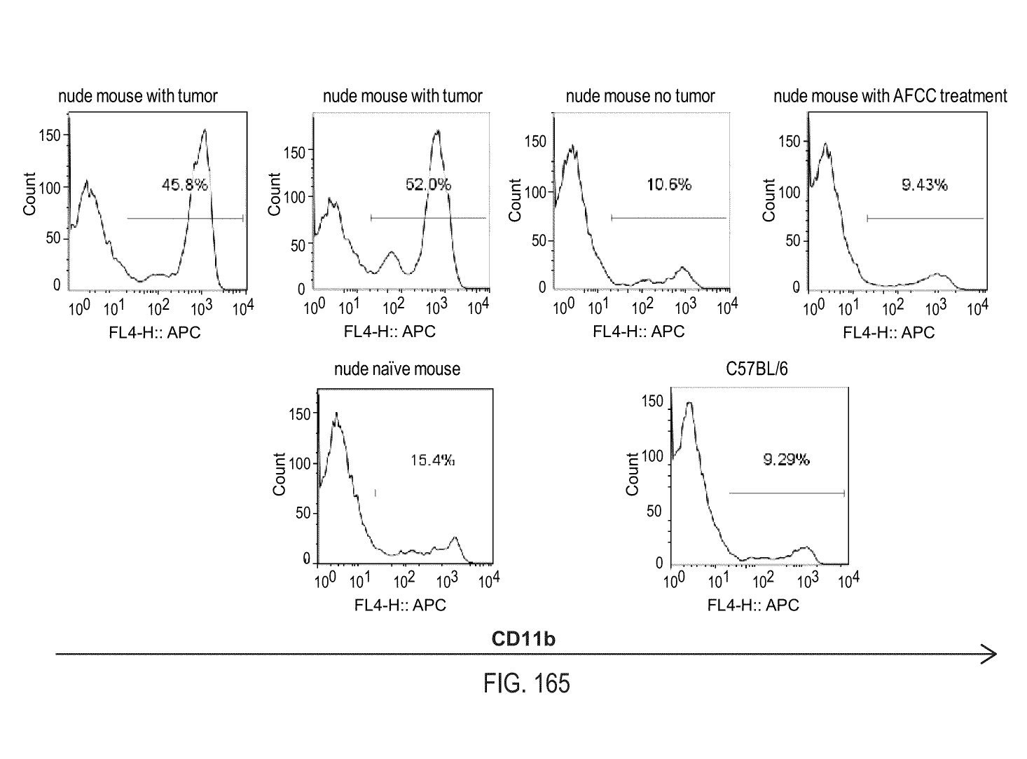

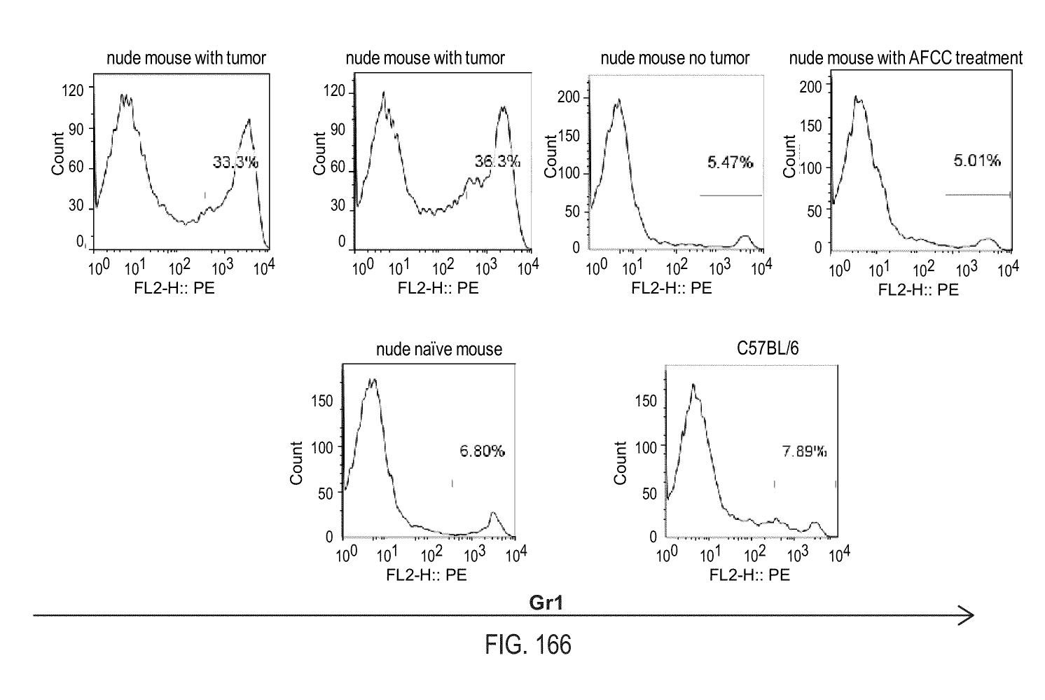

As shown by FIG. 9, AFCC treatment slightly increased CDJ′T cell population compared with that in nude mouse with tumor and nude mouse without tumor. After AFCC treatment, B cell population, on the other hand, increased to the similar percentage as seen in nude mouse no tumor, suggesting the potential effect of AFCC on B cell lineage (FIG. 10). Activated B cells also increased with AFCC treatment, which was illustrated in FIG. 12. 1\flacrophages and granulocytes dramatically decreased after AFCC treatment compared with those in nude mouse with tumor (FIG. 13 and FIG. 14}. Nude mouse no tumor and nude mouse with AFCC treatment had similar mDC and pDC percentage shown in FIG. 11.
FIG. 161
FIG. 162.
FIG. 163
FIG. 164
FIG. 165
FIG. 166

Cell Population in Draining LymJlh Nodes

Distinct cell lineage in draining lymph nodes suspension was further characterized by cell surface marker proteins. T cells, B cells, activated B cells, mDC, pDC, granulocytes, and monocytes/macrophages were included (FIG. 15 to FIG. 20).

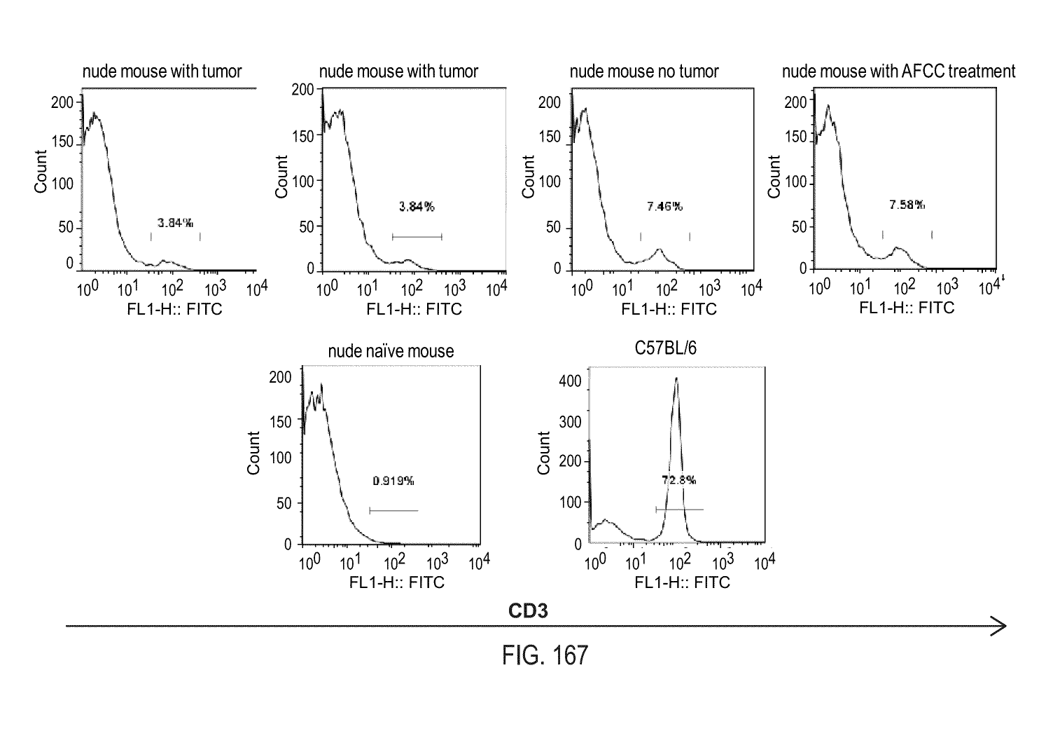

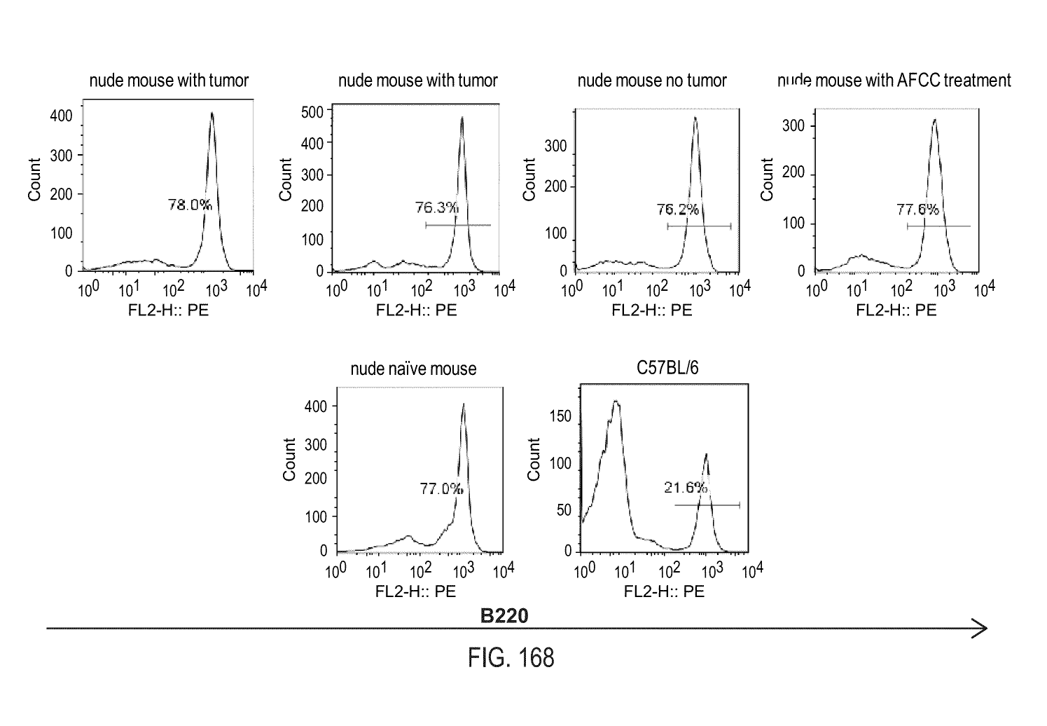

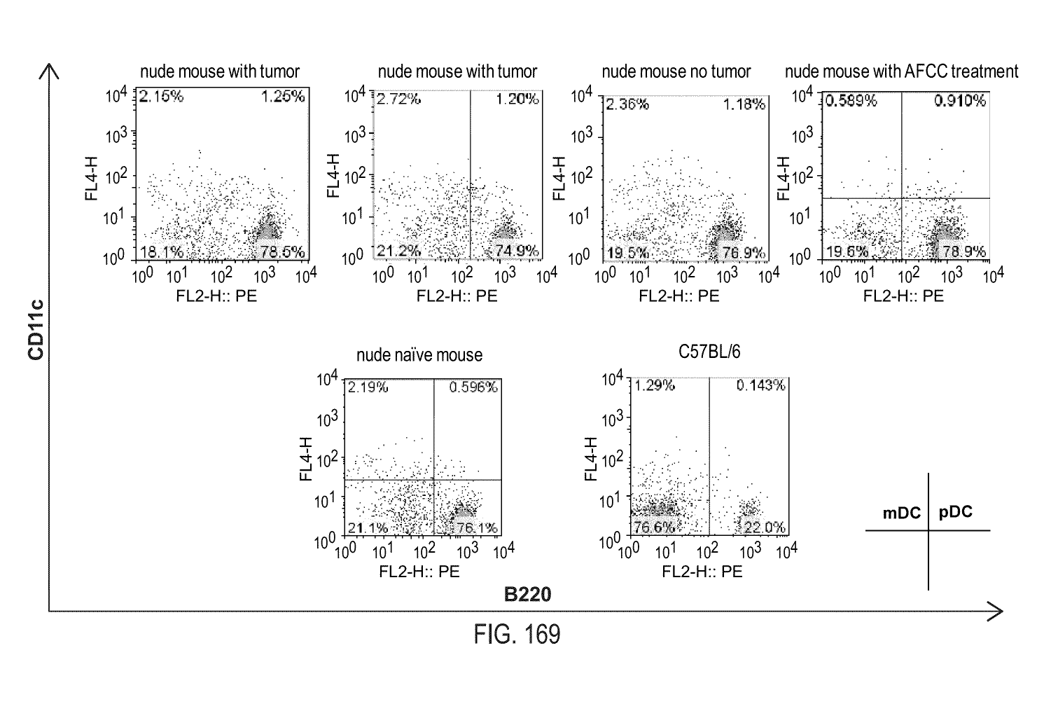

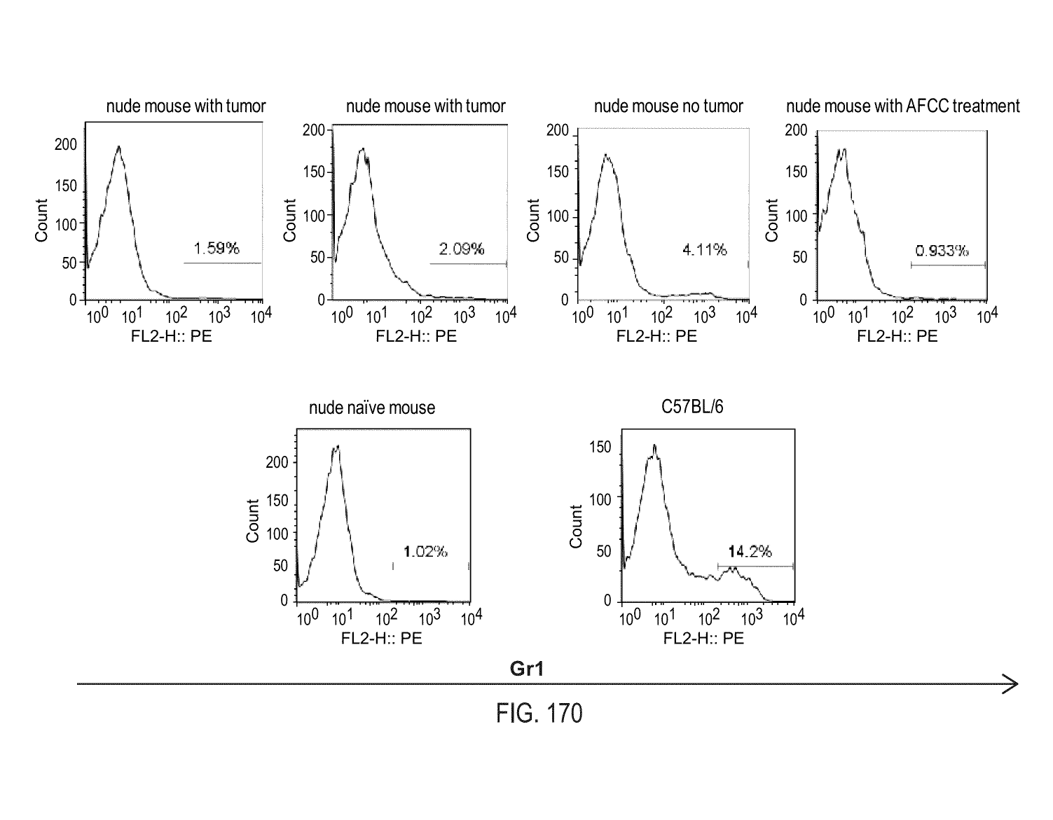

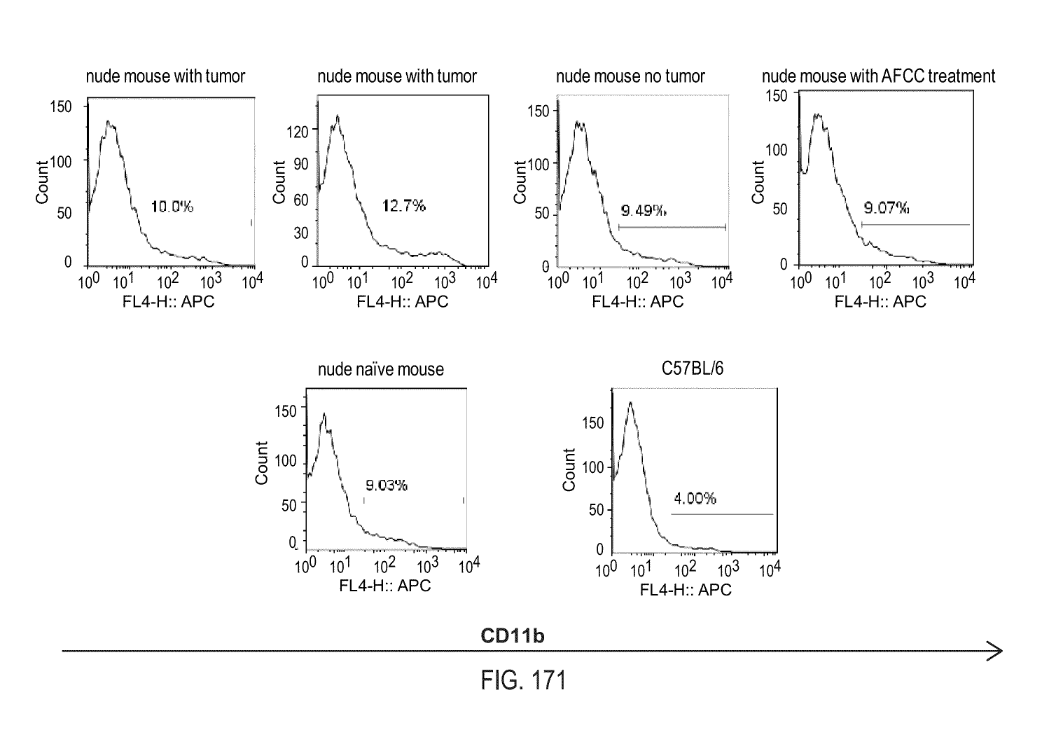

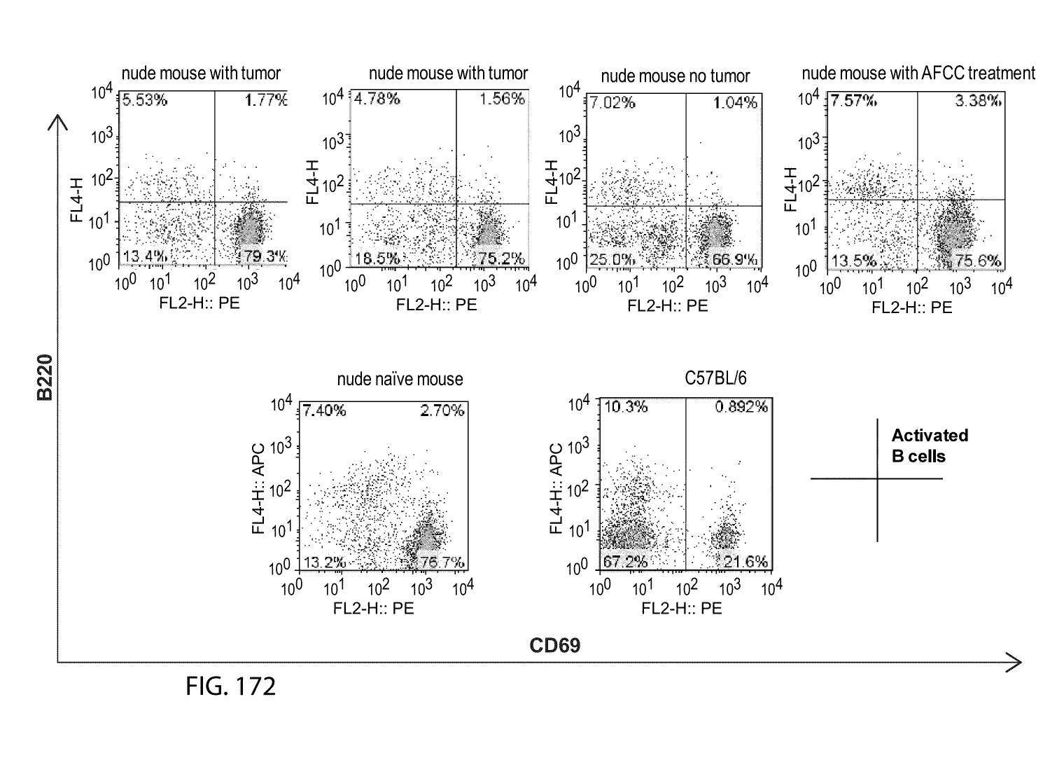

As shown by FIG. 15, AFCC treatment dramatically increased CD3_,_T cell population compared with that in nude mouse with tumor. T cells in nude mouse with AFCC treatment and mouse no tumor had the similar percentage (FIG. 15). After AFCC treatment, B cell population, on the other hand, increased to the similar percentage as seen in nude mouse no tumor, suggesting the potential effect of AFCC on B cell lineage (FIG. 16). Activated B cells also increased with AFCC treatment, which was illustrated in FIG. 20. Granulocytes dramatically decreased after AFCC treatment compared with those in nude mouse with tumor and na′ive nude mouse (FIG. 18). mDC and pDC also decreased in the presence of AFCC compared to those in nude mouse with or without tumor (FIG. 17). Macrophages still maintained the similar percentage with and without AFCC treatment (FIG. 19}.
FIG. 167
FIG. 168
FIG. 169
FIG. 170
FIG. 171
FIG. 172

7 Conclusions

The effect of AFCC on curing tumor through characterizing different cell lineage in lymphoid tissues and peripheral blood in nude mouse was investigated using staining with different marker proteins for distinct cell lineages followed by FACS. T cells, B cells, activated B cells, mDC, pDC, granulocytes, and monocytes/macrophages were characterized in 6 mice illustrated in FIG. 1 and FIG. 2.

FACS analysis showed that AFCC treatment had the effect on the population of major cell lineages in immune system. Increased CDJ′T cell population was found in nude mouse treated with AFCC compared with that in nude mouse with tumor in spleen and lymph nodes (FIG. 9, 15). B cells including activated B cells also increased compared with that in nude mice with tumor in spleen, lymph nodes, and peripheral blood (FIG. 4, 10, 1.6. 5, 10, 20}. Granulocytes and macrophages, however, were found to decrease after AFCC treatment in peripheral blood and spleen (FIGS. 7, 14, 18, 6, 1.3, and 19). The decrease as one of the lymphocytes, white blood cells. which are present in the peripheral blood of the nude mice with the breast cancer cell proves that the vehicle and positive control mice when the breast tumor grew the cancer cell have affected the peripheral blood. Even though the mice has not been metastasized. This make the inventor to believe that any cancer tumor grow the cancer cells are already in the peripheral blood.

KH good healthy cells 1—Send signals to the DAMAGED, SICK, AND BAD CELLS that triggers that synthesis of good proteins that transform these cells to become GOOD healthy cells; 2—Send signals to the other currently undarnaged cells to synthesis of good proteins to protect them from being DAMAGED, INFECTED and PRONE to DNA and other cellular alterations; 3 Send signals to the body to produce new cells that are healthy and forbid them from being affected by intra- and extracellular damaging signals in order to cure diseases, viruses infections, bacteria infections, auto immune disease neurological disorder, all type of solid and blood cancer, coagulation, diabetic, inhibitor, immune deficiency, muscle and nerve repair and restoration.

Macrophage have been found to decrease after AFCC treatrnent in peripheral blood and spleen. But it has not decreased in the vehicle and positive control mice. According to

the text books Macrophage is the big eater which consumes all bad and damaged cells and because of this they become sick or damaged. The level of Macrophage In the vehicle or positive control increase as they RNA of the bad damaged cells are synthesizing a bad protein that causes cancer. While KH good healthy cells synthesize good proteins against the breast cancer.

Taken together, this study suggests the effect of AFCC on curing tumor through changing the population of major cell lineages in immune system, including spleen, lymph nodes and peripheral blood.

Report: Antiviral efficacy of AFOD RAAS!R2 in an influenza H1N1 . . . infected mouse model

Report No: WX IFV05222012

Issue Date: Jun. 13, 2012

Study No: RAAS 05222012

Study Period: May″ 22I 2012 to Jun. 8, 2012

Objective

Infection with hurnan influenza virus (IFV) causes respiratory tract illness in human and animals including mice. Mouse model intranasally infected with IFV H1N1 is well recognized for antiviral compound screening against IFV infection. This study is designed to evaluate the compound AFOD RAAS2 from RAAS for its in vivo anti-IFV efficacy.

Study Method

This study was pe1 iormed in the following steps:

1) Infect mice with IFV by intranasal inoculation.

2) Treat the mice pre or post INF infection by iv/ip dosing of the AFOD RAAS2. 3) Daily record body weight of the mice.

4) Sacrifice survived mice and inspect their rnajor organs in the end of the study. Result Summary

One-week preventive treatment with RAAS-2 fully protected H1N1-challenged mice from death and body weight loss although one-week therapeutic treatment with RAAS-2 led to one mouse, out of 5 mice survived in this group to the end of the experiment. In the H1N1-challenged vehicle control group all mice died and their body weights dramatically dropped by 20% to 30% within 4-7 days post-IFV H1N1 challenge. In contrast with the vehicle group, all mice treated therapeutically with oseltamivir survived although their body weights dropped and recovered to some extent. This indicated that the mouse model worked successfully in current study.

For Study Protocol: RAAS 20120428.v.2

I. Method

Animals:

Female BALB/c mice (6-8 weeks, 17-22 g) were divided into defined study groups after a visual examination and a 3 to 5-day acclimation upon arrival.

Solution Preparation:

1. Sodium Pentobarbital: Freshly dissolved in saline for injection at 7.5 mg/ml prior to using.

2. Test article: human plasma derived protein 29% AFOD RAAS2 in sterile solutions for vein injection provided by the client.

3. Vehicle: PBS

4. Oseltamivir phosphate (prodrug): aqueous solution in PBS, 0.1 mg/ml

Experimental Procedure:

IFV Infection and Test Article Administration:

1, Frorn day-7 through day-1, 5 mice from group 4 are intravenously or intraperitoneally (iv/ip)

administrated daily for 7 days.

2. On the day of Influenza administration, mice are anesthetized by intraperitoneal injection of sodium pentobarbital (80 mg/kg).

3. Anesthetized mice are inoculated with 5×10″3 pfu/rnouse of Influenza H1N1 ANVSN/33 via the intranasal route in SFM medium.

4. Test article or vehicle is intravenously or intraperitoneally (iv/ip) administrated daily for 7 days. Oseltarnivir (1 mg/kg) is orally given twice daily for 8 days. First dosing for oseltarnivir or test article is executed 4 h pre H1N1 inoculation.

5. From day 1 through day 14 the infected mice are observed two times a day. Mortality and body weight are recorded daily.

6. On day 14, all living mice are sacrificed and dissected for the inspection of organ appearances.

II. Groups and Schedules:

[symbol]

TABLE 1
Action summary of the Study
Table 1 Action summary of the Study

| | | | 1FV | AFOD, | | | |
|---|---|---|---|---|---|---|---|
| Study | | | challenge, | iylip, | Oseltamivir, | po | mouse |
| Day | Date | Weighing | 14:00-16:00 | 10:00-12:00 | 10:00-10:20 | 19:50-20:10 | sacrifice |
| Day -7 | May 22, 2012 | -V | | N1 | | | |
| | | | | --------------------------- + | | | ---------------- I |
| Day -6 | May 23, 2012 | | [symbol] j | | | | |
| Day -5 | May 24, 2012 | [symbol] | | ..,1 | | | |
| Day -4 | May 25, 2012 | -V | | Ni | | | |
| | | | | --------------------------- + | | | ---------------- I |
| Day -3 | May 26, 2012 | [symbol] j | | ′ | | | |
| Day -2 | May 27, 2012 | | -V | | | | |
| Day -1 | May 28, 2012 | -V | | Ni | | | ---------------- I |
| | | | | ′ | | | |
| Day 0 | May 29, 2012 | [symbol] j | Ni | --------------------------- | Ni | | |
| Day 1 | May 30, 2012 | -V | | | [symbol] | | |
| Day 2 | May 31, 2012 | -V | | [symbol] | | N [symbol] | |
| | | | | ′ | | | ---------------- I |
| Day 3 | Jun. 01, 2012 | [symbol] j | | --------------------------- | Ni | | |
| Day 4 | Jun. 02, 2012 | -V | | ..,1 | [symbol] | [symbol] j | |
| Day 5 | Jun. 03, 2012 | -V | | v [symbol] | | | |
| | | | | --------------------------- | | N [symbol] | |
| Day 6 | Jun. 04, 2012 | ,j | | --------------------------- | Ni | | |
| Day 7 | Jun. 05, 2012 | -V | | | | | |
| Day 8 | Jun. 06, 2012 | -V | | | | | |
| Day 9 | Jun. 07, 2012 | ,,i | | ------------------ | | | ---------------- I |
| | | | | +----------------- | | | |
| Day 10 | Jun. 08, 2012 | -V | | | | | |
| Day 11 | Jun. 09, 2012 | -V | | | | | |
| Day 12 | Jun. 10, 2012 | | | _{d}+----------------- | | | ---------------- I |
| Day 12 | Jun. 11, 2012 | -V | | | | | |
| Day 13 | Jun. 12, 2012 | -V | | | | | I |
| | | | | ----------------- | | | v,----- |
| Day 14 | Jun. 13, 2012 | d | | | | | |

indicates ti⁻)at the action was taken. [symbol]
indicates data missing or illegible when filed

TABLE 2
ExperimentalDesign for the efficacy study

| | | | | | | 1^{st} | |
|---|---|---|---|---|---|---|---|
| | | | | Vol | Treatment | treatment | H1N1 |
| Group | Mice | Compound | Dose | (ml/kg) | Schedule | time | (PFU/mouse) |
| | 5 | Vehicle | 0.2/0.4 | — | Iv/ip, QD* | 4 hrs pre- | 5 × 1_0A3 |
| | | | mi/mouse | | | infection | |
| 2 | 5 | AFOD RAAS | nil/mouse | — | 0.2/0044 | hrs | 5 × 10A3 |
| | | 2 | | | Iviip, QUA | pre-infection | |
| 3 | 5 | Oseltamivir | 1 nig/kg | [symbol] | p0, BID″ | 4 | 5 × 1 0A3 |
| | | | | 10 | | | |
| 4 | | | | | | | |
| ----------- | 5 | AFOD RAAS | 0.2/0.4 | — | Iv/ip, QD* | 7 days | |
| + --------- | | | milmouse | | 5 × 10″′32 | pre-infection | |

Iv/ip, OD*: Iv/ip means that iv injection is carried out with the volume indicated in “dose” column on day 0, 1, 2, 4 and ip Injection is carried out on day 3;
QD: daily (QD) for 4 days after H1N1 inoculation;
**BID, twice daily.
Vehicle: PBS [symbol]
indicates data missing or illegible when filed

[symbol]

BI Adverse Events and Tolerability of Compounds:

1. On day 5 post H1N1 infection, hematuria occurred in group 2 of AFOD RAAS2 treatment. We stopped AFOD RAAS2 medication on the sixth day post H1N1 infection.

2. One mouse in the oseltamivir group died day 3 post H1N1 challenge. Its body dissection indicated that its esophagus was damaged probably due to harsh oral gavage. Therefore this rnouse was ruled out frorn the experiment

Result and Discussion

In the H1N1-challenged vehicle control group aII5 mice died and their body weights dramatically dropped by 20% to 30% within 4-8 days post-IFV H1N1 challenge (FIG. 1, FIG. 2, and Table 3). In contrast with the vehicle group, 4 out of 5 mice in the oseltamivir group survived to the end of experiment (FIG. 1, FIG. 2, and Table 3) although one mouse died accidentally of harsh oral gavage, which should be ruled out frorn the experiment as suggested early (see Part III, 2 in this report). The body weights in this group dropped by <15% days 5 to 8 post HI N1 challenge and recovered thereafter to some extent (FIG. 2). This indicated that the mouse model worked successfully in current study.

Impressively one-week preventive treatment with 0.2 ml/0.4 ml/mouse iv/ip QD of RAAS-2 totally protected HIN1-challenged mice from death and body weight loss till the end of this study (Fig I, FIG. 2 and Table 3). The protection of body weight loss by the preventive treatment of RAAS-2 is even better than that by oseltamivir treatment (FIG. 2). However the therapeutic treatment with 0.2 ml/0.4 ml iv/ip QD of RAAS-2 only protected one mouse out of 5 mice in the group from death and partial body weight loss of all 5 mice days 2 to 5 post H1N1 infection. Other 4 mice in this group died days 4 to 6 post H₁N1 infection. In addition, some of the mice in [symbol] le RAAS-2 [symbol] lerapeutl'c group (′G2) [symbol] ad [symbol] indicating that the dose used in this group was beyond mouse tolerance in H1N1 challenge statu8.

We don't understand why the RAAS-2 displayed such significant preventive efficacy on mouse death and body weight loss caused by H1N·1 challenge. We have a number of suggestions to fully establish and understand this efficacy. First, we need to expand the efficacy experirnent using a few rnore mice each group to confirrn the data due to the small experiment scale (5 mice each group only) in the current study. In addition, a longer term study should be designed to fully know how long the preventive efficacy of the blood-derived product RAAS-2 could last For example the mice should be challenged with H1N1 two weeks, three weeks, four weeks and even longer, respectively, post one week of preventive treatment of the RAAS-2. Some well designed mechanism studies should be carried out, such as in vivo H1N1 replication in infected rnouse lungs in the preventive treatment and control groups, detection of immunological markers to reflect immune system activation and other biomarker assays post preventive treatment and H1N1 challenge. Finally a dose-dependent observation should be carried out for the RAAS-2 preventive treatment.

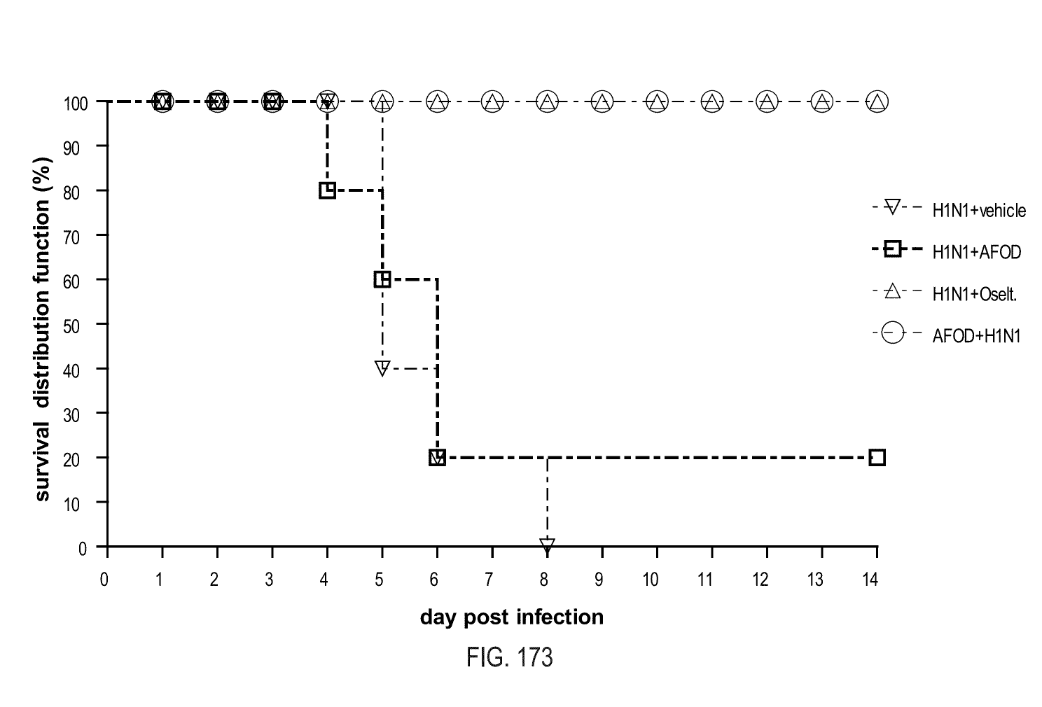

FIG. 173. Effect of AFOD RAAS2 on H1N1Mcaused mouse mortality

TABLE 3
Effect of AFOD RAAS2 or Oseltamivir on mean day to
death (MOD) of mice infected with H1N1 A/WSN/33

| | | | Survivor/ | Mean day to |
|---|---|---|---|---|
| Treatment | Dose | | total | death ± S.D. |
| H1N1 + Vehicle | 0.2/0.4 | nil/mouse | 0/5 | 4.8 ± 1.3 |
| H1N1 + | 1 | mg/kg | 1/5 | 6.2 ± 4.4 |
| AFOD RAAS2 | | | | |
| H1N1 + | 0.2/0.4 | ml/mouse | 4/4 | >14 ± 0.0*** |
| Oseltarnivir | | | | |
| AFOD RAAS2- | 0.2/0.4 | ramouse | 5/5 | >14 ± 0.0″* |
| 4 − H1N1 | | | | |

***P < :0.001 compared to the H1N1 + vehicle control

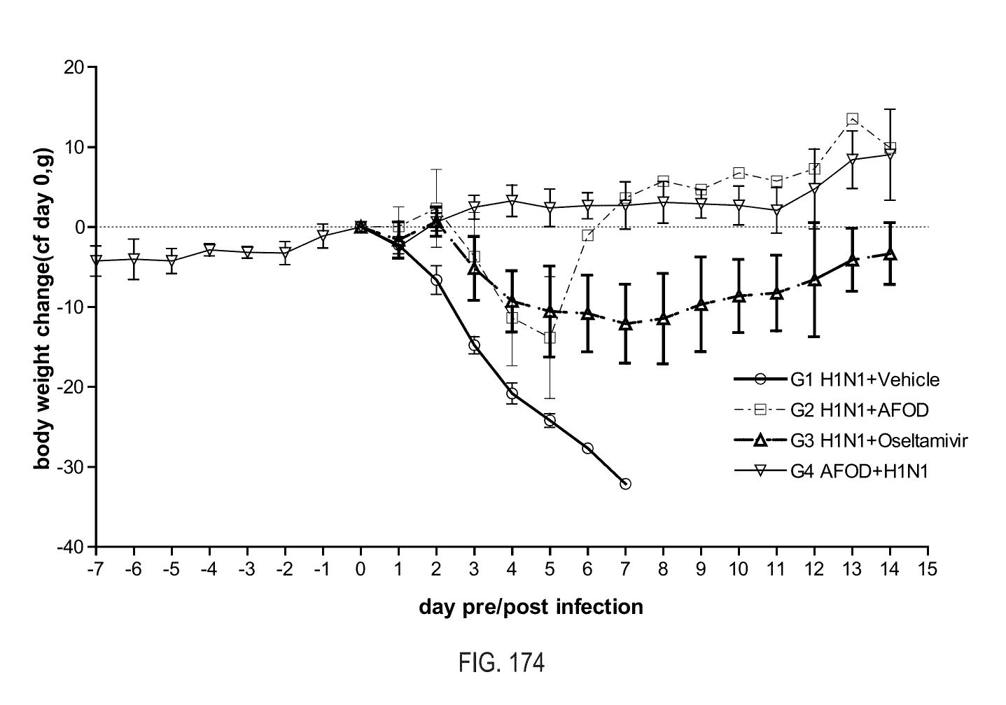

FIG. 174. The average body weight change in mice infected with H1N1 influenza

Appendixes:

The scanned primary in vivo experiment records of study RAAS 04242012 are attached. File name: Primary in vivo Experiment Record of Study RAAS 04242012

Effects of AFOD on 6-OHDA rat model of Parkinson's disease

I. General Information

Experiment requested by: Mr. Kieu Hoang from Shanghai
RAAS Project ID/code: RAAS/PD2k′11-01

Experimental objective: To study the effects of AFOD on 6-OHDA lesioned rat model of Parkinson's disease
Target start date: Jul. 18, 2011

II. Sample Information
Sample description: AFOD: Liquid, the concentration is 5%, store at 40 C

III. Introduction

The objective of this study was to determine if there were any neuroprotective or regeneration effects of AFOD on 6-OHDA lesioned rat model of Parkinson's disease. Behavioral tests (cylinder test, adjusting step test and rotation test) and tyrosine hydroxylase (TH) staining were used for evaluating the locomotive performance of the animals and survival of

dopaminergic neurons.

IV. Experimental Design

| | | | | Frequency | Drug tax in | | | | Behavioral | |
|---|---|---|---|---|---|---|---|---|---|---|
| | No. | | | (every 3 | relation | 6-OH DA | Dose | | tests & video | IHO TH) |
| Group | Animal | Sample | Route | days) | with lesion | lesion | (glkg) | | Recordings | ( |
| A | 10 rats | diluents | IV | Day 1, | Pre | Day 15 | 09/ | kg | 2 weeks | After |
| | | | | 4, 7, 10, 13 | | | | | after 6- | riehavioral |
| | | | | | | | | | OHDA | tests |
| B | 10 rats | AFOD | IV | Day 1, | Pre | Day 15 | 0.5 | g/kg | 2 weeks | After |
| | | | | 4, 7, 10, 13 | | | | | after 6- | behavioral |
| | | | | | | | | | OHDA | tests |
| C | 10 rats | AFOD | IV | Day 1, | Pre | Day 15 | 0.259/ | kg | 2 weeks | After |
| | | | | 4, 7, 10, 13 | | | | | after 6- | behavioral |
| | | | | | | | | | OHDA | tests |
| D | 10 rats | AFOD | IV | Day 1, | Pre | Day 15 | 0.125 | g/kg | 2 weeks | After |
| | | | | 4, 7, 10, 13 | | | | | after 6- | behavioral |
| | | | | | | | | | OHDA | tests |
| E | 10 rats | diluents | IV | Day 1, | post | Day 1 | O | g/kg | 2 weeks | After |
| | | | | 4, 7, 10, 13 | | | | | after 6- | behavioral |
| | | | | | | | | | OHDA | tests |
| F | 10 rats | AFOD | IV | Day 1, | post | Day 1 | 0.5 | g/kg | 2 weeks | After |
| | | | | 4, 7, 10, 13 | | | | | after 6- | behavioral |
| | | | | | | | | | OHDA | tests |
| | 10 rats | AFOD | IV | Day 1, | post | Day 1 | 0.25 | g/kg | 2 weeks | After |
| | | | | 4, 7, 10, 13 | | | | | after 6- | behavioral |
| | | | | | | | | | OHDA | tests |
| H | 10 rats | AFOD | IV | Day 1, | post | Day 1 | 0.1259/ | kg | 2 weeks | After |
| | | | | 4, 7, 10, 13 | | | | | after 6- | behavioral |
| | | | | | | | | | OHDA | tests |
| | 10 rats | diluents | IV | Day 1, | Pre + post | Day 15 | 09/ | kg | 2 weeks | After |
| | | | | 4, 7, 10, 13, 16 | | | | | after 6- | behavioral |
| | | | | 19, 22, 25, 28 | | | | | OHDA | tests |
| J | 10 rats | AFOD | IV | Day 1, | Pre + post | Day 15 | 0.59/ | kg | 2 weeks | After |
| | | | | 4, 7, 10, 13, 16 | | | | | after 6- | behavioral |
| | | | | 19, 22, 25, 28 | | | | | OHDA | tests |
| K | 10 rats | AFOD | IV | Day 1, | Pre + post | Day 15 | 0.25 | g/kg | 2 weeks | After |
| | | | | 4, 7, 10, 13, 16 | | | | | after 6- | behavioral |
| | | | | 19, 22, 25, 28 | | | | | OHDA | tests |
| L | 10 rats | AFOD | IV | Day 1, | Pre + post | Day 15 | 0.125 | g/kg | 2 weeks | After |
| | | | | 4, 7, 10, 13, 16 | | | | | after 6- | behavioral |
| | | | | 19, 22, 25, 28 | | | | | OHDA | tests |

V. Methods

1. Animals: rnale SO rats were purchased from Shanghai Laboratory Animal Center (SLAC). They were housed under 21-230C, with 12 h light-dark life cycle. Food and water were given ad libitum.

2. 6uOHDA lesion: Rats were anesthetized with 60 mg/kg sodium pentobarbital. They were stereotaxic injected with total dose of 20 pg of fresh prepared 6-OHDA (dissolved in saline containing 0.05% ascorbic acid, calculated as free base) into tvvo sites of the left striaturn, using the following coordinates (in mm relative to Bregma): AP+i 0.0, L −2.5, DV −5.0; AP −0.4, L −4.0, DV −5.5. The injection rate was i pi/min and a total of 2 iJI was injected at each site. The needle was left in place for 3 min before retracting.

3. Cylinder test: Rats were placed in a transparent cylinder (22 cm in diameter and 30 cm height). Animal would rear and support its body with one or both of its forelimbs. Numbers of left, right or both forelirnb(s) wall contacts were countered until total number of wall contact reached 20. Each behavioral was expressed as percent use of left, right or both limb(s) relative to the total number.

4. Adjusting step test The rats were held by the experimenter fixing the hindlimbs and slightly raising the hind pa1 of the body. The forelimb not to be tested was also fixed, with only the other forepaw touching the table. The rat was moved slowly sideways (90 cm in 5s), first in the forehand (defined as right paw to the left and left paw to the right) then in the backhand (defined as right paw to the right and left paw to left) direction. The number of adjusting steps of each left and right forelimbs on both directions was recorded individually.

5. Apomorphine induced rotation test After completing the above two tests, rats were placed in a round container of 40-cm diameter. After 10-min acclimation, they were injected s.c. with 0.25 mg/kg apomorphine which induced spontaneous contralateral rotations. The number of contralateral rotation was countered for 5 min.

6. TH staining: After the completion of behavioral tests, animals were sacrificed with an over dose of pentobarbital and transcardiac perfusion fixed with 4% paraformaldehyde in 0.1 M phosphate buffer (pH?0.4). Brains were removed and further fixed in the same fixative overnight at 4° C., they were transferred to 30% sucrose solution till sunk and then cut into 301Jm coronal sections on a cryostat microtome. Three sections of caudal, center and rostral part of the SN (bregma −5.5, −5.25 and −5.0 mm) were used for staining. The sections were incubated with prirnary antibody (TH, 1:1000, from Millipore) overnight at 4° C. followed by HRP-conjugated secondary antibody (Jackson Immunoresearch). The sections were developed using diaminobenzidine as the chromogen. Sections were digitally captured through an Olympus DP72 carnera connected to the microscope. Number of positively stained cells in the left and right sides of SN in each section was counted to make the summation. The ratio of left/right was calculated.

7. Statistic analysis: Data were expressed as mean±SEI\t1 and analyzed with ANOVA followed by Tukey test. Significance level was set at p<0.05.

VI. Results

The study of post groups was stopped after three injections following the sponsors request. There were one rat in pre control group, one in pre low dose group and two in pre-post control group died during lesion surgery. Other animals recovered well after lesion and continuous injection did not cause any obviously abnormal activities by normal clinical observation.

1. Effects of Pretreatment of AFOD on the Behavioral Performance

Rats were treated with vehicle or AFOD of three different doses for 2 weeks before the 6-OHDA lesion. Behavioral tests were performed 2 weeks after lesion. All the four groups showed significant decline of right forepaw step in forehand direction (FIG. 1A). In cylinder test, they also showed significant declined right forepaw use (FIG. 1C). Injection of apomorphine induced obvious rotation in control, moderate and high dose groups, however the rotation of low dose group \Nas slightly less (FIG. 1D).

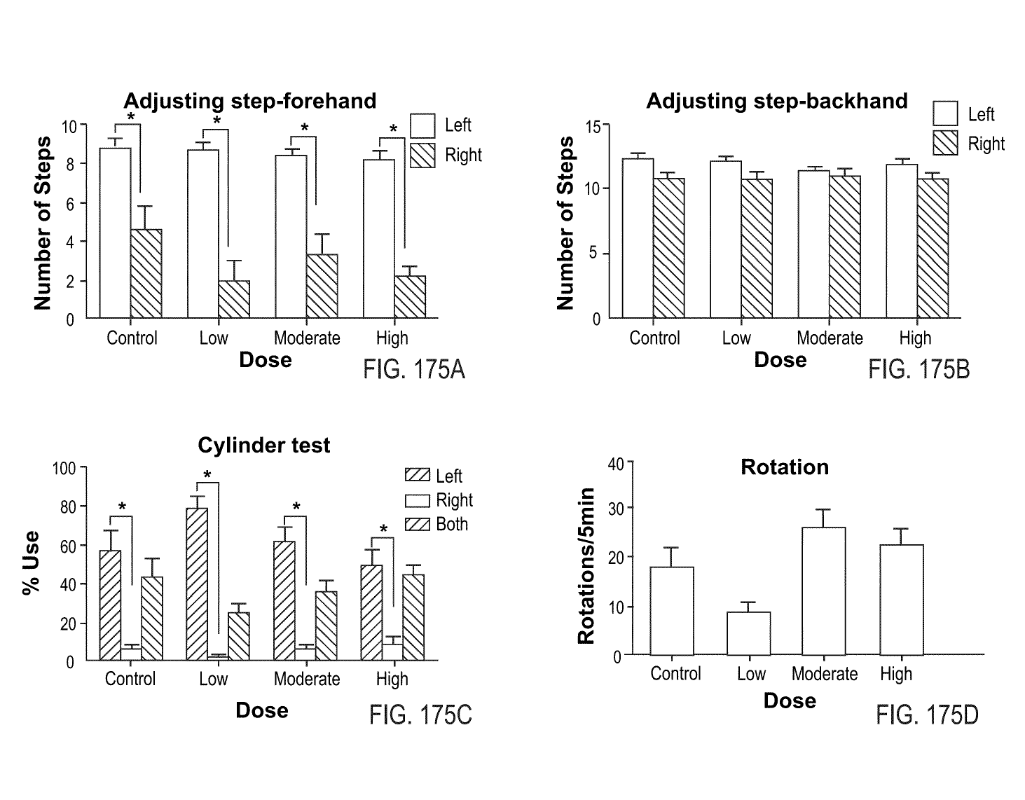

Data of the three tests were analyzed by ANOVA, there was no significant difference among groups.
FIG. 175. Effects of pretreatment of AFOD on the behavioral performance.

Rats were treated with vehicle or AFOD of three different doses for 2 weeks before the 6-OHDA lesion. Behavioral tests were performed 2 weeks after lesion. A. Adjusting step test forehand direction. B. Adjusting step test backhand direction. Number of steps was counted when the rats were moved sideways. C. Cylinder test. Rats were placed in a cylinder and number of left, right or both forelimb wall contacts was countered. The behavioral results were expressed as percent use relative to the total number. D. Apomorphine induced rotation. Rats were injected s.c. with 0.25 mg/kg apomorphine and rotation was counted for 5 min. Data were expressed as mean±SEM. *p<0.05.

2. Effects of Pretreatment+posHreatment of AFOD on the Behavioral Performance

Rats were treated with vehicle or AFOD of three different doses for 2 weeks before the 6-OHDA lesion. They were further treated for 2 weeks after lesion, and then behavioral tests were performed. All the four groups showed significant decline of right forepmN step in forehand direction (FIG. 2A). In cylinder test, they also showed significant declined right forepaw use (FIG. 2C). Injection of apomorphine induced obvious rotation in all the four groups (FIG. 2D).

Data of the three tests were analyzed by ANOVA, there was no significant difference among groups.

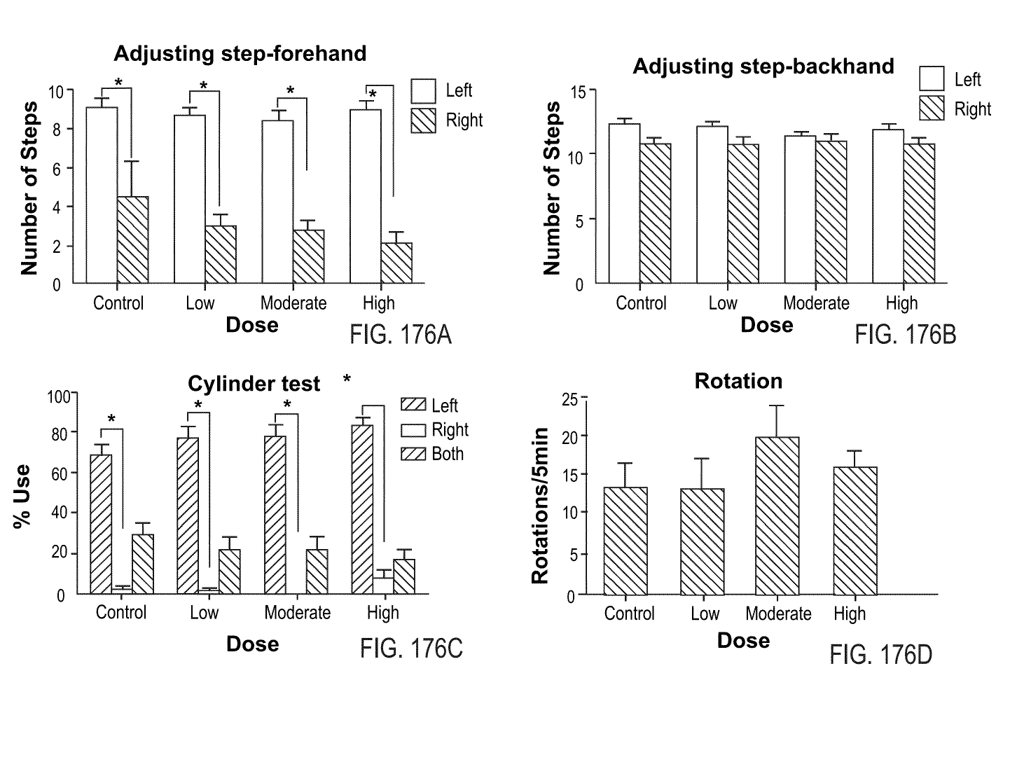

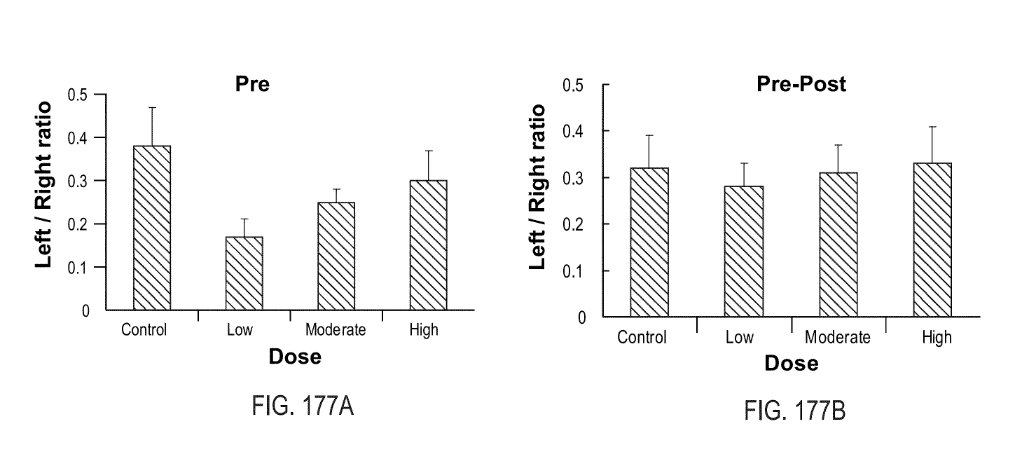

FIG. 176. Effects of pretreatment+post-treatment of AFOD on the behavioral performance.

Rats were treated with vehicle or AFOD of three different doses for 2 weeks before the 6-OHDA lesion. They were further treated for 2 weeks after lesion, and then behavioral tests were perfommed. A Adjusting step test forehand direction. B. Adjusting step test backhand direction. Rats were held and let one forelimb touch the table. Number of steps was counted when the rats were moved sideways. C. Cylinder test. Rats were placed in a cylinder and number of left, right or both forelimb wall contacts was countered. The behavioral results were expressed as percent use relative to the total number. D. Apomorphine induced rotation. Rats were injected s.c. with 025 mg/kg apomorphine and rotation was counted for 5 min. Data \Nere expressed as mean±SEM. *p<0.05.

3. TH Staining

To verify the neuron survival in the SN, five rats from each group (except pre low dose group that all the nine rats were sacrificed) were perfused for fixation after the behavioral tests and IHC staining of TH was performed. In control group, there was 30%-40% neurons survival in the lesion side (left side). Pre low dose group had less neurons remained in the lesion side, however there was no significant difference by ANOVA analysis.

Figure i 77. TH staining of the SN. Rats were perfused and the brains \Nere fixed for IHC study.

Three sections from caudal, center and rostral part of the SN (bregma −5.5, −5.25 and −5.0 mm) of each brain were used for staining. Cell number of each side was counted and the ratio of left/right was calculated. Data were expressed as mean±SEM.

4. Results from Daily Injected Rats

The rest of the rats of pre and pre/post groups were selected for further treatment of AFOD. The treatment protocol was shown in table 1:

Behavioral tests were conducted on October 8 and 9. After that, rat# A2-3, B1-2, B2-3, C1-1, C1-2, J1-1 and J2-5 were perfused for IHC staining of DA neurons. Ten negative control rats were also used for IHC staining.

4.1 Cylinder test: Since the rats were too big for cylinder test, they were not active and the number of wall contact was small, only raw data were shown here (Table 2).

TABLE 2
Number of wall contact in cylinder test

| | | | Numbe [symbol] | of | | | % | |
|---|---|---|---|---|---|---|---|---|
| Dose | Group | No. | [symbol] | [symbol] | Both | Left | Right | Both [symbol] |
| | B1 | 2 | 4 | 1 | 0 | 80.0 | 20.0 | 0.0 |
| | | | | 0 | 0 | 100.0 | 0.0 | 0.0 |
| | | 5 | 11 | 0 | | 100.0 | 0.0 | 0.0 |
| [symbol] /kg [symbol] + | B2 [symbol] | 2 | 6 | +kg 0 | 2 | 75.0 | 0.0 | 25.0 |
| 10 ml/kg | | | | | | | | |
| [symbol] 1 Omlikg | | 3 | 3 | 2 | 1 | 50.0 | [symbol] | [symbol] |
| sc | | | | | | | | |
| sc | | | | | | | | |
| | Cl | 1 | 12 | 3 | | 60.0 | 15.0 | 25.0 |
| | | 2 | 5 | 5 | 10 | 25.0 | 25.0 | +---------- |
| | | | | | | | | [symbol] |
| 8.3 m1/kg | 02 | 1 | 5 | 2 | 1 | 62.5 | 25.0 | 12.5 |
| iv -4- | | 2 | 10 | 0 | 0 | 100.0 | 0.0 | 0.0 |
| 8.3 rnl/kg | | 3 | [symbol] | 2 | 1 | 66.7 | 22.2 | 11.1 |
| | J1 | | | | | | | |
| | J2 | 1 | 1 | 0 | 0 | 100.0 | 0.0 | 0.0 |
| 6.7 m1/kg | | 2 | 0 | ., | | | | |
| iv + | | 4 | 0 | 0 | 0 | | --------------- | |
| 6.7 mllkg | | 5 | 7 | 0 | 0 | 100.0 | 0.0 | 0.0 |
| control | 11 | 3 | 1 | 1 | 2 | 25.0 | 25.0 | 50.0 |
| | | 4 | | | 2 | 77.8 | 0.0 | 22.2 |
| | 12 | 2 | | | | | | |
| control | 1 | 0 | 0 | 0 | | | | |
| | | 2 | 2 | 0 | 1 | 66.7 | 0.0 | 33.3 |
| | | 3 | 12 | 1 | 1 | 85.7 | 7.1 | 7.1 |
| | | 4 | 2 | 0 | 0 | 100.0 | 0.0 | 0.0 |

[symbol]

indicates data missing or illegible when filed

[symbol]

4.2 Adjusting Step Test

All the four groups showed significant declined right forepaw step in forehand direction, furthermore, control and high dose group had significant step decline in backhand direction (FIG. 4). There was no significant difference among groups analyzed by ANOVA.

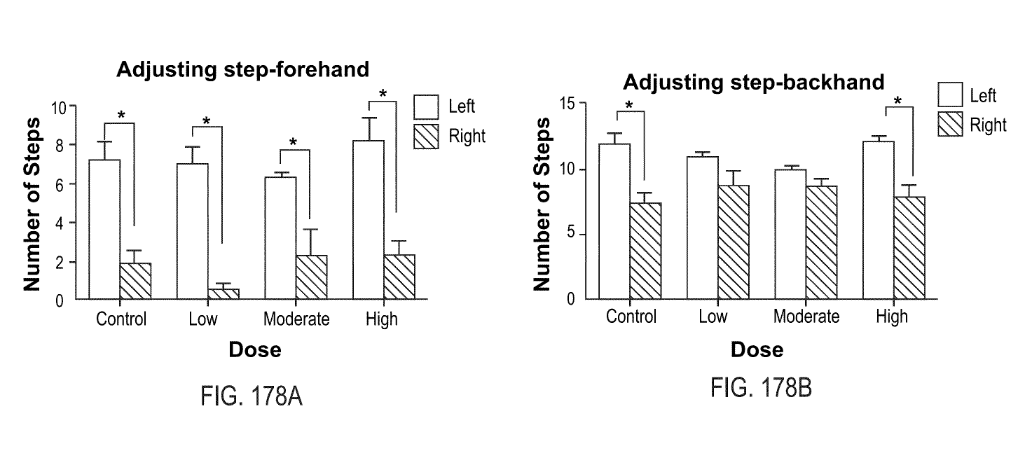

FIG. 178. Effects of daily injection of AFOD on adjusting step test. A. Forehand direction. 8. Backhand direction. Data were expressed as mean±SEM. *p<0.05.

4.3 Rotation Test

Number of apomorphine induced rotation was shown in FIG. 5. All the rats had obvious rotation after injection of apomorphine. There was no significant difference among groups.

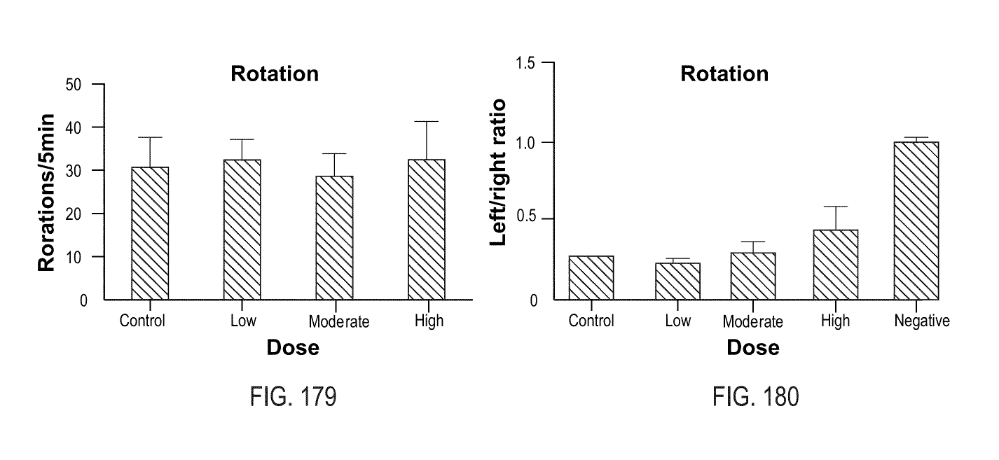

FIG. 179. Effects of daily injection of AFOD on rotation. Rats were injected s.c. with 0.25 mgikg apomorphine and rotation was counted for 5 min. Data were expressed as mean 1 SEM.

4.4 TH Staining

Rats were perfused for fixation and brain sections of SN were stained with TH antibody to show dopaminergic neurons. Data were shmNn in table 3 and FIG. 6.

TABLE 3
Nurnber of TH positive cell counting

| | | | | | | Neuron counting | | | | |
|---|---|---|---|---|---|---|---|---|---|---|
| | | Left | | | | Right | | | | ------------------ |
| Group | # | 1 | 2 | 3 | Sum | 1 | 2 | 3 | Sum | LIR ratio |
| Control | A2-3 | 32 | 43 | 47 | 122 | 126 | 170 | 152 | 448 | 0.27 |
| Low | J1-1 | 15 | 24 | 24 | 63 | 97 | 101 | 123 | 321 | 0,20 |
| | , ---------- | | | | | | | | | |
| | [symbol] | 27 | 28 | 38 | 93 | 117 | 139 | 108 | 364 | 0.26 |
| Moderate | 01-1 | 25 | 25 | 45 | 95 | 129 | 156 | 149 | 434 | 0.22 |
| | C1-2 | 74 | 45 | 85 | 204 | 169 | 182 | 221 | 572 | 0.36 |
| High | B1-2 | 91 | 63 | 111 | 265 | 141 | 133 | 179 | 453 | 0.58 |
| -----------------i | B2-3 | 59 | 25 | 50 | 134 | 129 | 163 | 178 | 470 | 0,29 |
| | 1 | 149 | 100 | 191 | 440 | 133 | 81 | 203 | 417 | 1.06 |
| | 2 | 96 | 79 | 217 | 392 | 125 | 107 | 170 | 402 | 0.98 |
| | 3 | 71 | 88 | 153 | 312 | 91 | 78 | 125 | 294 | 1,06 |
| | 4 | 17 | 207 | 151 | 485 | 102 | 154 | 140 | 396 | 1.22 |
| Negative | 5 | 76 | 112 | 118 | 306 | 61 | 120 | 110 | 291 | 1.05 |
| | 6 | 124 | 126 | 99 | 349 | 119 | 156 | 124 | 399 | 0,87 |
| | . . . | 116 | 114 | 195 | 425 | 101 | 148 | 204 | 453 | 0.94 |
| | 8 | 134 | 160 | 131 | 425 | 137 | -----------+ | 170 | ---------- + | 0.93 |
| | | | | | | | ------------ | | ------------ | |
| | 9 | 150 | 120 | 168 | 438 | 157 | 103 | 182 | 442 | 0.99 |
| | 10 | 112 | 135 | 193 | 440 | 154 | 187 | 141 | 482 | 0.91 |

[symbol]

indicates data missing or illegible when filed

[symbol]

FIG. 180. TH staining of the SN.

Rats were perfused and the brains were fixed for IHC study. Three sections from caudal, center and rostral part of the SN (bregma −55, −525 and −5.0 mm) of each brain were used for staining. Cell number of each side was counted and the ratio of left/right was calculated. Data were expressed as mean 1 SEM

5. Rotation Test for Post Groups

The rats in post groups were tested with apormorphine induced rotation on Oct. 10, 2011. The number of rotation was shown in Table 4. No further experiment was done on these rats.

TABLE 4
Number of rotation of post groups

| | | control | high | moderate | low |
|---|---|---|---|---|---|
| | rat # | E | F | G | H |
| cage 1 | 1 | 0 | 20 | 10 | 50 |
| | 2 | 30 | 4 | 11 | 0 |
| | 3 | 17 | 11 | 0 | |
| | | 0 | 16 | 11 | 14 |
| | 5 | 5 | 17 | 0 | 16 |
| cage 2 | 1 | 12 | 15 | 0 | 71 |
| | 2 | 20 | 11 | 6 | 8 |
| | 3 | 19 | 19 | 0 | 23 |
| | 4 | 16 | 0 | 10 | 11 |
| | 5 | 2 | 8 | 4 | 14 |

All the left rats were sacrificed on Nov. 22, 2011.

Conclusion:

The inventor ordered to abort the study for therapeutic as there was no statistical data to support a valid vehicle group before the surgical operation to remove the brain in order to count the neurons. The result of the cylinder test and the rotation test on the rat did not give a very convincing result for the controL However after the operation of the brain to count the neurons in the vehicle control, negative control and tested prophylactic group it showed the trend that using AFOD RAAS 1 reduce the damage caused by 6-OHDA lesion in the high and moderate groups to compare with the vehicle. Other studies are being conducted using 6-OHDA lethal dose in the rat

KH good healthy cells 1—Send signals to the DAMAGED, SICK, AND BAD CELLS that triggers that synthesis of good proteins that transform these cells to become GOOD healthy cells: 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being aff(cted by intra- and extracellular damaging signals.

Report Title: Antiviral efficacy of AFCC in an influenza H1N1 infected mouse model

Report No: WX IFV02162012

Issue Date: Apr. 11, 2012

Study No: RAAS-20120216B

Study Period: February 16! 201:2 to April 8! 201:2

Infection with hurnan influenza virus (IFV) causes respiratory tract illness in human and animals including mice. Mouse model infected Intranasally with IFV H1N1 is well recognized for anti-IFV compound screening. This study is designed to evaluate in vivo anti-IFV activity of a blood-derived product AFCC from RAAS in the mouse modeiJ L \LI I..I\ [symbol] LE1.\ UIQS.m g §.JL. tt LfLLU.\\?

Study

method

Study RAAS-201202168 was executed in the following steps:

1) Treat mice with RAAS blood product AFCC-KH.

1) Infect mice with IFV by intranasal inoculation.

2) Observe mice for 26 days.

3) Sacrifice mice in the end of the study. Result summary

Report for RAAS

20120216B L Method

Animals:

Female BALB/c mice (6-8 weeks, 17-22 g) \Nere divided into defined study groups after a visual examination and a 3 to 5-day acclimation upon arrivaL

Solution preparation:

1. Sodium Pentobarbital: Freshly dissolved in saline for injection at 8 mg/ml prior to using.

2. Test article: human plasma derived protein AFCC in sterile solutions for vein injection provided by the client

Experimental Procedure:

IFV Infection and Test Article Administration:

1. From day 1 to day 14, AFCC KH 1 is intravenously and/or intraperitoneally administrated for 14 days.

2. On day 15, mice are anesthetized by intraperitoneal injection of sodium pentobarbital (80 mg/kg). Mice are inoculated with 5×1QA3 pfu of Influenza H1N1 AiWSN/33 via the intranasal route in SFM medium.

3. From day 1 through day 40 mice are observed two times a day. Mortality and body weight are recorded daily”

4. On day 40, the experiment is terminated by sacrificing survived mice.

II. Groups and Schedules:

TABLE 1
Action summary of Study WX IFV02162012

| | | | IFV | ,FCC, | mouse |
|---|---|---|---|---|---|
| Study Day | Date | Weighing | challenge | iviip | sacrifice |
| Day 1 | Feb. 16, 2012 | • [symbol] | | ., | |
| Day 2 | Feb. 17, 2012 | Ni | | | |
| Day 3 | Feb. 18, 2012 | | | | |
| Day 4 | Feb. 19, 2012 | . [symbol] | | | |
| Day 5 | Feb. 20, 2012 | Ni | | N | |
| Day 6 | Feb. 21, 2012 | | | | |
| Day 7 | Feb. 22, 2012 | . [symbol] | | ., | |
| Day 8 | Feb. 23, 2012 | Ni | | | |
| Day 9 | Feb. 24, 2012 | | | | |
| Day 10 | Feb. 25, 2012 | . [symbol] | | | |
| Day 11 | Feb. 26, 2012 | Ni | | N | |
| Day 12 | Feb. 27, 2012 | | | | |
| Day 13 | Feb. 28, 2012 | . [symbol] | | ., | |
| Day 14 | Feb. 29, 2012 | Ni | | | |
| Day 15 | Mar. 01, 2012 | | | | |
| Day 16 | Mar. 02, 2012 | . [symbol] | | | |
| Day 17 | Mar. 03, 2012 | Ni | | | |
| Day 18 | Mar. 04, 2012 | | | | |
| Day 19 | Mar. 05, 2012 | . [symbol] | | | |
| Day 20 | Mar. 06, 2012 | Ni | | | |
| Day 21 | Mar. 07, 2012 | | | | |
| Day 22 | Mar. 08, 2012 | . [symbol] | | | |
| Day 23 | Mar. 09, 2012 | Ni | | | |
| Day 24 | Mar. 10, 2012 | . [symbol] | | | |
| | | , | | | |
| Day 25 | +Mar. 11, 2012 | , | | | |
| Day 26 | Mar. 12, 2012 | ,I | | | |
| Day 27 | Mar. 13, 2012 | . [symbol] | | | |
| Day 28 | +Mar. 14, 2012 | , | | | |
| Day 29 | Mar. 15, 2012 | ,I | | | |
| Day 30 | Mar. 16, 2012 | . [symbol] | | | |
| Day 31 | +Mar. 17, 2012 | , | | | |
| Day 32 | Mar. 18, 2012 | ,I | | | |
| Day 33 | Mar. 19, 2012 | . [symbol] | | | |
| Day 34 | +Mar. 20, 2012 | , | | | |
| Day 35 | Mar. 21, 2012 | ,I | | | |
| Day 36 | Mar. 22, 2012 | . [symbol] | | | |
| Day 37 | +Mar. 23, 2012 | , | | | |
| Day 38 | Mar. 24, 2012 | ,I | | | |
| Day 39 | Mar. 25, 2012 | . [symbol] | | | |
| Day 40 | Mar. 26, 2012 | | | | |

Indicates that the action was taken. [symbol]
indicates data missing or illegible when filed

[symbol]

TABLE 2
Experimental Design for the pilot experiment

| | | | |
|---|---|---|---|
| | AFCC-Kil | | H1N1 |
| Day | (m-_/iiouse) | animal number | WSN |

| | | | | |
|---|---|---|---|---|
| 1 | iv, 0.2 | 5 | | |
| 3 | ip, 0.6 | 5 | | |
| 5 | iv, 0.2 | 5 | | |
| 7 | ip, 0.6 | 5 | | |
| 9 | iv, 0.2 | 5 | | |
| 11 | ip, 0.6 | 5 | | |
| 13 | iv, 0.2 | 5 | | |
| 15 | ip, 0.6* | 5 | 5 | in, 5 × 10−3 |
| | | | | pfu/mouse |
| 17 | | | [symbol] | |
| | | | . .., | |
| 19 | | | [symbol] | |
| | | | . .., | |
| 21 | | | [symbol] | |
| | | | . ., | |
| 23 | | | [symbol] | |
| | | | . ., | |
| 25 | | | [symbol] | |
| | | | . ., | |
| 27 | | | [symbol] | |
| | | | . .., | |
| 29 | | | [symbol] | |
| | | | . .., | |
| 31 | | 5 | 2.-;- | |
| 33 | | 5 | 2.-;- | |
| 35 | | 5 | 2.-;- | |
| 37 | | 5 | 2.-;- | |
| 39 | | 5 | 2.-;- | |
| 40 | | 5 | :-.,,-;- | |

[symbol]

indicates data missing or illegible when filed

ill Adverse Events and Tolerability of Compounds:

1. In the AFCC treatmentgroup, --t4t.t--1-ae-t-.t-4, one mouse w;,:6 . . . 1, 2.012—the e--died of severe face end aeck demees on Ma/,2012 fexoerimenta de:117) due seHous fieht e:miong mice. This mouse was eliminated for final datass-s-ceeivais.

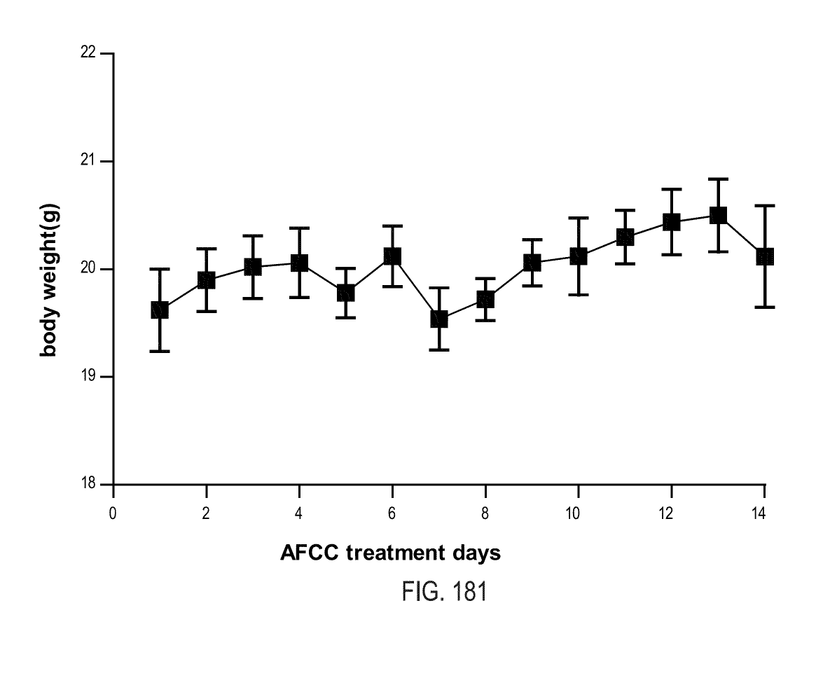

Results and Discussion
FIG. 181. Body weight changes caused with AFCC treatment in mice

TABLE 3
Effect of AFCC on mean day to death
of mice infected with H1N1 A/WSN/33

| Treatment | Survivor/total | Mean day to death ± S.D. |
|---|---|---|
| H1N1 + AFCC | 2/4 | 23.8 ± 3.0** |
| H1N1 + Vehicle | 0/5 | 5.8 ± 0.8 |

**P < 0.0i compared to the H1N1 + vehicle control

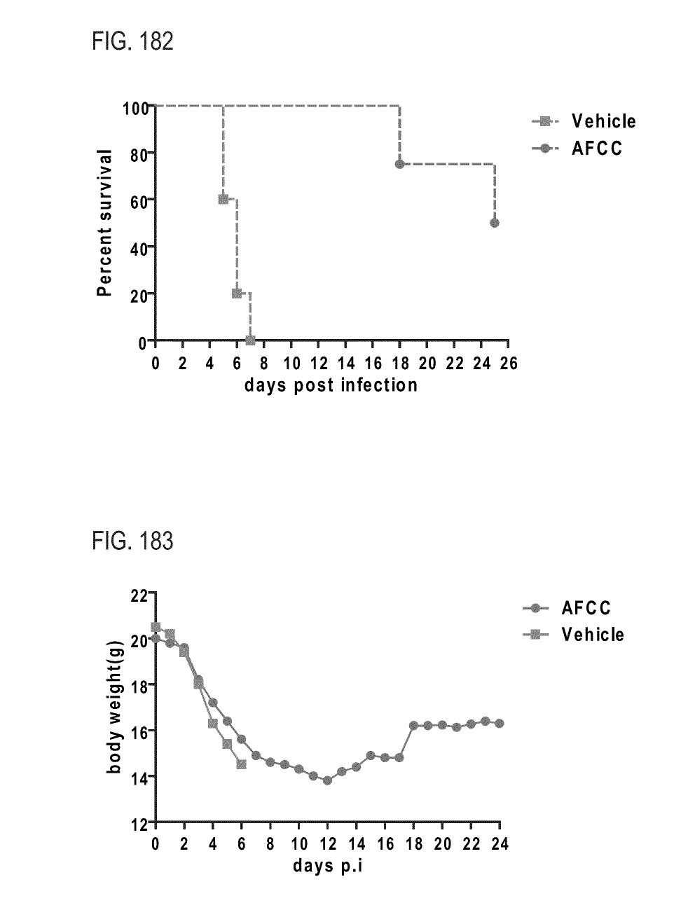

FIG. 182. Efficacy of AFCC on H1N1 WSNacaused mouse death

FIG. 183. Body weight changes caused by AFCC in mice infected with H1N1 (WSN) influenza

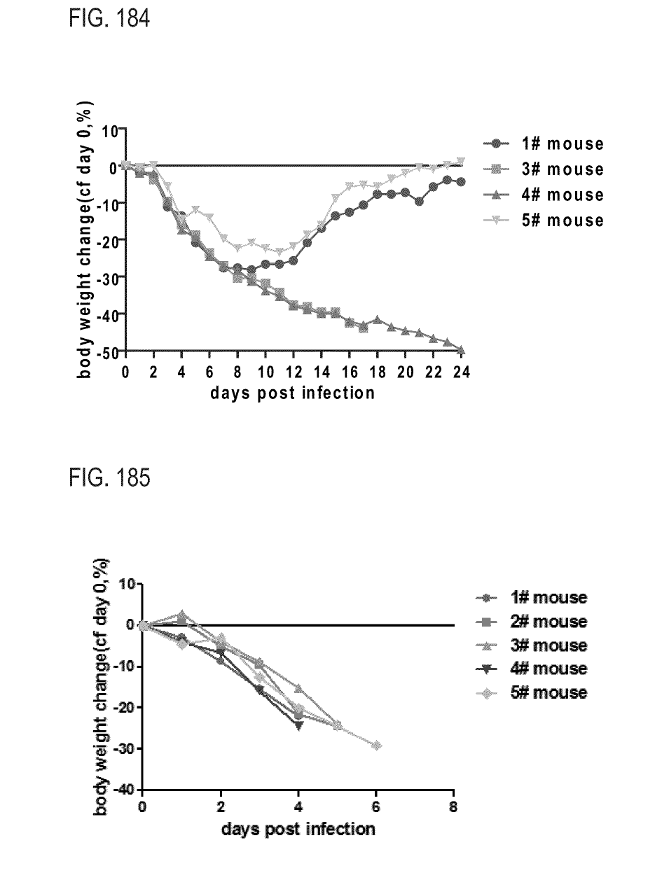

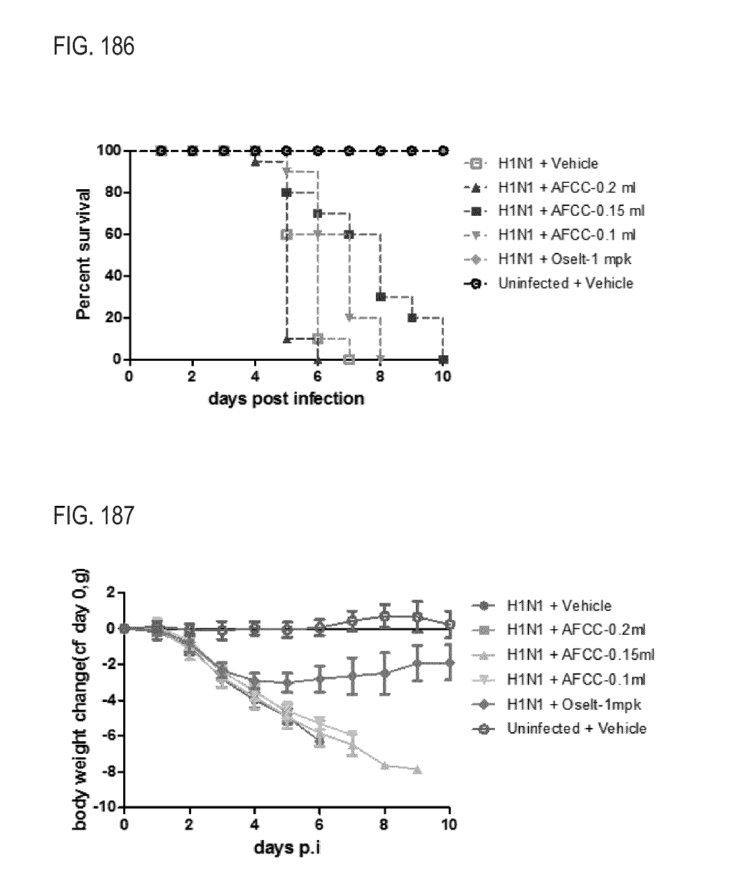

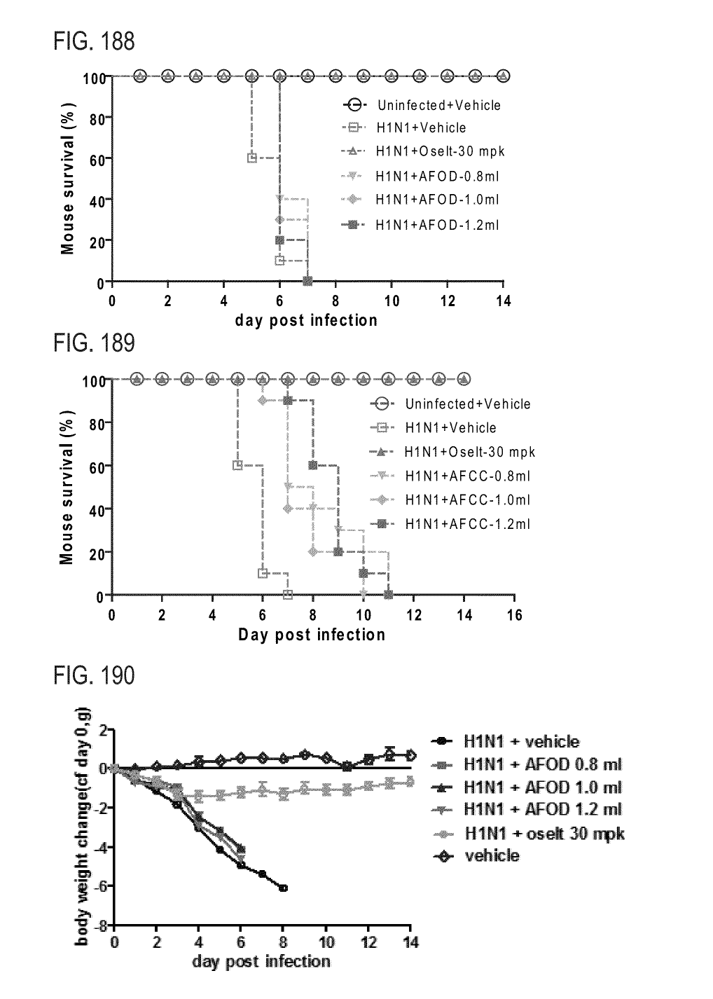

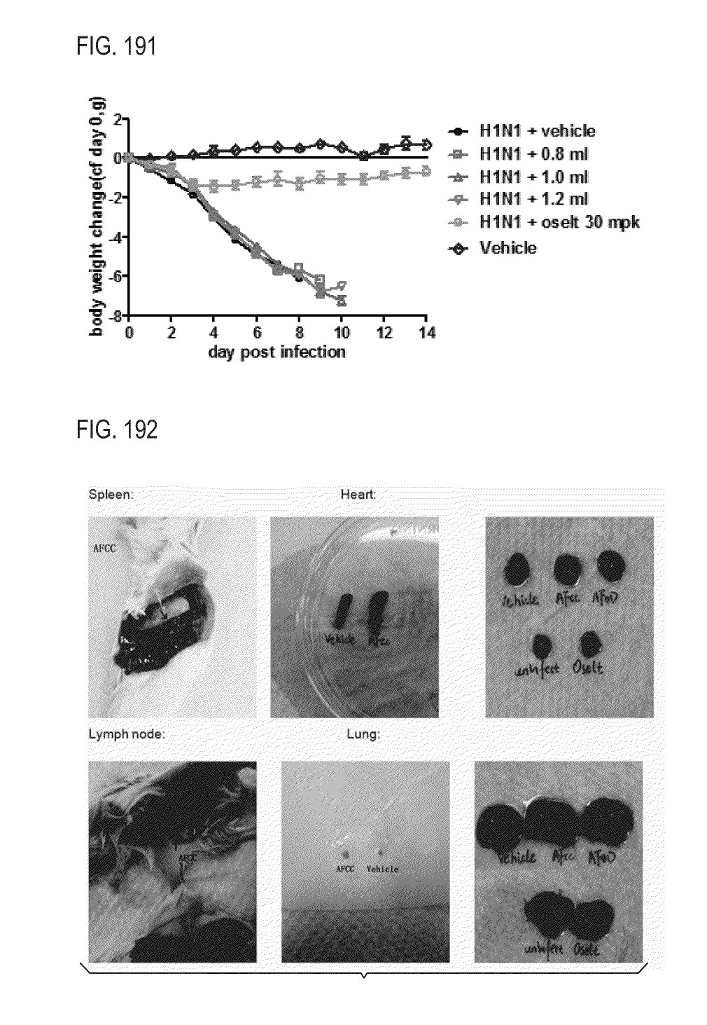

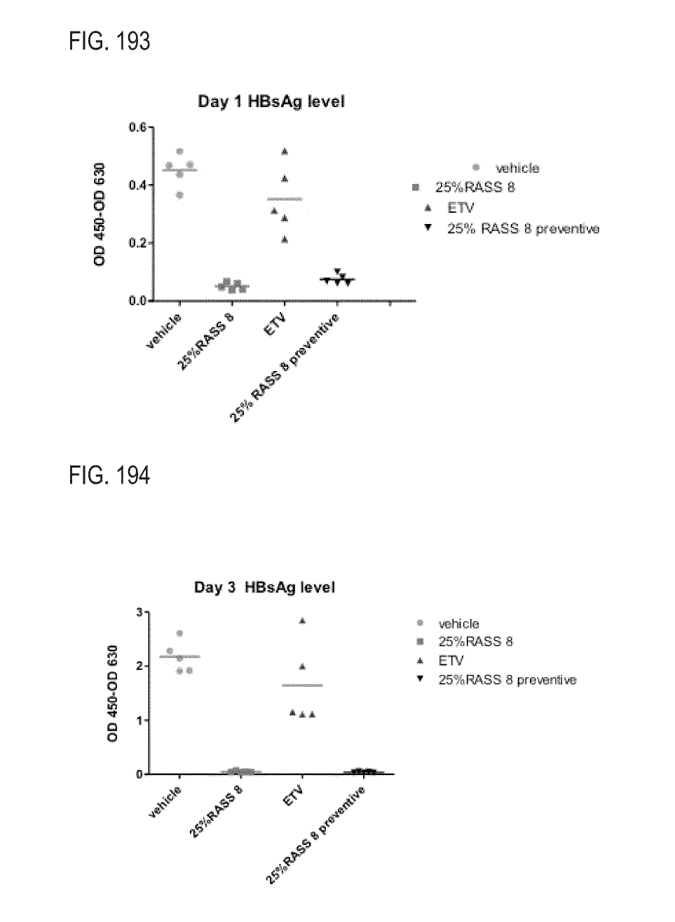

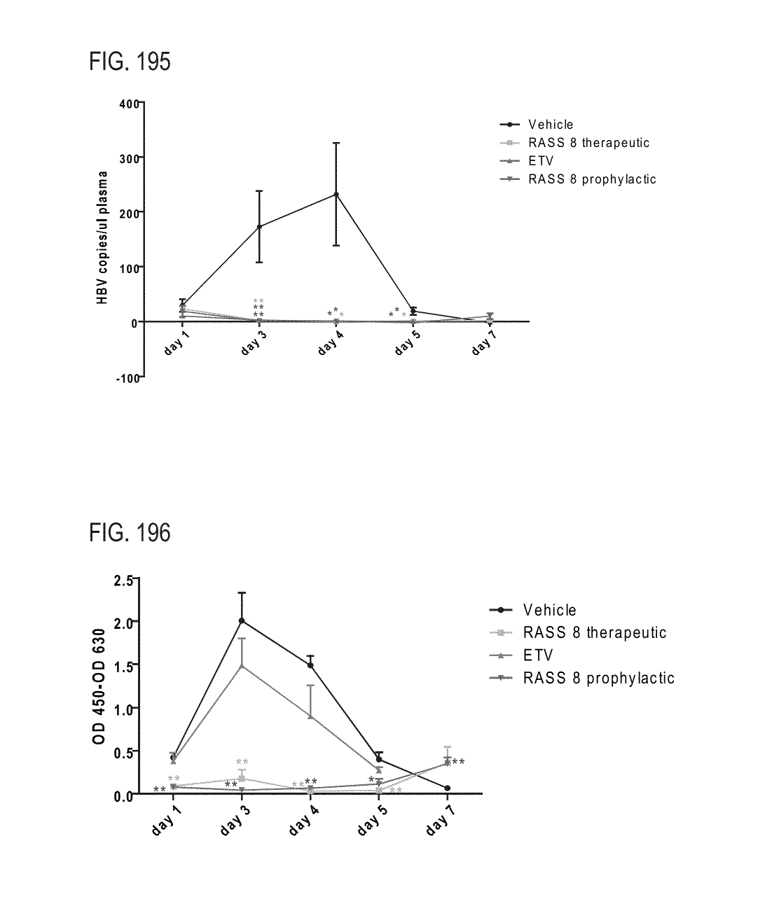

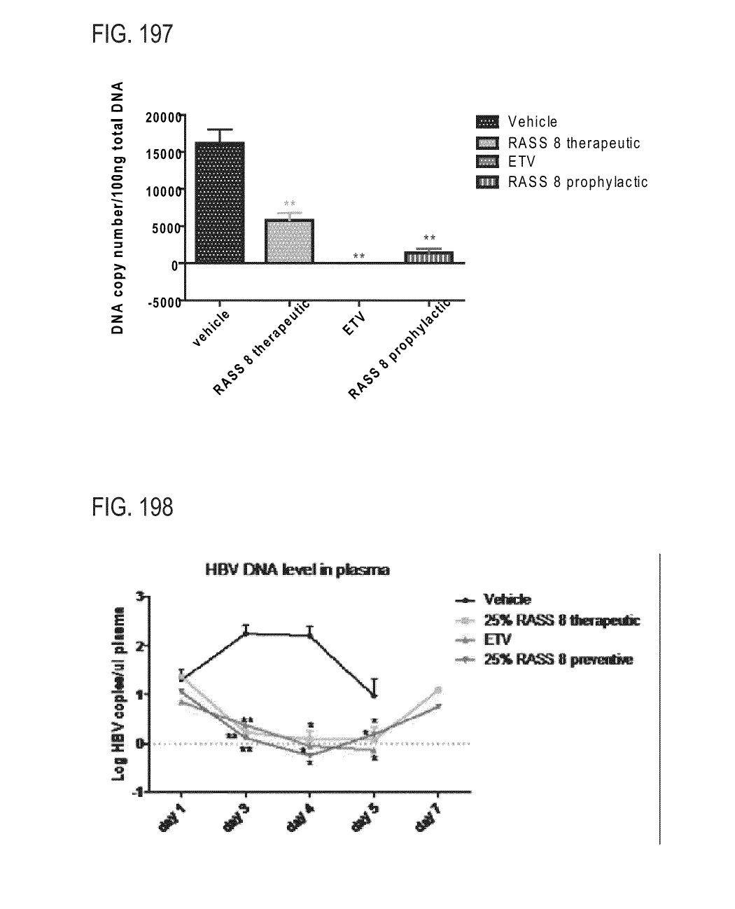

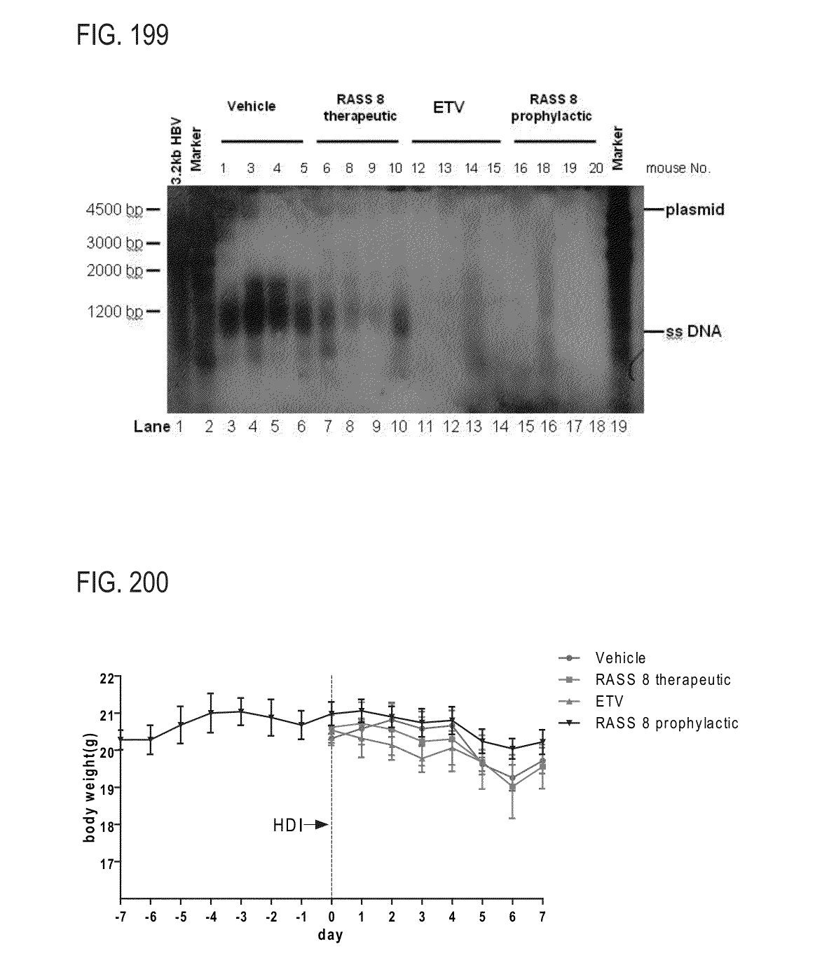

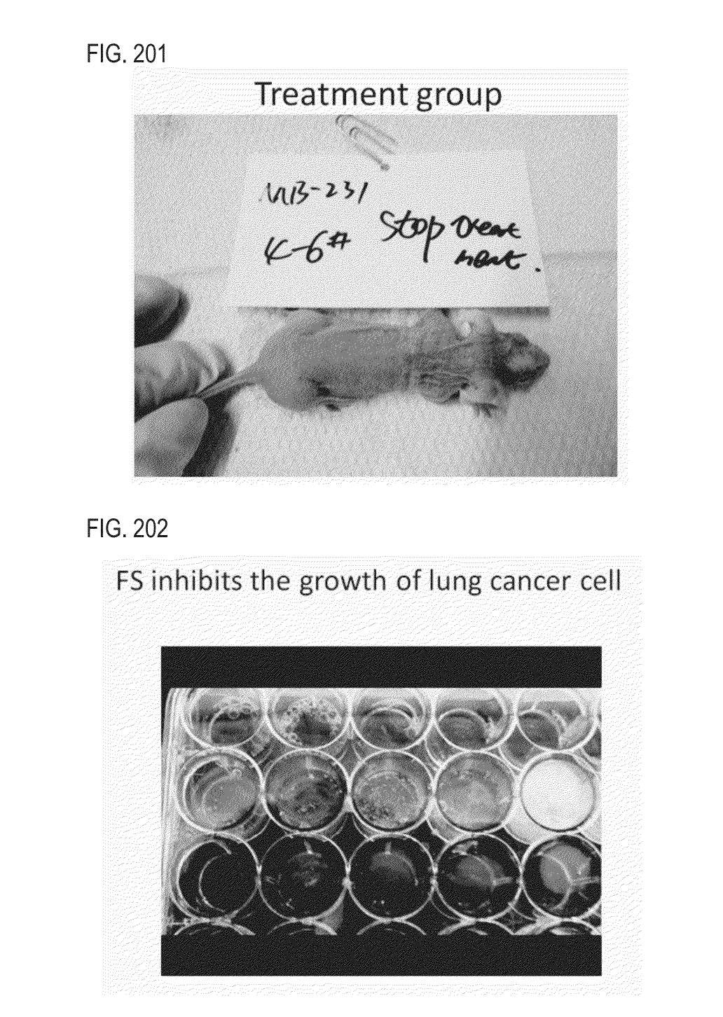

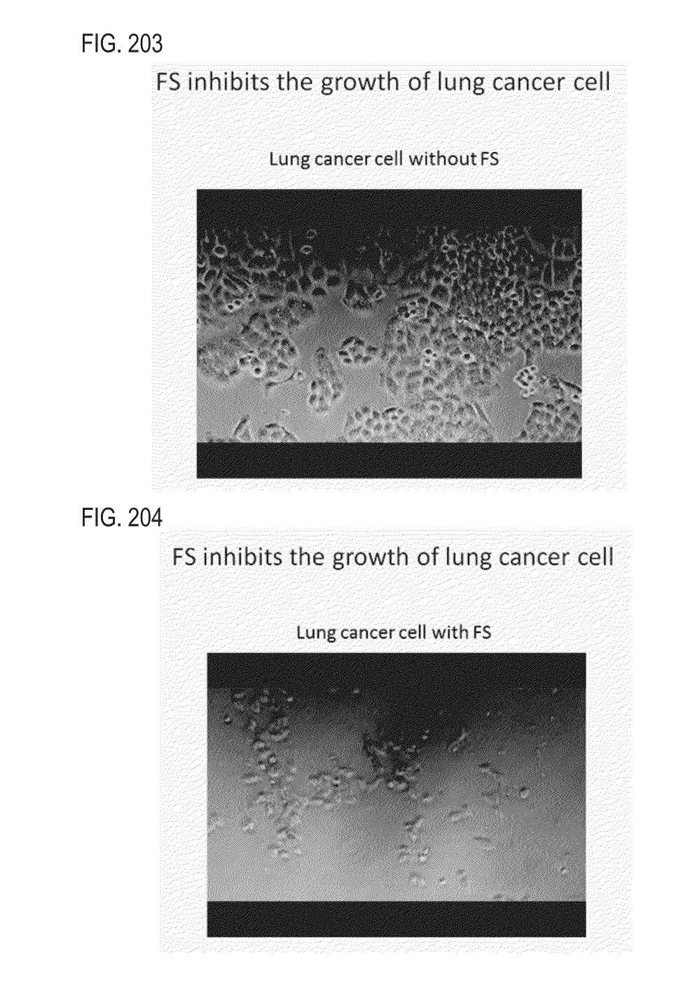

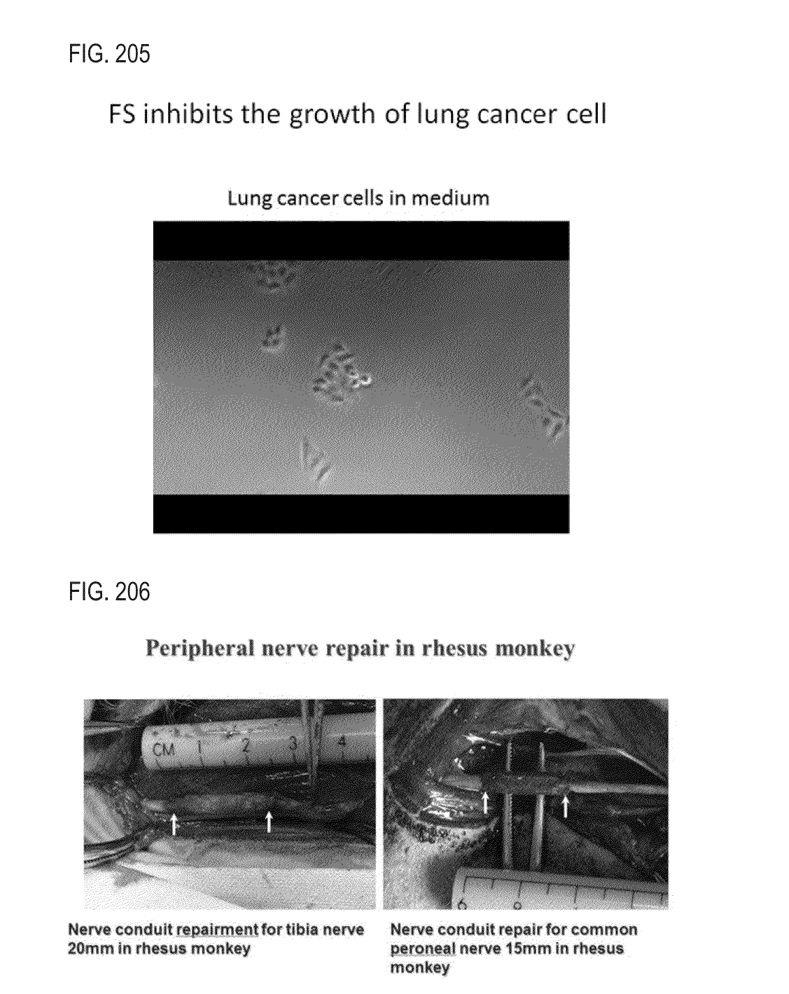

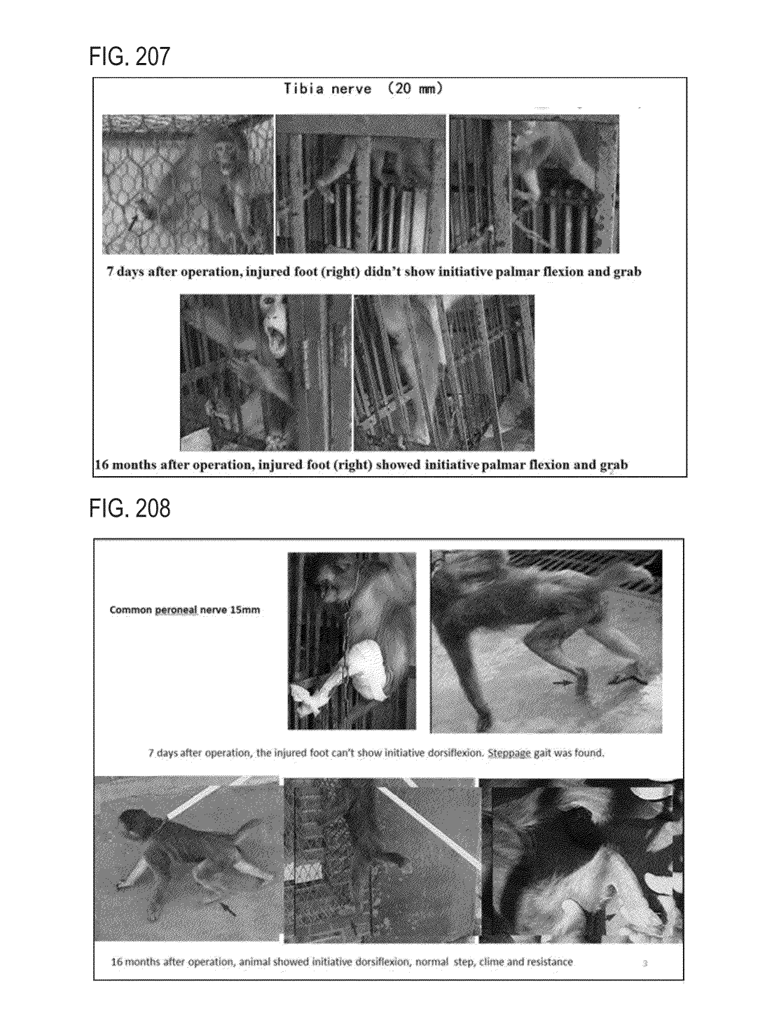

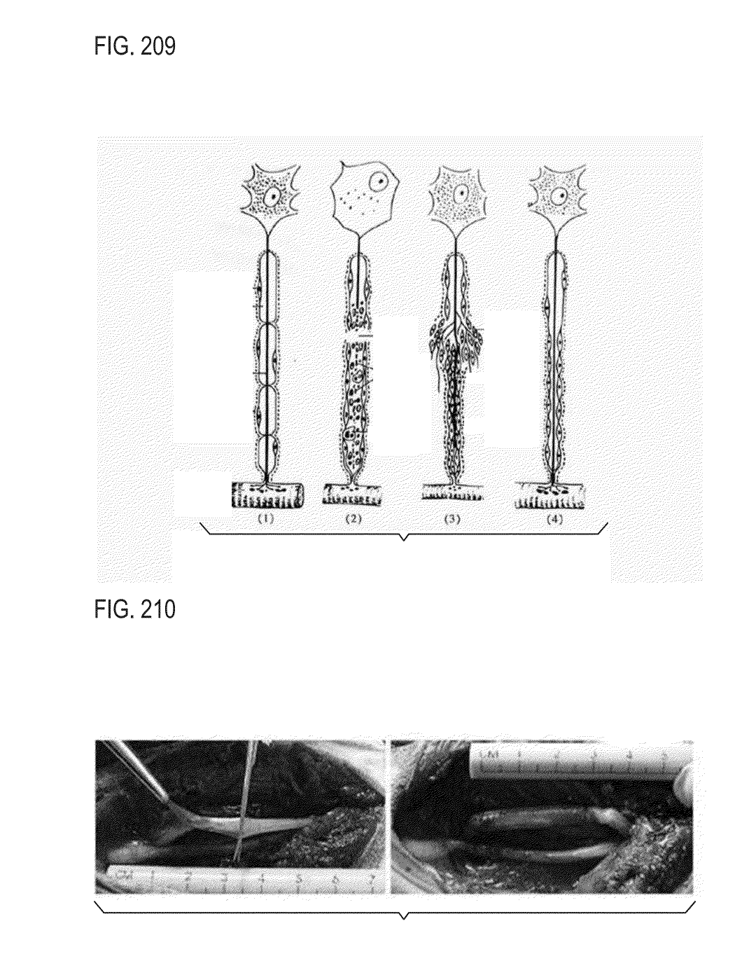

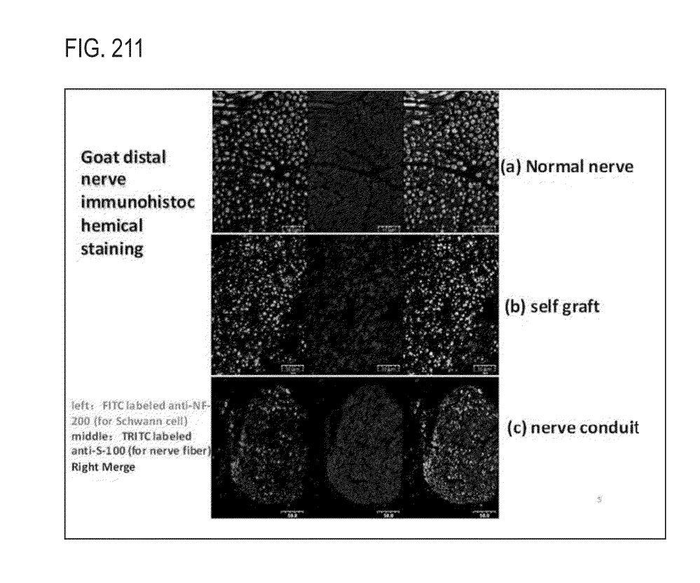

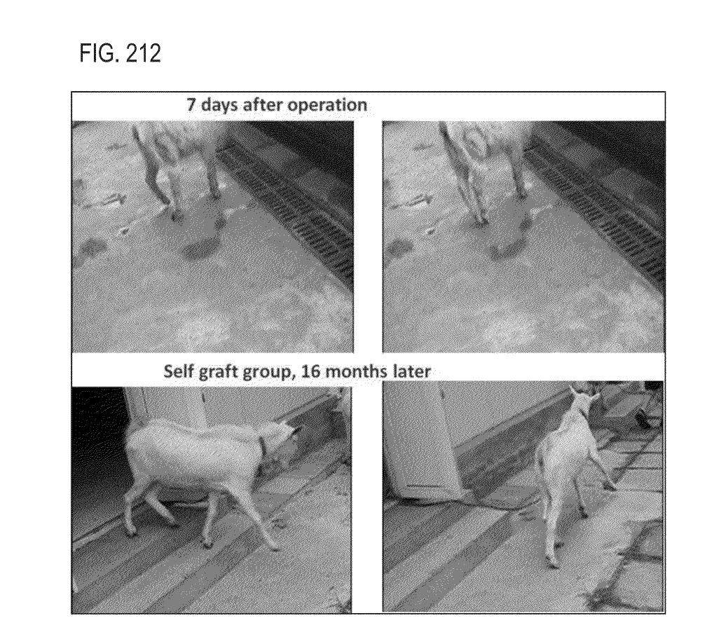

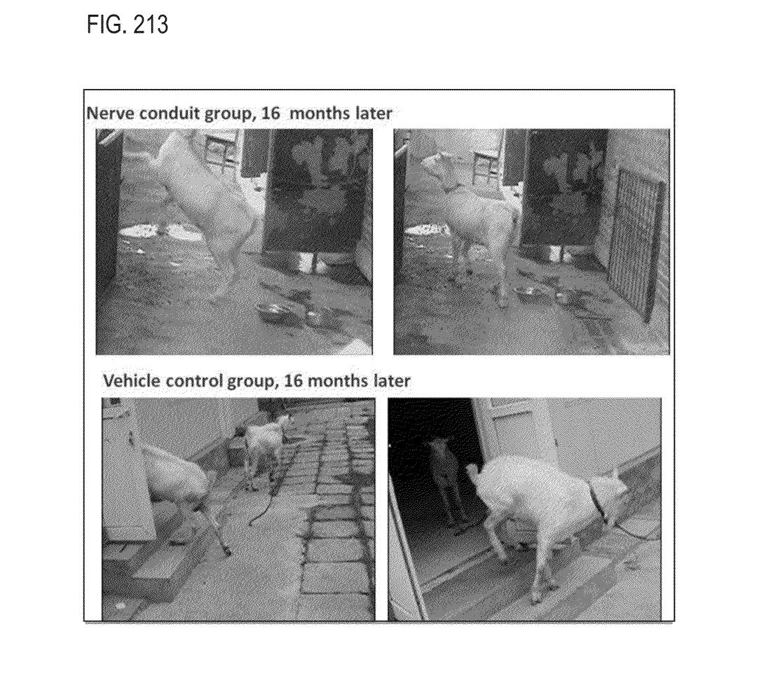

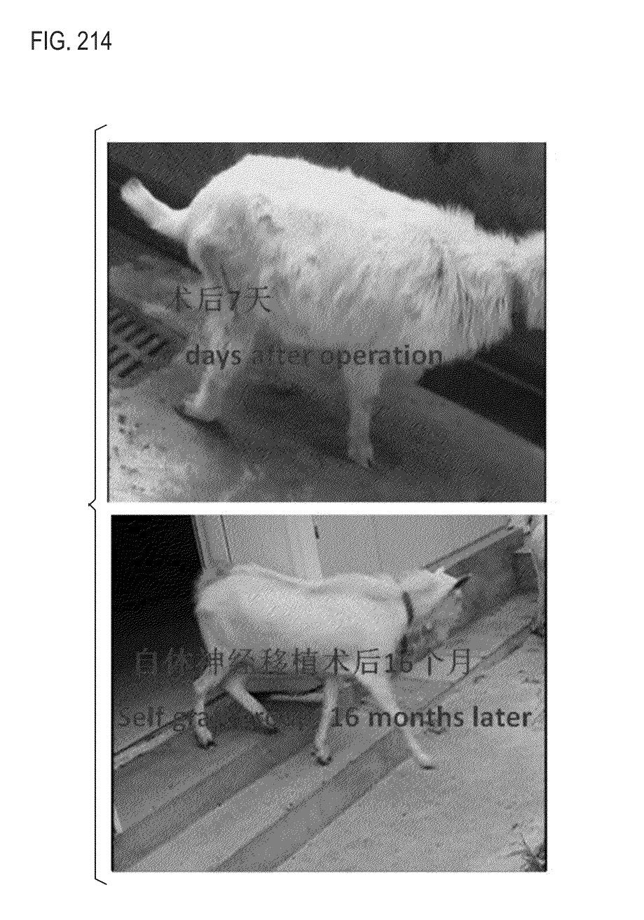

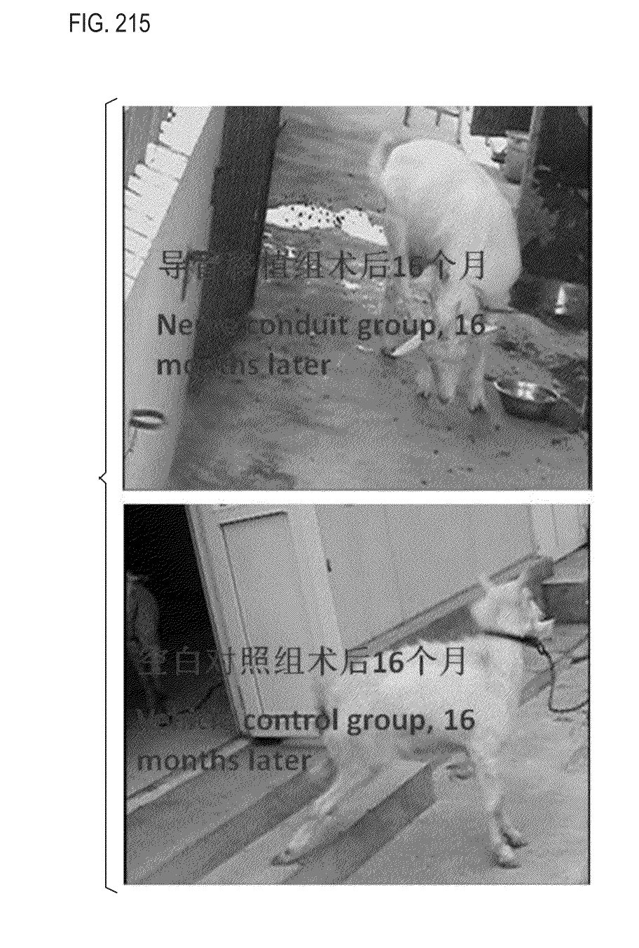

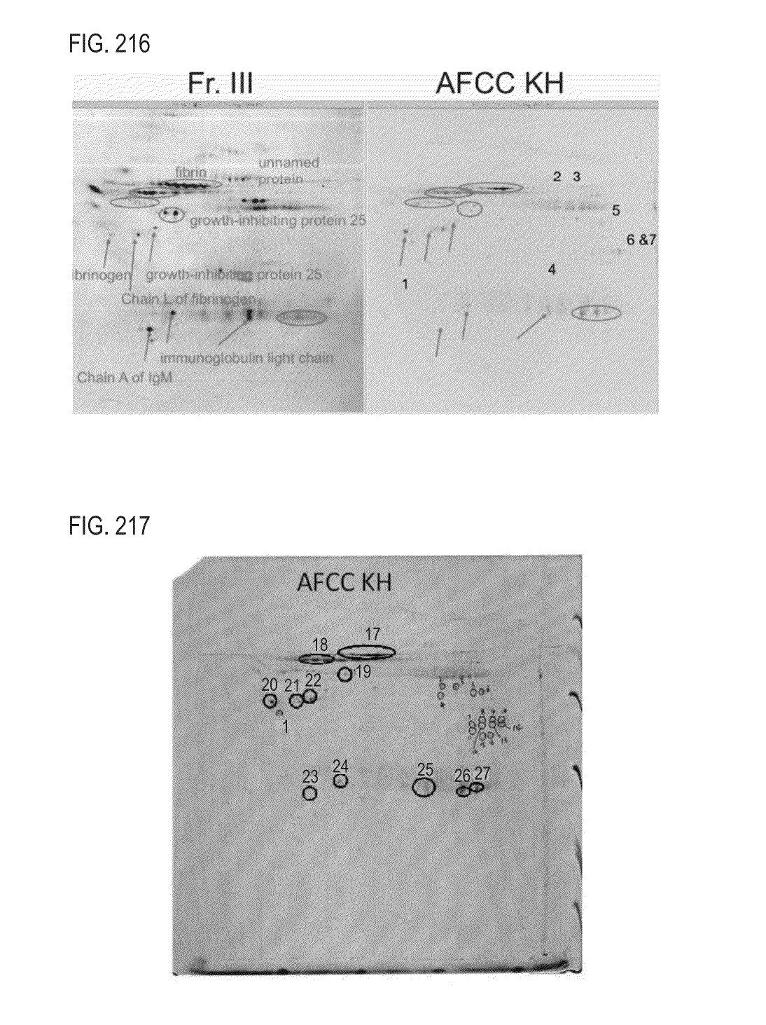

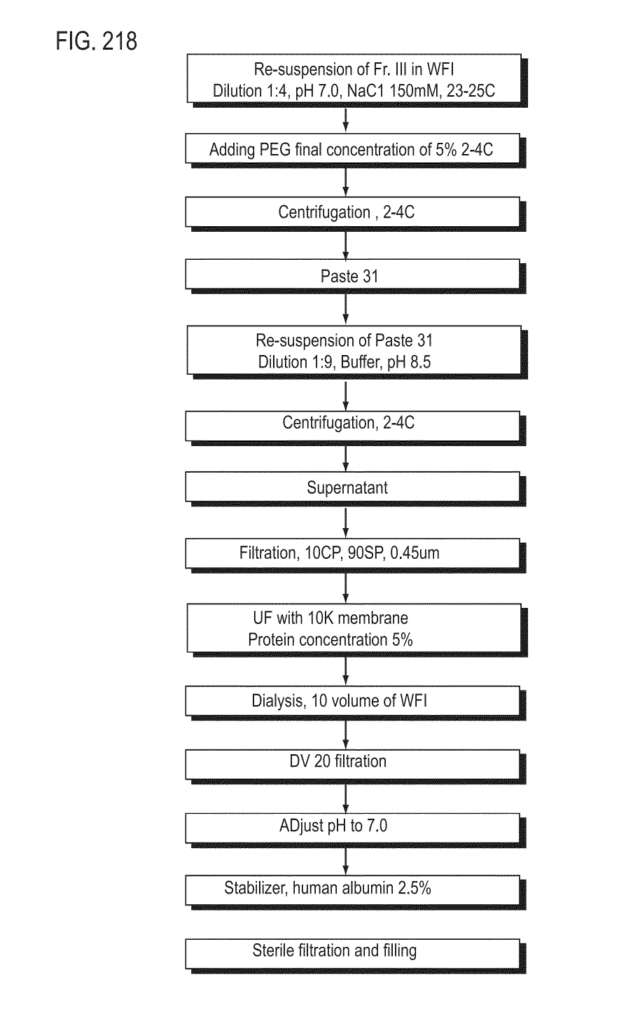

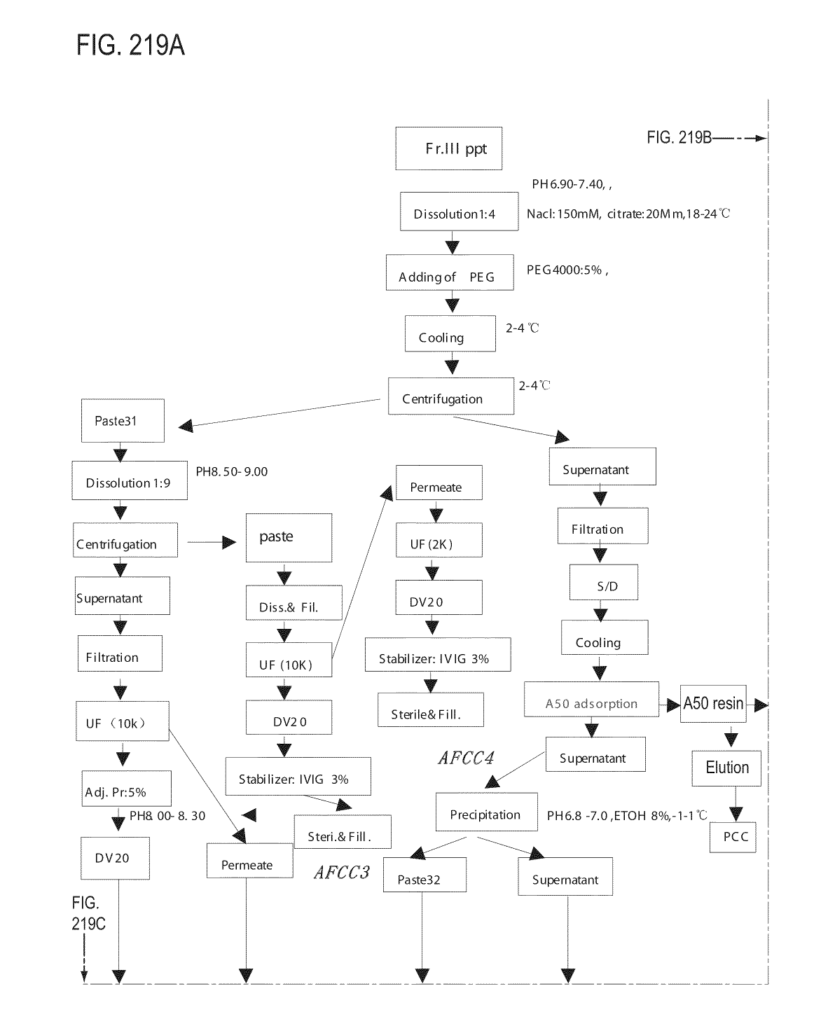

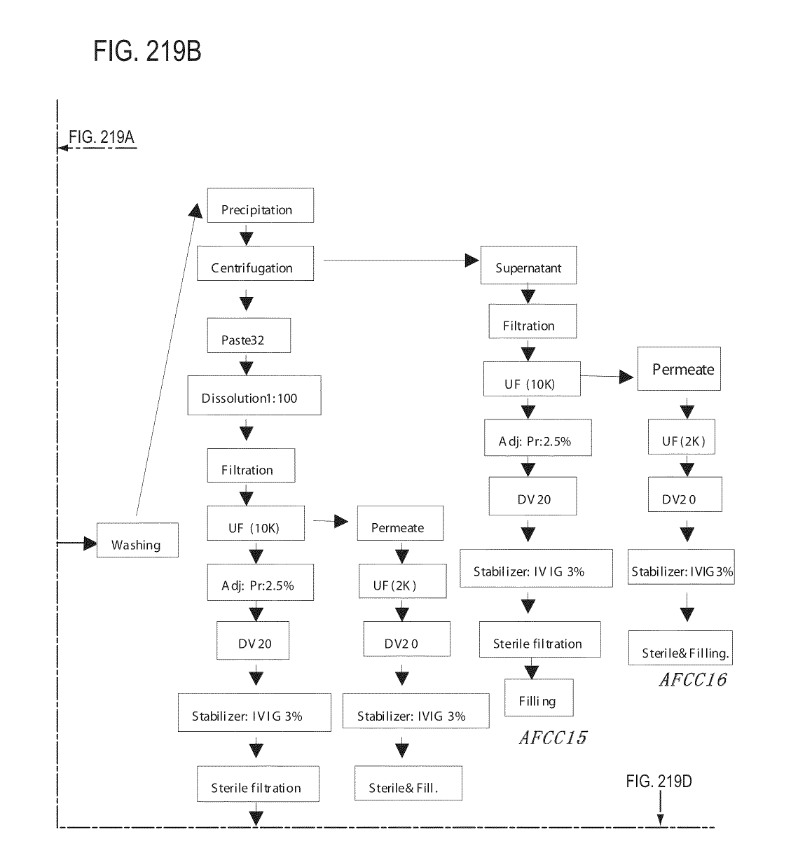

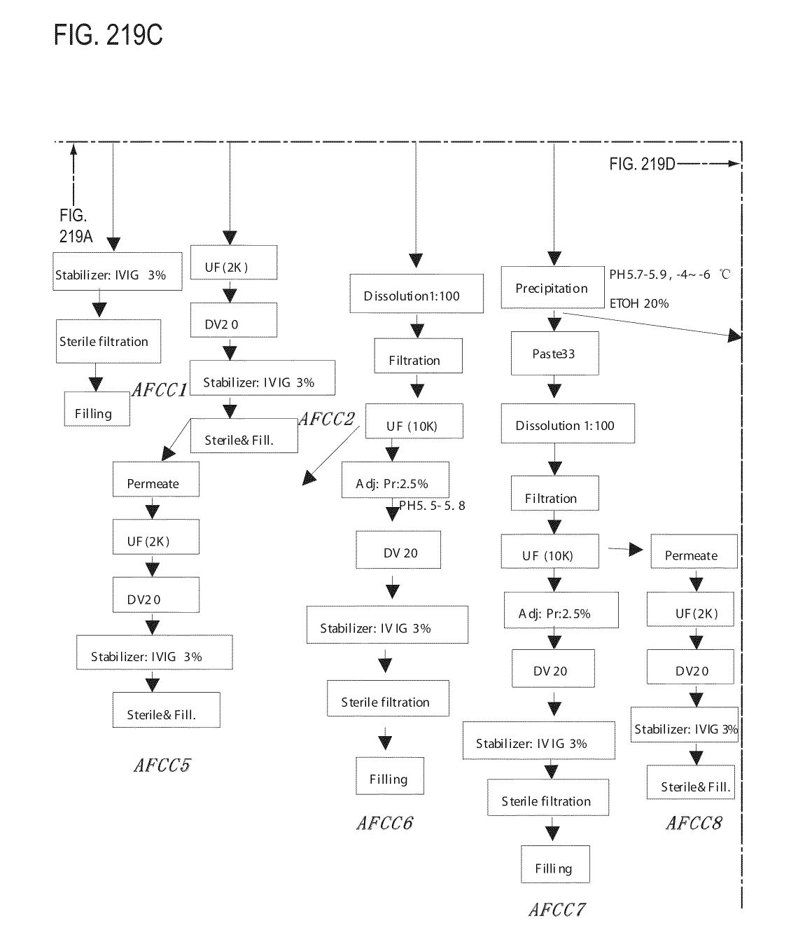

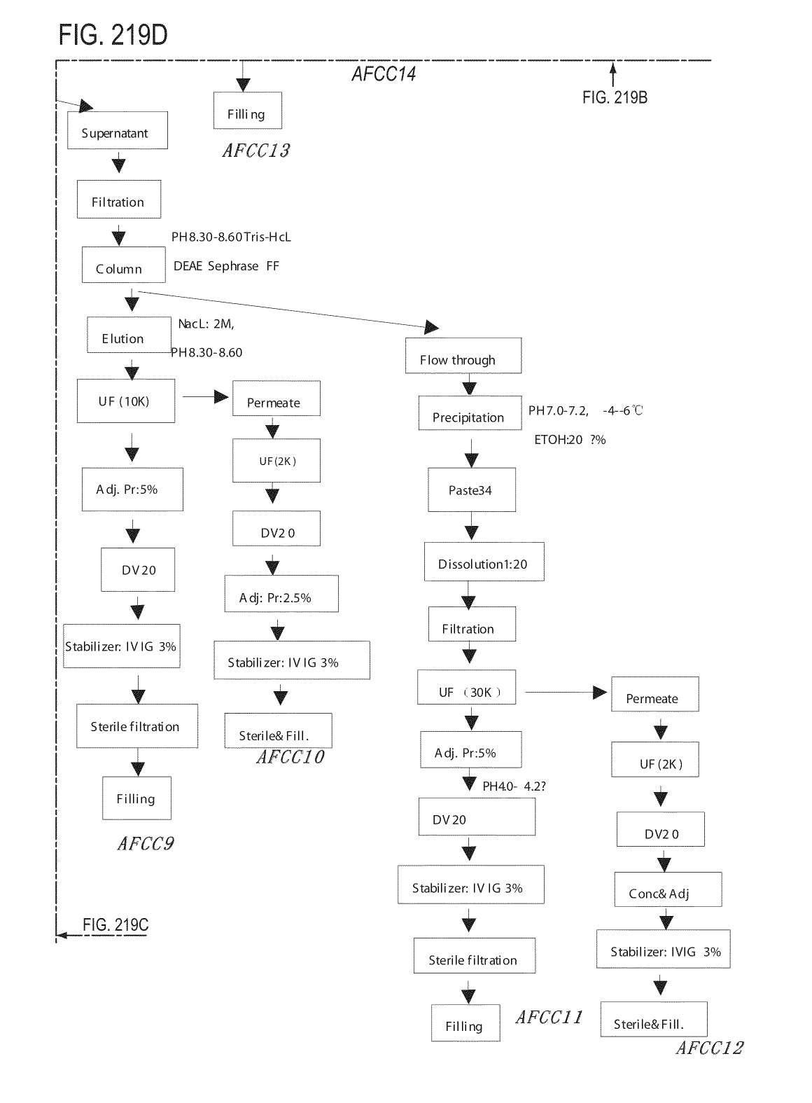

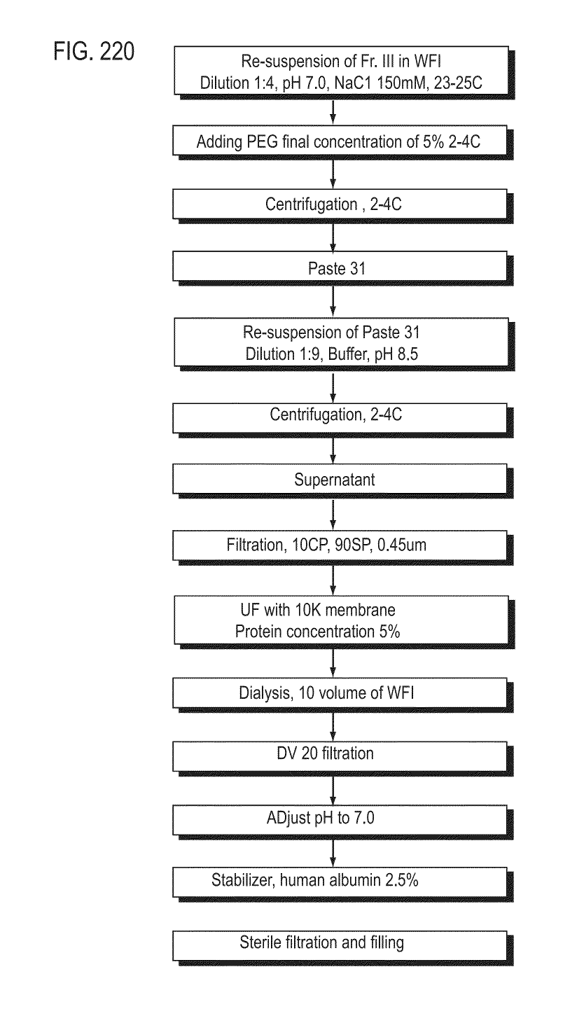

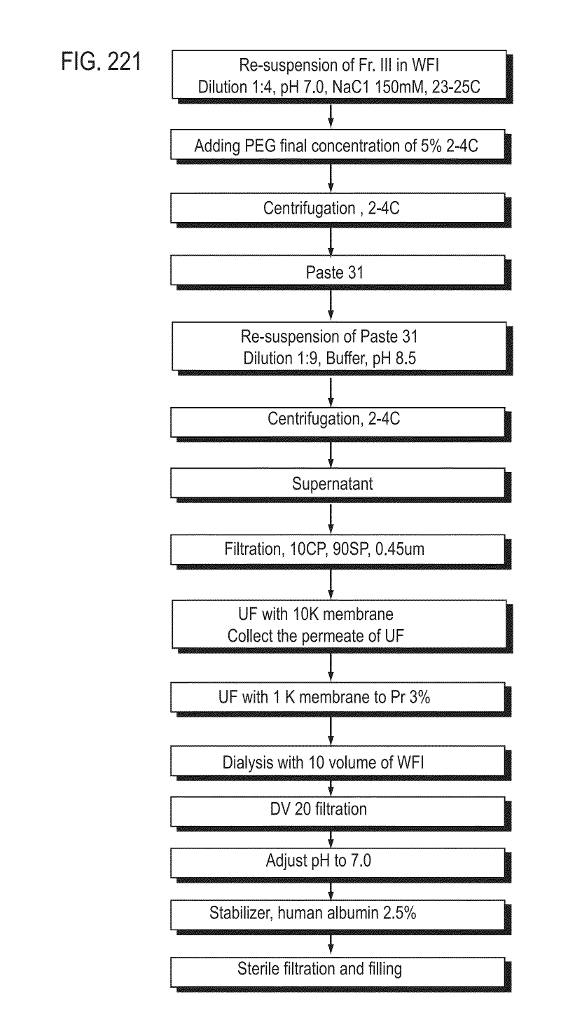

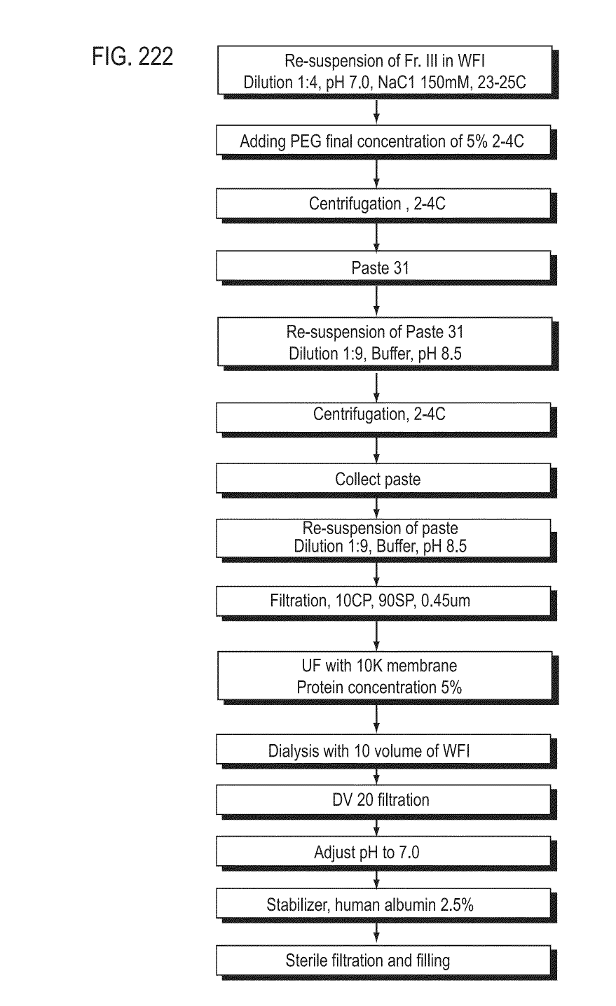

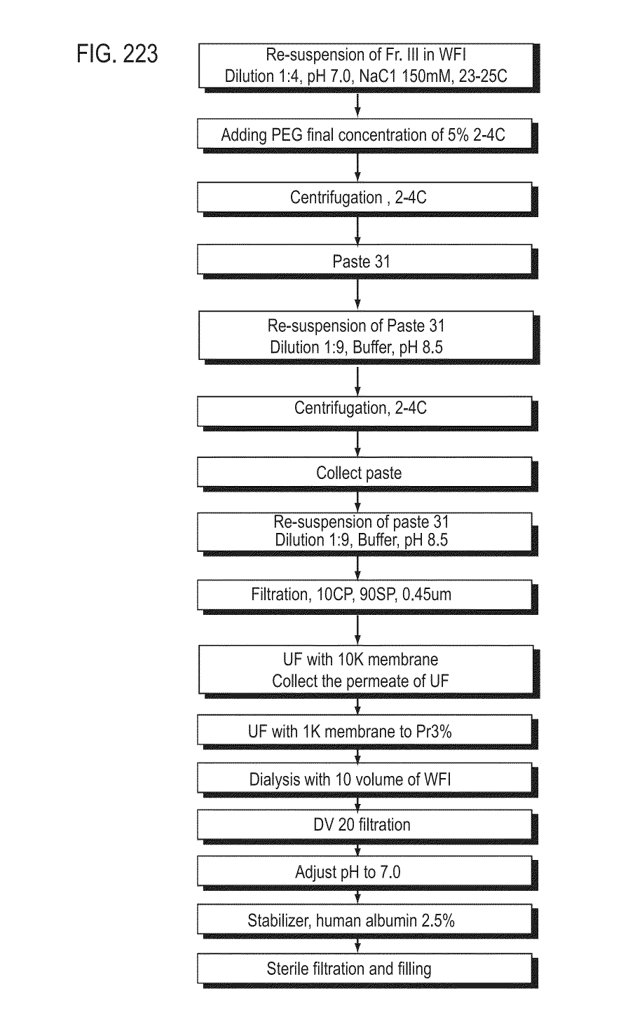

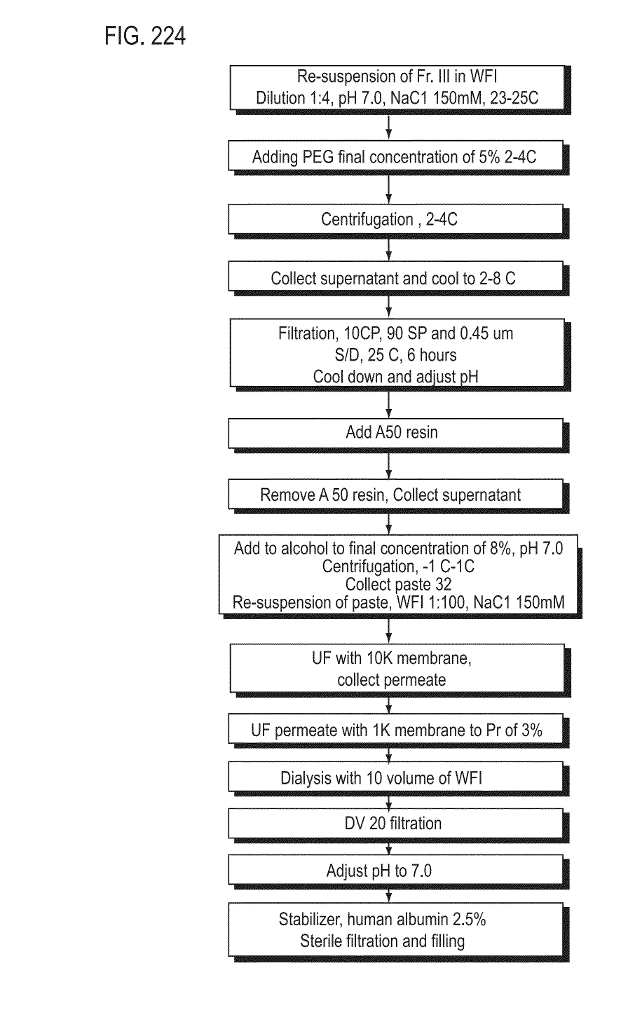

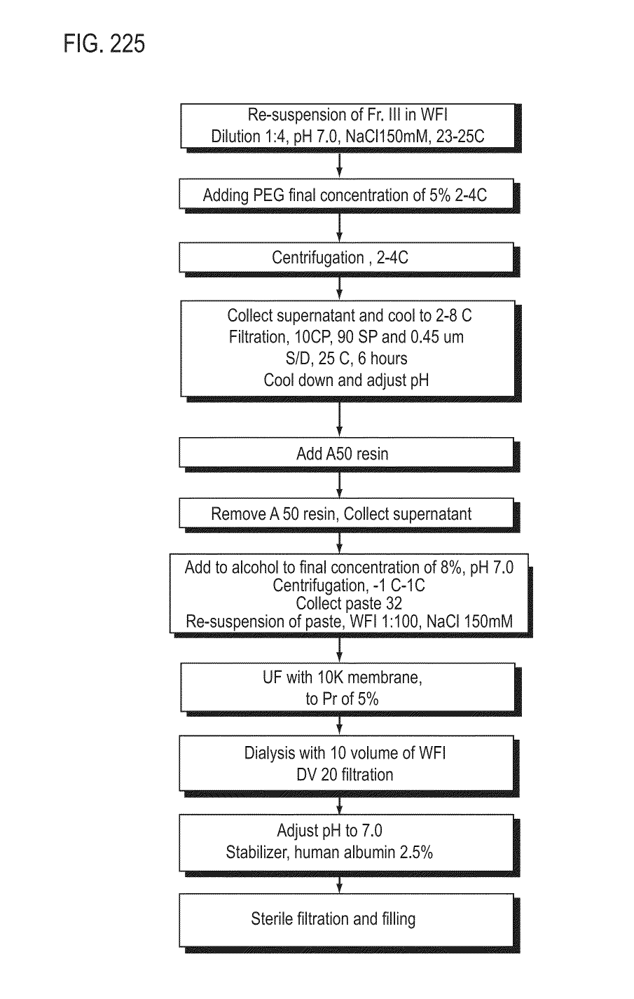

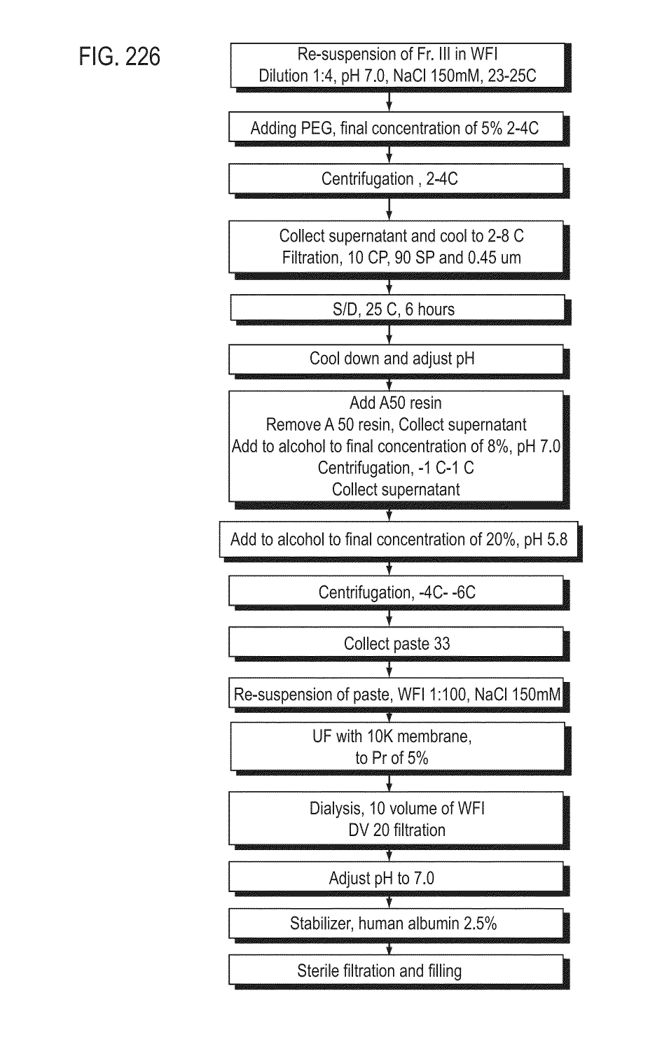

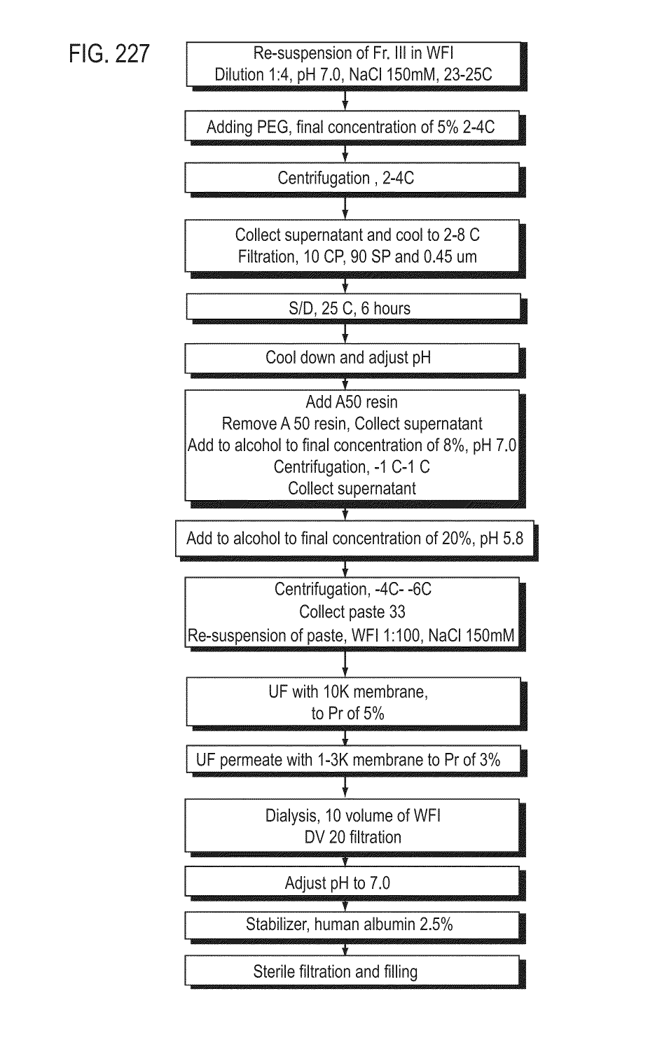

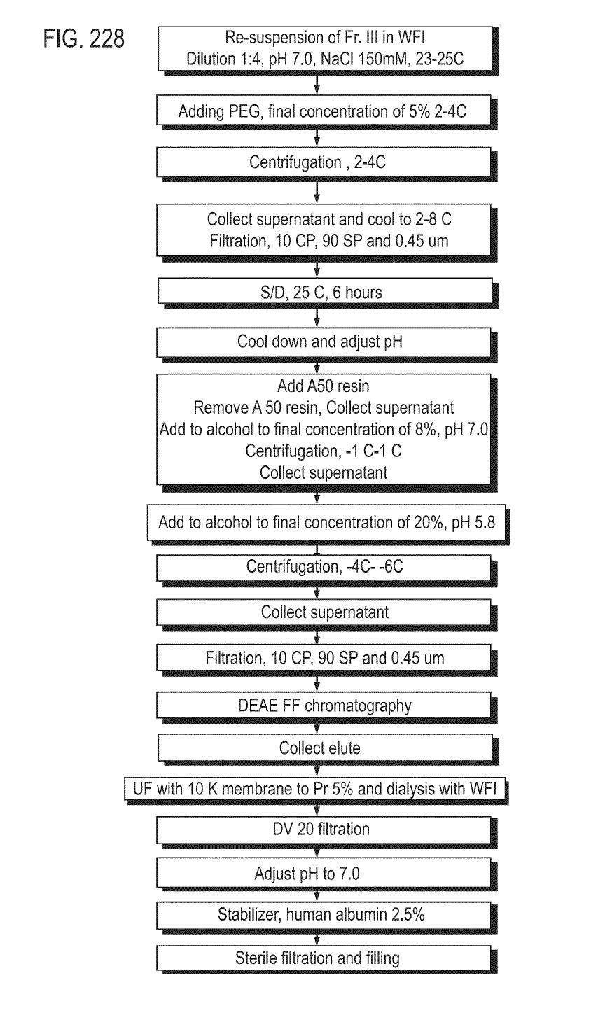

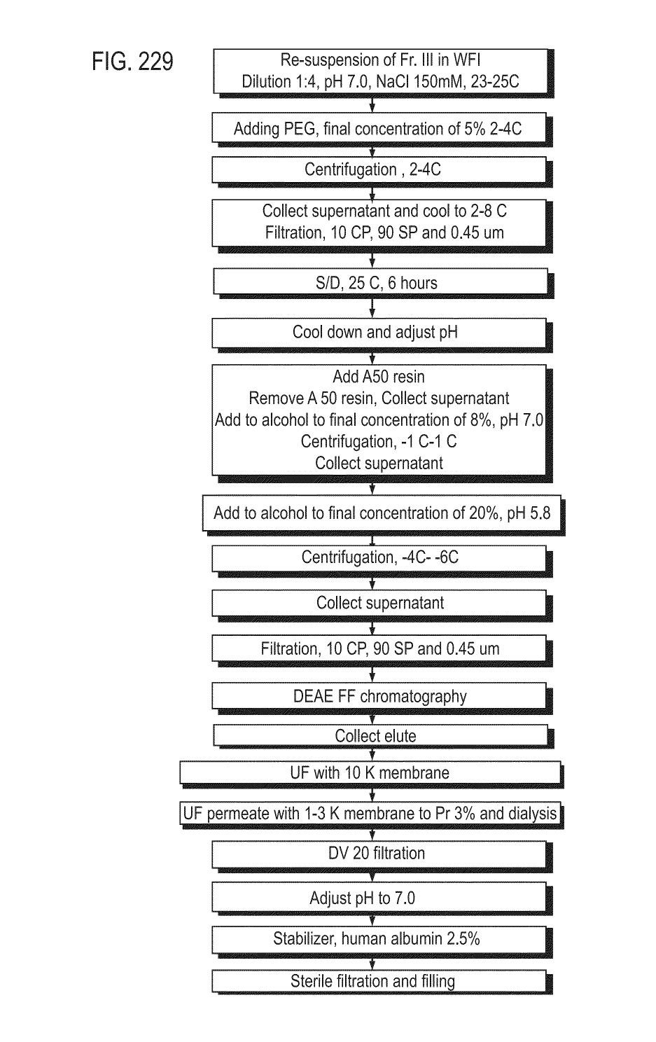

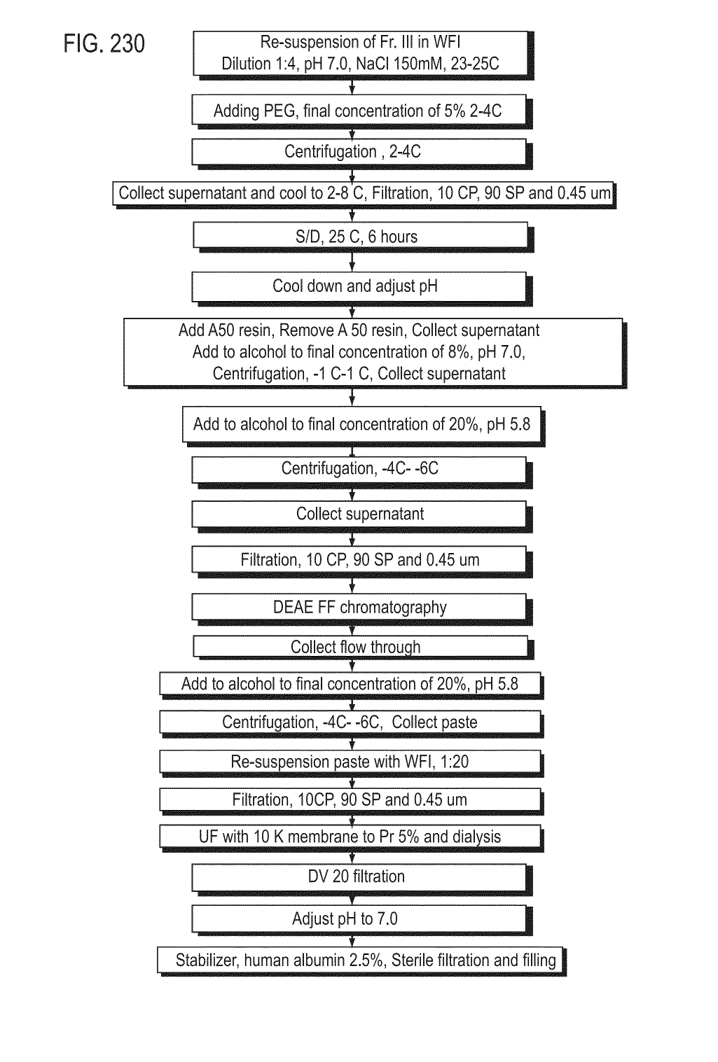

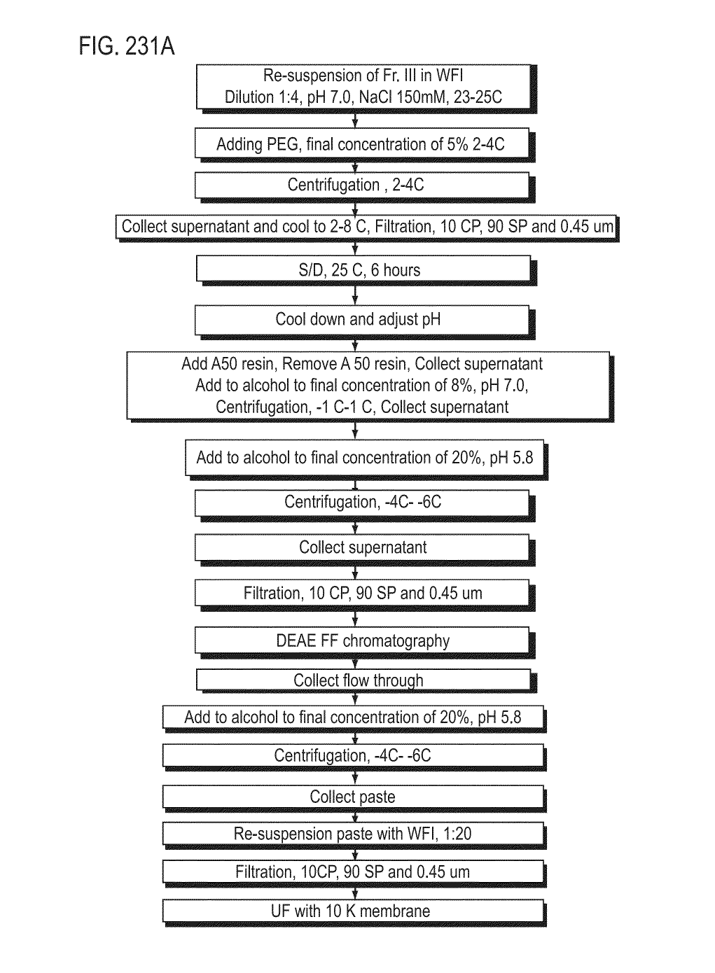

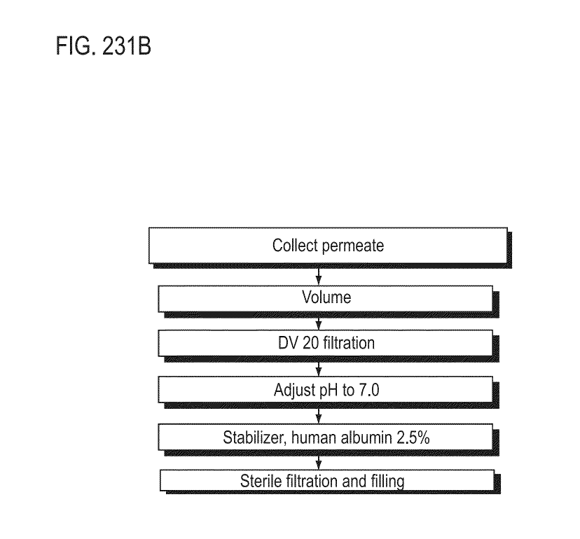

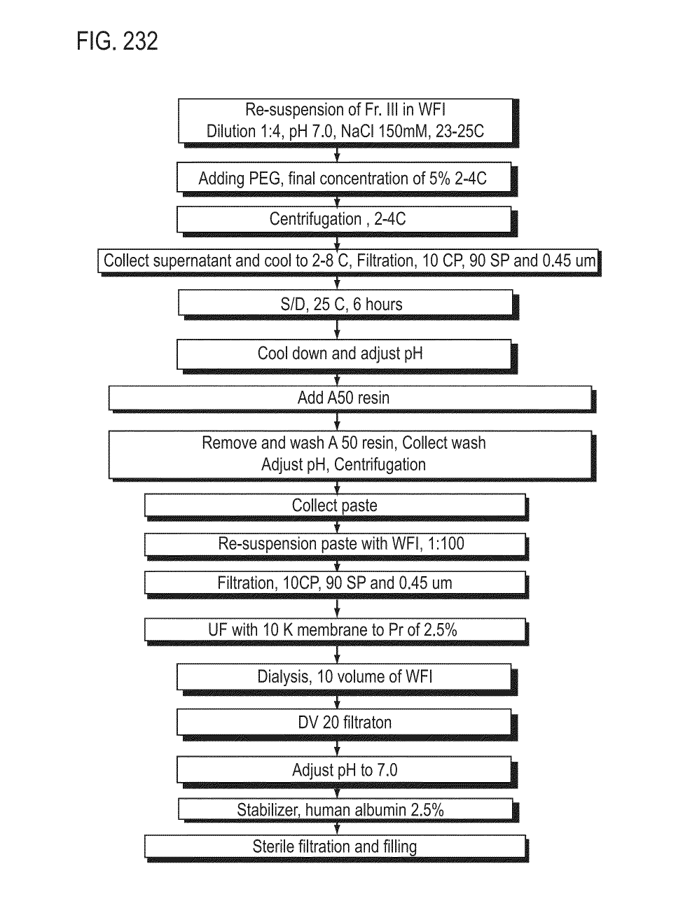

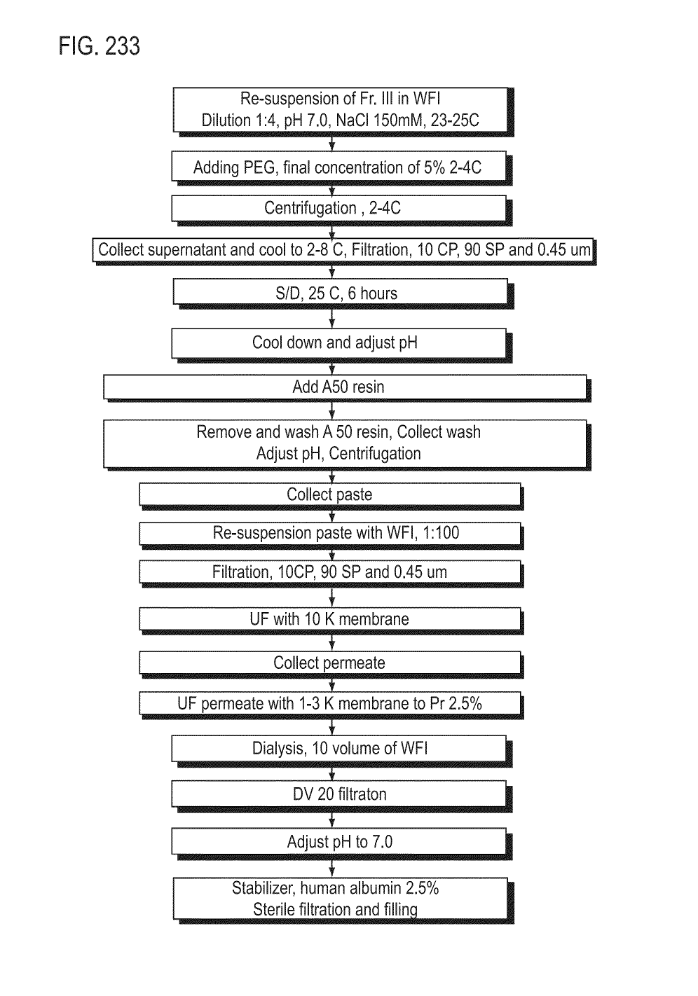

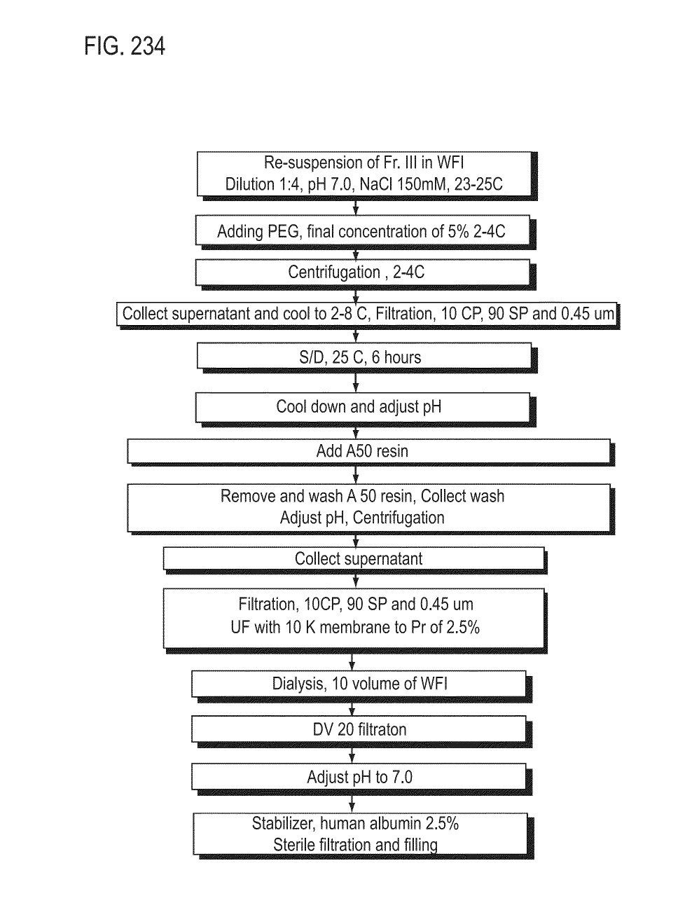

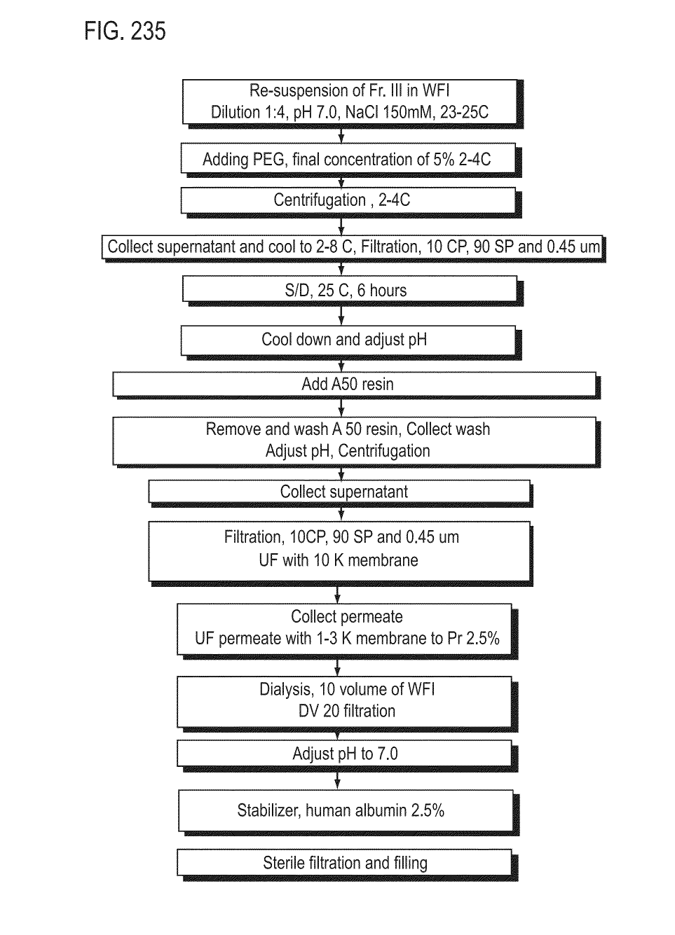

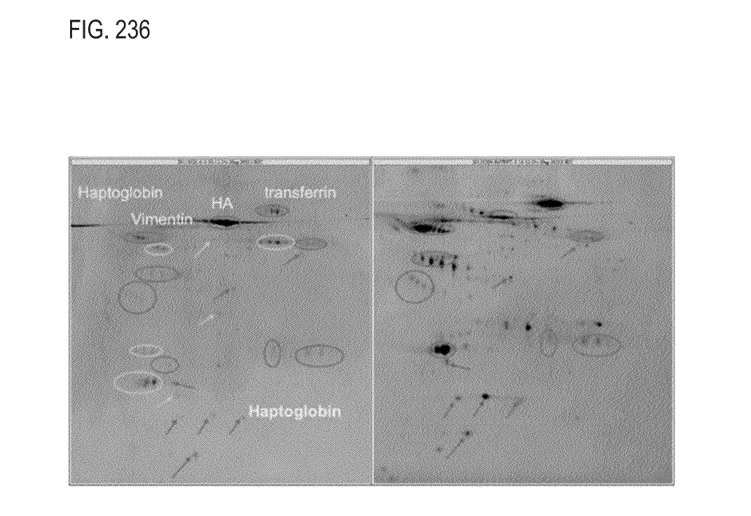

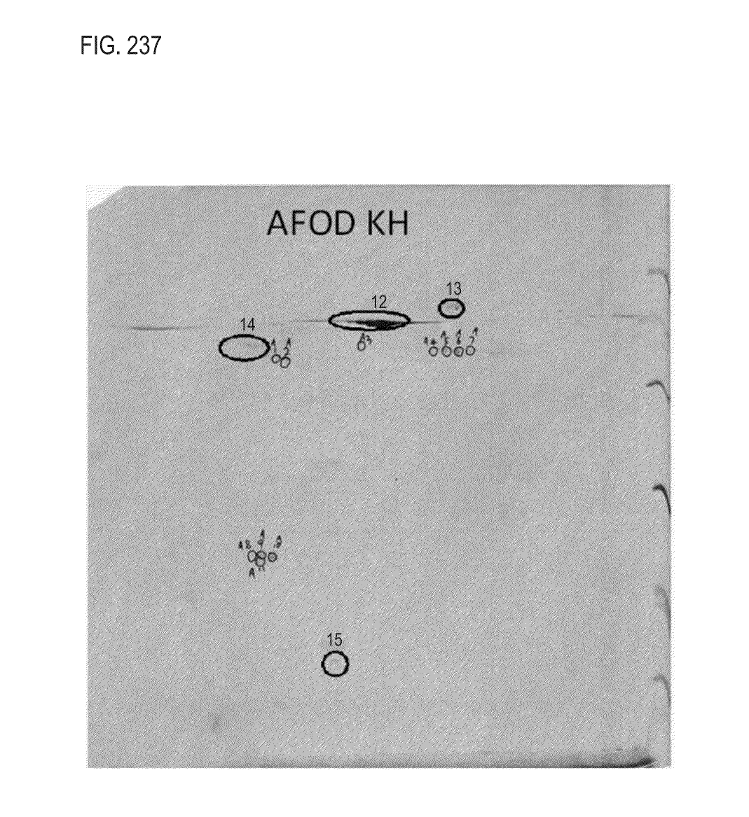

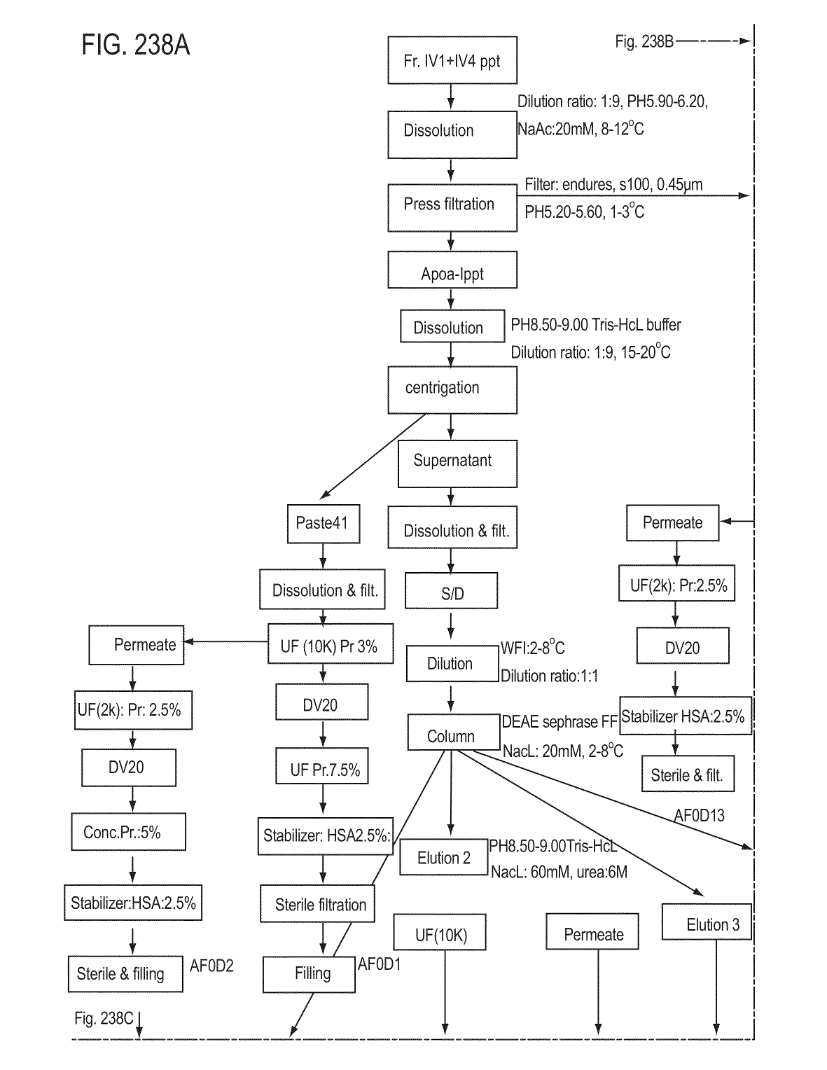

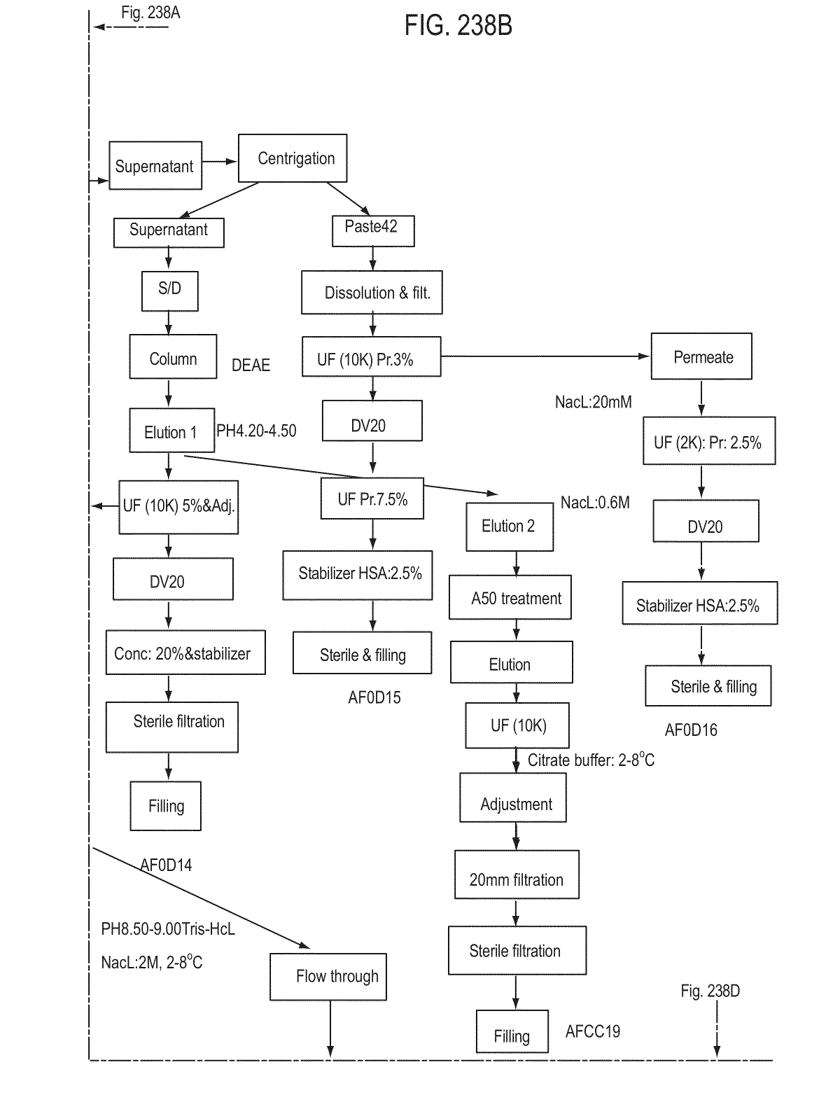

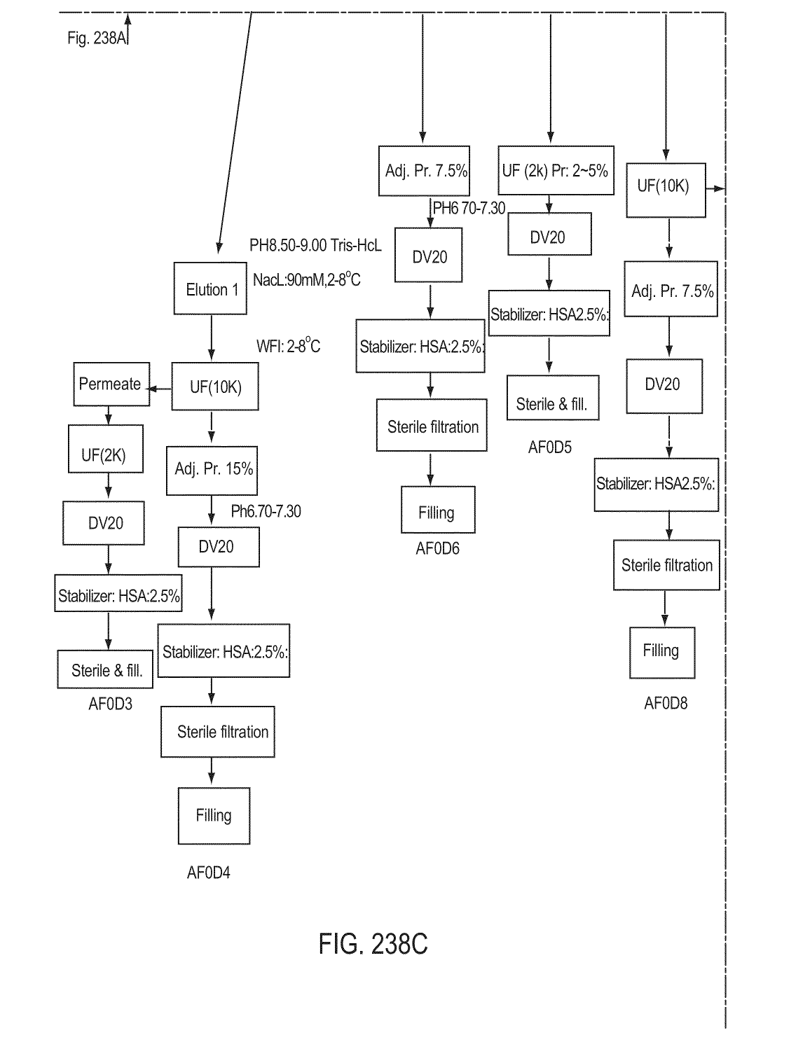

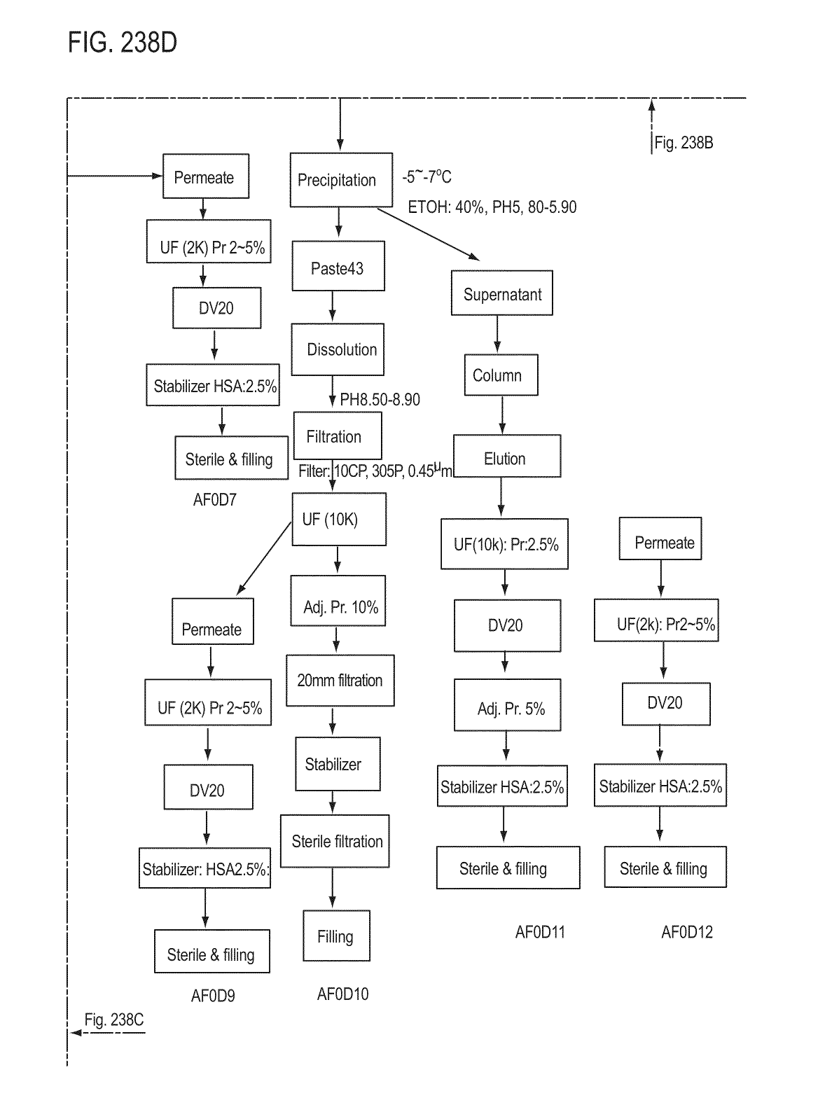

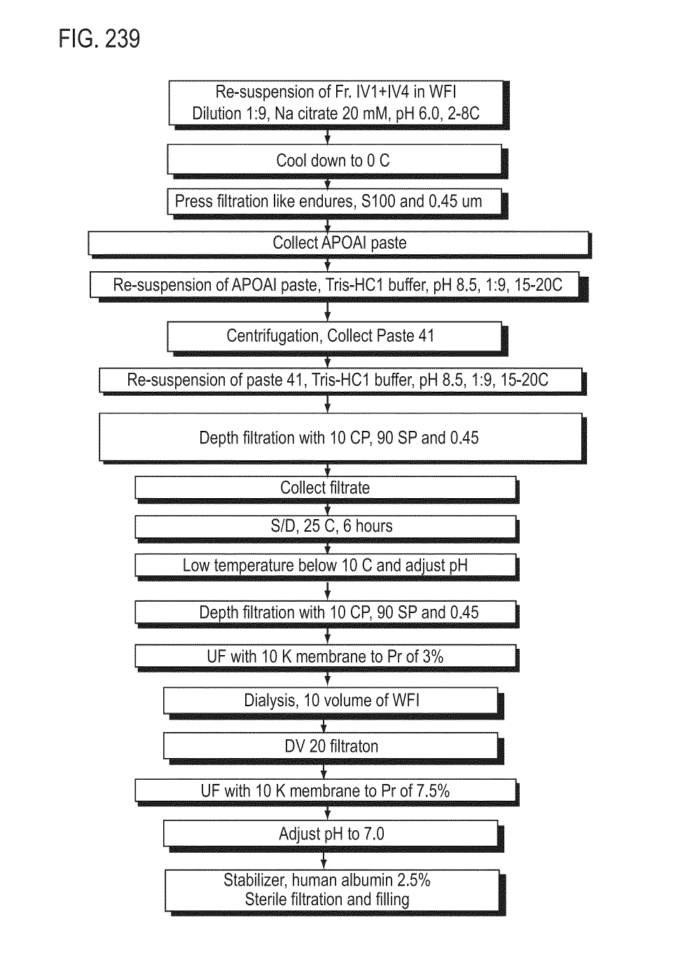

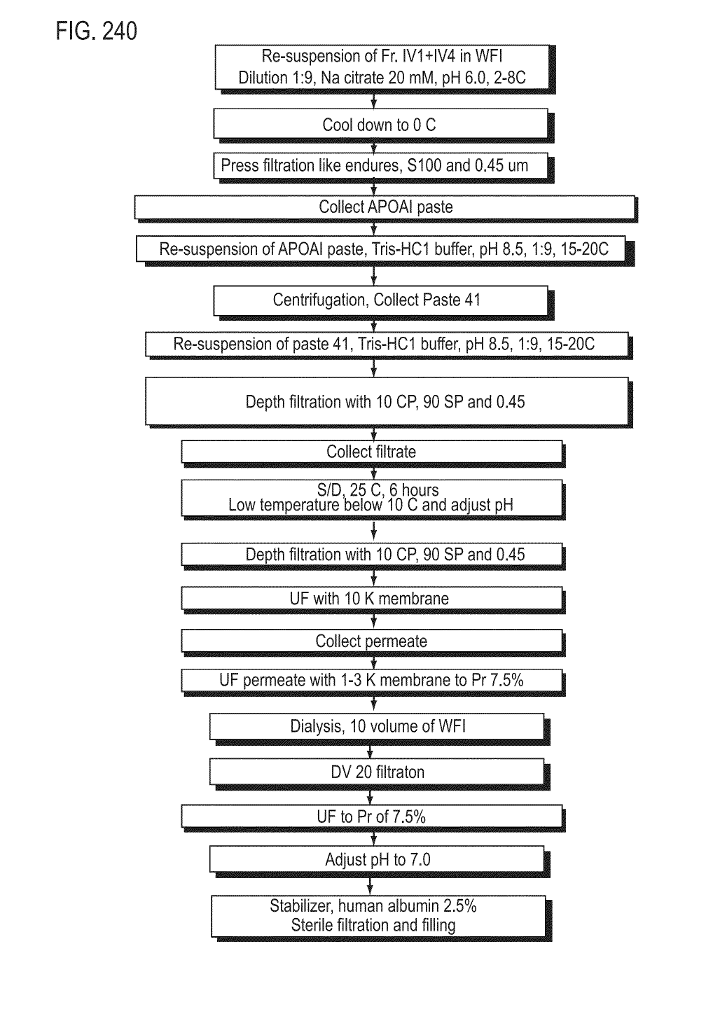

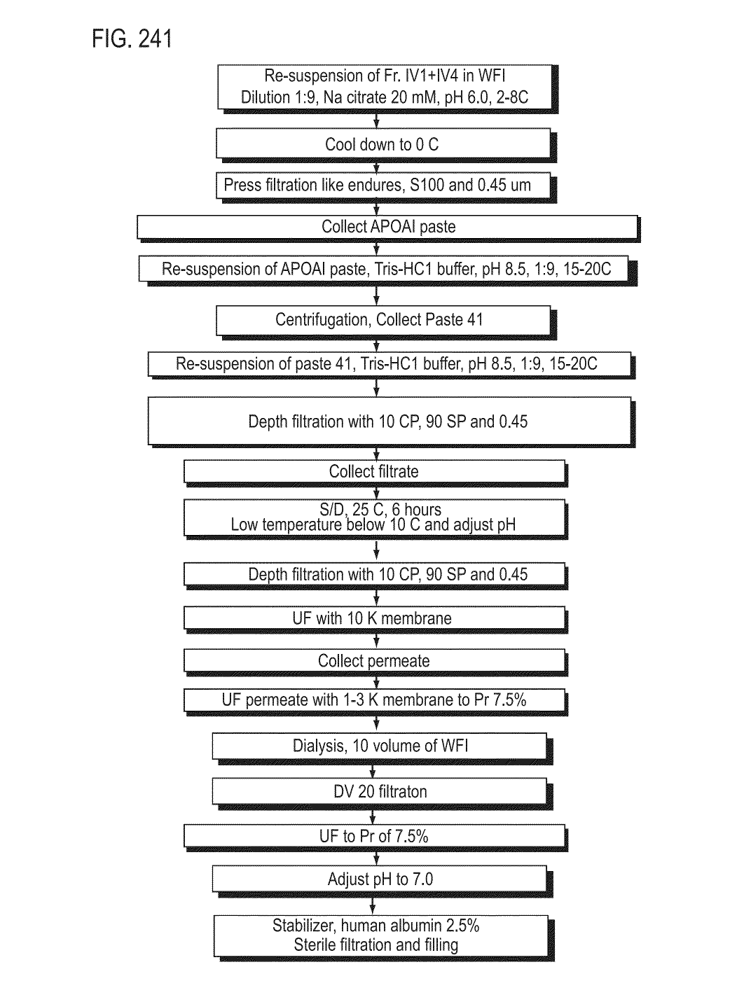

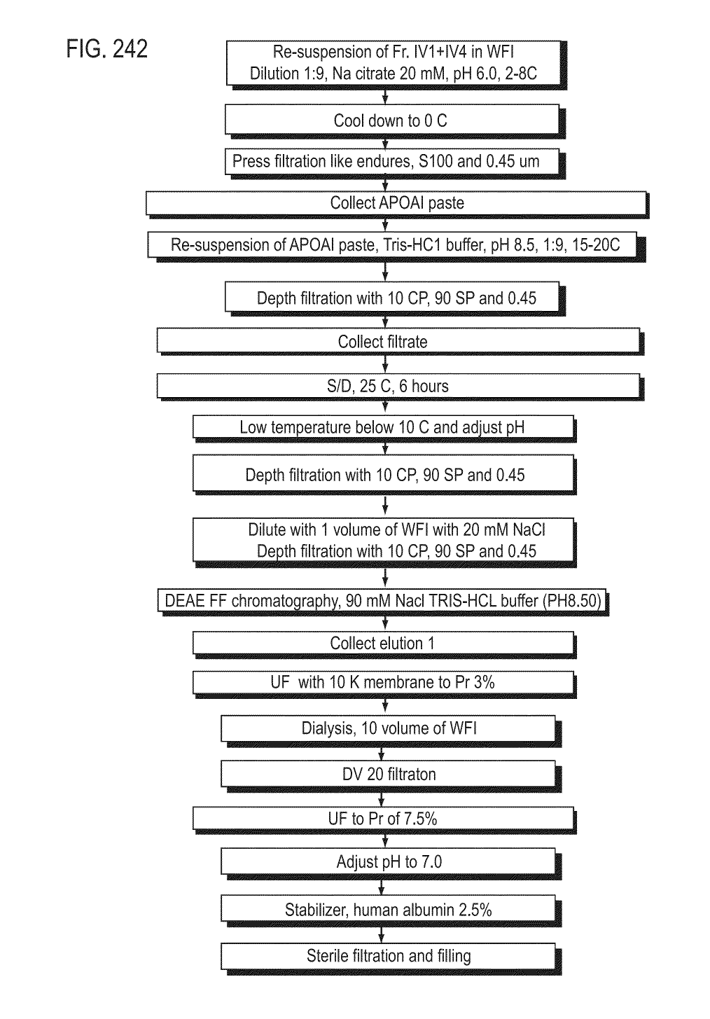

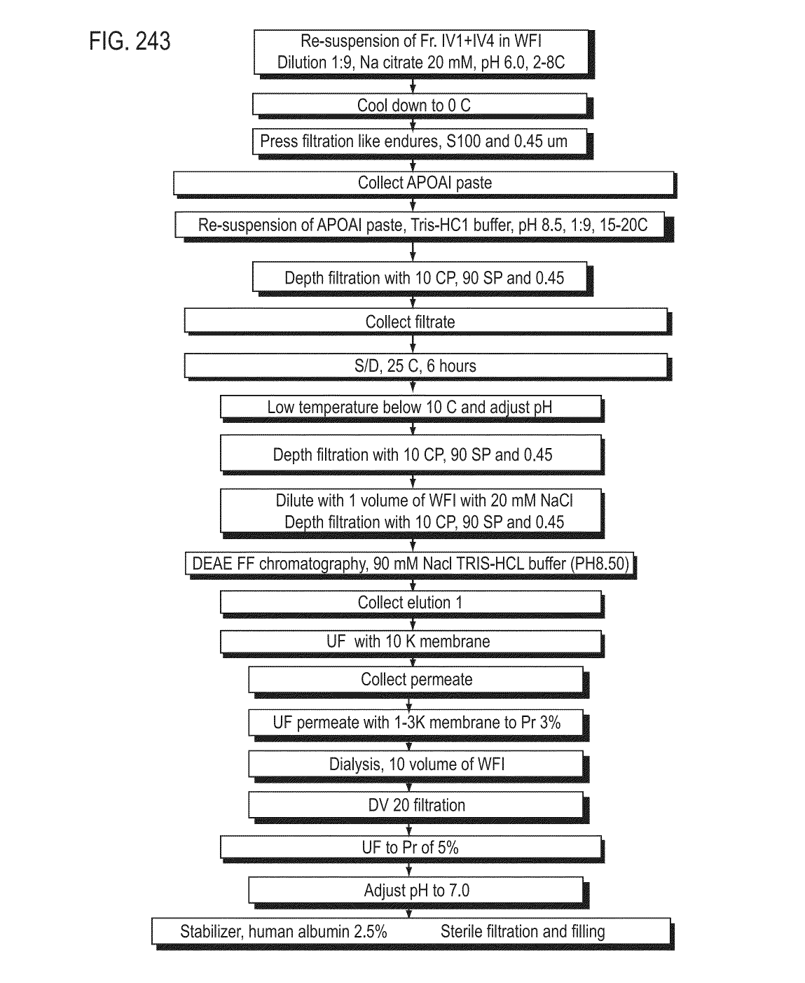

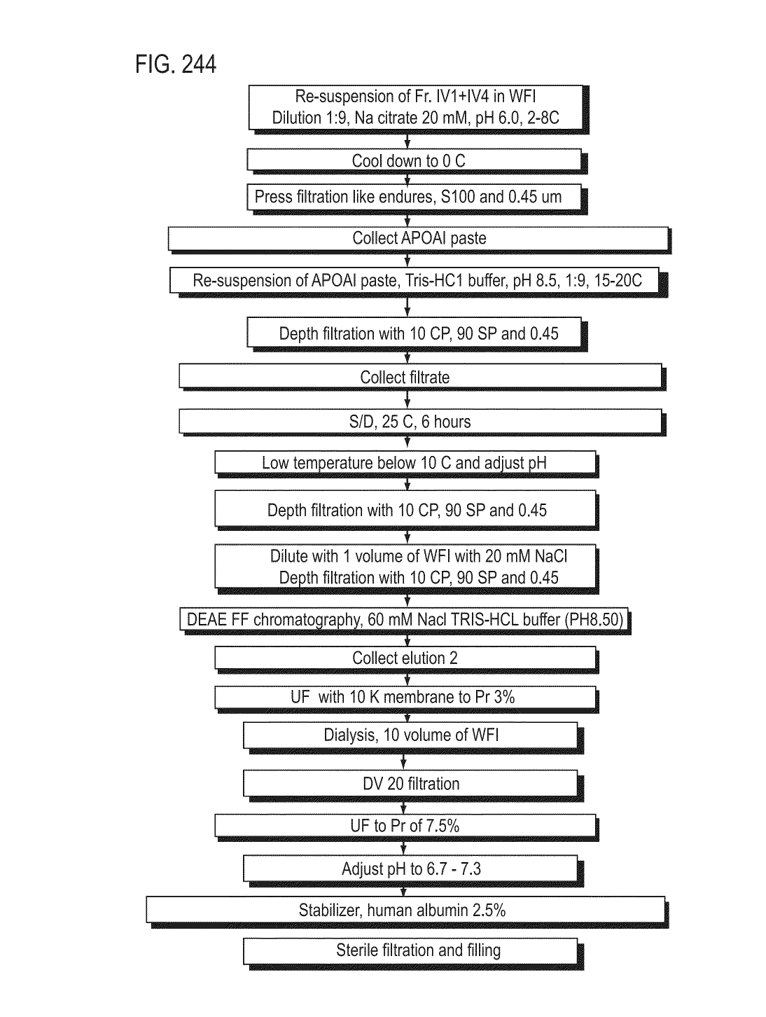

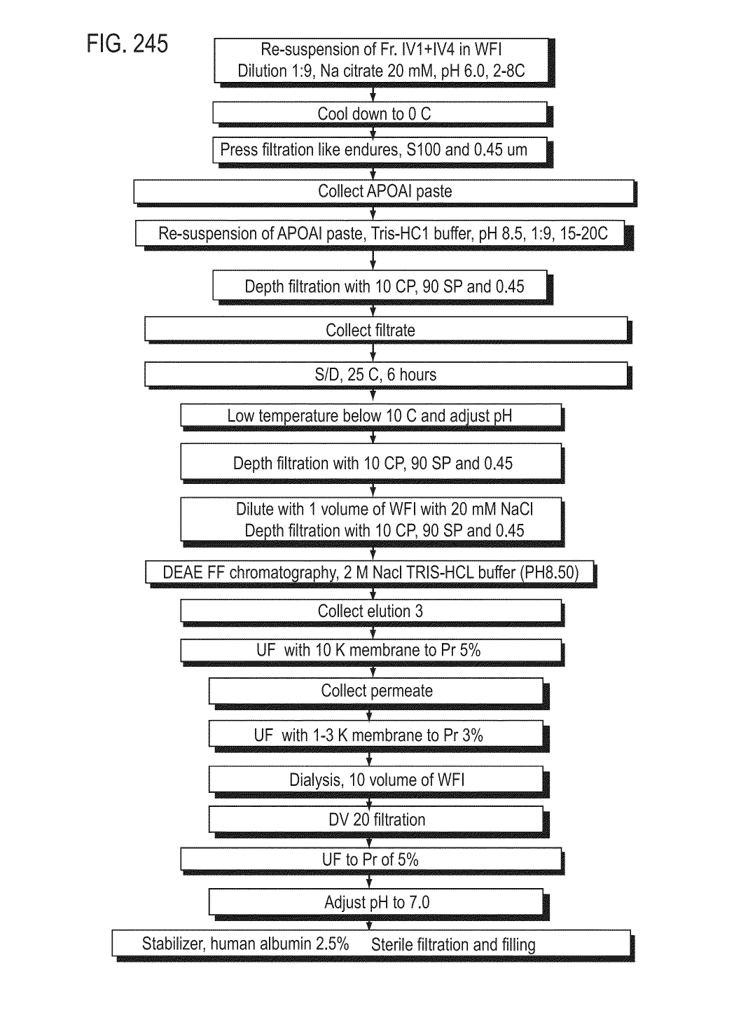

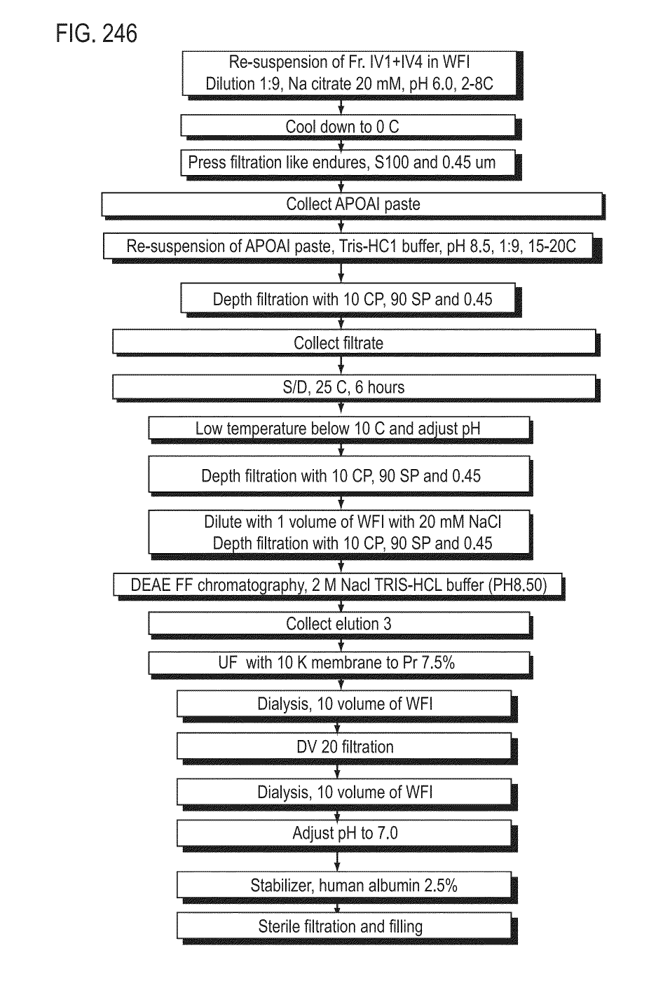

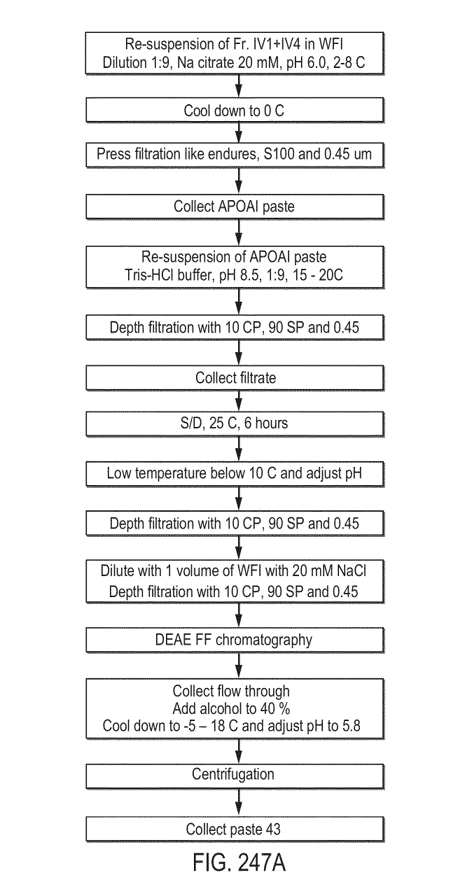

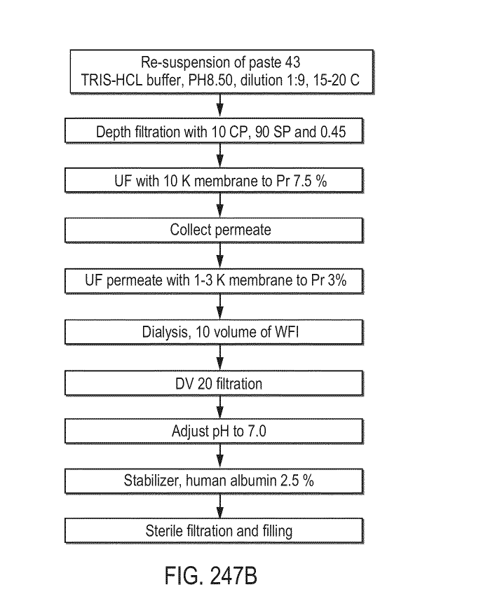

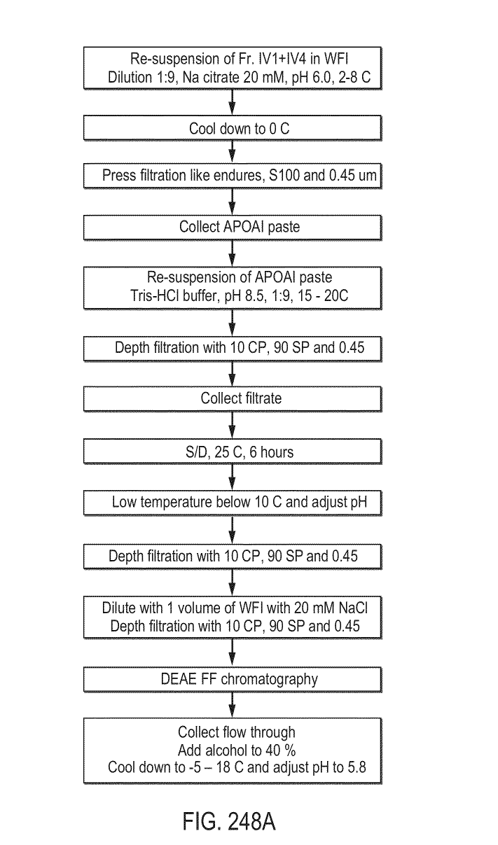

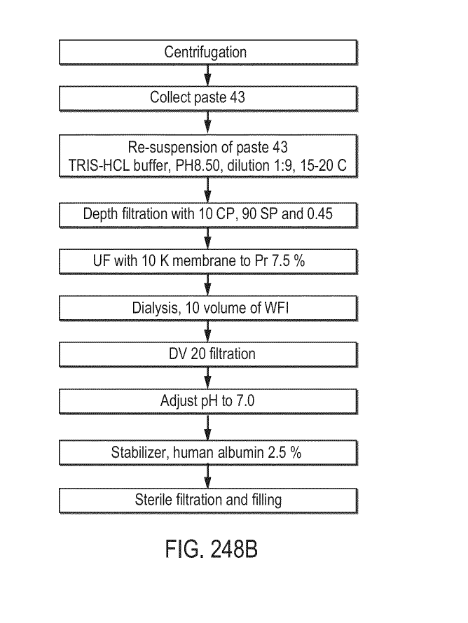

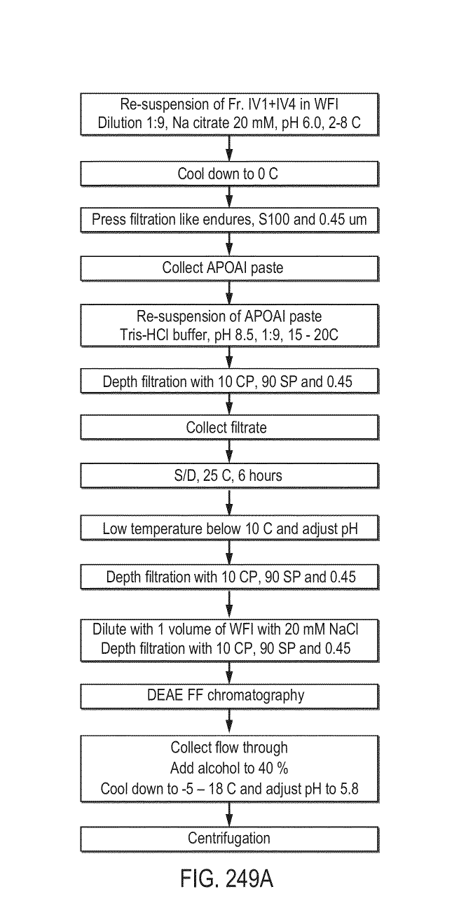

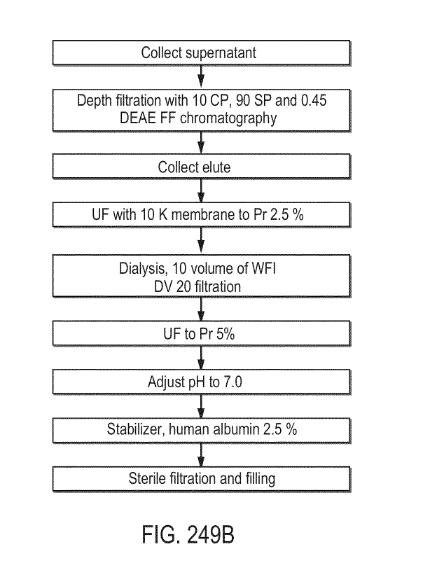

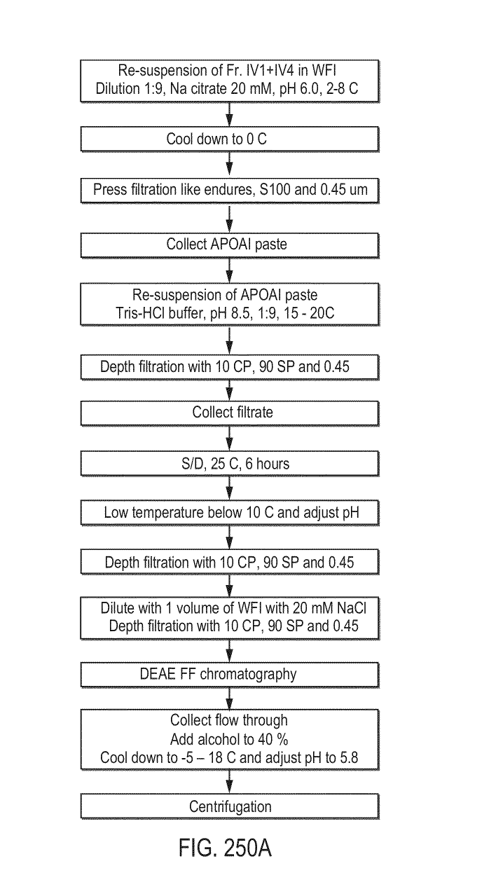

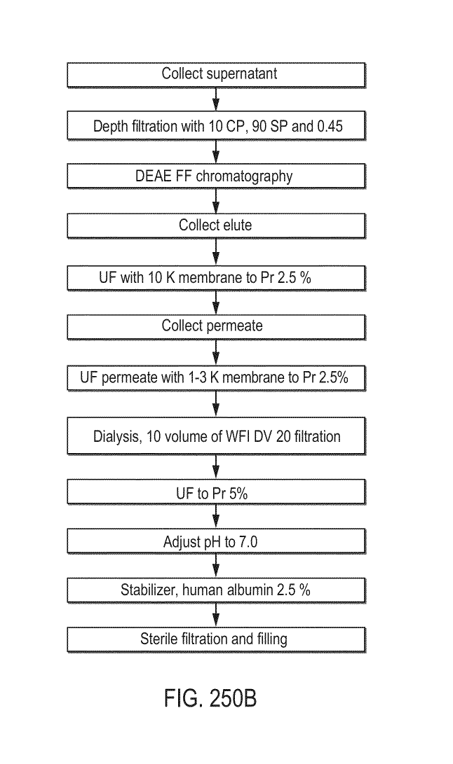

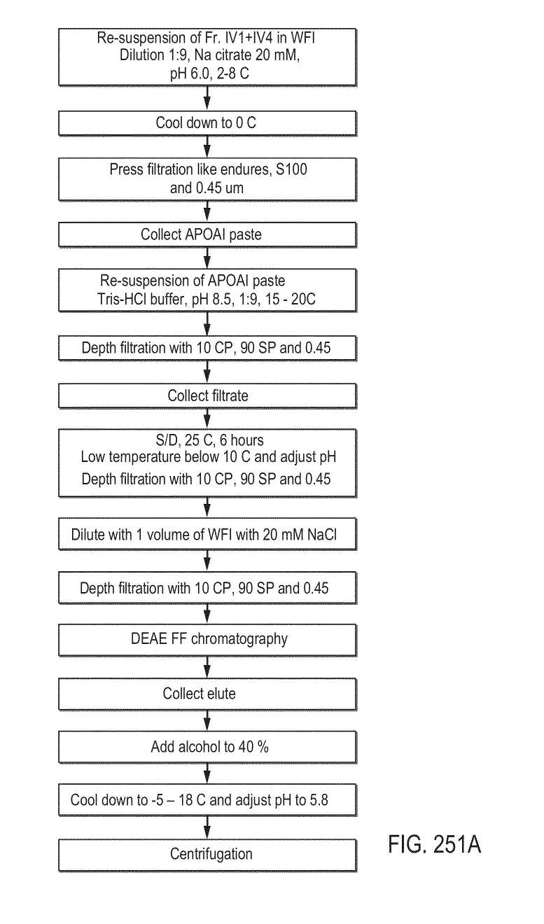

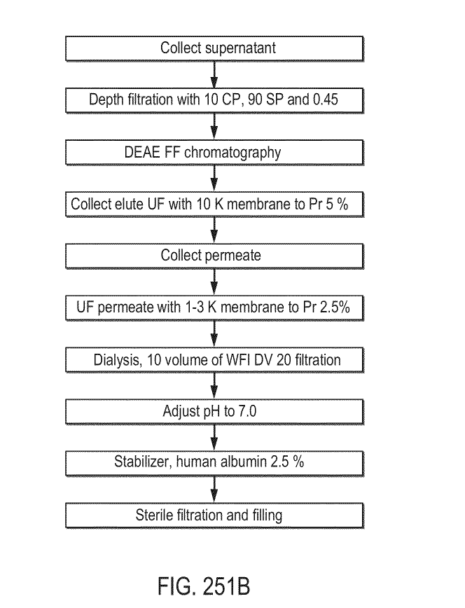

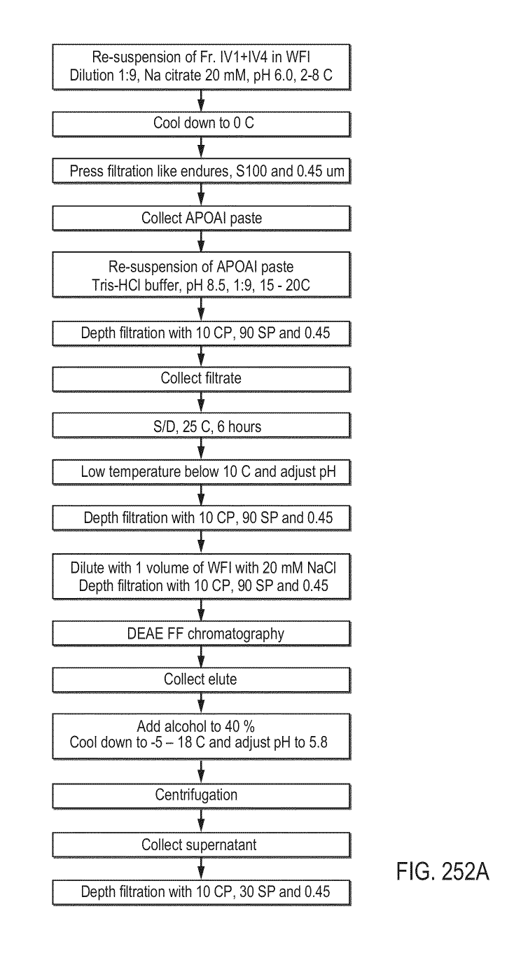

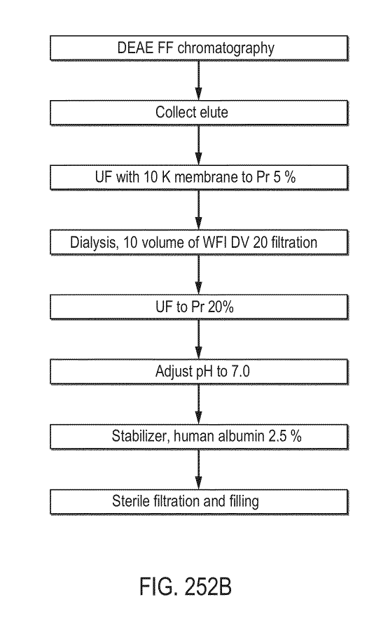

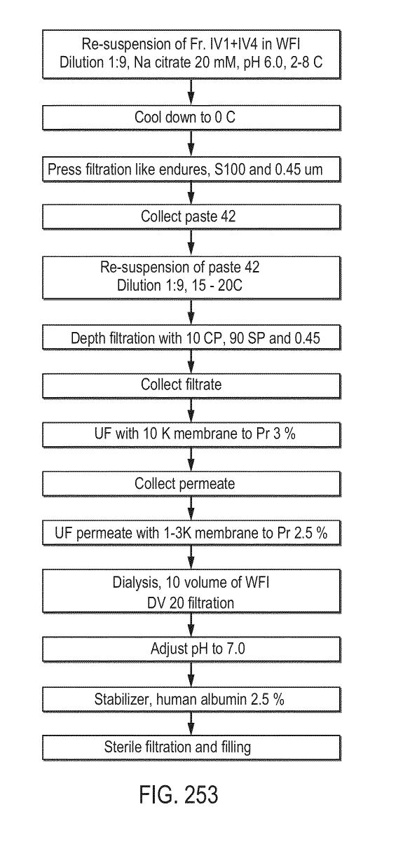

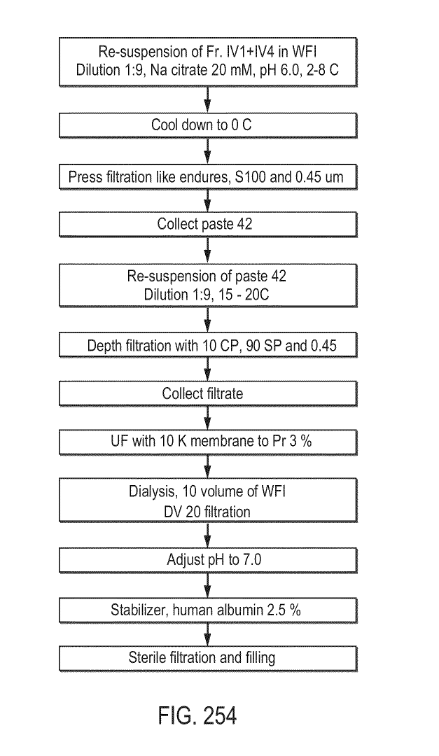

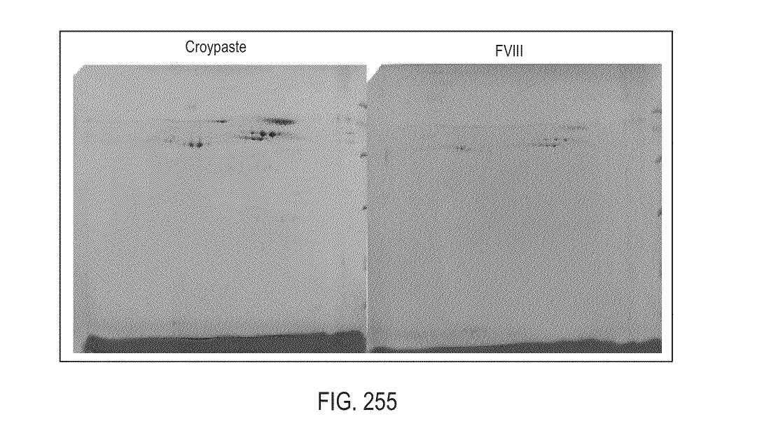

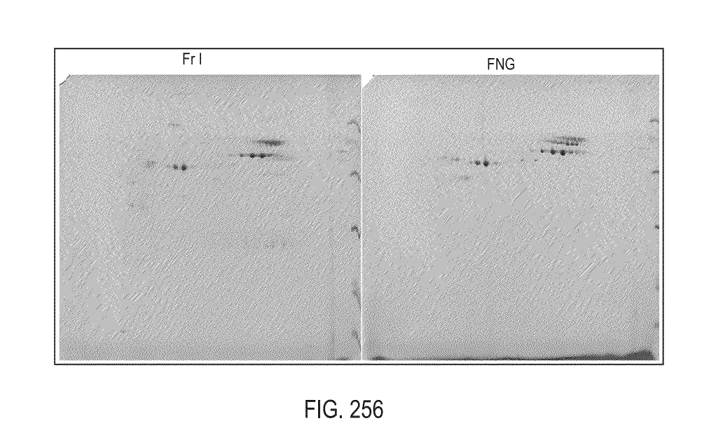

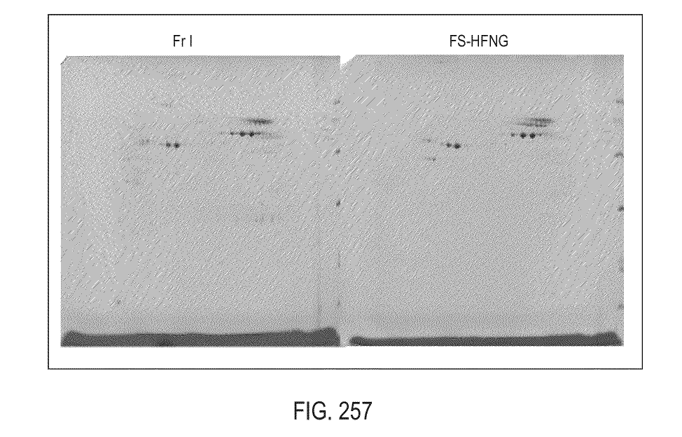

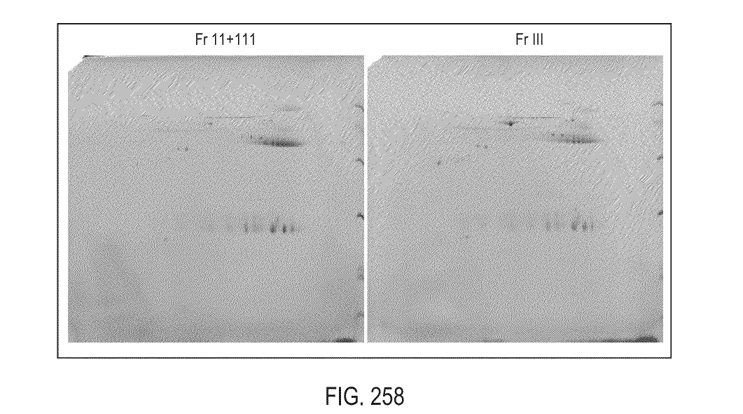

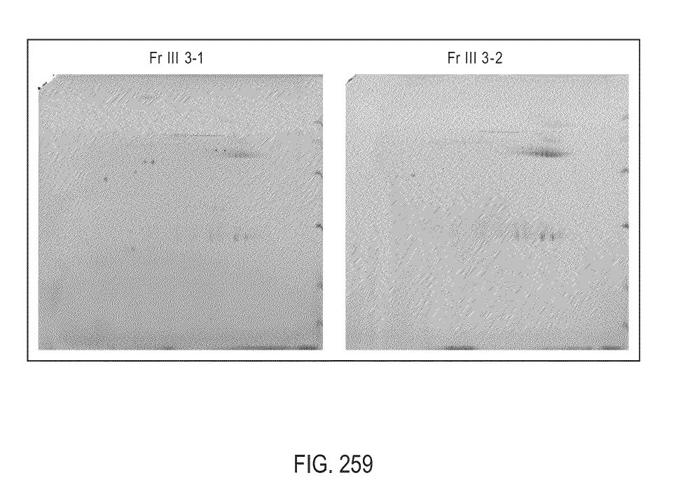

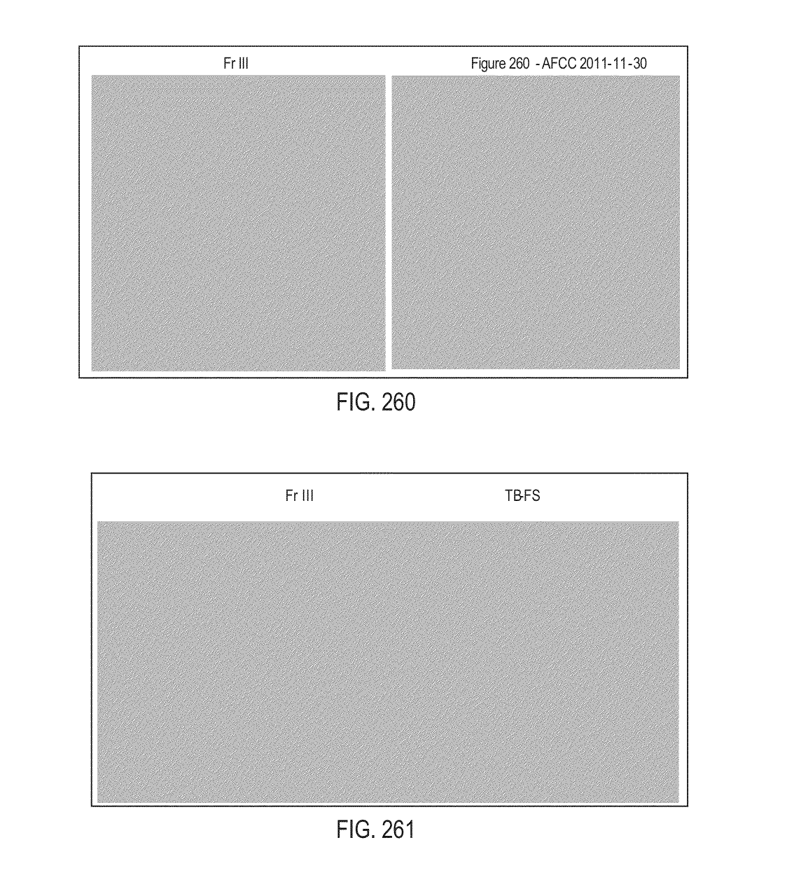

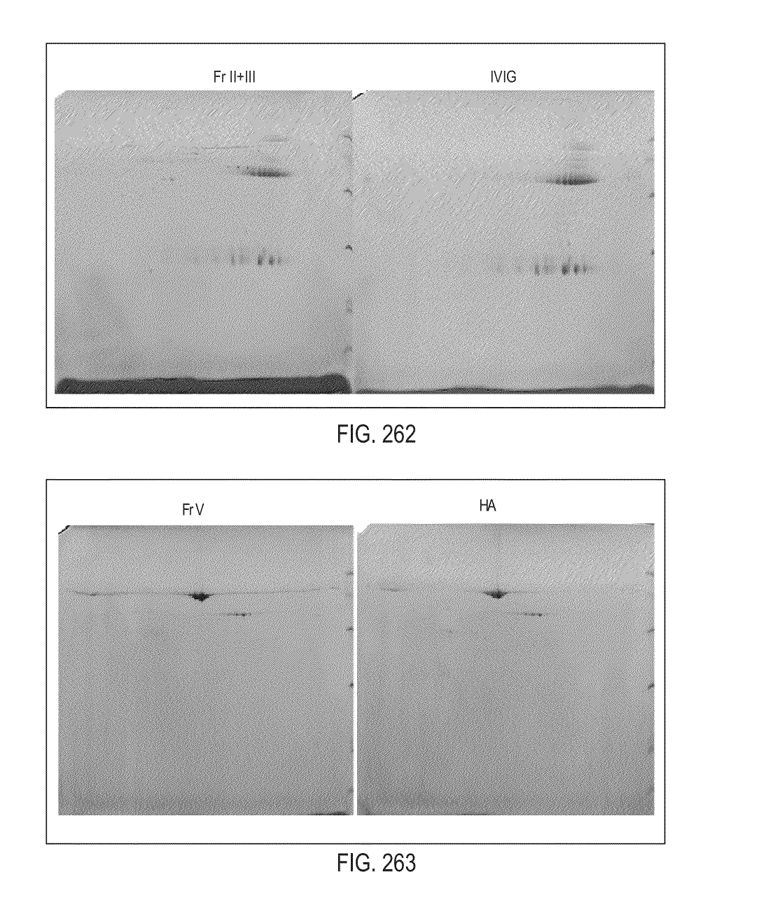

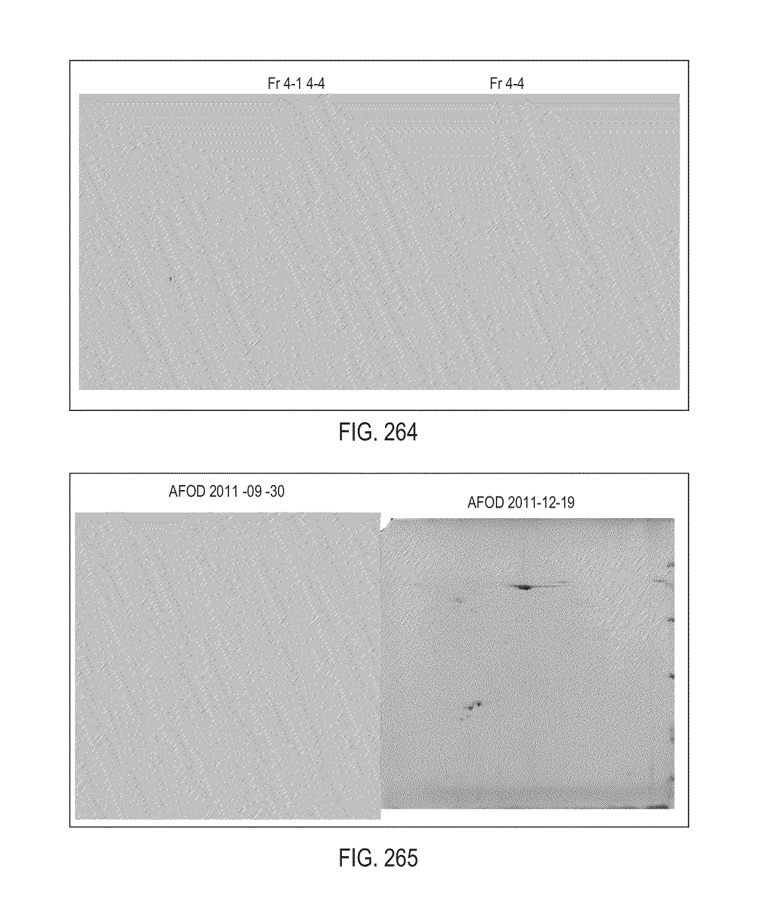

FIG. 184. Body weight change caused with AFCC treatment in mice infected with H1N1 (WSN) influenza

FIG. 185. Body weight change caused with Vehicle treatment in mice infected with H1N1

(WSN) influenza

1. The process of obtaining 30% or higher of a protein selected from the group consisting of Human Albumin protein, Human Albumin uncharacterized protein, HPR 31 kDa protein, AIBG isoform 1 of Alpha-1b-glycoprotein protein, HPR haptoglobin protein, ACTC1 Actin protein, Alpha cardiac muscle 1, KH51 protein, Immunoglobulin proteins from fraction II, 120/E19 IGHV4-31 protein, IGHG1 44 kDa protein, 191/H18 IGHV4-31 protein, IGHG1 32 kDa, IGHV4-31 protein, IGHG1 putative uncharacterized protein, KH 33 protein, KH 34 protein, KH 35 protein, KH 36 protein, KH37 protein, Hepatitis B immunoglobulin protein from fraction II, TF protein sequence#197/H24 protein, TF serotransferrin protein, Immunoglobulin protein from fraction III, 193/H20 TF serotransferrin protein, 194/H21 APOH beta2-glycoprotein 1 protein, 195/H22 cDNA FLJ5165 protein, beta-2-glycoprotein protein, 196/H23FCN3 isoform 1 of Ficolin-3 protein, KH 3 protein, KH 4 protein, KH 5 protein, KH 6 protein, KH 7 protein, KH 8 protein, KH 9 protein, KH 10 protein, KH 41 protein, KH 42 protein, KH 43 protein, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

2. The process of claim 1, wherein the protein is Human Albumin uncharacterized protein.

3. The process of claim 1, wherein the protein is HPR 31 kDa protein.

4. The process of claim 1, wherein the protein is AIBG isoform 1 of Alpha-1b-glycoprotein protein.

5. The process of claim 1, wherein the protein is HPR haptoglobin protein.

6. The process of claim 1, wherein the protein is ACTC1 Actin protein.

7. The process of claim 1, wherein the protein is Alpha cardiac muscle 1 protein.

8. The process of claim 1, wherein the protein is KH51 protein.

9. The process of claim 1, wherein the protein is any combination of any of the following proteins found in Human Albumin: Human Albumin uncharacterized, HPR 31 kDa, AIBG isoform 1 of Alpha-1b-glycoprotein, HPR haptoglobin, ACTC1 Actin, Alpha cardiac muscle 1 and KH51 protein.

10. The process of claim 1, wherein the protein is HPR 31 kDa, ACTC1 Actin, Alpha cardiac muscle 1 and KH51 protein can only be found in Human Albumin with trademark AIbuRAAS®.

11. The process of claim 1, wherein the protein is an Immunoglobulin protein from fraction II.

12. The process of claim 1, wherein the protein is 120/E19 IGHV4-31 protein.

13. The process of claim 1, wherein the protein is IGHG1 44 kDa protein.

14. The process of claim 1, wherein the protein is 191/H18 IGHV4-31 protein.

15. The process of claim 1, wherein the protein is IGHG1 32 kDa protein.

16. The process of claim 1, wherein the protein is IGHV4-31 protein.

17. The process of claim 1, wherein the protein is IGHG1 putative uncharacterized protein DKFZp686G11190 protein

18. The process of claim 1, wherein the protein is KH33 protein.

19. The process of claim 1, wherein the protein is KH34 protein.

20. The process of claim 1, wherein the protein is KH35 protein.

21. The process of claim 1, wherein the protein is KH36 protein.

22. The process of claim 1, wherein the protein is KH37 protein.

23. The process of claim 1, wherein the protein is any combination of any of the following proteins found in Immunoglobulin: 120/E19 IGHV4-31, IGHG1 44 kDa, 191/H18 IGHV4-31, IGHG1 32 kDa, IGHV4-31, IGHG1 Putative uncharacterized DKFZp686G11190, KH33, KH34, KH35, KH36 and KH37 proteins.

24. The process of claim 1, wherein the protein is KH33, KH34, KH35, KH36 and KH37 protein, that can only be found in Immunoglobulin with trademark GammaRAAS.

25. The process of claim 1, wherein the protein is Hepatitis B immunoglobulin protein.

26. The process of claim 1, wherein the protein is TF protein sequence#197/H24 protein.

27. The process of claim 1, wherein the protein is TF serotransferrin protein.

28. The process of claim 1, wherein the protein is any combination of any of the following proteins found in Hepatitis B Immunoglobulin: TF protein sequence#197/H24 and TF serotransferrin proteins.

29. The process of claim 1, wherein the protein is Immunoglobulin protein from fraction III.

30. The process of claim 1, wherein the protein is 193/H20 TF serotransferrin protein.

31. The process of claim 1, wherein the protein is 194/H21 APOH beta2-glycoprotein 1 protein.

32. The process of claim 1, wherein the protein is 195/H22 cDNA FU5165 protein.

33. The process of claim 1, wherein the protein is beta-2-glycoprotein protein.

34. The process of claim 1, wherein the protein is 196/H23FCN3 isoform 1 of Ficolin-3 protein.

35. The process of claim 1, wherein the protein is KH3 protein.

36. The process of claim 1, wherein the protein is KH4 protein.

37. The process of claim 1, wherein the protein is KH5 protein.

38. The process of claim 1, wherein the protein is KH6 protein.

39. The process of claim 1, wherein the protein is KH7 protein.

40. The process of claim 1, wherein the protein is KH8 protein.

41. The process of claim 1, wherein the protein is KH9 protein.

42. The process of claim 1, wherein the protein is KH10 protein.

43. The process of claim 1, wherein the protein is KH41 protein.

44. The process of claim 1, wherein the protein is KH42 protein.

45. The process of claim 1, wherein the protein is KH43 protein.

46. The process of claim 1, wherein the protein is any combination of any of the following proteins found in Immunoglobulin from fraction III: 193/H20 TF serotransferrin, 194/H21 APOH beta-2-glycoprotein, 195/H22 cDNA FLJ5165, beta-2-glycoprotein, 196/H₂₃FCN3 isoform 1 of Ficolin-3, KH3, KH4, KH5, KH6, KH7, KH8, KH9, KH10, KH41, KH42 and KH43 proteins.

47. The process of obtaining 80% or higher of Immunoglobulin from fraction II in combination with 20% Hepatitis B antibody proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

48. The process of obtaining 50% of Immunoglobulin from fraction II in combination with 50% Human Albumin from fraction V proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

49. The process of obtaining 10% or higher of a protein selected from the group consisting of 1 CP 98 kDa protein, Alpha 1 Antitrypsin protein, KH21 protein, KH22 protein, KH23 protein, KH24 protein, KH25 protein, KH26 protein, KH27 protein, KH48 protein, KH49 protein, KHSO protein, AntiThrombin III protein, and APOA1, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

50. The process of claim 49, wherein the protein is Alpha 1 Antitrypsin protein.

51. The process of claim 49, wherein the protein is KH21 protein.

52. The process of claim 49, wherein the protein is KH22 protein.

53. The process of claim 49, wherein the protein is KH23 protein.

54. The process of claim 49, wherein the protein is KH24 protein.

55. The process of claim 49, wherein the protein is KH25 protein.

56. The process of claim 49, wherein the protein is KH26 protein.

57. The process of claim 49, wherein the protein is KH27 protein.

58. The process of claim 49, wherein the protein is KH48 protein.

59. The process of claim 49, wherein the protein is KH49 protein.

60. The process of claim 49, wherein the protein is KHSO protein.

61. The process of claim 49, wherein the protein is any combination of any of the following proteins from fraction IV: Alpha 1 Antitrypsin, KH21, KH22, KH23, KH24, KH25, KH26, KH27, KH48, KH49 and KHSO proteins.

62. The process of claim 49, wherein the protein is AntiThrombin III protein.

63. The process of claim 49, wherein the protein is APOA1.

64. The process of obtaining 30% or higher of any combination of any of the following proteins from fraction IV: Human Albumin, KH21, KH22, KH23, KH24, KH25, KH26, KH27, KH48, KH49 and KHSO proteins, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

65. The process of claim 64, wherein the protein is Human Albumin from fraction IV protein.

66. The process of obtaining 30% or higher of a protein selected from the group consisting of Human Albumin from fraction III protein, KH19 protein, KH20 protein, KH38 protein, and KH40 protein, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

67. The process of claim 66, wherein the protein is KH19 protein.

68. The process of claim 66, wherein the protein is KH2O protein.

69. The process of claim 66, wherein the protein is KH38 protein.

70. The process of claim 66, wherein the protein is KH39 protein.

71. The process of claim 66, wherein the protein is KH40 protein.

72. The process of obtaining 30% or higher of any combinations of any of the following proteins from fraction III: Human Thrombin, Human Albumin, KH3, KH4, KH5, KH6, KH7, KH8, KH9, KH10, KH19, KH20, KH38, KH39, KH40, KH41, KH42, KH43, KH44, KH45, KH46, KH47, Human Prothrombin Complex, KH11, KH12, KH13, KH14, KH15, KH16, KH17 and KH18 in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

73. The process of obtaining a high percentage of Human Coagulation Factor VIII protein from Cryoprecipitate in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

74. The process of obtaining 30% or higher of protein from Cryoprecipitate selected from the group consisting Human Coagulation Factor VIII protein, KH1 protein, KH2 protein, KH28 protein, KH29 protein, KH30 protein, KH31 protein, KH32 protein and KH52 protein, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

75. The process of claim 74, wherein the protein is KH2 protein from Cryoprecipitate.

76. The process of claim 74, wherein the protein is KH28 protein from Cryoprecipitate.

77. The process of claim 74, wherein the protein is KH29 protein from Cryoprecipitate.

78. The process of claim 74, wherein the protein is any combination of any of the following proteins from cryoprecipitate: Human Factor VIII, KH1, KH2, KH28 and KH29 proteins.

79. The process of obtaining a high percentage of Human Fibrinogen protein from Cryoprecipitate or fraction I in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

80. The process of claim 74, wherein the protein is KH30 protein from Cryoprecipitate.

81. The process of claim 74, wherein the protein is KH31 protein from Cryoprecipitate.

82. The process of claim 74, wherein the protein is KH32 protein from Cryoprecipitate.

83. The process of claim 74, wherein the protein is any combination of any of the following proteins from cryoprecipitate: Human Fibrinogen, KH1, KH2, KH30, KH31 and KH32 proteins.

84. The process of obtaining a High Concentrate Human Fibrinogen protein from Cryoprecipitate or from fraction I in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

85. The process of claim 74, wherein the protein is KH52 protein from Cryoprecipitate.

86. The process of claim 74, wherein the protein is any combination of any of the following proteins from cryoprecipitate: High Concentrate Human Fibrinogen, KH1, KH2, KH30, KH31, KH32 and KH52 proteins.

87. The process of obtaining Human Thrombin protein from fraction III in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

88. The process of claim 72, wherein the protein is KH44 protein.

89. The process of claim 72, wherein the protein is KH45 protein.

90. The process of claim 72, wherein the protein is KH46 protein.

91. The process of claim 72, wherein the protein is KH47 protein.

92. The process of claim 72, wherein the protein is any combination of any of the following proteins from fraction III: Human Thrombin, KH44, KH45, KH46 and KH47 proteins.

93. The process of obtaining a high concentration of Human Prothrombin Complex proteins including factor II, factor VII, factor IX and factor X from fraction III in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

94. The process of claim 72, wherein the protein is KH11 protein.

95. The process of claim 72, wherein the protein is KH12 protein.

96. The process of claim 72, wherein the protein is KH13 protein.

97. The process of claim 72, wherein the protein is KH14 protein.

98. The process of claim 72, wherein the protein is KH15 protein.

99. The process of claim 72, wherein the protein is KH16 protein.

100. The process of claim 72, wherein the protein is KH17 protein.

101. The process of claim 72, wherein the protein is KH18 protein.

102. The process of claim 72, wherein the protein is any combination of any of the following proteins from fraction III: Human Prothrombin Complex, KH11, KH12, KH13, KH14, KH15, KH16, KH17 and KH18.

103. The process of claim 64, wherein the protein is any combination of any of the following proteins from fraction IV: KH21, KH22, KH23, KH24, KH25, KH26, KH27, KH48, KH49 and KHSO.

104. The process of obtaining a high concentration of any antibody protein in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

105. The process of claim 104, wherein the protein is a combination of at least two antibody proteins.

106. The process of claim 104, wherein the protein is a protein selected from the group consisting of Hepatitis A antibody protein, Cytomegalovirus antibody protein, Varicella zoster antibody protein, B19 Parvo antibody protein, Anti-D antibody protein, and C Esterase inhibitor antibody protein.

107. The process of claim 106, wherein the protein is Varicella zoster antibody protein.

108. The process of claim 106, wherein the protein is B19 Parvo antibody protein.

109. The process of claim 106, wherein the protein is Anti-D antibody protein.

110. The process of obtaining a high concentration of any protein from any source in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

111. The process of obtaining a High Concentrate Human Fibrinogen and Human Thrombin proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

112. The process of obtaining any recombinant DNA protein from any source in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

113. The process of claim 112, wherein the recombinant protein is recombinant KH1 protein.

114. The process of claim 112, wherein the recombinant protein is recombinant KH2 protein.

115. The process of claim 112, wherein the recombinant protein is recombinant KH3 protein.

116. The process of claim 112, wherein the recombinant protein is recombinant KH4 protein.

117. The process of claim 112, wherein the recombinant protein is recombinant KH5 protein.

118. The process of claim 112, wherein the recombinant protein is recombinant KH6 protein.

119. The process of claim 112, wherein the recombinant protein is recombinant KH7 protein.

120. The process of claim 112, wherein the recombinant protein is recombinant KH8 protein.

121. The process of claim 112, wherein the recombinant protein is recombinant KH9 protein.

122. The process of claim 112, wherein the recombinant protein is recombinant KH10 protein.

123. The process of claim 112, wherein the recombinant protein is recombinant KH11 protein.

124. The process of claim 112, wherein the recombinant protein is recombinant KH12 protein.

125. The process of claim 112, wherein the recombinant protein is recombinant KH13 protein.

126. The process of claim 112, wherein the recombinant protein is recombinant KH14 protein.

127. The process of claim 112, wherein the recombinant protein is recombinant KH15 protein.

128. The process of claim 112, wherein the recombinant protein is recombinant KH16 protein.

129. The process of claim 112, wherein the recombinant protein is recombinant KH17 protein.

130. The process of claim 112, wherein the recombinant protein is recombinant KH18 protein.

131. The process of claim 112, wherein the recombinant protein is recombinant KH19 protein.

132. The process of claim 112, wherein the recombinant protein is recombinant KH2O protein.

133. The process of claim 112, wherein the recombinant protein is recombinant KH21 protein.

134. The process of claim 112, wherein the recombinant protein is recombinant KH22 protein.

135. The process of claim 112, wherein the recombinant protein is recombinant KH23 protein.

136. The process of claim 112, wherein the recombinant protein is recombinant KH24 protein.

137. The process of claim 112, wherein the recombinant protein is recombinant KH25 protein.

138. The process of claim 112, wherein the recombinant protein is recombinant KH26 protein.

139. The process of claim 112, wherein the recombinant protein is recombinant KH27 protein.

140. The process of claim 112, wherein the recombinant protein is recombinant KH28 protein.

141. The process of claim 112, wherein the recombinant protein is recombinant KH29 protein.

142. The process of claim 112, wherein the recombinant protein is recombinant KH30 protein.

143. The process of claim 112, wherein the recombinant protein is recombinant KH31 protein.

144. The process of claim 112, wherein the recombinant protein is recombinant KH32 protein.

145. The process of claim 112, wherein the recombinant protein is recombinant KH33 protein.

146. The process of claim 112, wherein the recombinant protein is recombinant KH34 protein.

147. The process of claim 112, wherein the recombinant protein is recombinant KH35 protein.

148. The process of claim 112, wherein the recombinant protein is recombinant KH36 protein.

149. The process of claim 112, wherein the recombinant protein is recombinant KH37 protein.

150. The process of claim 112, wherein the recombinant protein is recombinant KH38 protein.

151. The process of claim 112, wherein the recombinant protein is recombinant KH39 protein.

152. The process of claim 112, wherein the recombinant protein is recombinant KH40 protein.

153. The process of claim 112, wherein the recombinant protein is recombinant KH41 protein.

154. The process of claim 112, wherein the recombinant protein is recombinant KH42 protein.

155. The process of claim 112, wherein the recombinant protein is recombinant KH43 protein.

156. The process of claim 112, wherein the recombinant protein is recombinant KH44 protein.

157. The process of claim 112, wherein the recombinant protein is recombinant KH45 protein.

158. The process of claim 112, wherein the recombinant protein is recombinant KH46 protein.

159. The process of claim 112, wherein the recombinant protein is recombinant KH47 protein.

160. The process of claim 112, wherein the recombinant protein is recombinant KH48 protein.

161. The process of claim 112, wherein the recombinant protein is recombinant KH49 protein.

162. The process of claim 112, wherein the recombinant protein is recombinant KHSO protein.

163. The process of claim 112, wherein the recombinant protein is recombinant KH51 protein.

164. The process of claim 112, wherein the recombinant protein is recombinant KH52 protein.

165. The process of obtaining any combination of the already discovered recombinant proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

166. The process of obtaining any monoclonal antibody protein in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

167. The process of claim 166, wherein the monoclonal antibody protein is a monoclonal antibody KH1 protein.

168. The process of claim 166, wherein the monoclonal antibody protein is a monoclonal antibody KH2 protein.

169. The process of claim 166, wherein the monoclonal antibody protein is a monoclonal antibody KH3 protein.

170. The process of claim 166, wherein the monoclonal antibody protein is a monoclonal antibody KH4 protein.

171. The process of claim 166, wherein the monoclonal antibody protein is a monoclonal antibody KH5 protein.

172. The process of claim 166, wherein the monoclonal antibody protein is a monoclonal antibody KH6 protein.

173. The process of claim 166, wherein the monoclonal antibody protein is a monoclonal antibody KH7 protein.

174. The process of claim 166, wherein the monoclonal antibody protein is a monoclonal antibody KH8 protein.

175. The process of claim 166, wherein the monoclonal antibody protein is a monoclonal antibody KH9 protein.

176. The process of claim 166, wherein the monoclonal antibody protein is a monoclonal antibody KH10 protein.

177. The process of claim 166, wherein the monoclonal antibody protein is a monoclonal antibody KH11 protein.

178. The process of claim 166, wherein the monoclonal antibody protein is a monoclonal antibody KH12 protein.

179. The process of claim 166, wherein the monoclonal antibody protein is a monoclonal antibody KH13 protein.

180. The process of claim 166, wherein the monoclonal antibody protein is a monoclonal antibody KH14 protein.

181. The process of claim 166, wherein the monoclonal antibody protein is a monoclonal antibody KH15 protein.

182. The process of claim 166, wherein the monoclonal antibody protein is a monoclonal antibody KH16 protein.

183. The process of claim 166, wherein the monoclonal antibody protein is a monoclonal antibody KH17 protein.

184. The process of claim 166, wherein the monoclonal antibody protein is a monoclonal antibody KH18 protein.

185. The process of claim 166, wherein the monoclonal antibody protein is a monoclonal antibody KH19 protein.

186. The process of claim 166, wherein the monoclonal antibody protein is a monoclonal antibody KH20 protein.

187. The process of claim 166, wherein the monoclonal antibody protein is a monoclonal antibody KH21 protein.

188. The process of claim 166, wherein the monoclonal antibody protein is a monoclonal antibody KH22 protein.

189. The process of claim 166, wherein the monoclonal antibody protein is a monoclonal antibody KH23 protein.

190. The process of claim 166, wherein the monoclonal antibody protein is a monoclonal antibody KH24 protein.

191. The process of claim 166, wherein the monoclonal antibody protein is a monoclonal antibody KH25 protein.

192. The process of claim 166, wherein the monoclonal antibody protein is a monoclonal antibody KH26 protein.

193. The process of claim 166, wherein the monoclonal antibody protein is a monoclonal antibody KH27 protein.

194. The process of claim 166, wherein the monoclonal antibody protein is a monoclonal antibody KH28 protein.

195. The process of claim 166, wherein the monoclonal antibody protein is a monoclonal antibody KH29 protein.

196. The process of claim 166, wherein the monoclonal antibody protein is a monoclonal antibody KH30 protein.

197. The process of claim 166, wherein the monoclonal antibody protein is a monoclonal antibody KH31 protein.

198. The process of claim 166, wherein the monoclonal antibody protein is a monoclonal antibody KH32 protein.

199. The process of claim 166, wherein the monoclonal antibody protein is a monoclonal antibody KH33 protein.

200. The process of claim 166, wherein the monoclonal antibody protein is a monoclonal antibody KH34 protein.

201. The process of claim 166, wherein the monoclonal antibody protein is a monoclonal antibody KH35 protein.

202. The process of claim 166, wherein the monoclonal antibody protein is a monoclonal antibody KH36 protein.

203. The process of claim 166, wherein the monoclonal antibody protein is a monoclonal antibody KH37 protein.

204. The process of claim 166, wherein the monoclonal antibody protein is a monoclonal antibody KH38 protein.

205. The process of claim 166, wherein the monoclonal antibody protein is a monoclonal antibody KH39 protein.

206. The process of claim 166, wherein the monoclonal antibody protein is a monoclonal antibody KH40 protein.

207. The process of claim 166, wherein the monoclonal antibody protein is a monoclonal antibody KH41 protein.

208. The process of claim 166, wherein the monoclonal antibody protein is a monoclonal antibody KH42 protein.

209. The process of claim 166, wherein the monoclonal antibody protein is a monoclonal antibody KH43 protein.

210. The process of claim 166, wherein the monoclonal antibody protein is a monoclonal antibody KH44 protein.

211. The process of claim 166, wherein the monoclonal antibody protein is a monoclonal antibody KH45 protein.

212. The process of claim 166, wherein the monoclonal antibody protein is a monoclonal antibody KH46 protein.

213. The process of claim 166, wherein the monoclonal antibody protein is a monoclonal antibody KH47 protein.

214. The process of claim 166, wherein the monoclonal antibody protein is a monoclonal antibody KH48 protein.

215. The process of claim 166, wherein the monoclonal antibody protein is a monoclonal antibody KH49 protein.

216. The process of claim 166, wherein the monoclonal antibody protein is a monoclonal antibody KHSO protein.

217. The process of claim 166, wherein the monoclonal antibody protein is a monoclonal antibody KH51 protein.

218. The process of claim 166, wherein the monoclonal antibody protein is a monoclonal antibody KH52 protein.

219. The process of obtaining any combination of the already discovered monoclonal antibodies proteins in combination with any of the following: KH1, KH2, KH3, KH4, KH5, KH6, KH7, KH8, KH9, KH10, KH11, KH12, KH13, KH14, KH15, KH16, KH17, KH18, KH19, KH20, KH21, KH22, KH23, KH24, KH25, KH26, KH27, KH28, KH29, KH30, KH31, KH32, KH33, KH34, KH35, KH36, KH37, KH38, KH39, KH40, KH41, KH42, KH43, KH44, KH45, KH46, KH47, KH48, KH49, KH50, KH51 and KH52 proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

220. The process of obtaining any desired protein by using cells selected from the group consisting of Human Albumin, Immunoglobulin, Human Factor VIII, Human Prothrombin Complex, Human Fibrinogen, Human Thrombin, High concentrate Human Fibrinogen, Hepatitis B antibody, Antithrombin III, Alpha 1 antitrypsin protein, CP kDa 98, APOA1 protein, Hepatitis A antibody, Cytomeglovirus antibody,Vericella zoster antibody, B 19 Parvo antibody, Anti-D antibody, C Esterase inhibitor antibody, KH1, KH2, KH3, KH4, KH5, KH6, KH7, KH8, KH9, KH10, KH11, KH12, KH13, KH14, KH15, KH16, KH17, KH18, KH19, KH20, KH21, KH22, KH23, KH24, KH25, KH26, KH27, KH28, KH29, KH30, KH31, KH32, KH33, KH34, KH35, KH36, KH37, KH38, KH39, KH40, KH41, KH42, KH43, KH44, KH45, KH46, KH47, KH48, KH49, KH50, KH51 and KH52 proteins, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

221. The process of claim 220, wherein the cells are from Immunoglobulin.

222. The process of claim 220, wherein the cells are from Human Factor VIII.

223. The process of claim 220, wherein the cells are from Human Prothrombin Complex.

224. The process of claim 220, wherein the cells are from Human Fibrinogen.

225. The process of claim 220, wherein the cells are from Human Thrombin.

226. The process of claim 220, wherein the cells are from High concentrate Human Fibrinogen.

227. The process of claim 220, wherein the cells are from Hepatitis B antibody.

228. The process of claim 220, wherein the cells are from Antithrombin III protein.

229. The process of claim 220, wherein the cells are from Alpha 1 antitrypsin protein.

230. The process of claim 220, wherein the cells are from CP kDa 98 protein.

231. The process of claim 220, wherein the cells are from APOA1 protein.

232. The process of claim 220, wherein the cells are from Hepatitis A antibody.

233. The process of claim 220, wherein the cells are from Cytomeglovirus antibody.

234. The process of claim 220, wherein the cells are from Varicella zoster antibody.

235. The process of claim 220, wherein the cells are from B19 Parvo antibody.

236. The process of claim 220, wherein the cells are from Anti-D antibody.

237. The process of claim 220, wherein the cells are from C Esterase inhibitor antibody.

238. The process of claim 220, wherein the cells are selected from the group consisting of KH1, KH2, KH3, KH4, KH5, KH6, KH7, KH8, KH9, KH10, KH11, KH12, KH13, KH14, KH15, KH16, KH17, KH18, KH19, KH20, KH21, KH22, KH23, KH24, KH25, KH26, KH27, KH28, KH29, KH30, KH31, KH32, KH33, KH34, KH35, KH36, KH37, KH38, KH39, KH40, KH41, KH42, KH43, KH44, KH45, KH46, KH47, KH48, KH49, KH50, KH51 and KH52 proteins.

239. The process of obtaining any desired protein by using the cells from any protein, antibody, any source, any substance or KH1, KH2, KH3, KH4, KH5, KH6, KH7, KH8, KH9, KH10, KH11, KH12, KH13, KH14, KH15, KH16, KH17, KH18, KH19, KH20, KH21, KH22, KH23, KH24, KH25, KH26, KH27, KH28, KH29, KH30, KH31, KH32, KH33, KH34, KH35, KH36, KH37, KH38, KH39, KH40, KH41, KH42, KH43, KH44, KH45, KH46, KH47, KH48, KH49, KH50, KH51 and KH52 proteins in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

240. The process of obtaining any desired protein from any animal source by using the cells from KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

241. The process of claim 240, wherein the desired protein is obtained from Bovine Albumin protein.

242. The process of claim 240, wherein the desired protein is obtained from Bovine Immunoglobulin protein.

243. The process of claim 240, wherein the desired protein is obtained from pig fibrinogen protein.

244. The process of claim 240, wherein the desired protein is obtained from any bird source protein.

245. The process of claim 240, wherein the desired protein is obtained from any canine source protein.

246. The process of claim 240, wherein the desired protein is obtained from any feline source protein.

247. The process of claim 240, wherein the desired protein is obtained from any Panda bear source protein.

248. The process of obtaining a protein from any animal source in combination with any protein from same or any animal source in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

249. Good healthy cells selected from the group consisting of good healthy T cells, good healthy B cells, good healthy Activated B cells, good healthy myeloid dendritic cells (mDC), good healthy plasmacytoid dendritic cells (pDC), good healthy Granulocytes cells, good healthy Monocytes cells, good healthy Macrophage cells, good healthy Neutrophil cells, good healthy Basophil cells, good healthy Eosonophil cells, good healthy CD3 T cells, in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

250. The good healthy cells of claim 249, wherein the good healthy cells are good healthy T cells.

251. The good healthy cells of claim 249, wherein the good healthy cells are good healthy B cells.

252. The good healthy cells of claim 249, wherein the good healthy cells are good healthy Activated B cells n.

253. The good healthy cells of claim 249, wherein the good healthy cells are good healthy myeloid dendritic cells (mDC).

254. The good healthy cells of claim 249, wherein the good healthy cells are good healthy plasmacytoid dendritic cells (pDC).

255. The good healthy cells of claim 249, wherein the good healthy cells are good healthy Granulocytes cells.

256. The good healthy cells of claim 249, wherein the good healthy cells are good healthy Monocytes cells.

257. The good healthy cells of claim 249, wherein the good healthy cells are good healthy Macrophage cells.

258. The good healthy cells of claim 249, wherein the good healthy cells are good healthy Neutrophil cells.

259. The good healthy cells of claim 249, wherein the good healthy cells are good healthy Basophil cells.

260. The good healthy cells of claim 249, wherein the good healthy cells are good Healthy Eosinophil cells.

261. The good healthy cells of claim 249, wherein the good healthy cells are good healthy CD3 T cells.

262. All existing discovered good healthy cells from Human, Animal, plant, recombinant, monoclonal, transgenic or any substance or form in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

263. All newly discovered or being discovered good healthy KH cells like Dragon, Double Ring, pixel, etc. in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

264. The process of claim 1, wherein the protein is Human Albumin protein.

265. The process of claim 49, wherein the protein is Alpha 1 Antitrypsin protein.

266. The process of claim 66, wherein the protein is Human Albumin from fraction Ill protein.

267. The process of claim 74, wherein the protein is KH1 protein from Cryoprecipitate.

268. The process of claim 74, wherein the protein is Human Coagulation Factor VIII protein.

269. The process of claim 106, wherein the protein is C Esterase inhibitor antibody protein.

270. The process of claim 106, wherein the protein is Cytomegalovirus antibody protein.

271. The process of claim 165, wherein the combination of the already discovered recombinant proteins is in combination with any of the following: KH1, KH2, KH3, KH4, KH5, KH6, KH7, KH8, KH9, KH10, KH11, KH12, KH13, KH14, KH15, KH16, KH17, KH18, KH19, KH20, KH21, KH22, KH23, KH24, KH25, KH26, KH27, KH28, KH29, KH30, KH31, KH32, KH33, KH34, KH35, KH36, KH37, KH38, KH39, KH40, KH41, KH42, KH43, KH44, KH45, KH46, KH47, KH48, KH49, KH50, KH51 and KH52 proteins in KH healthy cells.

272. The process of claim 220, wherein the cells are from Human Albumin.
